SEQUENCE LISTING (209 sequences)

SEQ ID NO: 1 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 3 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 223 aa, Homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 13 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 14 - protein, 112 aa, Homo sapiens
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 15 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 18 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 19 - protein, 266 aa, Homo sapiens [sequence omitted]

SEQ ID NO: 20 - protein, 266 aa, Homo sapiens [sequence omitted]

SEQ ID NO: 21 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 22 - protein, 266 aa, Homo sapiens [sequence omitted]

SEQ ID NO: 23 - protein, 266 aa, Homo sapiens [sequence omitted]

SEQ ID NO: 24 - protein, 266 aa, Homo sapiens [sequence omitted]

SEQ ID NO: 25 - protein, 266 aa, Homo sapiens [sequence omitted]

SEQ ID NO: 26 - protein, 266 aa, Homo sapiens
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQRRAEVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKMLSGVGGFVLGLLFLGAGLFIYFRNQKGHSGLQPTGFLS

SEQ ID NO: 27 - protein, 266 aa, Homo sapiens [sequence omitted]

SEQ ID NO: 28 - protein, 266 aa, Homo sapiens [sequence omitted]

SEQ ID NO: 29 - protein, 266 aa, Homo sapiens [sequence omitted]

SEQ ID NO: 30 - protein, 266 aa, Homo sapiens [sequence omitted]

SEQ ID NO: 31 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 32 - protein, 266 aa, Homo sapiens [sequence omitted]

SEQ ID NO: 33 - protein, 266 aa, Homo sapiens [sequence omitted]

SEQ ID NO: 34 - protein, 266 aa, Homo sapiens [sequence omitted]

SEQ ID NO: 35 - protein, 266 aa, Homo sapiens [sequence omitted]

SEQ ID NO: 36 - protein, 266 aa, Homo sapiens [sequence omitted]

SEQ ID NO: 37 - protein, 266 aa, Homo sapiens [sequence omitted]

SEQ ID NO: 38 - protein, 266 aa, Homo sapiens
MVCLRLPGGSCMAVLTVTLMVLSSPLALAGDTRPRFLEYSTSECHFFNGTERVRFLDRYFYNQEEYVRFDSDVGEFRAVTELGRPDEEYWNSQKDFLEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 39 - protein, 266 aa, Homo sapiens [sequence omitted]

SEQ ID NO: 40 - protein, 266 aa, Homo sapiens [sequence omitted]

SEQ ID NO: 41 - protein, 266 aa, Homo sapiens [sequence omitted]

SEQ ID NO: 42 - protein, 266 aa, Homo sapiens [sequence omitted]

SEQ ID NO: 43 - protein, 266 aa, Homo sapiens [sequence omitted]

SEQ ID NO: 44 - protein, 266 aa, Homo sapiens [sequence omitted]

SEQ ID NO: 45 - protein, 266 aa, Homo sapiens [sequence omitted]

SEQ ID NO: 46 - protein, 266 aa, Homo sapiens [sequence omitted]

SEQ ID NO: 47 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 48 - protein, 266 aa, Homo sapiens [sequence omitted]

SEQ ID NO: 49 - protein, 266 aa, Homo sapiens [sequence omitted]

SEQ ID NO: 50 - protein, 266 aa, Homo sapiens [sequence omitted]

SEQ ID NO: 51 - protein, 266 aa, Homo sapiens; identical to SEQ ID NO: 47

SEQ ID NO: 52 - protein, 266 aa, Homo sapiens; identical to SEQ ID NO: 47

SEQ ID NO: 53 - protein, 266 aa, Homo sapiens [sequence omitted]

SEQ ID NO: 54 - protein, 266 aa, Homo sapiens [sequence omitted]

SEQ ID NO: 55 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 56 - protein, 266 aa, Homo sapiens [sequence omitted]

SEQ ID NO: 57 - protein, 266 aa, Homo sapiens
MVCLKLPGGSSLAALTVTLMVLSSRLAFAGDTRPRFLELLKSECHFFNGTERVRFLERHFHNQEEYARFDSDVGEYRAVRELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSKMLSGVGGFVLGLLFLGAGLFIYFRNQKGHSGLQPTGFLS

SEQ ID NO: 58 - protein, 266 aa, Homo sapiens [sequence omitted]

SEQ ID NO: 59 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNSQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 60 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 61 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 62 - protein, 33 aa, Homo sapiens
RMKQIEDKIEEILSKIYHIENEIARIKKLIGER

SEQ ID NO: 63 - protein, 32 aa, Homo sapiens
LSSIEKKQEEQTSWLIWISNELTLIRNELAQS

SEQ ID NO: 64 - protein, 31 aa, Homo sapiens
LSSIEKKLEEITSQLIQISNELTLIRNELAQ

SEQ ID NO: 65 - protein, 31 aa, Homo sapiens
LSSIEKKLEEITSQLIQIRNELTLIRNELAQ

SEQ ID NO: 66 - protein, 31 aa, Homo sapiens
LSSIEKKLEEITSQLQQIRNELTLIRNELAQ

SEQ ID NO: 67 - protein, 31 aa, Homo sapiens
LSSLEKKLEELTSQLIQLRNELTLLRNELAQ

SEQ ID NO: 68 - protein, 31 aa, Homo sapiens
ISSLEKKIEELTSQIQQLRNEITLLRNEIAQ

SEQ ID NO: 69 - protein, 47 aa, Homo sapiens
LEIEAAFLERENTALETRVAELRQRVQRLRNRVSQYRTRYGPLGGGK

SEQ ID NO: 70 - protein, 31 aa, Homo sapiens
LKSVENRLAVVENQLKTVIEELKTVKDLLSN

SEQ ID NO: 71 - protein, 31 aa, Homo sapiens
LARIEEKLKTIKAQLSEIASTLNMIREQLAQ

SEQ ID NO: 72 - protein, 31 aa, Homo sapiens
VSRLEEKVKTLKSQVTELASTVSLLREQVAQ

SEQ ID NO: 73 - protein, 30 aa, Homo sapiens
IQSEKKIEDISSLIGQIQSEITLIRNEIAQ

SEQ ID NO: 74 - protein, 31 aa, Homo sapiens
LMSLEKKLEELTQTLMQLQNELSMLKNELAQ

SEQ ID NO: 75 - protein, 18 aa, Homo sapiens
VDLEGSTSNGRQCAGIRL

SEQ ID NO: 76 - protein, 27 aa, Homo sapiens
EDDVTTTEELAPALVPPPKGTCAGWMA

SEQ ID NO: 77 - protein, 27 aa, Homo sapiens
GHDQETTTQGPGVLLPLPKGACTGQMA

SEQ ID NO: 78 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 79 - protein, 64 aa, Homo sapiens
VTAFSNMDDMLQKAHLVIEGTFIYLRDSTEFFIRVRDGWKKLQLGELIPIPADSPPPPALSSNP

SEQ ID NO: 80 - protein, 178 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 81 - protein, 79 aa, Homo sapiens
VIIQAEFYLNPDQSGEFMFDFDGDEIFHVDMAKKETVWRLEEFGRFASFEAQGALANIAVDKANLEIMTKRSNYTPITN

SEQ ID NO: 82 - protein, 94 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 83 - protein, 199 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 84 - protein, 199 aa, Homo sapiens; identical to SEQ ID NO: 83

SEQ ID NO: 85 - protein, 94 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 86 - protein, 95 aa, Homo sapiens
GDTRCRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 87 - protein, 103 aa, Homo sapiens
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 88 - protein, 104 aa, Homo sapiens
YPEVTVYPAKTQPLQHHNLLVCSVNGFYPASIEVRWFRNGQEEKTGVVSTGLIQNGDCTFQTLVMLETVPRSGEVYTCQVEHPSLTSPLTVEWRARSESAQSKM

SEQ ID NO: 89 - protein, 197 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 90 - protein, 29 aa, Homo sapiens
MVCLKLPGGSSLAALTVTLMVLSSRLAFA

SEQ ID NO: 91 - protein, 94 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 92 - protein, 103 aa, Homo sapiens
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 93 - protein, 19 aa, Homo sapiens
MVCLKLPGGSCMAALTVTL

SEQ ID NO: 94 - protein, 105 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 95 - protein, 103 aa, Homo sapiens
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 96 - protein, 105 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRV

SEQ ID NO: 97 - protein, 102 aa, Homo sapiens
EPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 98 - protein, 19 aa, Homo sapiens
MVCLKLPGGSYMAKLTVTL

SEQ ID NO: 99 - protein, 5 aa, Artificial Sequence
  Peptide suitable for use as a linker.
GGGGS

SEQ ID NO: 100 - protein, 20 aa, Artificial Sequence
  Peptide suitable for use as a linker.
GCGGSGGGGSGGGGSGGGGS

SEQ ID NO: 101 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 102 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPSCVPQALEPLPIVYYVGRKPKVEQLSNMIVRSCKCS

SEQ ID NO: 103 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 104 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPSCVSQDLEPLTILYYIGKTPKIEQLSNMIVKSCKCS

SEQ ID NO: 105 - protein, 112 aa, Homo sapiens [sequence omitted]

SEQ ID NO: 106 - protein, 112 aa, Homo sapiens
ALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 107 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 108 - protein, 154 aa, Homo sapiens
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 109 - protein, 143 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 110 - protein, 129 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 111 - protein, 114 aa, Homo sapiens
TDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 112 - protein, 104 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 113 - protein, 111 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 114 - protein, 112 aa, Homo sapiens
SQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 115 - protein, 112 aa, Homo sapiens; identical to SEQ ID NO: 106

SEQ ID NO: 116 - protein, 133 aa, Homo sapiens [sequence omitted]

SEQ ID NO: 117 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 118 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 119 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 120 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 121 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 122 - protein, 227 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLNVSIKI

SEQ ID NO: 123 - protein, 124 aa, Homo sapiens
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 124 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 125 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 126 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 127 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 128 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 129 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 130 - protein, 5 aa, Artificial Sequence
  Peptide suitable for use as a linker.
GSGGS

SEQ ID NO: 131 - protein, 4 aa, Artificial Sequence
  Peptide suitable for use as a linker.
GGGS

SEQ ID NO: 132 - protein, 4 aa, Artificial Sequence
  Peptide suitable for use as a linker.
GGSG

SEQ ID NO: 133 - protein, 5 aa, Artificial Sequence
  Peptide suitable for use as a linker.
GGSGG

SEQ ID NO: 134 - protein, 5 aa, Artificial Sequence
  Peptide suitable for use as a linker.
GSGSG

SEQ ID NO: 135 - protein, 5 aa, Artificial Sequence
  Peptide suitable for use as a linker.
GSGGG

SEQ ID NO: 136 - protein, 5 aa, Artificial Sequence
  Peptide suitable for use as a linker.
GGGSG

SEQ ID NO: 137 - protein, 5 aa, Artificial Sequence
  Peptide suitable for use as a linker.
GSSSG

SEQ ID NO: 138 - protein, 5 aa, Artificial Sequence
  Peptide suitable for use as a linker.
GSSSS

SEQ ID NO: 139 - protein, 5 aa, Artificial Sequence
  Peptide suitable for use as a linker.
AAAGG

SEQ ID NO: 140 - protein, 5 aa, Artificial Sequence
  Peptide suitable for use as a linker. The EAAAK may be repeated 2 to 20 times.
EAAAK

SEQ ID NO: 141 - protein, 7 aa, Artificial Sequence
  Peptide suitable for use as a linker. The EAAAK subset may be repeated 2 to 20 times.
AEAAAKA

SEQ ID NO: 142 - protein, 16 aa, Artificial Sequence
  Peptide suitable for use as a linker. The EAAAK subset may be repeated independently 2 to 20 times.
AEAAAKALEAEAAAKA

SEQ ID NO: 143 - protein, 5 aa, Artificial Sequence
  Peptide suitable for use as a linker
EAAAK

SEQ ID NO: 144 - protein, 10 aa, Artificial Sequence
  Peptide suitable for use as a linker.
EAAAKEAAAK

SEQ ID NO: 145 - protein, 15 aa, Artificial Sequence
  Peptide suitable for use as a linker.
EAAAKEAAAKEAAAK

SEQ ID NO: 146 - protein, 46 aa, Artificial Sequence
  Peptide suitable for use as a linker.
AEAAAKEAAAKEAAAKEAAAKALEAEAAAKEAAAKEAAAKEAAAKA

SEQ ID NO: 147 - protein, 12 aa, Artificial Sequence
  Peptide suitable for use as a linker.
AEAAAKEAAAKA

SEQ ID NO: 148 - protein, 5 aa, Artificial Sequence
  Peptide suitable for use as a linker.
PAPAP

SEQ ID NO: 149 - protein, 8 aa, Artificial Sequence
  Peptide suitable for use as a linker.
APAPAPAP

SEQ ID NO: 150 - protein, 12 aa, Artificial Sequence
  Peptide suitable for use as a linker.
APAPAPAPAPAP

SEQ ID NO: 151 - protein, 16 aa, Artificial Sequence
  Peptide suitable for use as a linker.
APAPAPAPAPAPAPAP

SEQ ID NO: 152 - protein, 20 aa, Artificial Sequence
  Peptide suitable for use as a linker.
APAPAPAPAPAPAPAPAPAP

SEQ ID NO: 153 - protein, 4 aa, Artificial Sequence
  Peptide suitable for use as a linker.
KPKP

SEQ ID NO: 154 - protein, 8 aa, Artificial Sequence
  Peptide suitable for use as a linker.
KPKPKPKP

SEQ ID NO: 155 - protein, 12 aa, Artificial Sequence
  Peptide suitable for use as a linker.
KPKPKPKPKPKP

SEQ ID NO: 156 - protein, 16 aa, Artificial Sequence
  Peptide suitable for use as a linker.
KPKPKPKPKPKPKPKP

SEQ ID NO: 157 - protein, 20 aa, Artificial Sequence
  Peptide suitable for use as a linker.
KPKPKPKPKPKPKPKPKPKP

SEQ ID NO: 158 - protein, 4 aa, Artificial Sequence
  Peptide suitable for use as a linker.
EPEP

SEQ ID NO: 159 - protein, 8 aa, Artificial Sequence
  Peptide suitable for use as a linker.
EPEPEPEP

SEQ ID NO: 160 - protein, 12 aa, Artificial Sequence
  Peptide suitable for use as a linker.
EPEPEPEPEPEP

SEQ ID NO: 161 - protein, 16 aa, Artificial Sequence
  Peptide suitable for use as a linker.
EPEPEPEPEPEPEPEP

SEQ ID NO: 162 - protein, 20 aa, Artificial Sequence
  Peptide suitable for use as a linker.
EPEPEPEPEPEPEPEPEPEP

SEQ ID NO: 163 - protein, 15 aa, Artificial Sequence
  Linker
GCGASGGGGSGGGGS

SEQ ID NO: 164 - protein, 15 aa, Artificial Sequence
  Peptide suitable for use as a linker.
GCGGSGGGGSGGGGS

SEQ ID NO: 165 - protein, 17 aa, Homo sapiens
GAGSLQPLALEGSLQKR

SEQ ID NO: 166 - protein, 15 aa, Homo sapiens
GIVDQCCTSICSLYQ

SEQ ID NO: 167 - protein, 15 aa, Homo sapiens
GIVEQCCTSICSLYQ

SEQ ID NO: 168 - protein, 13 aa, Homo sapiens
NFFRMVISNPAAT

SEQ ID NO: 169 - protein, 13 aa, Homo sapiens
NFIRMVISNPAAT

SEQ ID NO: 170 - protein, 20 aa, Homo sapiens
SFYLKNVQTQETRTLTQFHF

SEQ ID NO: 171 - protein, 15 aa, Homo sapiens
SLQPLALEGSLQSRG

SEQ ID NO: 172 - protein, 18 aa, Homo sapiens
GSLQPLALEGSLQSRGIV

SEQ ID NO: 173 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 174 - protein, 18 aa, Homo sapiens
GAGSLQPLALEGSLQKRG

SEQ ID NO: 175 - protein, 15 aa, Homo sapiens
SLQPLALEGSLQKRG

SEQ ID NO: 176 - protein, 15 aa, Homo sapiens
SLQPLALEGSLQSRG

SEQ ID NO: 177 - protein, 13 aa, Homo sapiens
QPLALEGSLQKRG

SEQ ID NO: 178 - protein, 13 aa, Homo sapiens
QPLALEGSLQSRG

SEQ ID NO: 179 - protein, 9 aa, Artificial Sequence
  affinity domain - hemagglutinin
YPYDVPDYA

SEQ ID NO: 180 - protein, 8 aa, Artificial Sequence
  Epitope tag FLAG
DYKDDDDK

SEQ ID NO: 181 - protein, 10 aa, Artificial Sequence
  Epitope tag  c-myc
EQKLISEEDL

SEQ ID NO: 182 - protein, 5 aa, Homo sapiens
HHHHH

SEQ ID NO: 183 - protein, 6 aa, Homo sapiens
HHHHHH

SEQ ID NO: 184 - protein, 8 aa, Artificial Sequence
  Strep Tag
WSHPQFEK

SEQ ID NO: 185 - protein, 5 aa, Artificial Sequence
  Affinity Tag
RYIRS

SEQ ID NO: 186 - protein, 4 aa, Homo sapiens
FHHT

SEQ ID NO: 187 - protein, 17 aa, Homo sapiens
WEAAAREACCRECCARA

SEQ ID NO: 188 - protein, 111 aa, Homo sapiens; identical to SEQ ID NO: 113

SEQ ID NO: 189 - protein, 15 aa, Homo sapiens
SHLVEALYLVCGERG

SEQ ID NO: 190 - protein, 20 aa, Homo sapiens
YVVKSFDRSTKVIDFHYPNE

SEQ ID NO: 191 - protein, 17 aa, Homo sapiens
AMMIARFKMFPEVKEKG

SEQ ID NO: 192 - protein, 9 aa, Homo sapiens
VELGGGPGA

SEQ ID NO: 193 - protein, 14 aa, Homo sapiens
VYLKTNVFLGGGAS

SEQ ID NO: 194 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 195 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 196 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 197 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 198 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 199 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 200 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 201 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 202 - protein, 614 aa, Artificial Sequence
  Construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 203 - protein, 466 aa, Artificial Sequence
  Construct
ASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 204 - protein, 603 aa, Artificial Sequence
  Construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 205 - protein, 455 aa, Artificial Sequence
  Construct
ASTKGPSVFPLAPSSKSTSGGTAALGCLVKDYFPEPVTVSWNSGALTSGVHTFPAVLQSSGLYELVSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 206 - protein, 561 aa, Artificial Sequence
  Construct
PITAKAIAASVGGGGGSGGGGSGGGGSSECHFVVQFKGECYYTNGTQRIRLVTRYIYNREEYVRYDSDVGEYRAVTELGRPDAEYWNSQPEILERTRAEVDTACRHNYEGPETSTSLRRLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWSSGGGGSGGGGSGGGGSGGGGSGGGGSEDDIEADHVGFYGTTVYQSPGDIGQYTHEFDGDELFYVDLDKKKTVWRLPEFGQLILFEPQGGLQNIAAEKHNLGILTKRSNFTCATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSELTGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 207 - protein, 566 aa, Artificial Sequence
  Construct
ISQAVHAAHAEINEAGRGGGGSGGGGSGGGGSSECHFVVQFKGECYYTNGTQRIRLVTRYIYNREEYVRYDSDVGEYRAVTELGRPDAEYWNSQPEILERTRAEVDTACRHNYEGPETSTSLRRLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWSSGGGGSGGGGSGGGGSGGGGSGGGGSEDDIEADHVGFYGTTVYQSPGDIGQYTHEFDGDELFYVDLDKKKTVWRLPEFGQLILFEPQGGLQNIAAEKHNLGILTKRSNFTCATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSELTGGGGSGGGGSGGGGSGGGGSTVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 208 - protein, 106 aa, Artificial Sequence
  Construct
TVAAPSVFIFPPSDEQLKSGTASVVCLLNNFYPREAKVQWKVDNALQSGNSQESVTEQDSKDSTYSLLSSLTLSKADYEKHKVYACEVTHQGLSSPVTKSFNRGEC

SEQ ID NO: 209 - protein, 248 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

(19) United States
(12) Patent Application Publication
(10) Pub. No.: US 2024/0190933 A1
(43) Pub. Date: Jun. 13, 2024

(54) Antigen Presenting Polypeptide Complexes Bearing TGF-Beta and Methods of Use Thereof

(71) Applicant: Cue Biopharma, Inc., Boston, MA (US)

(72) Inventors: Ronald D. SEIDEL, III, Natick, MA (US); John F. ROSS, Arlington, MA (US); Chee Meng LOW, Cambridge, MA (US)

(21) Appl. No.: 18/287,754

(22) PCT Filed: Apr. 20, 2022
(86) PCT No.: PCT/US2022/025526

§ 371 Date: Oct. 20, 2023

Related U.S. Application Data
(60) Provisional application No. 63/177,954, filed on Apr. 21, 2021.

(51) Int. Cl. C07K 14/495; A61K 38/00; C07K 14/55; C07K 14/705; C07K 14/74

(52) U.S. Cl. CPC C07K 14/495; C07K 14/55; C07K 14/70532; C07K 14/70539; C07K 14/70575; A61K 38/00; C07K 2319/30

(57) ABSTRACT

The present disclosure provides Multimeric Antigen Presenting Polypeptides (MAPPs) that comprise a TID-associated peptide epitope and a reversibly masked TGF-β peptide capable of acting as an agonist of cellular TGF-β receptors. The MAPPs are capable of presenting the TID-associated peptide epitope in the context of a Class II MHC receptor to T cells. The present disclosure provides nucleic acids comprising nucleotide sequences encoding those MAPPs, as well as cells genetically modified with the nucleic acids encoding those MAPPs. MAPPs of the present disclosure are useful for selectively modulating activity of a T cells having T cell receptors that recognize the TID epitopes presented by the MAPP. Thus, the present disclosure provides compositions and methods for modulating the activity of T cells, as well as compositions and methods for treating persons who have TID.

This application contains a sequence listing submitted electronically via EFS-web, which serves as both the paper copy and the computer readable form (CRF) and consists of a file entitled “2910_29 PCT_seqlist_ST25”, which was created on Apr. 20, 2022, is 298,917 bytes in size, and which is herein incorporated by reference in its entirety.

### I. INTRODUCTION

An adaptive immune response involves the engagement of the T cell receptor (TCR), present on the surface of a T cell, with a small antigenic molecule non-covalently presented on the surface of an antigen presenting cell (APC) by a major histocompatibility complex (MHC; also referred to in humans as a human leukocyte antigen (“HLA”) complex). This engagement represents the immune system's targeting mechanism and is a requisite molecular interaction for T cell modulation (activation or inhibition) and effector function. In addition to epitope-specific cell targeting, the targeted T cells are activated through engagement of costimulatory proteins found, for example, on the APC with counterpart costimulatory proteins (e.g., receptors) on the T cells. Both signals—epitope/TCR binding and engagement of APC costimulatory proteins with T cell costimulatory proteins—are required to drive T cell specificity and activation or inhibition. The TCR is specific for a given epitope; however, costimulatory proteins are not epitope specific, and instead are generally expressed on all T cells or on subsets of T cells.

APCs generally serve to capture and break the proteins from foreign organisms, or abnormal proteins, into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived (infectious agent-derived), tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors. Conversely, the broad, non-specific activation of overly active T cell responses against self-antigens or shared antigens, such as those targeted in Type 1 Diabetes (T1D), can give rise to T cells that inappropriately attack and destroy healthy tissues or cells.

MHC proteins are referred to as human leukocyte antigens (HLA) in humans. HLA proteins are divided into two major classes, Class I and Class II proteins, which are encoded by separate loci. Unless expressly stated otherwise, for the purpose of this disclosure, references to MHC or HLA proteins are directed to Class II MHC or HLA proteins. HLA Class II proteins each comprise alpha and beta polypeptide chains encoded by separate loci. HLA Class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

Although the immune system is designed to avoid the development of immune responses to proteins and other potentially antigenic materials of the body, in some instances the immune system develops T cells with specificity for self-antigens leading to autoimmune diseases, and in particular Type 1 diabetes (T1D).

Transforming growth factor beta (TGF-β) is a cytokine belonging to the transforming growth factor superfamily that includes three mammalian (human) isoforms, TGF-β1, TGF-β2, and TGF-β3. TGF-βs are synthesized as precursor molecules containing a propeptide region in addition to the TGF-β sequences that homodimerize as an active form of TGF-β. TGF-β is secreted by macrophages and other cell types in a latent complex in which it is combined with two other polypeptides-latent TGF-β binding protein (LTBP) and latency-associated peptide (LAP). The latent TGF-β complex is stored in the extra cellular matrix (ECM), for example, bound to the surface of cells by CD36 via thrombospondin-1 (where it can be activated by plasmin) or to latent transforming growth factor beta binding proteins 1, 2, 3, and/or 4 (LTBP1-4).

The biological functions of TGF-β are seen after latent TGF-β activation, which is tightly regulated in response to ECM perturbations. TGF-β may be activated by a variety of cell or tissue specific pathways, or pathways observed in multiple cell or tissue types; however, the full mechanisms behind such activation pathways are not fully known. Activators include, but are not limited to, proteases, integrins, pH, and reactive oxygen species (ROS). In effect, the cell/tissue bound latent TGF-β complex functions, senses and responds to environmental perturbations releasing active TGF-β in a spatial and/or temporal manner. The released TGF-β acts to promote or inhibit cell proliferation depending on the context of its release. It also recruits stem/progenitor cells to participate in the tissue regeneration/re-modeling process. Aberrations in TGF-β ligand expression, bioavailability, activation, receptor function, or post-transcriptional modifications disturb the normal function, and can lead to pathological consequences associated with many diseases, such as through the recruitment of excessive progenitors (e.g., in osteoarthritis or Camurati-Engelmann disease), or by the trans-differentiation of resident cells to unfavorable lineages (e.g., in epithelial to mesenchymal transition during cancer metastasis or tissue/organ fibrosis). Xu et al Bone Research, 6 (Article No. 2) (2018).

A number of approaches to regulate TGF-β action at the level of the protein by sequestering it to effectively neutralize its action have been described in the literature, and are sometimes referred to as “TGF-β traps.” For example, monoclonal antibodies such as Metelimumab (CAT192) that is directed against TGF-β1, and Fresolimumab directed against multiple isoforms of TGF-β have been developed to bind, sequester, and neutralize TGF-β in vivo. In addition, receptor traps that tightly bind and sequester TGF-β thereby sequestering and neutralizing it have also been developed (see, e.g., Swaagrtra, et al., Mol Cancer Ther; 11(7): 1477-87 (2012) and U.S. Pat. Pub. No. 2018/0327477).

### II. SUMMARY

The present disclosure provides multimeric antigen-presenting polypeptide complexes (“MAPP” singular and “MAPPs” plural) that are at least heterodimeric and include at least one framework polypeptide and at least one dimerization polypeptide. Framework polypeptides comprise one or more polypeptide dimerization sequences that permit specific binding with other polypeptides (dimerization polypeptides) having a counterpart dimerization sequence thereby forming at least a heterodimer (see, e.g., FIGS. 1A and 1B). Framework polypeptides also comprise a multimerization sequence(s) that permit two or more framework polypeptides to associate, thereby forming a higher order structure (e.g., a duplex of the two or more heterodimers, a “duplex MAPP” see, e.g., FIGS. 1A and 1B). Neither the dimerization sequence nor the multimerization sequence of the framework polypeptide (or the counterpart dimerization sequence) comprises an MHC Class II (e.g., HLA) α chain or β chain polypeptide sequence; and as such, interaction brought about by those sequences are not consider dimerization or multimerization of framework and/or dimerization peptides.

The present disclosure also provides an immunomodulatory polypeptide (“MOD”) comprising a TGF-β aa sequence that is reversibly masked by a peptide with affinity for the TGF-β sequence (a “masking sequence”), that taken together are termed “masked TGF-β MOD.” In addition to framework and dimerization polypeptides, the MAPPs described herein further comprise either or both the TGF-β sequence and the masking sequence of the masked TGF-β MOD. Individual MAPPs may comprise a complete masked TGF-β MOD where both the TGF-β sequence and masking sequence are present on the same polypeptide (i.e. placed in “cis,” see, e.g., FIG. 1C at (c) and (d)). Alternatively, an individual MAPP may comprise a complete masked TGF-β MOD where the TGF-β sequence and masking sequence are present in “trans” located on separate polypeptides of the MAPP (e.g., the framework and dimerization polypeptides). When the masking sequence and TGF-β sequence are placed in trans, they may be part polypeptides present in separate MAPPs (e.g., the framework polypeptides of two different MAPPs) and a complete masked TGF-β MOD is formed when those polypeptides are brought together in a higher order MAPP complex (e.g., duplex MAPP, see e.g., FIG. 1C at (a)). Where the masking sequence and the TGF-β sequence are part polypeptides in different MAPP, pairing between a TGF-β sequence and a masking sequence in the higher order MAPP (duplex) can be obtained by using interspecific multimerization sequences (sec, e.g., FIG. 1C at (a) and (b)).

Unlike the TGF-β traps and related molecules discussed above designed to bind and sequester the TGF-β and that act as antagonists to TGF-β action, masked TGF-β MODs provide active TGF-β polypeptides (e.g., TGF-β signaling pathway agonists). The TGF-β polypeptides and a masking polypeptide (e.g., a TGF-β receptor fragment) of masked TGF-β MODs interact with each other to reversibly mask the TGF-β polypeptide sequence permitting the TGF-β polypeptide sequence to interact with its cellular receptor. In addition, the masking sequence competes with cellular receptors that can scavenge TGF-β, such as the non-signaling TβRIII, thereby permitting the MAPP to effectively deliver active TGF-β agonist to target cells. While the MAPP construct permits epitope-specific/selective presentation of a reversibly masked TGF-β to a target cell, it also provides sites for the presentation of one or more additional MODs. The ability of the MAPP construct to include one or more additional MODs thereby permits the combined presentation of TGF-β and the additional MOD(s) to direct a target T cell's response in a substantially epitope-specific/selective manner.

Accordingly, the framework and dimerization peptide containing MAPPs, duplex MAPPs, and MAPPs of higher order (e.g., triplex MAPPs) described herein provide a means by which epitope-presenting peptides (“peptide epitopes” or simply “epitopes”) may be presented in the context of a Class II MHC (e.g., Class II HLA) to a target T cell displaying a TCR specific for the epitope, while at the same time permitting for the flexible presentation of at least one masked TGF-β MOD″, and optionally one or more additional MODs to the target T cell in order to modulate the target T cell. The MAPPs and higher order MAPP complexes thereby permit delivery of one or more masked TGF-β MODs in an epitope selective (e.g., dependent/specific) manner that permits (i) formation of an active immune synapse with a target T cell, such as a CD4+ cell selective for the epitope, and (ii) modulation (e.g., control/regulation) of the target T cell's response to the epitope.

The terms “MAPP” and “MAPPs” as used herein will be understood to refer in different contexts to the heterodimer comprising a framework and dimerization peptide structure as well as higher order complexes of those MAPP heterodimers, such as duplexes (duplex MAPPs). Where reference to both a MAPP and higher order complex is made, it is done for the purpose of emphasis. It will be clear to the skilled artisan when specific reference to only higher order structures are intended (e.g., by reference to duplex MAPPs etc.).

The framework and dimerization peptide containing MAPPs, duplex MAPPs, and MAPPs of higher order (e.g., triplex MAPPs) described herein provide a means by which epitope-presenting peptides (“peptide epitopes” or simply “epitopes”) may be presented in the context of a Class II MHC (e.g., Class II HLA) to a target T cell displaying a TCR specific for the epitope, while at the same time permitting for the flexible presentation of at least one masked TGF-β MOD″, and optionally one or more additional MODs. The MAPPs and higher order MAPP complexes thereby permit delivery of one or more masked TGF-β MODs in an epitope selective (e.g., dependent/specific) manner that permits (i) formation of an active immune synapse with a target T cell, such as a CD4+ cell selective for the epitope, and (ii) modulation (e.g., control/regulation) of the target T cell's response to the epitope.

Peptide epitope presentation by a MAPP to a target T cell is accomplished via a moiety that comprises MHC Class II polypeptides and the peptide epitope. Such moieties may be either (i) a single polypeptide chain, or (ii) a complex comprising two or more polypeptide chains.

Where the peptide epitope. MHC Class II polypeptides, and optionally one or more MODs are provided in a single polypeptide chain, it is termed a “presenting sequence” See, e.g., FIG. 15. The presenting sequences may be integrated into a MAPP as part of a framework polypeptide or a dimerization polypeptide. A MAPP may have presenting sequences as part of either or both of framework or dimerization polypeptide. Compare, for example. FIG. 9 structures A-D and FIG. 10 structures A-D.

As an alternative to utilizing a single polypeptide to present an epitope, the MHC components (e.g., α1, α2, β1 and β2 domain sequences) and the epitope may be divided among two separate polypeptide sequences, which together are denoted herein as a “presenting complex.” See, e.g., FIGS. 17 to 20. A presenting complex is integrated into a MAPP by having a presenting complex first amino acid sequence (“presenting complex 1^{st} sequence”) as part of a framework or dimerization polypeptide. The remaining MHC sequence(s) are part of a polypeptide termed the presenting complex second amino acid sequence (“presenting complex 2^{nd} sequence”). The peptide epitope and any independently selected MODs that are present may be part of the polypeptide comprising either the presenting complex 1^{st} sequence or the presenting complex 2^{nd} sequence. The presenting complex 1^{st} sequence and presenting complex 2^{nd} sequence generally associate through non-covalent interactions between the α chain and β chain polypeptide sequence, and may be stabilized by disulfide bonds between either the MHC sequences or peptide/polypeptide linkers attached to the N- or C-t terminus of the MHC sequences. The presenting complex 1^{st} sequence and presenting complex 2^{nd} sequence may also associate through dimerization or interspecific dimerization sequences if present in those polypeptides.

Although an individual MAPP may not comprise a presenting sequence or presenting complex, for the purpose of this disclosure the MAPPs are, unless stated otherwise, understood to comprise at least one presenting sequence or presenting complex.

MAPPs that comprise a presenting sequence typically contain one or two presenting sequences. Duplex MAPPS thus typically comprise two, three or four presenting sequences, but also may comprise one presenting sequence (e.g., if one of the MAPPS does not comprise a presenting sequence). MAPPs and duplex MAPPs may comprise more presenting sequences depending on, for example, the number of dimerization sequences in the framework polypeptide. The presenting sequences may be integrated into a MAPP as part of a framework polypeptide, a dimerization polypeptide, or both. Compare, for example, FIG. 9 structures A-D and FIG. 10 structures A-D.

Likewise, MAPPs with presenting complexes typically contain one or two presenting complexes, and accordingly, duplex MAPPs with presenting complexes typically comprise two, three or four presenting complexes, but also may comprise one presenting complex (e.g., if one of the MAPPs does not comprise a presenting complex). As discussed above, MAPPs and duplex MAPPs may comprise more presenting complexes depending on, for example, the number of dimerization sequences in the framework polypeptide.

A MAPP of the present disclosure may have a structure comprising:
a framework polypeptide comprising (e.g., from N-terminus to C-terminus) a dimerization sequence and a multimerization sequence and;
a dimerization polypeptide comprising a counterpart dimerization sequence complementary to the dimerization sequence of the framework polypeptide, and dimerizing therewith through covalent (e.g., disulfide bonds) and/or non-covalent interactions to form a heterodimer; and
at least one (e.g., at least two) presenting sequence and/or presenting complex,
wherein each presenting sequence comprises a T1D peptide epitope, and MHC class II α1, α2, β1, and β2 domain polypeptide sequences;
wherein each presenting complex comprises a presenting complex 1^{st} sequence and a presenting complex 2^{nd} sequence that together comprise a peptide epitope and MHC class II α1, α2, β1, and β2 domain polypeptide sequences, where the peptide epitope is part of the presenting complex 1^{st} sequence or presenting complex 2^{nd} sequence along with at least one of the α1, α2, β1, and β2 domain polypeptide sequences;
wherein at least one or both of the dimerization polypeptide and/or the framework polypeptide (e.g., either the framework polypeptide, dimerization polypeptide, or both polypeptides) comprise a presenting sequence or a presenting complex 1^{st} sequence (e.g., located on the N-terminal side of the framework polypeptide's dimerization sequence, or the N-terminal side of the dimerization polypeptide’ counterpart dimerization sequence);
wherein the framework polypeptide, dimerization polypeptide, presenting sequence, or presenting complex comprises (i) a TGF-β sequence, (ii) a masking sequence, or (iii) at least one (e.g., at least two) masked TGF-β immunomodulatory polypeptide (masked TGF-β MOD), each masked TGF-β MOD comprising a masking sequence and TGF-β sequence;
wherein the framework polypeptide, or dimerization peptide (including the presenting sequence(s) or presenting complex(s) that are present) optionally comprise at least one (e.g., at least two, at least three or more) additional MOD (wt. and/or variant) or pair of additional MODs in tandem (both wt., both variant, or one wt. and one variant) (e.g., located at the N-terminus or C-terminus of the dimerization polypeptide or framework polypeptide, and/or on the C-terminal side of the dimerization sequences); and
wherein the framework polypeptide, dimerization polypeptide, presenting sequence, presenting complex 1^{st} sequence and/or presenting complex 2^{nd} sequence optionally comprise one or more linker sequences that are selected independently. (Sec, e.g., FIGS. 1A and 1B).

Such MAPPs may be complexed to form a duplex or higher order MAPP comprising at least a first MAPP heterodimer and a second MAPP heterodimer wherein:
(i) the first MAPP comprises a first framework polypeptide having a first multimerization sequence and a first dimerization sequence, and a first dimerization polypeptide having first counterpart dimerization sequence complementary to the first dimerization sequence; and
(ii) the second MAPP comprises a second framework polypeptide having a second multimerization sequence and a second dimerization sequence, and a second dimerization polypeptide having second counterpart dimerization sequence complementary to the second dimerization sequence; wherein the first and second framework polypeptides are associated by binding interactions between the first and second multimerization sequences optionally including one or more interchain covalent bonds (e.g., one or two disulfide bonds), and the multimerization sequences are not the same (e.g., not the same type and/or not identical to), and do not substantially associate with or bind to, the dimerization sequences or counterpart dimerization sequences. See e.g., the duplexes in FIGS. 19 to 23; and
wherein the duplex or higher order MAPP comprises at least one masked TGF-β MOD, wherein the masking sequence and the TGF-β sequence are present in cis or in trans.

It is understood that the dimerization sequence and multimerization sequences are different polypeptide sequences and do not bind in any substantial manner to each other, e.g., the framework polypeptides do not, to any substantial extent, form hair pin structures, self-polymerize, or self-aggregate. The MAPPs of the present disclosure may be subject to the proviso that neither the dimerization sequence nor the multimerization sequence of the framework polypeptide comprises an MHC-Class II polypeptide sequence having at least 85% (e.g., 90%, 95% or 98%) sequence identity to at least 20 (e.g., at least 30, 40, 50, 60 or 70) contiguous aas of a MHC-Class II polypeptide in any of FIGS. 4 through 18B. It is also understood that none of the α1, α2, β1 and β2 domain polypeptide sequences include a transmembrane domain, or a portion thereof, that will anchor the MAPP in a cell membrane.

While the term MAPP(s) as used in the present disclosure refers to MAPPs comprising a framework polypeptide and dimerization polypeptide heterodimer, the term MAPP, and its plural MAPPs, also refer to their higher order complexes comprising two or more copies of the heterodimer. Where specific forms of higher order complexes are being referred to, e.g., duplexes of the heterodimer, the are specified as duplex, triplex, etc. Accordingly, unless specified otherwise, where the term terms MAPP or MAPPs are used, the terms include higher order complexes, such as duplexed MAPPs, particularly where therapeutic applications and treatments are involved. MAPPs, and accordingly their higher order complexes (duplexes, triplexes etc.), comprise MHC Class II polypeptide sequences and a T1D peptide epitope for presentation to a TCR, may present peptides to T cells (e.g., CD4+ T cells) that have a TCR specific for the epitope. Once engaged with the TCR of a T cell, the effect of a TGF-β MOD-containing MAPP on the T cell depends on which additional MODs (e.g., IL-2 MOD polypeptides), if any, are present as part of the MAPP.

The masked TGF-β MOD-containing MAPPs of the present disclosure can function as a means of producing TGF-β driven T cell responses. For example, TGF-β by itself can inhibit the development of effector cell functions of T cells, activate macrophages, and/or promote tissue the repair after local immune and inflammatory action subside. Accordingly, the TGF-β MOD-containing MAPPs may be employed in vitro or in vivo, including as a therapeutic to induce any of those functions. TGF-β also regulates the differentiation of functional distinct subsets of T cell. TGF-β in the presence of IL-1 and/or IL-6 promotes the development of cells of the Th17 lineage, particularly in the absence of either IL-2 or an IL-2 agonist (e.g., an antibody binding to and acting as an agonist of the IL-2 receptor).

The TGF-β MOD containing MAPPs, and particularly those comprising one or more IL-2 MODs (e.g., variant MODs) or co-administered with an IL-2 or an IL-2 agonist, can bring about the induction and/or proliferation and/or maintenance (survival) of CD4⁺FOXP3⁺ T reg cells specific/selective for the epitope presented by the MAPP. Contacting T cells with a combination of a MAPP and IL-2 (either as an IL-2 MOD, an IL-2R agonist or IL-2) in vitro or in vivo potently inhibits effector T helper (Th) cell differentiation into cells of the Th1, Th2, and/or Th17 lineages. Accordingly, the masked TGF-β MOD-containing MAPPs (e.g., those bearing an IL-2 MOD) are capable of suppressing the immune response to the MAPP-included epitope through, for example, the induction, proliferation, and/or maintenance of T reg cells induced/produced in response to the MAPPs, and any downstream effects of those T reg cells including suppression of CD8+ T cells (activity and/or proliferation) and/or suppression B cells (e.g., antibody production and/or proliferation). MAPPs (e.g., duplex MAPPs) therefore may provide methods of suppressing T cell and B cell activity in vitro and in vivo, and the use of MAPPs (e.g., duplex MAPPs) as therapeutics for in vivo or in vitro methods of treatment. Thus, the present disclosure provides methods of modulating activity of T cells and/or B cells in vitro and in vivo, in disorders related to immune dysregulation/disfunction including the autoimmune disease type 1 diabetes (“T1D”).

In addition to the foregoing. MOD-containing MAPPs, including the masked TGF-β MODs, can function as a means of selectively delivering the MODs to T cells specific for the MAPP associated epitope, thereby resulting in MOD-driven responses to those MAPPs (e.g., the reduction in number and/or suppression of CD4+ effector T cells reactive with MAPP-associated epitopes). Depending on the chosen MOD, the incorporation of one or more MODs with increased affinity for their cognate receptor on T cells (“co-MOD”) may reduce the specificity of MAPPs and duplex MAPPs for epitope specific T cells where MOD-co-MOD binding interactions significantly compete with MHC/epitope binding to target cell TCR. Conversely, and again depending on the chosen MOD, the inclusion of MODs with reduced affinity for their co-MOD(s), and the affinity of the epitope for a TCR, may provide for enhanced selectivity of MAPPs and duplex MAPPs, while retaining the desired activity of the MODs. Where a MOD already possesses a relatively low affinity for its cognate receptor, mutations that reduce the affinity may be unnecessary and/or undesirable for their incorporation into a MAPP.

The ability of MAPPs (e.g., duplex MAPPs) of the present disclosure to modulate T cells provides methods of modulating T cell activity in vitro and in vivo, and accordingly the MAPPs (e.g., duplex MAPPs) are useful as therapeutics are useful in methods of treating T1D.

The present disclosure provides nucleic acids comprising nucleotide sequences and vectors encoding individual MAPP polypeptides and MAPPs (e.g., all polypeptides of a MAPP), as well as cells genetically modified with the nucleic acids and vectors for producing individual MAPP polypeptides and/or MAPP proteins (e.g., duplex MAPPs). The present disclosure also provides methods of producing MAPPs, duplex MAPPs, and higher order MAPPs utilizing such cells.

### III. BRIEF DESCRIPTION OF THE DRAWINGS

FIG. 1A is provided to illustrate of the terminology used to describe MAPPs and duplex MAPPs with presenting sequences. The peptides are oriented from N-terminus (left) to C-terminus (right). The figure shows first and second framework polypeptides, which in this case are different and, in this instance, have specific multimerization sequences comprising a knob and counterpart hole. Such “knob-in-hole” configurations may include knob-in-hole configurations without a stabilizing disulfide bond (herein “KiH”) or with a stabilizing disulfide bond (herein “KiHs-s”). Also shown are first and second dimerization polypeptides having an N-terminal peptide epitope and counterpart dimerization sequences. The dashed circles indicate five potential locations for the addition of polypeptide sequences, including MOD sequences (discussed below). The figure depicts the formation of a first and second heterodimer MAPPs, each comprising a framework polypeptide and dimerization polypeptide. The heterodimers may interact through the multimerization sequence to form a multimer (a duplex MAPP as shown). The use of knob-in-hole sequences permit the assembly of an asymmetric interspecific duplex MAPP where, for example, different MOD sequences are provided at positions 1 and 1′ and/or positions 3 and 3′. While interactions between polypeptide chains through peptide interaction sequences may initially be non-covalent in nature, interchain disulfide bond formation reactions may occur thereby providing covalently linked polypeptides at, for example, either dimerization sequences or multimerization sequences. Throughout the figures, lines connecting various elements of MAPP polypeptides are optional amino acids serving as linkers (e.g., peptide linkers).

FIG. 1B parallels FIG. 1A and is provided to illustrate of the terminology used to describe MAPPs and duplex MAPPs with presenting complexes (the epitope is not shown). The word “sequence” may be abbreviated by “seq.”.

FIG. 1C shows MAPPs with interspecific multimerization sequences, epitope presenting sequences, and at least one masked TGF-β MOD. In (a) and (b) the mask polypeptide sequence and the TGF-β sequence of the masked TGF-β MOD are placed in trans at the 3 and 3′ position of the MAPP. In (c) and (d) the MAPP has two masked TGF-β MODs at the 3 and 3′ positions of the MAPP, with the mask polypeptide sequence and the TGF-β sequence of each masked TGF-β MOD placed in cis. In (a) and (c) the masked TGF-β MODs are shown in the “closed” position. In (b) and (d) the masked TGF-β MODs are shown in the “open” position. Additional MODs, such as IL-2 can be placed at other positions such as positions 1 and 1′.

FIG. 1D parallels FIG. 1C, however each MAPP shown has epitope presenting complexes instead of presenting sequences. The word “sequence” may be abbreviated by “seq.”.

FIGS. 2A-2H provide amino acid sequences of immunoglobulin polypeptides including their heavy chain constant regions (“Ig Fc” or “Fc”, e.g., the CH2-CH3 domain of IgG1) (SEQ ID NOs: 1-13).

FIG. 2I provides the sequence of an Ig CH1 domain (SEQ ID NO:14).

FIG. 2J provides the sequence of a human Ig-J chain (SEQ ID NO:78).

FIG. 3A provides the sequence of an Ig κ chain (kappa chain) constant region (SEQ ID NO:15).

FIG. 3B provides the sequence of an Ig λ chain (lambda chain) constant region (SEQ ID NO:16).

FIG. 4 provides an amino acid sequence of a HLA Class II DRA (sometimes referred to as DRA1) α chain (SEQ ID NO:17).

FIG. 5 provides amino acid sequences of HLA Class II DRB1 β chains (SEQ ID NOs:18-54).

FIG. 6 provides amino acid sequences of HLA Class II DRB3 β chains (SEQ ID NOs:55-58).

FIG. 7 provides an amino acid sequence of a HLA Class II DRB4 β chain (SEQ ID NOs:59-60).

FIG. 8 provides an amino acid sequence of a HLA Class II DRB5 β chain (SEQ ID NO:61).

FIG. 9 provides a series of duplex MAPP structures based on framework polypeptides having both (i) a multimerization sequence, and (ii) first and second dimerization sequences that may be the same or different. The structure is shown generically in A with locations 1-5 and 1′-5′ indicating locations for additional peptide sequences (e.g., MOD polypeptide sequences). The MHC/epitope moiety is illustrated generically, and can be either a presenting sequence (see, e.g., FIGS. 15-16), or a presenting complex (sec FIGS. 17-22). Locations 4 and 4′ are shown at the N-terminus of a presenting sequence or the N-terminus of a presenting complex polypeptide, and locations 5 and 5′ are shown at the C-termini of those polypeptides. Locations 1 and 1′ are shown at the N-terminus of the framework peptide and locations 3 and 3′ at the C-terminus of the framework polypeptide. In A and C, the framework polypeptides are multimerized to form a duplex of heterodimers via non-covalent binding between the multimerization sequences. In B and D, the framework polypeptides are multimerized to form a duplex of heterodimers using an immunoglobulin Fc region knob-in-hole motif, although other methods of covalently bonding the multimerization sequences may be used. In C the duplexes contain heterodimers in which two different asymmetric interspecific dimerization sequences bind together the framework peptides and their associated dimerization peptides. In D the framework peptides are joined together by a knob-in-hole Fc motif and the dimerization peptide and framework peptide are joined together by different dimerization sequences to form a duplex of heterodimers.

FIG. 10 provides in A to D a series of MAPP structures as in FIG. 9, with the addition of presenting sequences or presenting complexes at the N-terminus of the framework peptides. Positions 4 and 4′ may still serve as locations for peptide addition (e.g., MOD polypeptide addition).

FIG. 11 provides in A to D a series of MAPP structures as in FIG. 9, where the dimerization sequences are Ig CH1 sequences (CH1) that pair with Ig light chain sequences (CL). The framework peptides are multimerized (dimers in this instance) through the interaction of Ig Fc (e.g., CH2 and CH3) regions, with the structures in B and D having knob-in-hole motifs to permit heteroduplexes to be formed. The peptides are also joined by disulfide bonds (e.g., those that form between Ig Fc region peptides).

FIG. 12 provides a series of MAPP structures as in FIG. 11, with the addition of presenting sequences or presenting complexes at the N-terminus of the framework peptides. Positions 4 and 4′ may still serve as locations for peptide addition (e.g., MOD polypeptide addition).

FIG. 13 provides in A to H a series of MAPP structures as in FIG. 11. In each instance, a presentation sequence lacking a MOD sequence is present on the dimerization peptide (marked as a single chain MHC and epitope). Locations 2, 2′, 4, 4′, 5 and 5′ are unfiled and not shown numbered. Locations 1 and 1′ are substituted with one or more MODs, e.g., for illustration purposes wild-type (wt.) and/or variants of IL-2, PD-L1, and 4-1BBL, although other MODs may be used, e.g., wild-type and/or variant CD80 or CD86. In A-D Positions 3 and 3′ are used to present a masked TGF-β MOD with the masking sequence and the TGF-β polypeptide sequence in trans (on different polypeptides of the MAPP duplex). Positions 3 a, 3′ in E-H parallel those of A-D respectively, however, positions 3 and 3′ in E-H each bear a masked TGF-β with the masking sequence and TGF-β sequence in cis. The Fc CH2, CH3 sequences in E-H may be replaced by interspecific sequences such as the KiH Fc sequences shown in A-D. In each instance the masked TGF-β MOD is in the closed position with the mask engaging the TGF-β polypeptide sequence so that it cannot effectively act as an agonist of a cellular TGF-β receptor.

FIG. 14 shows four MAPP heterodimer constructs as structures A-D that can form duplex MAPPs. The polypeptide sequences of structures A to D where the “MOD” is PDL1 are provided in FIGS. 34 to 37, however, other MODs such as IL-2 may be utilized. Masking sequence and TGF-β sequence in cis or trans may be incorporated into, for example, the carboxy terminal end (3 or 3′ position) of the framework peptides (the Fc sequence in A-C or the CH1 sequence in D).

FIG. 15 shows in A to C three different MHC Class II presenting sequences (from the epitope at the N-terminus to C-terminus. The sequences optionally comprise one or more independently selected MODs (including two or more MODs in tandem) at the indicated locations.

FIG. 16 shows in A to I nine different embodiments of MHC Class II presenting sequences (from left to right N-terminus to C-terminus).

FIGS. 17 to 22 show a series of MHC Class II presenting complexes from left to right N- to C-terminus. The sequence bearing the symbol “-//-” is the presenting complex 1^{st} sequence. The other sequence is its associated presenting complex 2^{nd} sequence. The symbol “-//-” denotes the point of attachment of the complex to the remainder of the dimerization or framework peptide. In FIG. 20A-L, the presenting complex 1^{st} sequence and its associated presenting complex 2^{nd} sequence include dimerization sequences to unite the peptides (shown as an Ig Fc region associated with the Ig light chain constant region Cκ (kappa chain), although other sequences could be utilized). In FIGS. 21A-F and 22A-F, the presenting complex 1^{st} sequence and its associated presenting complex 2^{nd} sequence include dimerization sequences to unite the peptides (shown as a leucine zipper pair, although other sequences could be utilized).

FIG. 23 provides a table showing examples of HLA Class II alleles. MODs, and T1D epitopes that may be incorporated into a MAPP for T1D therapy.

FIG. 24 the sequences of three different isoforms of TGF-β as preproproteins and the mature form of TGF-β3 along with the C77S mutant of the mature protein.

FIG. 25 provides an alignment of TGF-β isoforms 1-3 with the residues corresponding to the mature form of TGF-β2 bolded, except aa residues Lys 25, Cys 77, Ile 92, and Lys 94 of TGF-β2 and their corresponding residues in the other forms of TGF-β isoforms 1 and 3 that are underlined and italicized but not bolded. TGF-β1 (NP_000651.3 and P01137). TGF-β2 (AAA50405.1), and TGF-β-3 isoform 1 (NP_0013168.1).

FIG. 26A provides the sequences of a type 1 TGF-β receptor (TβRI) and its ectodomain.

FIG. 26B provides the sequences of a type 2 TGF-β receptor (TβRII), its ectodomain, and fragments of the ectodomain. The locations indicated in bold and underlining in the isoform B are aas F30, D32, S52, E55 and D118 of the mature polypeptide, any of which may be substituted with an aa other than the naturally occurring aa. The ectodomain fragments are based upon NCBI Ref. Seq. NP_003233.4, and UniProtKB Ref. P37173; with the ectodomain sequence corresponding to aas 49 to 159 of those sequences. The substitution at aspartic acid “D119” of the mature protein with an alanine “A” (bolded, italicized, and underlined) is marked as a “D118A” substitution for consistency with the literature describing that substitution when signal peptide is understood to be 23 aas in length as opposed to 22 aas in the NCBI record. The aa D119 numbering assignment is based on the mature protein, and accordingly, it is D141 of the precursor protein when the 22 aa signal sequence is included. The location of D32, sometimes substituted with asparagine (D32N), corresponds to D55 in the precursor protein. The corresponding aas in mature isoform A lacking its signal sequence are F55, D57, S77, E80, and D143 (see e.g., SEQ ID NO:198).

FIG. 26C provides the sequences of a type 3 TGF-β receptor (TβRIII).

FIG. 27 provides at (a) the structure of the MAPPs of Example 1 having the masking sequence and the TGF-β polypeptide on the C termini of the framework polypeptides (position 3 and 3′). The masked TGF-β is shown in the closed position with the mask engaging the TGF-β polypeptide. The structure could have also been shown in the open position where the TGF-β is available for interaction with TGF-β receptors. At (b) the figure shows an SDS PAGE gel eight different MAPPs (in lanes 1-8) and four control proteins lanes 9-12, after expression and purification. An optional disulfide bond holding the epitope (circle in the Class II pHLA structure) in the MHC/HLA binding cleft (e.g., between the linker connected to the epitope and an MHC α chain) is not shown.

FIG. 28 provides the amino acid sequences of the polypeptides forming the eight MAPP and four control proteins of Example 1.

### IV. DETAILED DESCRIPTION

### A. Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids, which unless stated otherwise are the naturally occurring proteinogenic L-amino acids that are incorporated biosynthetically into proteins during translation in a mammalian cell. Furthermore, as used herein, a “polypeptide” and “protein” include modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References to a specific residue or residue number in a known polypeptide, e.g., position 72 or 75 of human DRA MHC Class II polypeptide, are understood to refer to the amino acid at that position in the wild-type polypeptide (i.e. I72 or K75). To the extent that the sequence of the wild-type polypeptide is altered, either by addition or deletion of one or more amino acids, the specific residue or residue number will refer to the same specific amino acid in the altered polypeptide (e.g., in the addition of one amino acid at the N-terminus of a peptide reference as position 172, will be understood to indicate the amino acid, Ile, that is now position 73). Substitution of an amino acid at a specific position is denoted by an abbreviation comprising, in order, the original amino acid, the position number, and the substituted amino acid, e.g., substituting the Ile at position 72 with a cysteine is denoted as I72C.

A nucleic acid or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including blast.ncbi.nlm.nih.gov/Blast.cgi for BLAST+2.10.0, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10. Unless otherwise indicated, the percent sequence identities described herein are those determined using the BLAST program.

As used herein amino acid (“aa” singular or “aas” plural) means the naturally occurring proteogenic amino acids incorporated into polypeptides and proteins in mammalian cell translation. Unless stated otherwise: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acid also includes the amino acids hydroxyproline and selenocysteine, which appear in some proteins found in mammalian cells, however, unless their presence is expressly indicated they are not understood to be included.

As used herein the term “in vivo” refers to any process or procedure occurring inside of the body, e.g., of a T1D patient.

As used herein, “in vitro” refers to any process or procedure occurring outside of the body.

The term “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

The term “binding” refers to a direct association between molecules and/or atoms, due to, for example, covalent, electrostatic, hydrophobic, and ionic and/or hydrogen-bond interactions, including interactions such as salt bridges and water bridges.

“Covalent bonding.” or “covalent binding” as used herein, refers to the formation of one or more covalent chemical bonds between two different molecules. The term “binding.” as used with reference to the interaction between a MAPP and a T cell receptor (TCR) on a T cell, refers to a non-covalent interaction between the MAPP and TCR.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen. As used herein, the term “avidity” refers to the resistance of a complex of two or more agents to dissociation after dilution.

“T cell” includes all types of immune cells expressing CD3, including T-helper cells (CD4+ T-helper cells), cytotoxic T cells (CD8+ cells), T-regulatory cells (Treg), and NK-T cells.

The term “immunomodulatory polypeptide” (also referred to as a “costimulatory polypeptide” or, as noted above, “MOD”), as used herein includes a wild-type or variant of a polypeptide or portion thereof that can specifically bind a cognate co-immunomodulatory polypeptide (“co-MOD”) present on a T cell, and provide a modulatory signal to the T cell when the TCR of the T cell is engaged with an MHC-epitope moiety that is specific for the TCR. Unless stated otherwise the term “MOD” includes wild-type and/or variant MODs, and statements including reference to both wild-type and variant MODs are made to emphasize that one, the other, or both are being referenced. The signal provided by the MOD engaging its co-MOD mediates (e.g., directs) a T cell response. Such responses include, but are not limited to, proliferation, activation, differentiation, suppression/inhibition of proliferation, activation and/or differentiation, and the like.

“Heterologous.” as used herein, means a nucleotide or polypeptide that is not found in the native nucleic acid or protein, respectively.

“Recombinant.” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector.” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual.” “subject.” “host.” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “purifying”. “isolating”, and the like, refer to the removal of a desired substance, e.g., a MAPP, from a solution containing undesired substances, e.g., contaminates, or the removal of undesired substances from a solution containing a desired substance, leaving behind essentially only the desired substance. In some instances, a purified substance may be essentially free of other substances, e.g., contaminates. As will be understood by those of skill in the art, generally, components of the solution itself, e.g., water or buffer, or salts are not considered when determining the purity of a substance.

Where a range of values is provided, it is understood that each intervening value between the upper and lower limit of that range to a tenth of the lower limit of the range is encompassed within the disclosure along with any other stated or intervening value in the range. The upper and lower limits of these smaller ranges may independently be included in smaller ranges, that are also encompassed within the disclosure subject to any specifically excluded limit in the stated range. Where the stated range a value (e.g., an upper or lower limit), ranges excluding those values are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a.” “an.” and “the” include plural referents unless the context clearly dictates otherwise. Thus, for example, reference to “a Treg” includes a plurality of such Tregs and reference to “the MHC Class II alpha chain” includes reference to one or more MHC Class II alpha chains and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### B. Description

### 1. MAPP Structure and the Role of Framework and Dimerization Peptides

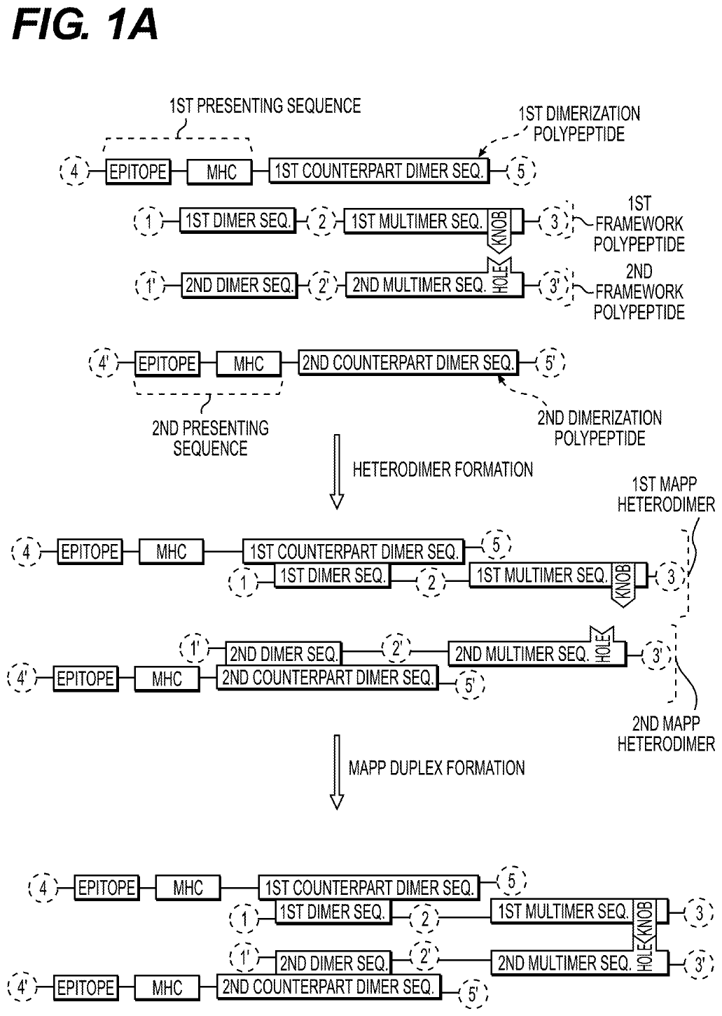

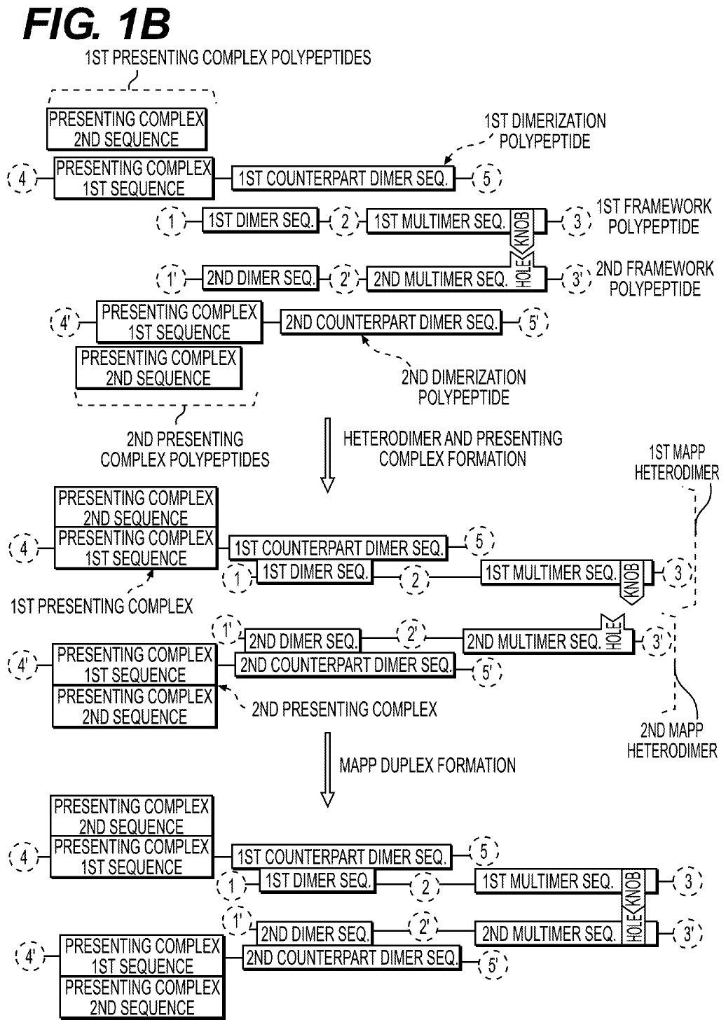

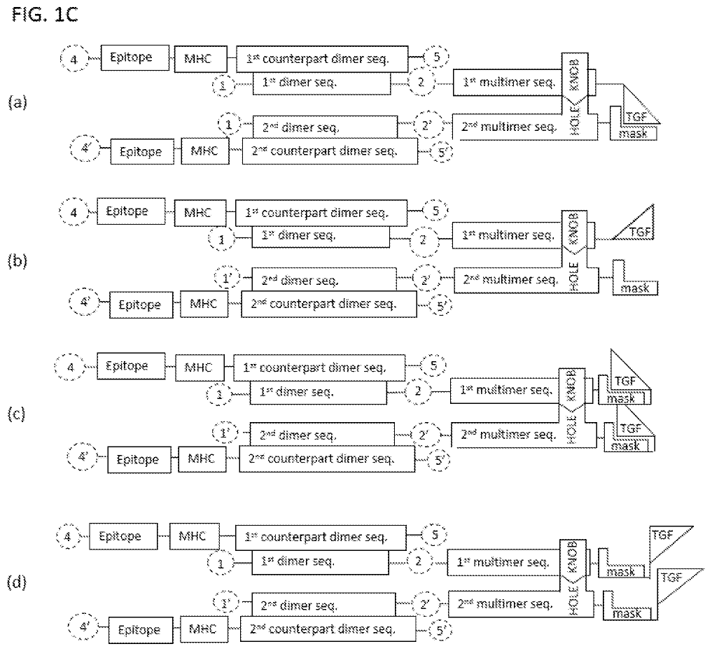

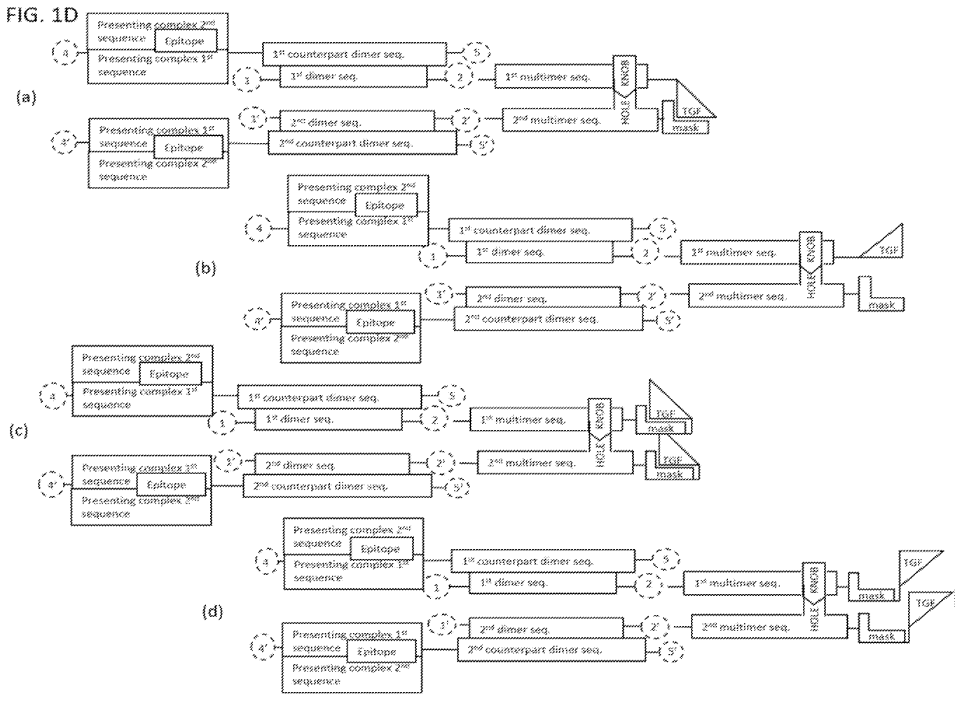

The present disclosure provides MAPPs for use in the treatment of T1D. As discussed above, the MAPPs include at least one framework polypeptide and at least one dimerization polypeptide. Framework polypeptides comprise one or more polypeptide dimerization sequence that permits specific binding with other polypeptides (dimerization polypeptides) having a counterpart dimerization sequence thereby forming at least a heterodimer (See FIGS. 1A and 1B). Framework polypeptides also comprise a multimerization sequence(s) that permits two or more framework polypeptides to associate, thereby forming a higher order structure (e.g., a duplex of the two or more heterodimers, as a “duplex MAPP.” see, e.g., FIGS. 1A and 1B). Neither the dimerization sequence nor the multimerization sequence of the framework polypeptide (or the counterpart dimerization sequence) comprises an MHC (e.g., HLA) Class II α chain or β chain polypeptide sequence; and as such, interaction brought about by those sequences are not consider dimerization or multimerization of framework and/or dimerization peptides. Accordingly, the framework polypeptides provide a structure upon which other polypeptides can be organized by interactions at the dimerization sequences, and which can interact with other framework polypeptides by way of multimerization sequences.

FIGS. 1C and 1D show as examples a series of duplex MAPPs with masked TGF-β MODs in the close or open positions. In (a) and (b) the mask polypeptide sequence and the TGF-β sequence of the masked TGF-β MOD are placed in trans at the 3 and 3′ position of the MAPP. In (c) and (d) the MAPP has two masked TGF-β MODs at the 3 and 3′ positions of the MAPP, with the mask polypeptide sequence and the TGF-β sequence of each masked TGF-β MOD placed in cis. Because the masking sequence and the TGF-β sequence of each MOD in (c) and (d) are placed in cis, the first and second multimerization sequences of the framework polypeptides need not be an interspecific pair. In (a) and (c) the masked TGF-β MODs are shown in the “closed” position with the TGF-β sequence engaged by the masking polypeptide and unavailable to interact with cellular receptors. In (b) and (d) the masked TGF-β MODs are shown in the “open” position with the TGF-β sequence available to interact with cellular receptors. Additional MODs, such as IL-2 can be placed at other positions such as positions 1 and 1′.

The MAPPs, duplex MAPPs, and MAPPs of higher order (e.g., triplex MAPPs) discussed above provide a means by which peptide epitopes may be delivered in the context of an MHC (e.g., HLA) and masked TGF-β MOD(s) to a target T cell displaying a TCR specific for the epitope, while at the same time permitting for the flexible presentation of one or more MODs in addition to the masked TGF-β MOD(s). The MAPPs, duplex MAPPs, and higher order MAPPs thereby permit deliver of one or more MODs in an epitope selective (e.g., dependent/specific) manner that permits formation of an active immune synapse with a target T cell selective for the epitope, and control/regulation of the target T cell's response to the epitope. The target T cell's response to the MAPP depend on the MODs and epitope presented by the MAPP. Accordingly, where MAPPs comprise stimulatory or activating MODs (e.g., IL-2, CD80, CD86, and/or 4-1BBL) that increase T cell proliferation and/or effector functions in an epitope selective manner. In contrast, where MAPPs comprise suppressive/inhibitory MODs (e.g., FasL and/or PD-L1) they generally decrease T cell activation, proliferation, and/or effector functions in an epitope selective manner. The MAPPs, particularly when comprising one or more masked TGF-β MOD and one or more IL-2 MOD polypeptide sequences may function to increase the induction or proliferation of Tregs in an epitope selective manner. MAPPs of the present disclosure, which bear at least one masked TGF-β MOD alone or in combination with one or more IL-2 MOD polypeptide sequence may also be combined with additional MOD such as PD-L1 or 4-1BBL to provide additional modulatory signals.

The framework/dimerization polypeptide architecture of MAPPs and their higher order structures may also be understood to provide flexibility in locating MODs and epitope presenting complexes or epitope presenting sequences. Duplex MAPP and higher order MAPP structures can be particularly useful when both the MOD and the epitope presenting complexes (or epitope presenting sequences) are positioned so as to provide the desired biological activity as well as other desired properties of the MAPP. e.g., thermal stability and manufacturability. In some cases, acceptable combinations of properties may be obtained when the MOD and presenting complex or presenting sequence are positioned at the N-terminus of a polypeptide, e.g., each may be located at the N-terminus of different framework and/or dimerization polypeptide sequences. In some cases, acceptable combinations of properties may be obtained when the MOD and presenting complex or presenting sequence are positioned at the C-terminus of a polypeptide, e.g., each may be located at the C-terminus of different framework and/or dimerization polypeptide sequences. In some cases, acceptable combinations of properties may be obtained when the MOD and presenting complex or presenting sequence are positioned at the N-terminus and C-terminus of a polypeptide, respectively, e.g., the MOD may be located at the N-terminus and the presenting complex or presenting sequence may be located at the C-terminus of different framework and/or dimerization polypeptide sequences. In some cases, acceptable combinations of properties may be obtained when the MOD and presenting complex or presenting sequence are positioned at the C-terminus and N-terminus of a polypeptide, respectively, e.g., the MOD may be located at the C-terminus and the presenting complex or presenting sequence may be located at the N-terminus of different framework and/or dimerization polypeptide sequences.

Examples of masked TGF-β MOD placement include locating a masked TGF-β MOD that comprises the masking sequence and the TGF-β sequence in cis at the carboxy terminal end of MAPP framework polypeptides or dimerization polypeptides (e.g., See e.g., FIGS. 1A and 1B positions 3, 3′, 5 or 5′). Masked TGF-β MODs that comprise the masking sequence and the TGF-β sequence in cis may also be located at, for example, the amino terminal end of MAPP framework polypeptides or dimerization polypeptides (e.g., See e.g., FIG. 1 positions 1, 1′, 4 or 4′). Other positions of the MAPP may also be utilized including the N- or C-terminus of the presenting complex second sequences.

Placement of TGF-β MODs where the masking sequence and TGF-β sequence are in trans (on different polypeptides of the MAPP) is exemplified by having the masking sequence and the TGF-β sequence both at the carboxyl ends or both at N-terminal ends of MAPP polypeptides. By way of example, in a duplex MAPP the masking sequence and the TGF-β sequence may both be at the carboxyl terminus of the framework peptides (sec e.g., structures (a) and (b) of FIGS. 1C and 1D). Masked TGF-β MODs that comprise the masking sequence and the TGF-β sequence in trans may also be located at, for example, the amino terminal end of MAPP framework polypeptides (positions 1 and 1′).

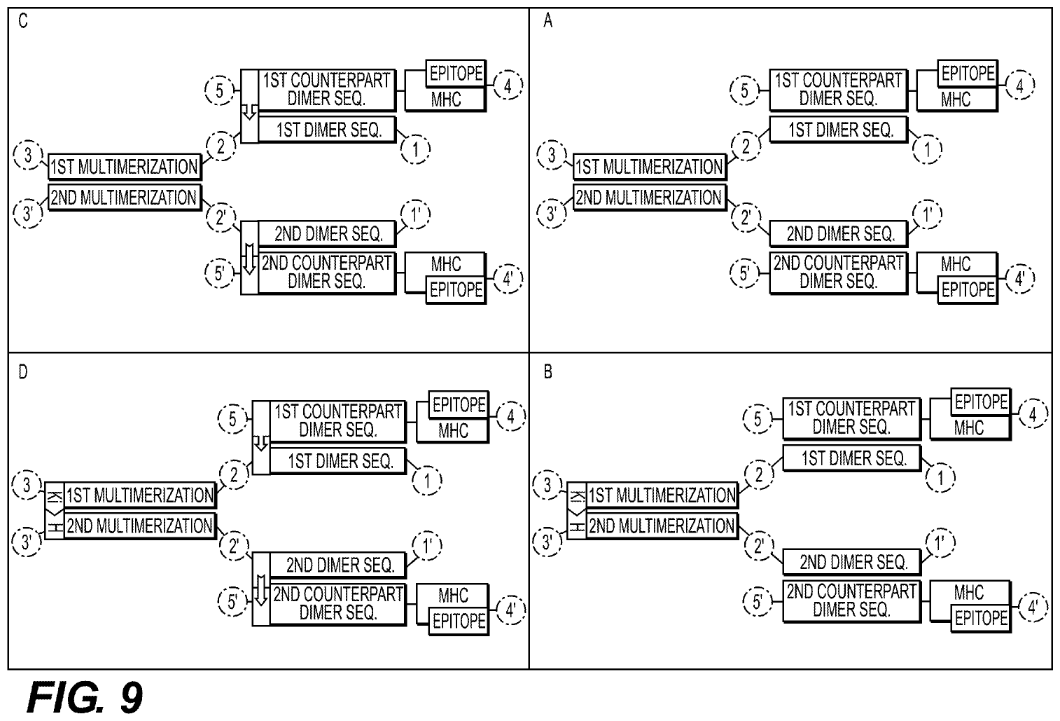

The structure of MAPPs, and particularly higher order MAPPs such as duplexes, may be specified by the use of pairs of polypeptides having different sequences that specifically pair with each other. Multimerization of framework polypeptides results from interactions between multimerization sequences, and dimerization (the interaction of a framework and dimerization polypeptide) results from the interaction of a dimerization sequence on the framework polypeptide and a counterpart dimerization on a dimerization polypeptide. For example, in a duplex MAPP the multimerization sequences may be Ig Fc heavy chain (e.g., CH2-CH3) sequences, and the dimerization sequence and counterpart dimerization sequences may be the same (e.g., all leucine zipper sequences). An additional degree of control may be obtained by utilizing non-identical peptide sequences that specifically/selectively pair with each other that are referred to herein generally as “interspecific sequences,” in the case of dimerization sequences “interspecific dimerization sequences.” or in the case of multimerization sequences “interspecific multimerization sequences.” and which give rise to asymmetric interspecific pairs of sequences. The structure of MAPPs thus permits diverse and effective placement of each polypeptide into the MAPP structure (see, e.g., FIGS. 9-13). Interspecific sequences include Ig heavy chain Fc (e.g., CH2-CH3) region modified with, for example, knob-in-hole variations; and Fos peptide sequences paired with Jun peptide sequences. Accordingly, MAPP structures include, but are not limited to, MAPPs where each, or some, of the dimerization sequences are different (permit different peptide pairings). For example, duplex MAPPs where each multimerization and dimerization sequence is different and provides separate peptide pairings.

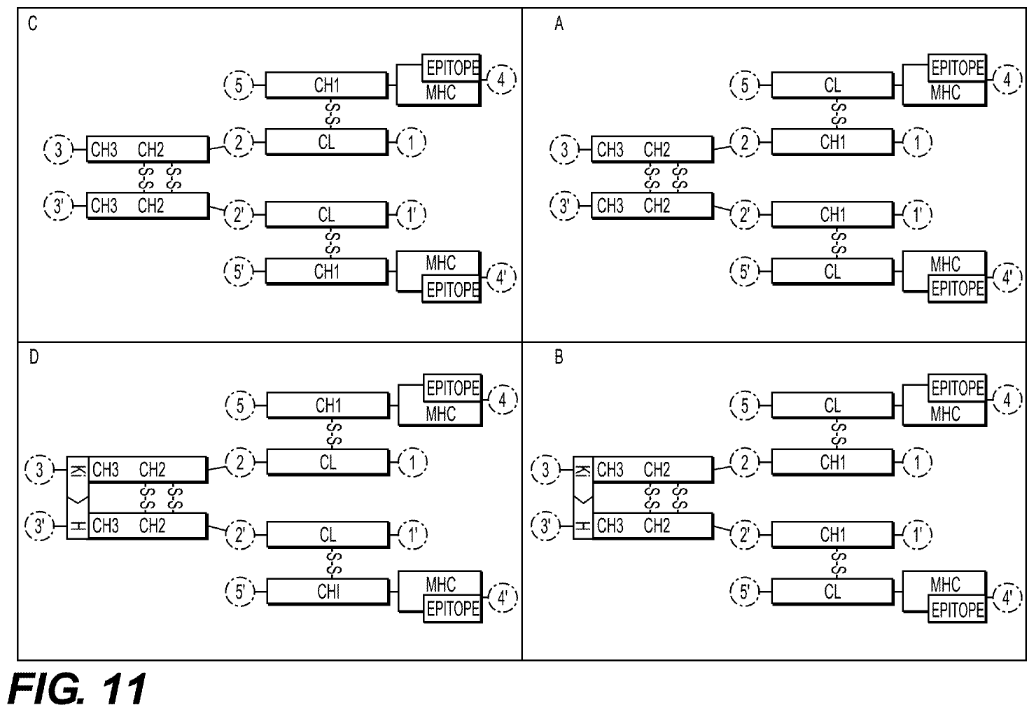

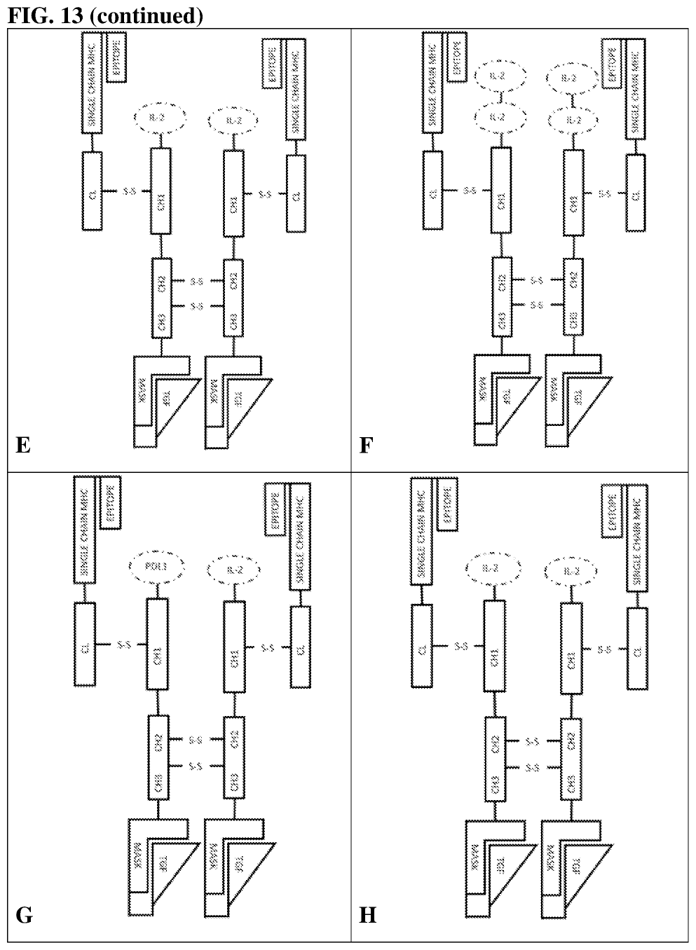

In an embodiment, the framework peptide multimerization sequence is an Ig Fc heavy chain region (optionally and interspecific Fc sequence such as a knob-in hole Fc sequence) and the dimerization sequences are the same (e.g., Ig CH1 sequences paired with light chain λ or κ constant region sequences) (see, for example, FIGS. 11 and 22, structures A to D). In another embodiment, the framework peptide multimerization sequence is an Ig Fc heavy chain region (optionally and interspecific Fe sequence such as a knob-in hole Fe sequence) and the dimerization sequences are selected to be different (e.g., a dimerization sequence pair comprising an Ig CH1 paired with light chain λ or κ sequence and a dimerization sequence comprising a leucine zipper pair, see for example, FIG. 13, structures E to H). For example, in a duplex MAPP the multimerization sequences may be a knob-in-hole Ig sequence, one dimerization sequence and its counterpart dimerization sequence may be leucine zipper sequences, and second dimerization sequence and its counterpart dimerization sequence may be an Ig CH1 and Ig CL λ domain pair.

MAPPs and accordingly their higher order complexes (duplexes, triplexes etc.) comprise MHC Class II polypeptide sequences that bind an epitope for presentation to a TCR, and accordingly may present peptides to T cells (e.g., CD4⁺ T cells). The effect of MAPPs on T cells with TCRs specific to the epitope depends on which, if any. MODs in addition to the masked TGF-β MOD(s) that are present in the MAPP. As noted above. MAPPs, duplex MAPPS and higher order MAPPs comprising MOD(s) permit MOD delivery to T cells in an epitope selective manner and the MODs principally dictate the effect of MAPP-T cell engagement in light of the specific cell type stimulated and the environment. While not wishing to be bound by any particular theory, the effect of MAPP (e.g., duplex MAPP) presentation of MOD(s) and epitope to a T cells in some cases may be enhanced relative to the situation encountered in antigen presenting cells (APC) where epitope can diffuse away from the MHC (e.g., HLA) complex and any MODs the APC is presenting. This cannot occur with a MAPP where the epitope and MOD are part of the MAPP polypeptide(s) and cannot diffuse away even if the epitope's affinity for the MHC complex would normally permit it to leave the comparable cell complex. The inability of epitope to diffuse away from MHC and MOD components of a MAPP or higher order MAPP complex may be further limited where the polypeptide(s) of the MAPP (e.g., framework, dimerization sequence, and if present, the presenting complex 2^{nd} sequence) are covalently attached to each other (e.g., by disulfide bonds). Consequently. MAPPs and their higher order structures may be able to prolong delivery of MOD(s) to T cells in an epitope selective manner relative to systems where epitopes can diffuse away from the presenting MHC.

Incorporation of one or more MODs with affinity for their cognate receptor on T cells (“co-MOD”) can reduce the specificity of MAPPs (e.g., duplex MAPPs) for epitope selective/specific T cells. The reduction in epitope selectivity/specificity of the MAPPs becomes more pronounced where MOD/co-MOD binding interactions increase in strength (binding energy) and significantly compete with MHC/epitope binding to target cell TCR. The inclusion of variant MODs, including TGF-β MODs, with reduced affinity for their co-MOD(s) thus may provide a lower contribution of MOD binding energy, thereby permitting MHC-epitope interactions in which the TCR dominates the binding and provides epitope selective interactions with T cells while retaining the activity of the MODs. Variant MODs with one or more substitutions (or deletions or insertions) that reduced the affinity of the MOD for their co-MOD may be incorporated into MAPPs and their higher order complexes alone or in combination with wild-type MODs polypeptide sequences. Wild-type and variant MODs are described further below. Inclusion of masking sequences that bind tightly to the TGF-β polypeptide sequence effectively reduces the apparent affinity of the TGF-β polypeptide sequence for the cellular receptors, thereby decreasing the contribution of TGF-β polypeptide to cellular TβR bind in MAPP association with a T cells, which permits MHC-epitope interactions with the TCR to dominate the T-cell binding interactions and effect epitope specific/selective T cell interactions and epitope specific/selective delivery of the masked TGF-β MOD and any other MODs on the MAPP to the target T cell.

The ability of MAPPs to modulate T cells in an epitope selective/specific manner thus provides methods of modulating activity of a T cell in vitro, and in vivo, and accordingly, methods of treating autoimmune T1D diabetes resulting from immune dysregulation/disfunction.

The present disclosure provides nucleic acids comprising nucleotide sequences encoding MAPP polypeptides, cells genetically modified with the nucleic acids and capable of producing the MAPP, and methods of producing MAPPs and their higher order complexes utilizing such cells.

Each presenting sequence or presenting complex present in a MAPP comprises MHC Class II alpha and beta chain polypeptide sequences (e.g., human MHC Class II sequences) sufficient to bind a peptide epitope and present it to a TCR. MHC Class II peptides, may include sequence variations that are designed to stabilize the MHC, stabilize the MHC-peptide epitope complex, and/or stabilize the MAPP. Sequence variations may also serve to enhance cellular expression of MAPPs prepared in cell-based systems as well as the stability (e.g., thermal stability) of MAPPs and their higher order complexes such as duplex MAPPs. Some MHC Class II sequences suitable for use in MAPPs are described below.

As indicated in the description of the drawings. MAPPs may comprise one or more independently selected peptide sequences or (one or more “linker” or “linkers”) between any two or more components of the MAPP, which in the figures may be shown as a line between peptide and/or polypeptide elements of the MAPPs. The same sequences used as linkers may also be located at the N- and/or C-termini of the MAPP peptides to prevent, for example, proteolytic degradation. Linker sequences include but are not limited to polypeptides comprising: glycine; glycine and serine; glycine and alanine; alanine and serine; and glycine, alanine and serine; any one which may comprise a cysteine for formation of an intra or interpolypeptide disulfide bond. Various linkers are described in more detail below.

### 2. Exemplary MAPP Structures

MAPPs of the present disclosure comprise (i) framework polypeptides with a multimerization sequence and at least one dimerization sequence, and (ii) dimerization polypeptides with a counterpart dimerization sequence that binds with the framework polypeptide's dimerization sequence. As discussed above. MAPPs of the present disclosure further comprise one, two, or more masked TGF-β MODs, and either one or more epitope presenting sequences or one or more epitope presenting complexes. Exemplary structures for such MAPPs appear in FIG. 1A to 1D, and FIGS. 9-13 in some instances at least one MOD is shown as a masked TGF-β MOD. The structure depicted in FIG. 13 represents MAPPs with at least one masked TGF-β MOD, multimerizing framework polypeptides and epitope presenting sequences (the “Single Chain MHC” with the “Epitope”). In FIGS. 1A and 1n FIGS. 9-12, the structures represent MAPPs with multimerizing framework polypeptides where the epitope MHC combination represents either epitope presenting sequences or epitope presenting complexes.

Interactions of MHC (e.g., HLA) sequences are not considered herein to result in multimerization and/or dimerization. In an embodiment, neither the dimerization sequence nor the multimerization sequence of the framework polypeptide, nor the counterpart dimerization sequence of the dimerization polypeptide comprises a Class II MHC polypeptide sequence having at least 90% (e.g., 95% or 98%) sequence identity to at least 15 (e.g., at least 20, 30, 40, 50, 60 or 70) contiguous aas of a MHC Class II polypeptide (e.g., a polypeptide in any of FIGS. 4 to 8). In embodiments. MAPPs comprise at least one, or at least two, dimerization peptides that comprise an epitope presenting sequence. See, e.g., FIGS. 1A and 1B.

One group of masked TGF-β MOD-containing having epitope presenting sequences, comprise in addition to the masked TGF-β MOD: a multimerizing framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptide and the framework polypeptide comprise a presenting sequence located on the N-terminal side of their dimerization or counterpart dimerization sequences. In such a MAPP the presenting sequence may comprise a peptide epitope and one or more MHC polypeptide sequences, with the peptide epitope sequence located: (i) at or within 10 aa, 15 aa, 20 aa, or 25 aa of the N-terminus of the presenting sequence, or (ii) in a polypeptide located at the N-terminus of the presenting sequence comprising, from N-terminus to C-terminus, a MOD, one or more optional linkers, and the peptide epitope; optionally at least one (e.g., one, two or each) of the framework polypeptide, dimerization peptide, and presenting sequence comprises one or more independently selected MODs located at their N-terminus and/or C-terminus (or on the N-terminal or C-terminal side of the dimerization or counterpart dimerization sequences); wherein the MHC polypeptide sequences are MHC Class II polypeptide sequences they comprise MHC Class II α1, α2, β1, and β2 polypeptide sequences (e.g., human MHC Class II sequences). In an embodiment, neither the dimerization sequence nor the multimerization sequence of the framework polypeptide comprises a Class II MHC peptide sequence having at least 90% (e.g., 95% or 98%) sequence identity to at least 15 (e.g., at least 20, 30, 40, 50, 60 or 70) contiguous aas of a MHC Class II polypeptide in any of FIGS. 4 to 18B.

Another group of MAPPs, those having epitope presenting complexes, comprise a multimerizing framework polypeptide having, from N-terminus to C-terminus, a dimerization sequence and multimerization sequence; and a dimerization polypeptide comprising a counterpart dimerization sequence complementary to the dimerization sequence of the framework polypeptide and dimerizing therewith through covalent and/or non-covalent interactions to form a heterodimer; wherein at least one (e.g., one, or both) of a dimerization polypeptides and/or at least one (e.g., one or both) of the framework polypeptide comprise a presenting complex 1^{st} sequence located on the N-terminal side of their dimerization sequence. A presenting complex 2^{nd} sequence is associated with the presenting complex 1^{st} sequence (e.g., non-covalently or covalently such as by one or two interchain disulfide bonds) to form a presenting complex. In such a MAPP each of the presenting complex 1^{st} sequence and its associated presenting complex 2^{nd} sequence are comprised of one or more MHC polypeptide sequences, with one of the sequences further comprising the peptide epitope. The peptide epitope may be located (i) at or within 10 aa, 15 aa, 20 aa, or 25 aa of the N-terminus of the presenting complex 1^{st} sequence or presenting complex 2^{nd} sequence, or (ii) in a polypeptide located at the N-terminus of the presenting complex 1^{st} sequence or presenting complex 2^{nd} sequence, with the polypeptide comprising, from N-terminus to C-terminus, a MOD, one or more optional linkers, and the peptide epitope. Optionally, at least one (e.g., one, two or each) of the framework polypeptide, dimerization peptide, or the peptides of a presenting complex comprise one or more independently selected MODs located at their N-terminus or C-terminus (or on the N-terminal or C-terminal side of the dimerization sequences).

MAPPs may be constructed such that neither the dimerization sequence nor the multimerization sequence of the framework polypeptide comprises a Class II MHC peptide sequence having at least 90% (e.g., 95% or 98%) sequence identity to at least 15 (e.g., at least 20, 30, 40, 50, 60 or 70) contiguous aas of a MHC Class II polypeptide in any of FIGS. 4 to 18B.

As discussed above, a dimerization sequence of a framework polypeptide may interact with dimerization peptides to form heterodimers. The multimerization sequence of the framework polypeptide may associate with another framework polypeptide multimerization sequence forming a duplex (or higher order structure, such as a triplex, quadraplex or pentaplex) of the heterodimers. Where the multimerization sequences are interspecific (e.g., a knob-in-hole Fc peptide pair), and at least one heterodimer comprises an interspecific dimerization and counterpart dimerization pair, two different heterodimers may be formed. When the different heterodimers are combined to form a duplex MAPP, any one or more component (e.g., MODs) may differ (e.g., in type or location) between the two heterodimers.

### C. MAPP Components

### 1. Framework Polypeptides and Dimerization Polypeptides

As may be understood from the preceding sections, framework polypeptides serve as the structural basis or skeleton of MAPPs, permitting the organization of other elements in the MAPP complex. Framework peptides interact with other peptides through binding interactions, principally at dimerization and multimerization sequences. Interactions at dimerization sequences permit association of non-framework peptides (e.g., dimerization peptides) with framework peptides. In contrast, multimerization sequences are involved in the interaction of two or more framework peptides.

The framework polypeptide(s) of MAPPs comprise at least one multimerization sequence, and at least one independently selected dimerization sequence that is not identical to, or of the same type (e.g., not both leucine zipper variants) as, the multimerization sequence. By utilizing different types of sequences for the interactions at multimerization and dimerization sequences, it becomes possible to control the interactions of the framework polypeptide with other framework polypeptides and with dimerization polypeptides. In an embodiment, framework polypeptides comprise one multimerization sequence and one dimerization sequence. In an embodiment, framework polypeptides comprise at least one multimerization sequence and at least two independently selected dimerization sequences. Framework peptides may contain peptide sequences (e.g., linker sequences and/or MOD sequences) between any of the elements of the framework polypeptide or at the ends of the framework polypeptide including the multimerization sequences and dimerization sequences.

In addition to providing for the structural organization of MAPPS through their multimerization and dimerization sequences, framework peptides, and particularly their N- and C-termini, may also serve as locations for placement of elements such as MOD sequences, an epitope, presenting sequences, and/or a presenting complex 1^{st} sequences (one polypeptide of an epitope presenting complex, see FIG. 1B). When placed at the N- and/or C-termini of a framework polypeptide, such polypeptide elements are part of the framework polypeptide (e.g., a single translation product formed in a cell).

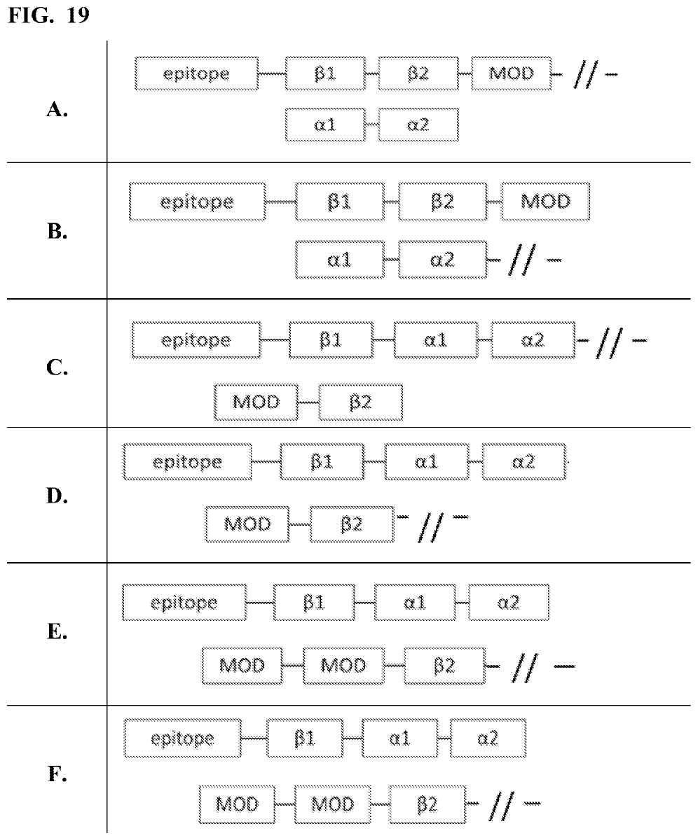

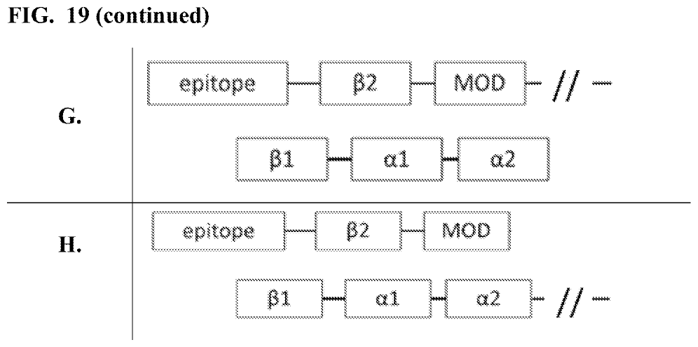

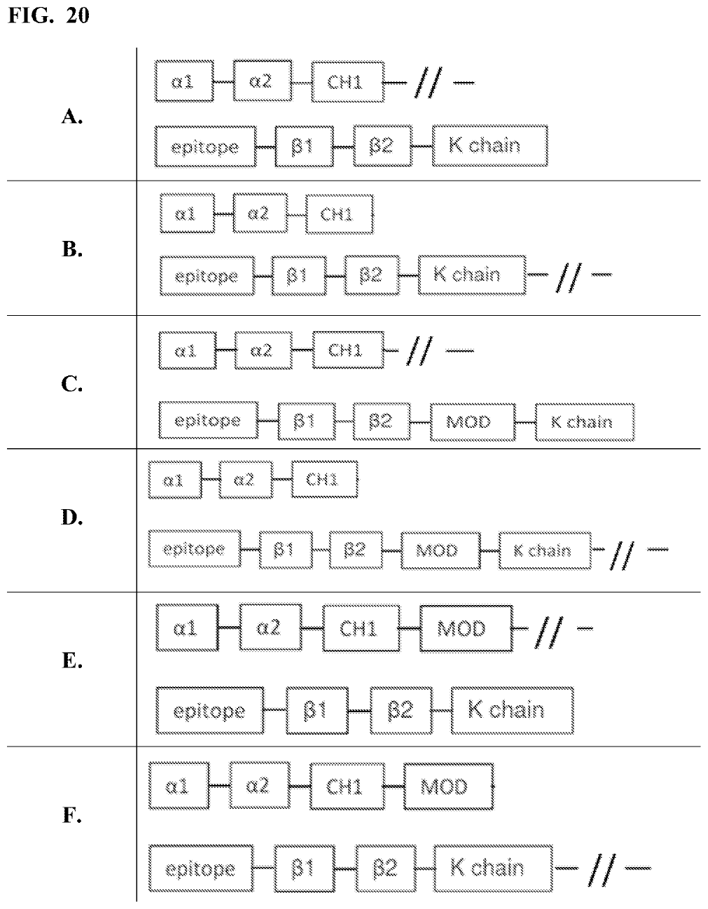

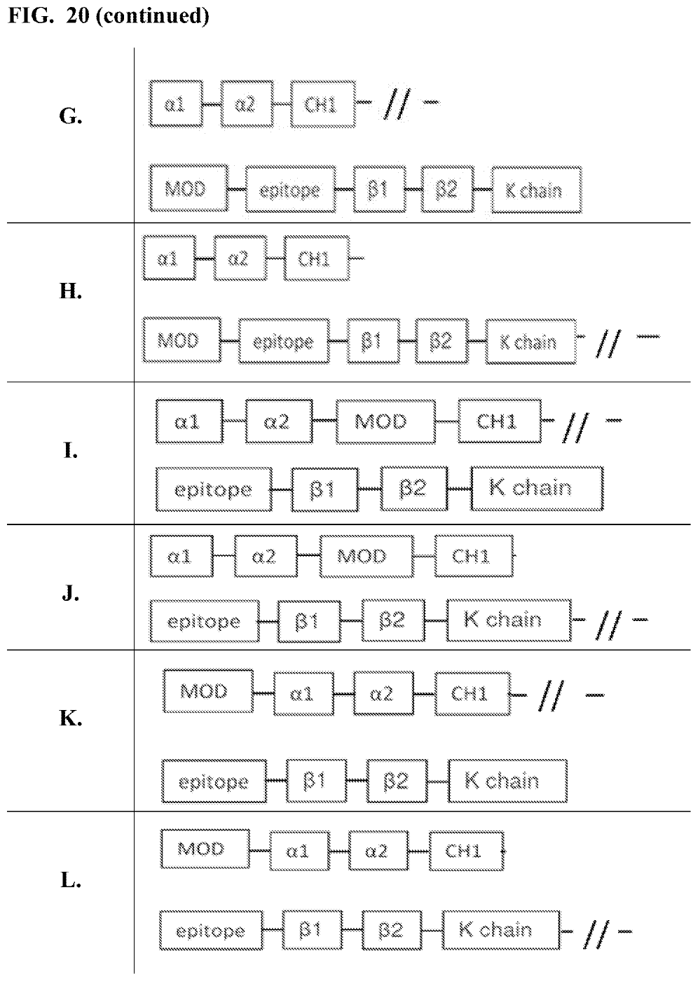

Within a MAPP all of the dimerization sequence may be non-interspecific (such as leucine zipper pairs) while the multimerization sequences is either interspecific or non-interspecific (See, e.g., structures A & B of FIGS. 19 and 20). For example, in a duplex MAPP with first and second framework polypeptides, the multimerization sequences may be a non-interspecific (e.g., an IgFc sequence such as CH2, CH3 domain sequences or leucine zippers) or the multimerization sequences may be an interspecific knob-in-hole sequence pair; with the dimerization sequences of the first and second framework polypeptide as a non-interspecific leucine zipper polypeptides. Where an Fc polypeptide is employed it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region.

Within a MAPP, all of the dimerization sequences may be interspecific, while the multimerization sequences are not interspecific (See, e.g., FIG. 13A). For example, in a duplex MAPP with first and second framework polypeptides, the multimerization sequences may be an IgFc sequence, with the a ZW1 sequence or its counterpart employed as the dimerization sequence of the first framework polypeptide and an Ig CH1 domain or its counterpart Ig C_{L} κ sequence as the dimerization sequence of the second framework polypeptide.

All of the dimerization sequences or all of the dimerization and multimerization sequences, in a MAPP may differ in that they bind only specific binding partners present in the MAPP (e.g., each are part of a different interspecific sequence pair). For example, in a duplex MAPP with first and second framework polypeptides, the multimerization sequences may be a pair of knob-in-hole IgFc sequences, with the a ZW1 sequence or its counterpart employed as the dimerization sequence of the first framework polypeptide, and a Ig CH1 or its counterpart Ig C_{L} sequence as the dimerization sequence of the second framework polypeptide.

### 2. Multimerization and Dimerization Polypeptide Sequences

Amino acid sequences that permit polypeptides to interact may be utilized as dimerization sequences or counterpart dimerization sequences when they are involved in the formation of dimers between a framework polypeptide and a dimerization polypeptide. The same type of aa sequences may be utilized as multimerization sequences when they are used to form duplex or higher order structures (trimers, tetramers, pentamer, etc.) between framework polypeptides. In any given MAPP, sequences that can interact with each other are not utilized as both dimerization and multimerization sequences. Stated another way, the same aa sequence pair may serve as either dimerization or multimerization sequences depending on whether they: bring together two or more framework peptides, in which case they are multimerization sequences; or they bring together a dimerization and multimerization sequence, in which case they are designated as dimerization sequences.

Where dimerization or multimerization sequences employ identical sequences that pair or multimerize (e.g., some leucine zipper sequences), they can form symmetrical pairs or multimers (e.g., homodimers) as shown in FIG. 9 structure A. In contrast, where dimerization or multimerization sequences that pair are not identical and require a specific complementary counterpart sequence to form a dimer, they are interspecific binding sequences and can form asymmetric pairs. Both immunoglobulin (e.g., IgFc) and non-immunoglobulin polypeptides can be interspecific or non-interspecific in nature. For example, both Fos/Jun binding pairs and Ig CH1 polypeptide sequences and light chain constant region C_{L} sequences form interspecific binding pairs. Natural Ig Fc regions tend to be non-interspecific, but, as discussed below, can be made to form interspecific pairs (e.g., KiH pairs). Coiled-coil sequences, including leucine zipper sequences, can be either interspecific leucine zipper or non-interspecific leucine zipper sequences. See, e.g., Zeng et al., (1997) PNAS (USA) 94:3673-3678; and Li et al., (2012), Nature Comms. 3:662.

Interspecific binding sequences may in some instances form some amount of homodimers, but preferentially dimerize by binding more strongly with their counterpart interspecific binding sequence. Accordingly, specific heterodimers tend to be formed when an interspecific dimerization sequence and its counterpart interspecific binding sequence are incorporated into a pair of polypeptides. By way of example, where an interspecific dimerization sequence and its counterpart are incorporated into a pair of polypeptides, they may selectively form greater than 70%, 80%, 90%, 95%, 98% or 99% heterodimers when an equimolar mixture of the polypeptides are combined (for example in PBS buffer at 20° C.). The remainder of the polypeptides may be present as monomers or homodimers, which may be separated from the heterodimer. See, for example, FIG. 9, structure B, with an interspecific multimerization sequence and structure C with two different interspecific dimerization sequences. Moreover, because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences.

Sequence are considered orthogonal to other sequences when they do not form complexes (bind) with each other's counterpart sequences. See FIG. 9 structure D where the MAPP comprises an interspecific multimerization sequence and two independently selected interspecific dimerization sequences, all of which are orthogonal to each other. Any of the MAPPS described herein may have two or more (e.g., three, four or more) orthogonal dimerization sequences. In an embodiment, MAPPs with multimerizing framework peptides may have orthogonal multimerization and dimerization domains (where the dimerization domains may or may not be orthogonal to each other).

Some sequences permitting polypeptides to interact with sufficient affinity to be used as dimerization and/or multimerization sequences are provided for example in U.S. Patent Publication No. 2003/0138440. The sequences may be of relatively compact size (e.g., such as less than about 300, 250, 225, 200, 175, 150, 125, 100, 75, 60, 50, 40, or 30 aa). In an embodiment, at least one (e.g., at least two or all) of the dimerization and/or multimerization sequences are less than 300 aa. In an embodiment, at least one (e.g., at least two or all) of the dimerization and/or multimerization sequences are less than 200 aa. In an embodiment, at least one (e.g., at least two or all) of the dimerization and/or multimerization sequences are less than 100 aa. In an embodiment, at least one (e.g., at least two or all) of the dimerization and/or multimerization sequences are less than 75 aa. In another embodiment, at least one (e.g., at least two or all) of the dimerization and/or multimerization sequences are less than are less than 50 aa. In an embodiment, at least one (e.g., at least two or all) of the dimerization and/or multimerization sequences are less than 30 aa.

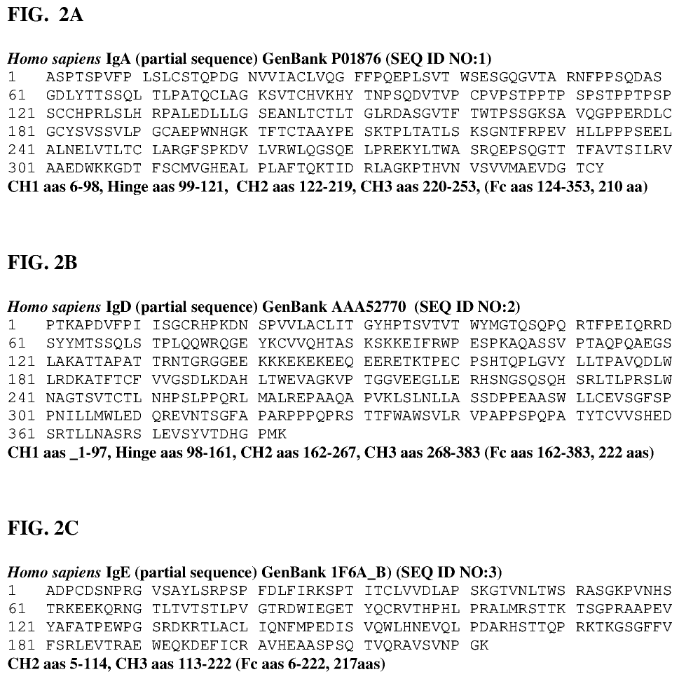

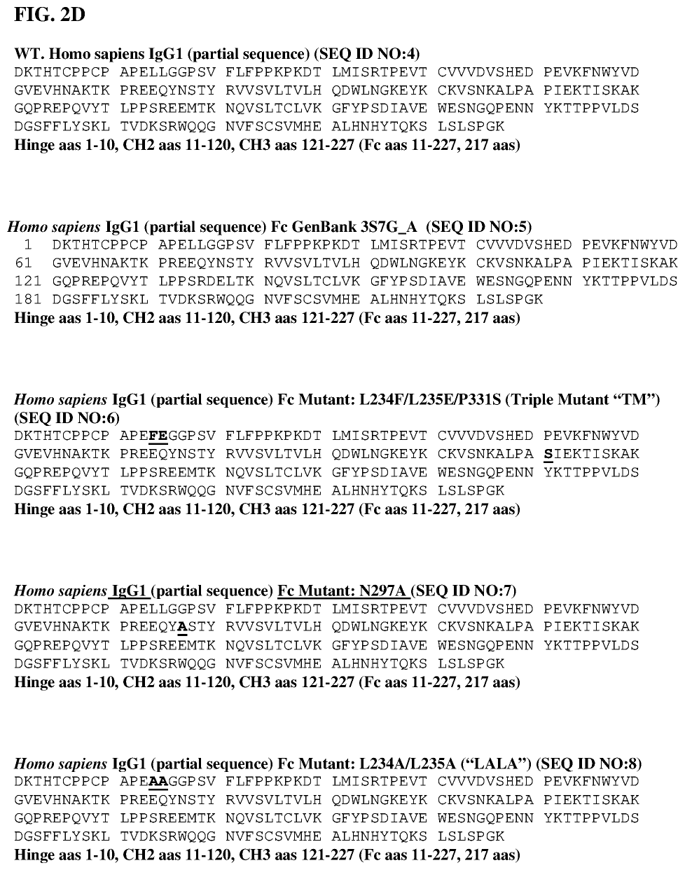

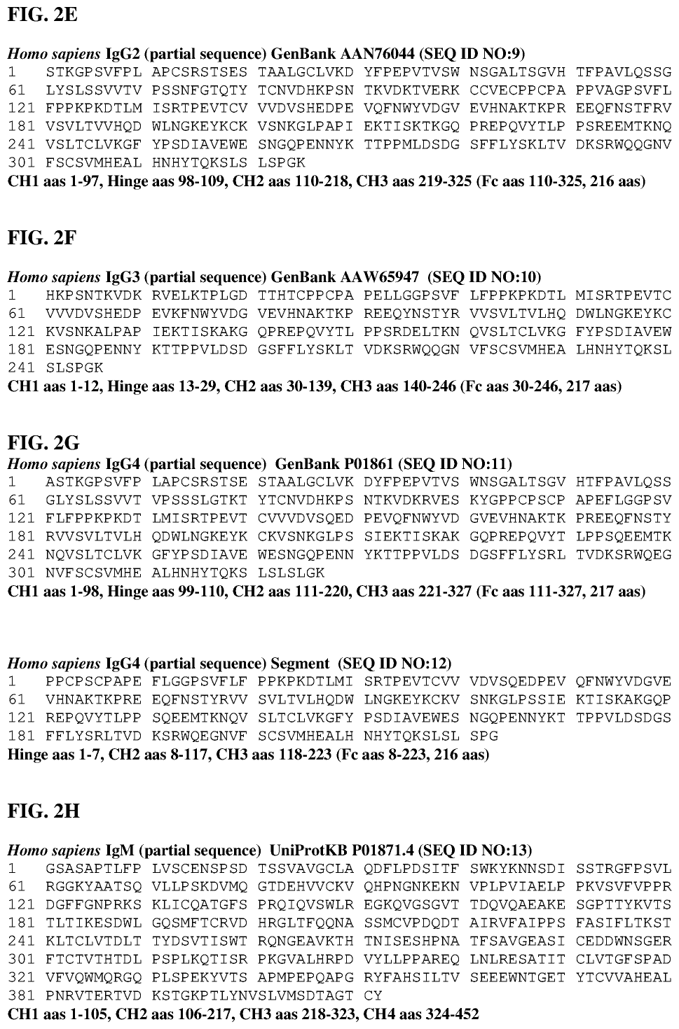

Dimerization/multimerization sequences include but are not limited to: immunoglobulin heavy chain constant region (Ig Fc) polypeptide sequences (e.g., sequences comprising CH2-CH3 regions of immunoglobulins such as those provided in FIGS. 2A-2H and SEQ ID NOs: 1 to 13); polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; interspecific Ig Fc heavy chain constant regions (such as knob-in-hole sequences described in more detail below); Fos/Jun binding pairs; immunoglobulin heavy chain constant region (CH2-CH3) sequences, and; Ig CH1 and light chain constant region CL sequences (Ig CH1/CL pairs such as a Ig CH1 sequence paired with a Ig C_{L} κ or λ light chain constant region sequence).

Framework and/or dimerization polypeptides of a MAPP may comprise an immunoglobulin heavy chain constant region (e.g., CH2-CH3 domains) polypeptide sequence that functions as a dimerization or multimerization sequence. Where the framework polypeptide comprises an IgFc multimerization sequence, and a CH1 dimerization sequence it may comprise all or part a native or variant immunoglobulin sequence set forth in any of FIGS. 2A to 2H that comprise the CH1, CH2 and CH3 domains and any hinge sequences that may be present. An Ig Fe sequence, or any one or more of the CH1, CH2, and CH3 domains, may have at least about 70%, at least about 80%, at least about 90%, at least about 95%, at least about 98%, at least about 99%, or 100% aa sequence identity to an aa sequence of an Fc region depicted in FIGS. 2A-2H. In particular, the C-terminal lysine provided in some of the sequences provided in FIGS. 2A-2H (e.g., the IgG sequences in FIGS. 2D, 2E, 2F, and 2G) may be removed during cellular processing of MAPPs and may not be present on some or all of the MAPP molecules as expressed. See, e.g., van den Bremer et al. (2015) mAbs 7:4; and Sissolak et al. (2019) J. Industrial Microbiol. & Biotechnol. 46:1167.

Such immunoglobulin sequences can covalently link the polypeptides of MAPP complex together by forming one or two interchain disulfide bonds, thereby stabilizing MAPPs, particularly where a pair of interspecific Ig sequence such as knob-in-hole polypeptide pairs are employed. Where an Fc polypeptide sequence, alone or in combination with a CH1 polypeptide sequence, is employed as a multimerization or dimerization sequence it may be, for example, from an IgA, IgD, IgE, IgG, or IgM, which may be a human polypeptide sequence, a humanized polypeptide sequence, a Fc region polypeptide of a synthetic heavy chain constant region, or a consensus heavy chain constant region. As discussed below, the Ig Fc region can further contain substitutions that can substantially remove the ability of the Ig Fc to effect complement-dependent cytotoxicity (CDC) or antibody-dependent cell cytotoxicity (ADCC).

Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgA Fc sequence depicted in FIG. 2A (SEQ ID NO:1). Framework and/or dimerization polypeptides of a MAPP may comprise a sequence that has at least about 70%, at least about 80%, at least about 90%, at least about 95%, at least about 98%, at least about 99%, or 100% aa sequence identity to at least 150 contiguous aas (at least 175, at least 200, at least 225, at least 250, at least 275, at least 300, at least 325, or at least 350 contiguous aas), or all aas, of the IgD Fc sequence depicted in FIG. 2B (SEQ ID NO:2). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (at least 150, at least 175, or at least 200 contiguous aas), or all aas, of the IgE Fc sequence depicted in FIG. 2C (SEQ ID NO:3).

A MAPP may comprise one or more IgG Fe sequences as dimerization and/or multimerization sequences. The Fc polypeptide of a MAPP can be a human IgG1 Fc, a human IgG2 Fc, a human IgG3 Fc, a human IgG4 Fc, etc. In some cases, the Fc sequence has at least about 70%, at least about 80%, at least about 90%, at least about 95%, at least about 98%, at least about 99%, or 100% aa sequence identity to an aa sequence of an Fc region depicted in FIG. 2D-2G. Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 contiguous aas (e.g., at least 150, at least 175, at least 200, or at least 220 contiguous aas), or all aas, of the wt. IgG1 Fc polypeptide sequence depicted in FIG. 2D (SEQ ID NO: 4). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 225) contiguous aas, or all aas, of the IgG2 Fc polypeptide sequence depicted in FIG. 2E (SEQ ID NO:9). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, at least 225, or at least 240) contiguous aas, or all aas, of the IgG3 Fe sequence depicted in FIG. 2F (SEQ ID NO: 10). Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (e.g., at least 150, at least 175, at least 200, or at least 220) contiguous aas, or all aas, of the IgG4 Fe sequence depicted in FIG. 2G (SEQ ID NO:11 or 12).

Framework and/or dimerization polypeptides of a MAPP may comprise a sequence that has at least about 70%, at least about 80%, at least about 90%, at least about 95%, at least about 98%, at least about 99%, or 100% aa sequence identity to at least 80 (at least 90, at least 100, at least 110, or at least all 112) contiguous aas, or all aas, of the IgG1 CH1 sequence provided in FIG. 2I.

Framework and/or dimerization polypeptides of a MAPP may comprise a sequence that has at least about 70%, at least about 80%, at least about 90%, at least about 95%, at least about 98%, at least about 99%, or 100% aa sequence identity to at least 125 (at least 150, at least 175, at least 200, at least 225, or at least 250) contiguous aas, or all aas, of the IgM Fe polypeptide sequence depicted in FIG. 2H (SEQ ID NO:13)

Framework and/or dimerization polypeptides a MAPP comprising immunoglobulin sequences (e.g., depicted in FIGS. 2A-2H) can be covalently linked together by formation of at least one or at least two interchain disulfide bonds between cysteines that are adjacent to the immunoglobulin hinge regions. Such disulfide bonds can stabilize the interaction of framework and dimerization polypeptide heterodimers, or, for example, duplexes of such heterodimers when the disulfide bonds are between framework multimerization sequences.

A framework or dimerization polypeptide may comprise an aa sequence having 100% aa sequence identity to the wt. human IgG1 Fc polypeptide depicted in FIG. 2D. A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, that includes a substitution of N297 (N77 as numbered in FIG. 2D, SEQ ID NO:7) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in complement dependent cytotoxicity (“CDC”). K322 (e.g., K322A) substitutions shows a substantial reduction in reduction in FcγR binding affinity and ADCC, with the C1q binding and CDC functions substantially or completely eliminated. Hezareh et al., (2001) J. Virol. 75:12161-168.

Amino acid L234 and other aas in the lower hinge region (e.g., aas 234 to 239, such as L235, G236, G237, P238, S239) which correspond to aas 14-19 of SEQ ID NO:8) of IgG are involved in binding to the Fc gamma receptor (FcγR), and accordingly, mutations at that location reduce binding to the receptor (relative to the wt. protein) and result in a reduction in antibody-dependent cellular cytotoxicity (or alternatively antibody-dependent cell-mediated cytotoxicity, “ADCC”). Hezarch et al., (2001) have demonstrated that the double mutant (L234A, L235A) does not effectively bind either FcγR or C1q, and both ADCC and CDC functions were substantially or completely abolished. A framework or dimerization polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, that includes a substitution of L234 (L14 of the aa sequence depicted in FIG. 2D) with an aa other than leucine.

In view of the foregoing, a framework or dimerization polypeptides, and in particular Ig Fc sequences used as multimerization or dimerization sequences, may comprise substitutions that reduce or substantially eliminate ADCC and/or CDC responses. The framework or dimerization polypeptides, and in particular Ig Fc sequences used as multimerization or dimerization sequences, may also comprise substitutions that reduce or substantially eliminate ADCC and/or CDC.

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, that includes a substitution of L235 (L15 of the aa sequence depicted in FIG. 2D) with an aa other than leucine. In some cases, the framework and/or dimerization polypeptide present in a MAPP with substitutions in the lower hinge region includes L234A and L235A (“LALA”) substitutions (the positions corresponding to positions 14 and 15 of the wt. aa sequence depicted in FIG. 2D; sec, e.g., SEQ ID NO:8).

A framework or dimerization polypeptide with a substitution in the lower hinge region may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, that includes a substitution of P331 (P111 of the aa sequence depicted in FIG. 2D) with an aa other than proline. Substitutions at P331, like those at N297, lead to reduced binding to C1q relative to the wt. protein, and thus a reduction in complement dependent cytotoxicity (CDC). In one embodiment, the substitution is a P331S substitution. In another embodiment, the substitution is a P331A substitution.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, and include substitutions of D270, K322, and/or P329 (corresponding to D50, K102, and P109 of SEQ ID NO:4 in FIG. 2D) that reduce binding to C1q protein relative to the wt. protein.

A framework or dimerization polypeptide may comprise an aa sequence (e.g., as a multimerization sequence) having at least about 70% (e.g., at least about 80%, 90%, 95%, 98%, or 99%) aa sequence identity to the wt. human IgG1 Fc polypeptide depicted in FIG. 2D, including substitutions at L234 and/or L235 (L14 and/or L15 of the aa sequence depicted in FIG. 2D) with aas other than leucine such as L234A and L235A, and a substitution of P331 (P111 of the aa sequence depicted in FIG. 2D) with an aa other than proline such as P331S. In one instance, a framework or dimerization polypeptide present in a MAPP comprises the “Triple Mutant” aa sequence (SEQ ID NO:6) depicted in FIG. 2D (human IgG1 Fc) having L234F, L235E, and P331S substitutions (corresponding to aa positions 14, 15, and 111 of the aa sequence depicted in FIG. 2D).

Where an asymmetric pairing between two polypeptides of a MAPP is desired, a framework or dimerization polypeptide present in a MAPP may comprise, consist essentially of, or consist of an interspecific binding sequence. Interspecific binding sequences favor formation of heterodimers with their cognate polypeptide sequence (i.e., the interspecific sequence and its counterpart interspecific sequence), particularly those based on immunoglobulin Fc (Ig Fc) sequence variants. Such interspecific polypeptide sequences include KiH, KiHs-s, HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon immunoglobulin sequences are summarized in the table that follows, with cross reference to the numbering of the aa positions as they appear in the wt. IgG1 sequence (SEQ ID NO:4) set forth in FIG. 2D shown in brackets “{ }”.

TABLE 1
Interspecific immunoglobulin sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain S—S |
| | | | bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 modified from Ha et al., Frontiers in Immunol. 7: 1-16 (2016).
*aa forms a stabilizing disulfide bond.

In addition to the interspecific pairs of sequences in Table 1, framework and/or dimerization polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

A framework or dimerization polypeptide found in a MAPP may comprise an interspecific binding sequence or its counterpart interspecific binding sequence selected from the group consisting of: KiH; KiHs-s; HA-TF; ZW-1; 7.8.60; DD-KK; EW-RVT; EW-RVTs-s; A107; or SEED sequences.

A MAPP may comprise a framework or dimerization polypeptide comprising an IgG1 KiH or KiHs-s sequence with a T146W sequence substitution, and its counterpart interspecific KiH or KiHs-s binding partner polypeptide comprises an IgG1 sequence having T146S, L148A, and Y187V sequence substitutions, where the framework and/or dimerization polypeptides comprises a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D. One or both of the framework, or both of dimerization polypeptides optionally comprising substitutions at one of more of: L234 and L235 (e.g., L234A/L235A “LALA” or L234F/L235E); N297 (e.g., N297A); P331 (e.g., P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g., P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 2D.

A MAPP may comprise a framework or dimerization polypeptide comprising an IgG1 sequence with a T146W KiH sequence substitution, and its counterpart interspecific binding partner polypeptide comprises an IgG1 sequence having T146S. L148A, and Y187V KiH sequence substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a T146W and S134C KiHs-s substitutions, and its counterpart interspecific binding partner polypeptide comprises an IgG1 sequence having T146S. L148A, Y187V and Y129C KiHs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a S144H and F185A HA-TF substitutions, and its counterpart interspecific binding partner polypeptide comprises an IgG1 sequence having Y129T and T174F HA-TF substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise framework or dimerization polypeptide comprising an IgG1 sequence with a T130V. L131Y. F185A, and Y187V ZW1 substitutions, and its counterpart interspecific binding partner polypeptide comprises an IgG1 sequence having T130V, T146L, K172L, and T174W ZW1 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140D, D179M, and Y187A 7.8.60 substitutions, and its counterpart interspecific binding partner polypeptide comprises an IgG1 sequence having T130V E125R, Q127R, T146V, and K189V 7.8.60 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2Ds

A MAPP may comprise a framework or dimerization polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D. One or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

A MAPP may comprise a framework or dimerization polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the framework and/or dimerization polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both of the framework and/or dimerization polypeptide sequences may comprise additional substitutions such as L14 and/or L15 substitutions (e.g., “LALA” substitutions L234A and L235A), and/or N77 (N297 e.g., N297A or N297G).

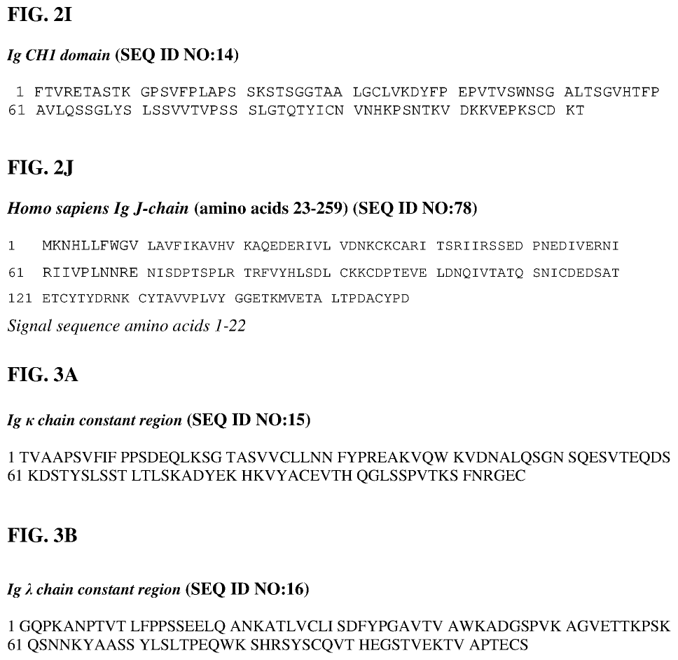

As an alternative to the use of immunoglobulin CH2 and CH3 heavy chain constant regions as dimerization or multimerization sequences, immunoglobulin light chain constant regions (See FIGS. 3A and 3B) can be paired with Ig CH1 sequences (See FIG. 2I) as multimerization or dimerization sequences and their counterpart sequences of a framework polypeptide.

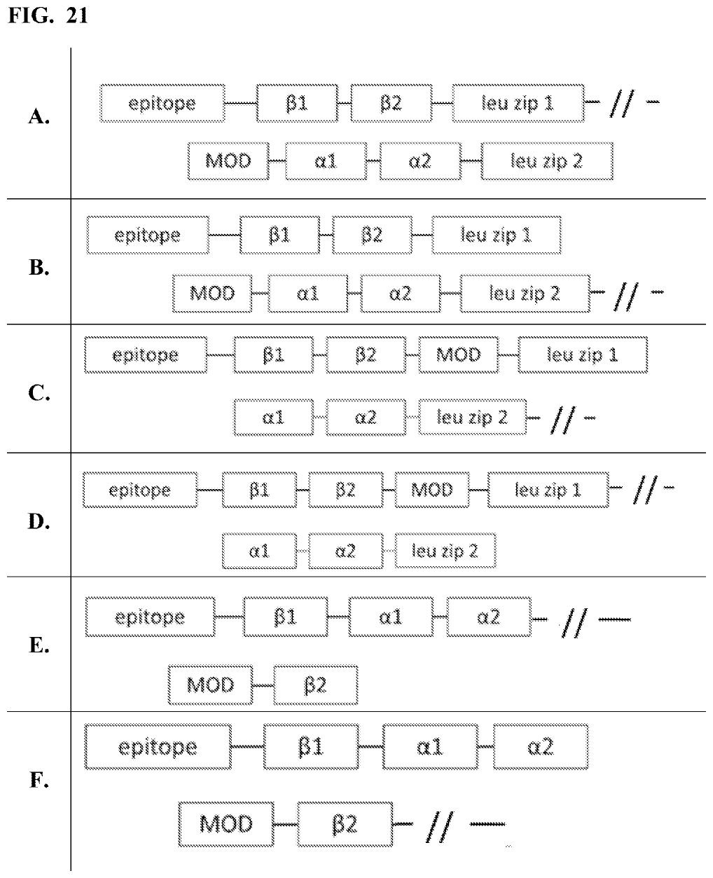

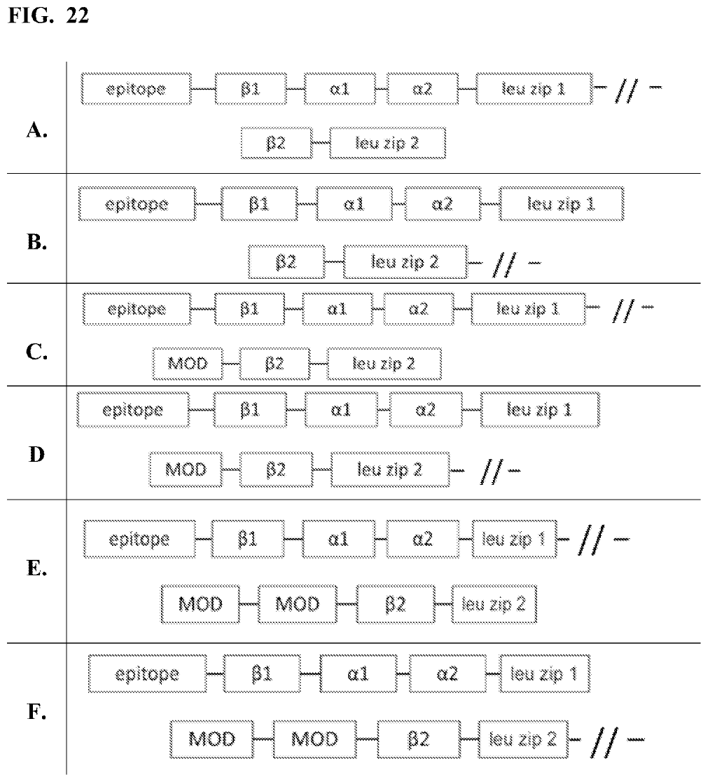

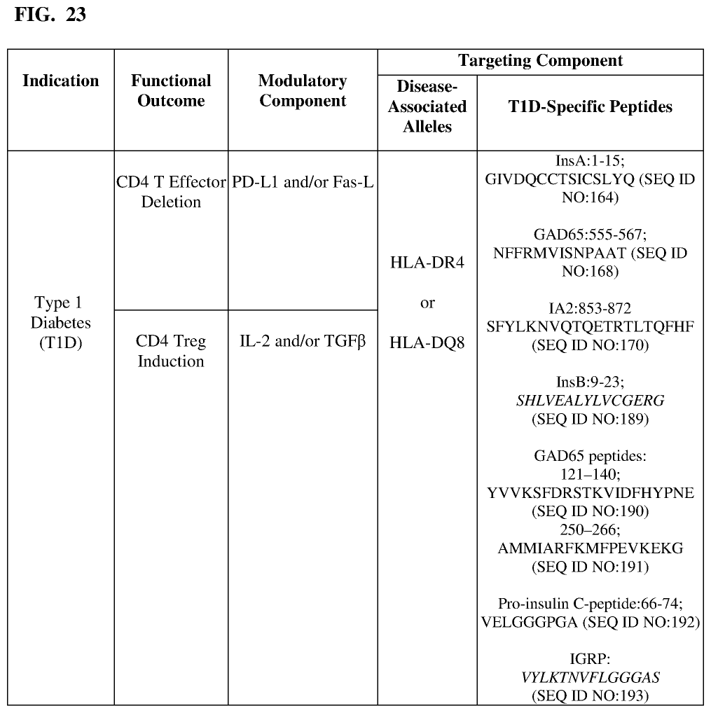

A MAPP framework or dimerization polypeptide may comprise an Ig CH1 domain (e.g., the polypeptide of FIG. 2I), and the sequence with which it will form a complex (its counterpart binding partner) comprises an Ig K chain constant region sequence, where the framework or dimerization polypeptide comprise a sequence having at least 80%, 85%, 90%, 95%, 98%, 99%, or 100% sequence identity to at least 70, at least 80, at least 90, at least 100, or at least 110 contiguous aas of SEQ ID NOs: 14 and/or 15 respectively. See FIGS. 21 and 3A. The Ig CH1 and Ig κ sequences may be modified to increase their affinity for each other, and accordingly the stability of any heterodimer formed utilizing them as a dimerization or multimerization sequences. Among the substitutions that increase the stability of CH1-Ig κ heterodimers are those identified as the MD13 combination in Chen et al., MAbs, 8(4):761-774 (2016). In the MD13 combination two substitutions are introduced into to each of the IgCH1 and Ig κ sequences. The Ig CH1 sequence is modified to contain S64E and S66V substitutions (S70E and S72V of the sequence shown in FIG. 2I). The Ig K sequence is modified to contain S69L and T71S substitutions (S68L and T70S of the sequence shown in FIG. 3A).

A framework or dimerization polypeptide of a MAPP may comprise an Ig CH1 domain (e.g., the polypeptide of FIG. 2I SEQ ID NO: 14), and its counterpart sequence comprises an Ig λ chain constant region sequence such as is shown in FIG. 3B (SEQ ID NO: 16), where the framework or dimerization polypeptide comprises a sequence having at least 80%, 85%, 90%, 95%, 98%, 99%, or 100% sequence identity to at least 70 (e.g., at least 80, at least 90, or at least 100) contiguous aas of the sequences shown in FIG. 3B.

Framework and/or dimerization polypeptides of a MAPP may each comprise a leucine zipper polypeptide as a dimerization or multimerization sequence. The leucine zipper polypeptides bind to one another to form dimer (e.g., homodimer). Non-limiting examples of leucine-zipper polypeptides include a peptide comprising any one of the following aa sequences: RMKQIEDKIEEILSKIYHIENEIARIKKLIGER (SEQ ID NO:62); LSSIEKKQEEQTSWLIWISNELTL-IRNELAQS (SEQ ID NO:63); LSSIEKKLEEITSQLIQISNELTLIRNELAQ (SEQ ID NO:64); LSSIE-KKLEEITSQLIQIRNELTLIRNELAQ (SEQ ID NO:65); LSSIEKKLEEITSQLQQIRNELTLIRNELAQ (SEQ ID NO:66); LSSLEKKLEELTSQLIQLRNELTLLRNELAQ (SEQ ID NO:67); ISSLEKKIEE-LTSQIQQLRNEITLLRNEIAQ (SEQ ID NO:68). In some cases, a leucine zipper polypeptide comprises the following aa sequence: LEIEAAFLERENTALETRVAELRQRVQRLRNRVSQYRTRYGPLGGGK (SEQ ID NO:69). Additional leucine-zipper polypeptides are known in the art, a number of which are suitable for use as multimerization or dimerization sequences.

The framework and/or dimerization polypeptides of a MAPP may comprise a coiled-coil polypeptide that forms a dimer. Non-limiting examples of coiled-coil polypeptides include, for example, a peptide of any one of the following aa sequences: LKSVENRLAVVENQLKTVIEELKTVKDLLSN (SEQ ID NO:70); LARIEEKLKTIKAQLSEIASTLNMIREQLAQ (SEQ ID NO:71); VSRLEEKVKT-LKSQVTELASTVSLLREQVAQ (SEQ ID NO:72); IQSEKKIEDISSLIGQIQSEITLIRNEIAQ (SEQ ID NO:73); and LMSLEKKLEELTQTLMQLQNELSMLKNELAQ (SEQ ID NO:74).

A MAPP may comprise a pair of two framework polypeptides and/or a framework and dimerization polypeptide that each have an aa sequence comprising at least one cysteine residue that can form a disulfide bond permitting homodimerization or heterodimerization of those polypeptides stabilized by disulfide bond between the cysteine residues. Examples of such aa sequences include: VDLEGSTSN-GRQCAGIRL (SEQ ID NO:75); EDDVTTTEELAPALVPPPKGTCAGWMA (SEQ ID NO:76); and GHDQETTTQGPGVLLPLPKGACTGQMA (SEQ ID NO:77).

Some aa sequences suitable as multimerization (oligomerization) sequences permit formation of MAPPs capable of forming structures greater than duplexes of a heterodimers comprising a framework and dimerization polypeptide. In some instances, triplexes, tetraplexes, pentaplexes may be formed. Such aa sequences include, but are not limited to. IgM constant regions (See, e.g., FIG. 2H) which forms hexamer, or pentamers (particularly when combined with a mature j-chain peptide lacking a signal sequence such as that provided in FIG. 2J (SEQ ID NO:78). Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times, such as 10-20, 20-30, or 30-40 times). In such sequences. Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times, such as 10-20, 20-30, or 30-40 times. A collagen oligomerization peptide can comprise the following aa sequence:

(SEQ ID NO: 79)
VTAFSNMDDMLQKAHLVIEGTFIYLRDSTEFFIRVRDGWKKLQLGEL
IPIPADSPPPPALSSNP.

Suitable framework polypeptides (e.g., those with an Ig Fc multimerization sequence) will, in some cases, be half-life extending polypeptides. Thus, in some cases, a suitable framework polypeptide increases the in vivo half-life (e.g., the serum half-life) of the MAPPs, compared to a control MAPP having a framework polypeptide with a different aa sequence. For example, in some cases, a framework polypeptide increases the in vivo half-life (e.g., the serum half-life in a mammal such as a human) of the MAPP, compared to a control MAPP having a framework polypeptide with a different aa sequence. The half-life may be extended by at least about 10%, at least about 15%, at least about 25%, at least about 50%, at least about 100%, at least about 2-fold, at least about 5-fold, at least about 10-fold, at least about 25-fold, at least about 50-fold, at least about 100-fold, or more than 100-fold. As an example, in some cases, an Ig Fc polypeptide sequence (e.g., utilized as a multimerization sequence to form a duplex of MAPP heterodimers comprising a framework and dimerization polypeptide) increases the stability and/or in vivo half-life (e.g., the serum half-life) of a MAPP duplex, compared to a control MAPP duplex lacking the Ig Fc polypeptide sequence by at least about 10%, at least about 15%, at least about 25%, at least about 50%, at least about 100%, at least about 2-fold, at least about 2.5-fold, at least about 5-fold, at least about 10-fold, at least about 25-fold, at least about 50-fold, at least about 100-fold, or more than 100-fold.

### 3. Presenting Sequence and Presenting Complexes

As discussed in more detail below. Class II MHC polypeptides, include two types of polypeptide chains, α-chain and β-chain. More specifically. MHC Class II α-chain polypeptides include α1 and α2 domains, and β-chain polypeptides include β1 and β2 domains. Presenting sequences and presenting complexes comprise MHC Class II polypeptides sufficient to bind and present an epitope to a TCR. Presenting sequences and complexes may also comprise additional protein (peptide) elements including one or more independently selected MODs and/or one or more independently selected linkers (e.g., linkers placed between various domains). As discussed herein, unless stated otherwise, neither presenting sequences nor presenting complexes comprise an MHC transmembrane domain (or intracellular domain such as a cytoplasmic tail) sufficient to anchor MAPP molecules (e.g., more than 50% of the MAPP molecules) in a mammalian cell membrane (e.g., a CHO cell membrane) when expressed therein.

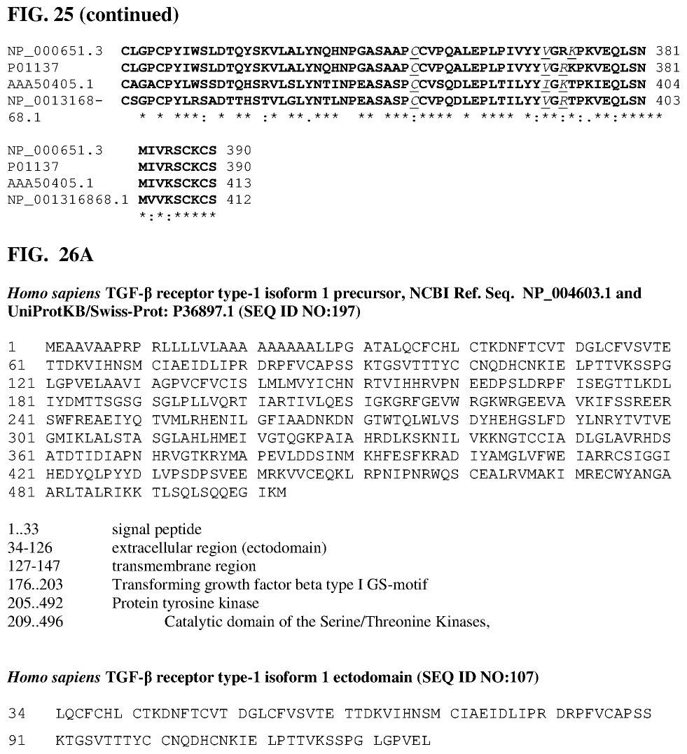

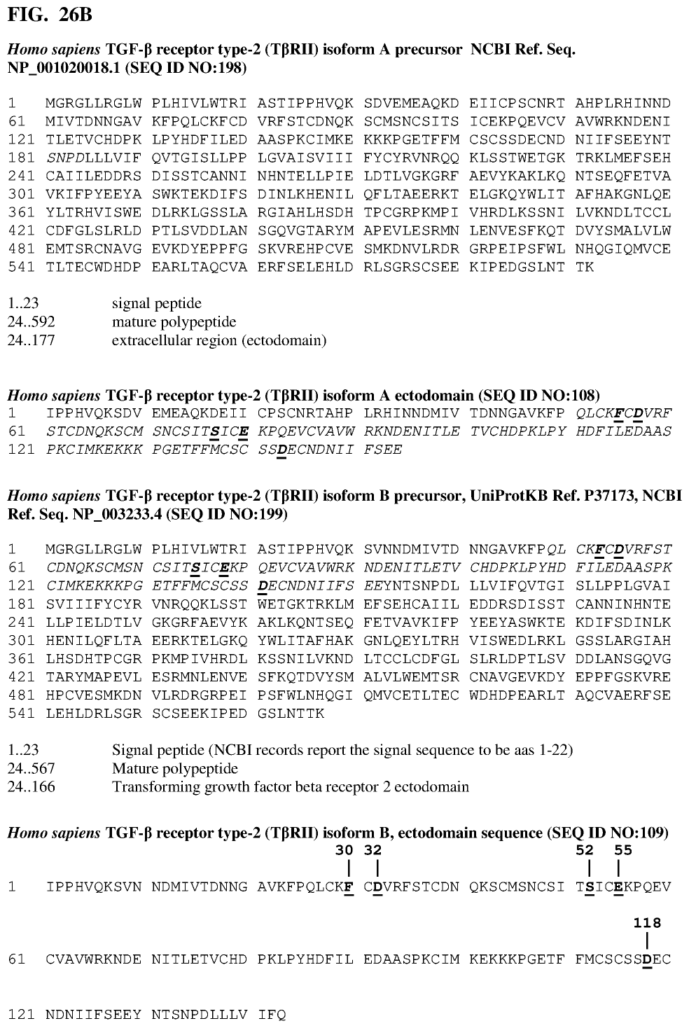

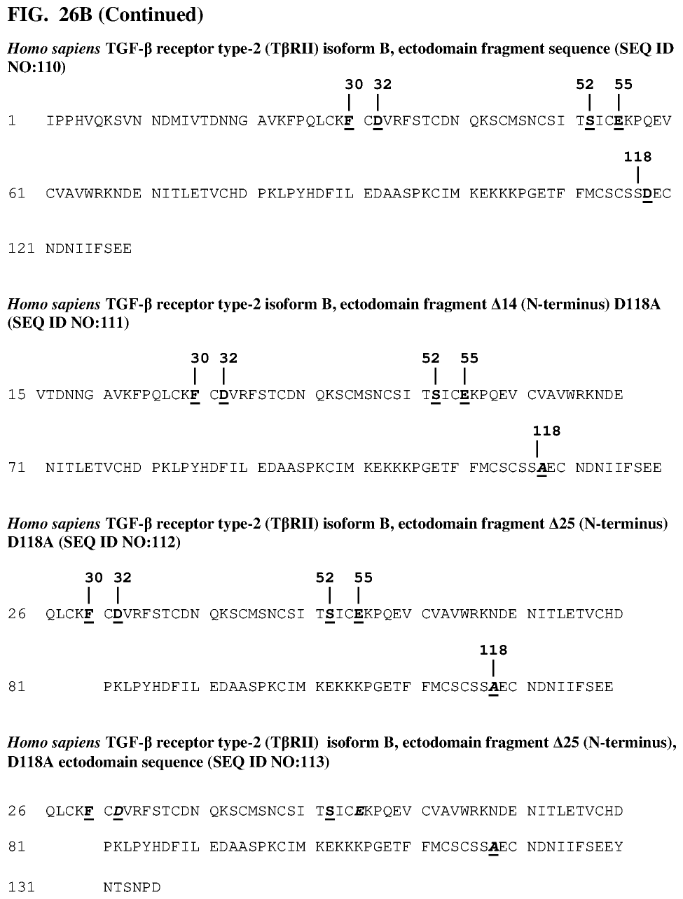

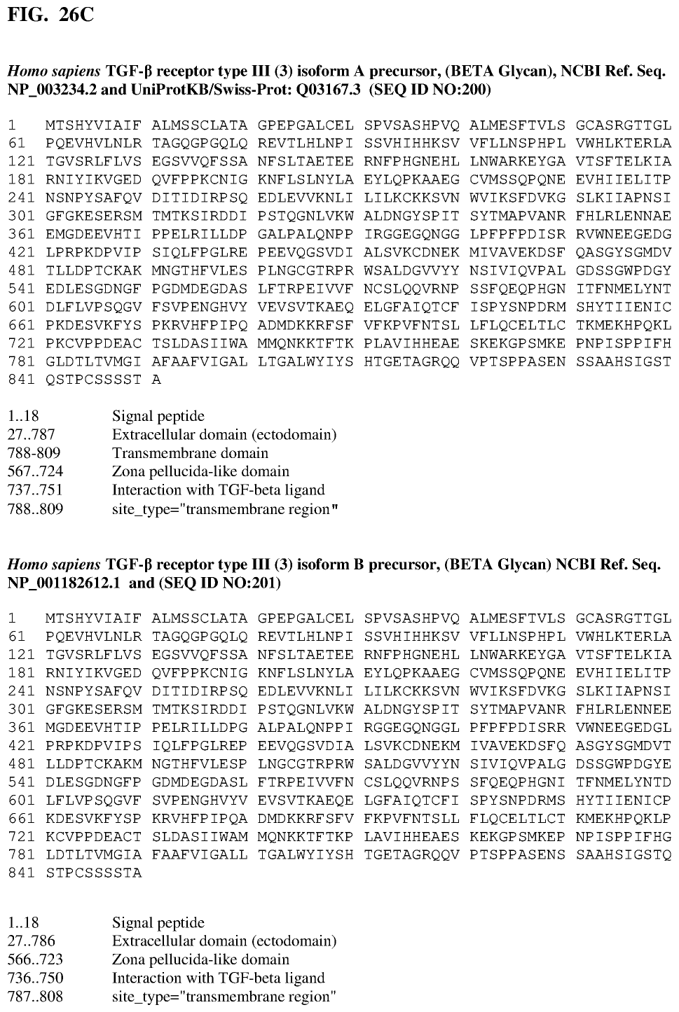

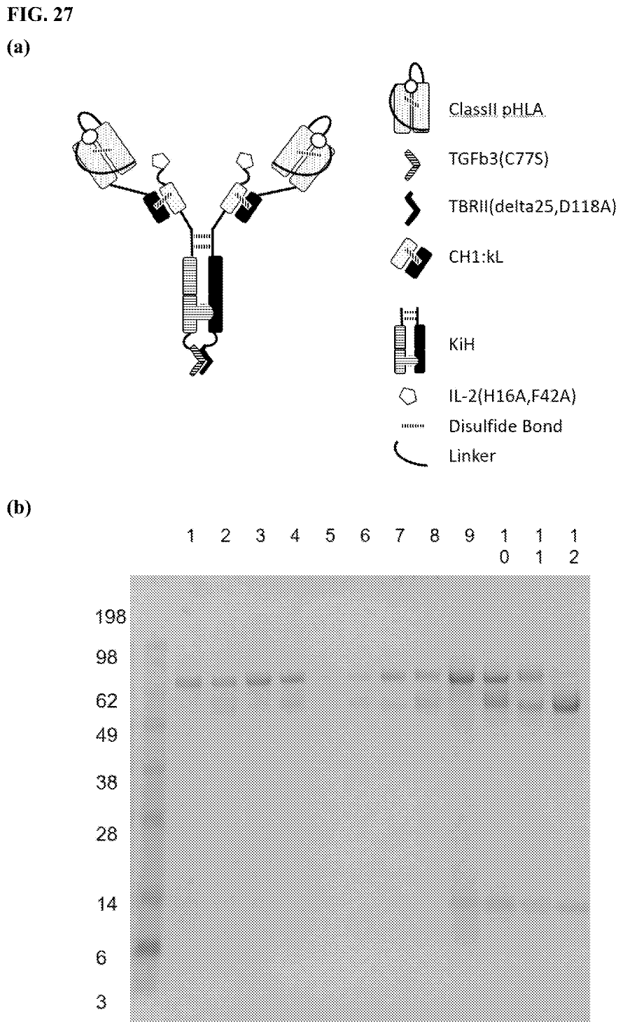

Conceptually, each of the presenting sequences and presenting complexes may be considered a “soluble MHC” that is fully capable of binding and presenting a peptide epitope. Unless stated otherwise, in presenting sequences all of the MHC α1, α2, β1, and β2 domain sequences, as well as the epitope polypeptide, are present in a single polypeptide chain (single linear sequence of aas produced by translation). See, e.g., FIGS. 25 and 26.

Where the MHC α1, α2, β1, and β2 domain sequences are divided among two or more polypeptide chains, the “soluble MHC” is termed a presenting complex. The presenting complex has one chain that is part of a framework peptide or dimerization peptide, referred to as a “presenting complex 1^{st} sequence.” The second chain of the presenting complex is termed the “presenting complex 2^{nd} sequence.” The presenting complex 2^{nd} sequence may be associated non-covalently with the MHC components present in the presenting complex 1^{st} sequence (through binding interactions between MHC-Class II α1, α2, β1, and β2 domain components as in FIGS. 27 to 29), in addition, one or more disulfide bonds between the presenting complex 1^{st} sequence and the presenting complex 2^{nd} sequence. Alternatively, the presenting complex 2^{nd} sequence may be associated non-covalently with the MHC components present in the presenting complex 1^{st} sequence through binding interactions between MHC-Class II α1, α2, β1, and β2 domain components and through binding sequences (e.g., such as interspecific binding sequences as in FIGS. 30, 31 structures A-E, and 32) in the presence or absence of one or more disulfide bonds between the presenting complex 1^{st} sequence and the presenting complex 2^{nd} sequence.

In some cases, one or more presenting sequence of a MAPP comprises all of the Class II components required for binding and presenting the epitope of interest to a TCR; e.g., the α1, α2, β1, and β2 domain and epitope in a single polypeptide sequence. In MAPPs comprising presenting complexes, the peptide epitope may be part of the presenting complex 1^{st} sequence or the presenting complex 2^{nd} sequence.

As noted above, presenting sequences and complexes typically will comprise a peptide epitope that is part of a MAPP polypeptide chain. It is possible, however, to make MAPPS that comprise the MHC components, but which do not comprise a peptide epitope that is part of a MAPP polypeptide chain. In such embodiments, the epitope, which is non-covalently loaded into the MHC pocket, may be a separate peptide (e.g., phosphopeptide, lipopeptide, glycosylated peptide, etc.) or non-peptide epitope, and may be subject to dissociation from the MAPPs.

### 4. MHC Class II Polypeptides

As noted above, the epitope containing MAPPs include MHC Class II polypeptides of various species, including human MHC polypeptides (HLA polypeptides), rodent (e.g., mouse, rat, etc.) MHC polypeptides, and MHC polypeptides' of other mammalian species (e.g., lagomorphs, non-human primates, canines, felines, ungulates (e.g., equines, bovines, ovines, caprines, etc.)), and the like.

For the purpose of this disclosure the term “MHC polypeptide” is meant to include Class II MHC polypeptides, including the α- and β-chains or portions thereof. More specifically, MHC Class II polypeptides include the α1 and α2 domains of Class II MHC α chains, and the β1 and 2 domains of Class II MHC β chains, which represent all or most of the extracellular Class II protein required for presentation of an epitope. In an embodiment, both the α and β Class II MHC polypeptide sequences in a MAPP are of human origin.

MAPPs and their higher order complexes (e.g., duplex MAPPs) are intended to be soluble in aqueous media under physiological conditions (e.g., soluble in human blood plasma at therapeutic levels). Unless expressly stated otherwise, as noted above, the MAPPs described herein are not intended to include membrane anchoring domains (such as transmembrane regions of MHC Class II α and β chains) or a part thereof sufficient to anchor MAPP molecules (e.g., more than 50% of the MAPP molecules), or a peptide thereof, in the membrane of a cell (e.g., a eukaryotic cell such as a mammalian cell such as a Chinese Hamster Ovary or “CHO” cell) in which the MAPP is expressed. Similarly, unless expressly stated otherwise, the MAPPs described herein do not include the leader and/or intracellular portions (e.g., cytoplasmic tails) that may be present in some naturally-occurring MHC Class II proteins.

a. T1D and its Risk-Associated Alleles and Haplotypes

Certain alleles and haplotypes of MHC Class II have been associated with disease, e.g., increased risk of developing T1D. See, e.g., Erlich et al. (2008) Diabetes 57:1084; Gough and Simmonds (2007) Curr. Genomics 8:453; Mitchell et al. (2007) Robbins Basic Pathology Philadelphia: Saunders, 8th ed.; Margaritte-Jeannin et al. (2004) Tissue Antigens 63:562; and Kurko et al. (2013) Clin. Rev. Allergy Immunol. 45:170.

### (i) MHC Class II Polypeptides in Type 1 Diabetes Mellitus (T1D)

Alleles/isoforms showing increased association with T1D represent suitable sources of MHC II α1, α2, β1, and β2 polypeptide sequences for incorporation into MAPPs directed to the treatment of T1D. T1D is associated with alleles belonging to the HLA-DR3 and HLA-DR4 haplotypes/serotypes, with the strongest risk associated with the HLA-DQ8, (e.g., HLA-DQB1*03:02) and alleles of the HLA-DQ2 serotype. Some high and moderate risk haplotypes and their association with various DR serotypes are shown in Table 2 adopted from Kantárová and Buc, Physiol. Res. 56: 255-266 (2007).

TABLE 2

| | | | | |
|---|---|---|---|---|
| DR | DRB | DQ | DQA | DQB |
| serotype | allele | serotype | allele | allele |

High risk TID haplotypes

| | | | | |
|---|---|---|---|---|
| DR3 | DRB1*0301 | DQ 2.5 | DQA1*0501 | DQB1*0201 |
| DR4 | DRB1*0401 | DQ 8.1 | DQA1*0301 | DQB1*0302 |
| DR4 | DRB1*0402 | DQ 8.1 | DQA1*0301 | DQB1*0302 |
| DR4 | DRB1*0405 | DQ 8.1 | DQA1*0301 | DQB1*0302 |

Moderate risk TID haplotypes

| | | | | |
|---|---|---|---|---|
| DR1 | DRB1*01 | DQ 5 | DQA1*0101 | DQB1*0501 |
| DR8 | DRB1*0801 | | DQA1*0401 | DQB1*0402 |
| DR9 | DRB1*0901 | | DQA1*0301 | DQB1*0303 |

The serotypically defined DR3 and DR4 protein isoforms/haplotypes of the DRB1 gene are associated with increased risk that an individual expressing such alleles will develop T1D. The DR3 serotype includes the alleles encoding the DRB1*03:01, *03:02, *03:03, and *03:04 proteins, with the HLA-DRB1*0301 allele often found associated with a predisposition to T1D. The DR4 serotype includes the alleles encoding the DRB1*04:01, *04:02, *04:03, *04:04, *04:05, *04:06, *04:07, *04:08, *04:09, *04:10, *04:11, *04:12, and *04:13 proteins. Certain HLA-DR4 (e.g., HLA-DRB1*0401 and HLA-DRB1*0405) predispose individuals to T1D, whereas HLA-DRB1*04:03 allele/isoform may afford protection. DRB1*16:01 also show an increased frequency in diabetic children relative to healthy controls (Deja, et al., Mediators of Inflammation 2006:1-7 (2006)). Alleles/isoforms showing increased association with T1D represent suitable sources of MHC II α1, α2, β1, and β2 polypeptide sequences.

The above-mentioned alleles associated with an increased risk of T1D represent suitable candidates from which the α1, α2, β1, and/or β2 polypeptide sequences present in a MAPP may be taken.

b. MHC Class II α and β Polypeptides

As discussed above, MAPPs comprise Class II MHC polypeptides. Naturally occurring Class II MHC polypeptides comprise an α chain and a β chain (e.g., HLA α- and β-chains). While MHC Class II polypeptides include MHC Class II DP α and β polypeptides. DM α and β polypeptides. DO α and β polypeptides. DQ α and β polypeptides, and DR α and β polypeptides. The polypeptides of central interest for the treatment of T1D are DQ α and β polypeptides and DR α and β polypeptides. As used herein, the term “Class II MHC polypeptide” refers to a Class II MHC α chain polypeptide, a Class II MHC β chain polypeptide, or only a portion of a Class II MHC α and/or β chain polypeptide, or combinations of the foregoing. For example, the term “Class II MHC polypeptide” as used herein can be a polypeptide that includes: i) only the α1 domain of a Class II MHC α chain; ii) only the α2 domain of a Class II MHC α chain; iii) only the α1 domain and an α2 domain of a Class II MHC α chain; iv) only the β1 domain of a Class II MHC β chain; v) only the β2 domain of a Class II MHC β chain; vi) only the β1 domain and the β2 domain of a Class II MHC β chain; vii) the α1 domain of a Class II MHC α chain, the β1 domain of a Class II MHC β chain, and the β2 domain of a Class II MHC; and the like.

The human MHC or HLA locus is highly polymorphic in nature, and thus as used herein, the term “Class II MHC polypeptide” includes allelic forms of any known Class II MHC polypeptide. See. e.g., the HLA Nomenclature site run by the Anthony Nolan Research Institute, available on the world wide web at hla.alleles.org/nomenclature/index.html, which indicates that there are numerous DRA alleles. DRB1 alleles, DRB3 alleles, DRB4 alleles, DRB5 alleles, DRB6 alleles, DRB7 alleles, DRB9 alleles, DQA1 alleles, DQB1 alleles, DPA1, DPB1 alleles, DMA alleles, DMB alleles, DOA alleles and DOB alleles.

In some cases, a MAPP comprises a Class II MHC α chain, without the leader, transmembrane, and intracellular portions (e.g., cytoplasmic tails) that may be present in a naturally-occurring Class II MHC α chain. Thus, in some cases, a MAPP comprises only the α1 and α2 portions of a Class II MHC α chain; and does not include the leader, transmembrane, and intracellular portions (e.g., cytoplasmic tails) that may be present in a naturally-occurring Class II MHC α chain.

In some cases, a MAPP comprises a Class II MHC β chain, without the leader, transmembrane, and intracellular portions (e.g., cytoplasmic tails) that may be present in a naturally-occurring Class II MHC β chain. Thus, in some cases, a MAPP comprises only the β1 and β2 portions of a Class II MHC β chain; and does not include the leader, transmembrane, and intracellular portions (e.g., cytoplasmic tails) that may be present in a naturally-occurring Class II MHC β chain.

### (i) MHC Class II Alpha Chains

MHC Class II alpha chains comprise an α1 domain and an α2 domain. In some cases, the α1 and α2 domains present in an antigen-presenting cell are from the same MHC Class II α chain polypeptide. In some cases, the α1 and α2 domains present in an antigen-presenting cell are from two different MHC Class II α chain polypeptides.

MHC Class II alpha chains suitable for inclusion in a presenting sequence or complex of a MAPP may lack a signal peptide. An MHC Class II alpha chain suitable for inclusion in a MAPP can have a length of from about 60 aas to about 200 aas; for example, an MHC Class II alpha chain suitable for inclusion in a MAPP can have a length of from about from about 60 amino acids to about 80 amino acids, 80 aas to about 100 aas, from about 100 aas to about 140 aas, from about 140 aas to about 170 aas, from about 170 aas to about 200 aas. An MHC Class II α1 domain suitable for inclusion in a MAPP can have a length of from about 30 aas to about 95 aas; for example, an MHC Class II α1 domain suitable for inclusion in a MAPP of the present disclosure can have a length of from about 30 aas to about 50 aas, from about 50 aas to about 70 aas, or from about 70 aas to about 95 aas. In an embodiment a MHC Class II α1 domain of a MAPP is from about 70 aas to about 95 aas. An MHC Class II α2 domain suitable for inclusion in a MAPP can have a length of from about 30 aas to about 95 aas; for example, an MHC Class II α2 domain suitable for inclusion in a MAPP can have a length of from about 30 aas to about 50 aas, from about 50 aas to about 70 aas, or from about 70 aas to about 95 aas. In an embodiment, an MHC Class II α2 domain of a MAPP is from about 70 aas to about 95 aas.

### (a) DRA Polypeptides

A suitable MHC Class II DRA polypeptide for inclusion in a MAPP may have at least 60%, at least 70%, at least 80%, at least 90%, at least 95%, at least 98%, at least 99%, or 100% aa sequence identity with at least 150, at least 160, or at least 170 contiguous amino acids of the aa sequence from aa 26 to aa 203 (the α1 and α2 domain region) of the DRA aa sequence depicted in FIG. 4 or a -naturally occurring allelic variant thereof. In some cases, the DRA polypeptide has a length of about 178 aas (e.g., 175, 176, 177, 178, 179, or 180 aas).

As used herein, the term “DRA polypeptide” includes allelic variants, e.g., naturally occurring allelic variants. Thus, in some cases, a suitable DRA polypeptide comprises aas 26-203 of DRA*01:02:01 (see FIG. 4), or an allelic variant thereof. In some cases, the allelic variant is the DRA*01:01 polypeptide (e.g., from the DRA*01:01:01:01 allele) that differs from DRA*01:02 by having a valine in place of the leucine at position 242 (see FIG. 4).

A suitable DRA for inclusion in a MAPP polypeptide can have at least 70%, at least 80%, at least 90%, at least 95%, at least 98%, at least 99%, or 100%, aa sequence identity with at least 160, at least 170, or at least 180 contiguous aas of the sequence from aa 26 to aa 216 of the DRA*01:02 sequence depicted in FIG. 4. A “DRA polypeptide” includes allelic variants, e.g., naturally occurring allelic variants.

Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNS PVELREPNVL ICFIDKFTPP VVNVTWLRNG KPVTTGVSET VFLPREDHLF RKFHYLPFLP STEDVYDCRV EHWGLDEPLL KHW (SEQ ID NO:80, amino acids 26-203 of DRA*01:02, see FIG. 4), or an allelic variant thereof. In some cases, the allelic variant is the DRA*01:01 allelic variant that differs from DRA*01:02 polypeptide by having a valine in place of the leucine at position 242 of the sequence in FIG. 4. In some cases, a DRA polypeptide suitable for inclusion in a MAPP comprises an amino acid substitution, relative to a wild-type DRA polypeptide, where the amino acid substitution replaces an amino acid (other than a Cys) with a Cys (e.g., for forming a disulfide bond that stabilizes the MAPP).

In some cases, a MAPP comprises a variant DRA polypeptide that comprises a non-naturally occurring Cys residue (e.g., for forming a disulfide bond that stabilizes the MAPP). For example, in some cases, a MAPP comprises a variant DRA polypeptide that comprises at least one aa substitution selected from E3C, E4C, F12C, G28C, D29C, I72C, K75C, T80C, P81C, 182C, T93C, N94C, and S95C (see, e.g., FIG. 4 SEQ ID NO: 17).

A suitable DRA α1 domain for inclusion in a MAPP polypeptide, including naturally occurring allelic variants thereof, may comprise an aa sequence having at least 70%, at least 80%, at least 90%, at least 95%, at least 98%, at least 99%, or 100% aa sequence identity to the following aa sequence: VIIQAEFYLN PDQSGEFMFD FDGDEIFHVD MAKKETVWRL EEFGRFASFE AQGALANIAV DKANLEIMTK RSNYTPITN (SEQ ID NO:81); and can have a length of about 84 aas (e.g., 80, 81, 82, 83, 84, 85, or 86 aas).

A suitable DRA α2 domain for inclusion in a MAPP polypeptide, including naturally occurring allelic variants thereof, may comprise an aa sequence having at least 70%, at least 80%, at least 90%, at least 95%, at least 98%, at least 99%, or 100% aa sequence identity to the following aa sequence: V PPEVTVLTNSPVELREPNVL ICFIDKFTPP VVNVTWLRNG KPVTTGVSET VFLPREDHLF RKFHYLPFLP STEDVYDCRV EHWGLDEPLL KHW (SEQ ID NO:82); and can have a length of about 94 aas (e.g., 90, 91, 92, 93, 94, 95, 96, 97, or 98 aas).

### (b) DQA Polypeptides

A suitable MHC Class II a DQA polypeptide for inclusion in a MAPP may have at least 60%, at least 70%, at least 80%, at least 90%, at least 95%, at least 98%, at least 99%, or 100% aa sequence identity with the α1, α2, β1, and/or the β2 domain(s) of DQA1*0101, DQA1*0301, or DQA1*0401. In some cases, the DQA polypeptide has a length of about 178 aas (e.g., 175, 176, 177, 178, 179, or 180 aas). The sequences of those HLA polypeptides are available, for example on the world wide web at hla.alleles.org/nomenclature/index.html, which is run by the Anthony Nolan Research Institute, and at www.ncbi.nlm.nih.gov (National Center for Biotechnical Information or “NCBI”) operated by the U.S. National Library of Medicine.

### (ii) MHC Class II Beta Chains

MHC Class II beta chains comprise a β1 domain and a β2 domain. In some cases, the β1 and β2 domains present in an antigen-presenting cell are from the same MHC Class II β chain polypeptide. In some cases, the β1 and β2 domains present in an antigen-presenting cell are from two different MHC Class II β chain polypeptides.

MHC Class II beta chains suitable for inclusion in a MAPP (e.g., a higher order MAPP construct such as a duplex MAPP) lack a signal peptide. An MHC Class II beta chain suitable for inclusion in a MAPP can have a length of from about 60 aas to about 210 aas; for example, an MHC Class II beta chain suitable for inclusion in a MAPP can have a length of from about 60 aas to about 90 aas, from about 90 aas to about 120 aas, from about 120 aas to about 150 aas, from about 150 aas to about 180 aas, from about 180 aas to 210 aas. An MHC Class II β1 domain suitable for inclusion in a MAPP can have a length of from about 30 aas to about 105 aas; for example, an MHC Class II β1 domain suitable for inclusion in a MAPP can have a length of from about 30 aas to about 50 aas, from about 50 aas to about 70 aas, from about 70 aas to about 90 aas, from about 90 aas to about 105 aas. An MHC Class II β2 domain suitable for inclusion in a MAPP can have a length of from about 30 aas to about 105 aas; for example, an MHC Class II β2 domain suitable for inclusion in a MAPP can have a length of from about 30 aas to about 50 aas, from about 50 aas to about 70 aas, from about 70 aas to about 90 aas, from about 90 aas to about 105 aas.

An MHC Class II β chain polypeptide suitable for inclusion in a MAPP may comprise an aa substitution, relative to a wild-type MHC Class II β chain polypeptide, where the aa substitution replaces an aa (other than a Cys) with a Cys (e.g., for forming a disulfide bond that stabilizes the MAPP). For example, in some cases, the MHC Class II β chain polypeptide is a variant DRB1 MHC Class II polypeptide that comprises an aa substitution selected from the group consisting of P5C, F7C, Q10C, N19C, G20C, H33C, G151C, D152C, and W153C. In some cases, the MHC Class II β chain polypeptide is a variant DRB1 polypeptide comprising an aa sequence having at least 60%, at least 70%, at least 80%, at least 90%, at least 95%, at least 98%, or at least 99%, aa sequence identity to the following mature DRB1 aa sequence lacking the signal peptide: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPA SIEVRWFRNGQEEKTGVVSTGLIQNGDWTFQTLVMLETVPRSGEVYTCQVEHPSLTSPLTVEWR ARSESAQSKM (SEQ ID NO:83), and comprising an cysteine substitution at one or more (e.g., two or more) aas selected from the group consisting of P5C, F7C, Q10C, N19C, G20C, H33C, G151C, D152C, and W153C. In some cases, the MHC Class II β chain polypeptide is a variant of a mature DRB3 polypeptide, mature DRB4 polypeptide, or mature DRB5 polypeptide (lacking their signal sequences) comprising a cysteine substitution at one or more (e.g., two or more) of positions 5, 7, 10, 19, 20, 33, 151, 152, and 153 (e.g., P5C, F7C, Q10C, N19C, G20C, N33C, G151C, D152C, and/or W153C substitutions).

### (a) DRB Polypeptides

### (1) DRB1 Polypeptides

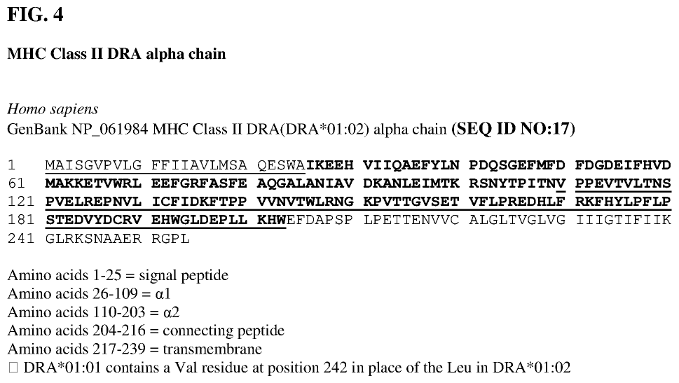

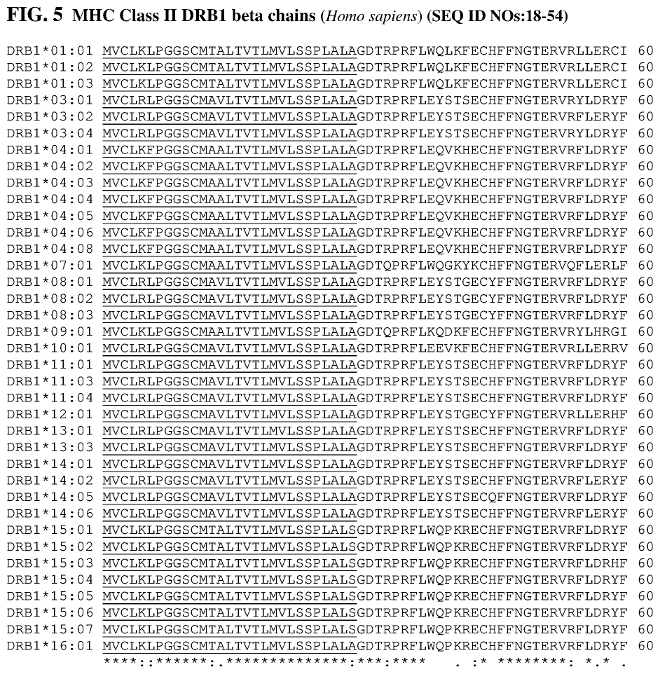

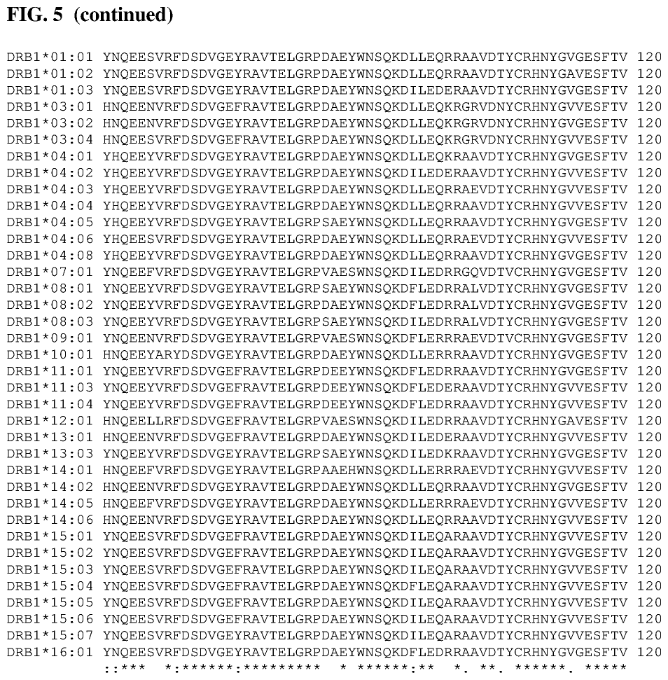

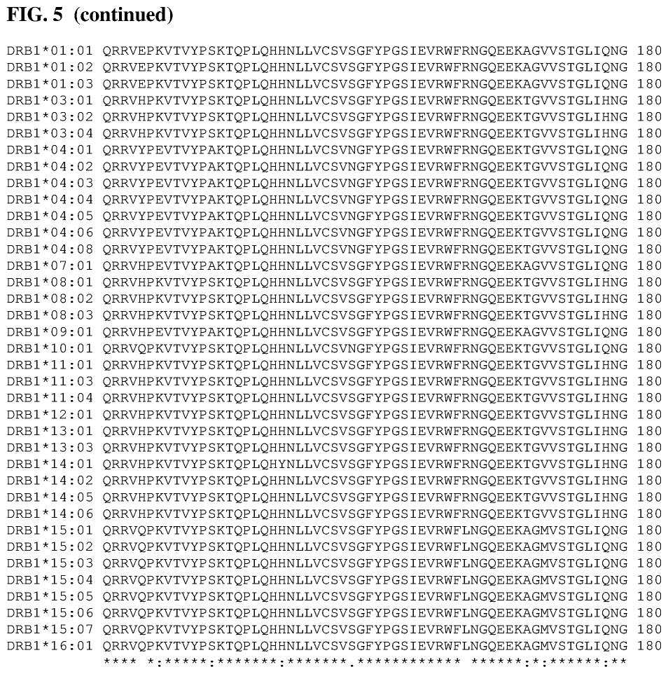

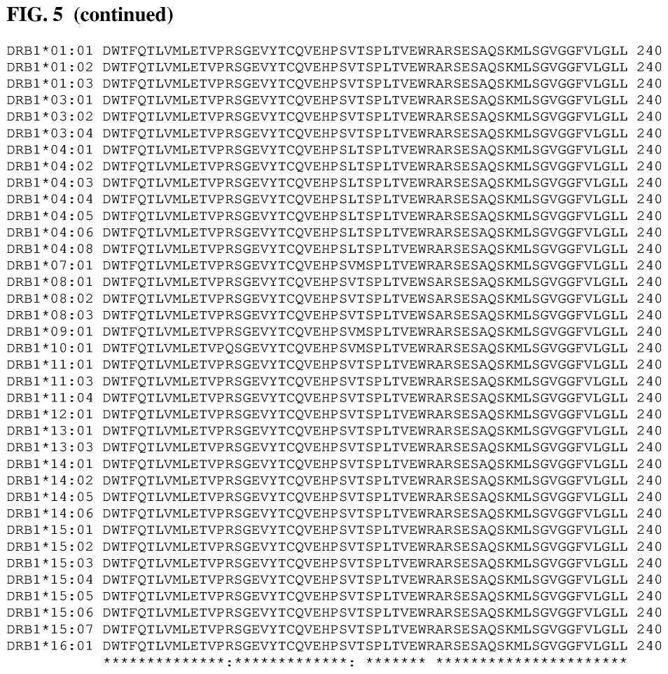

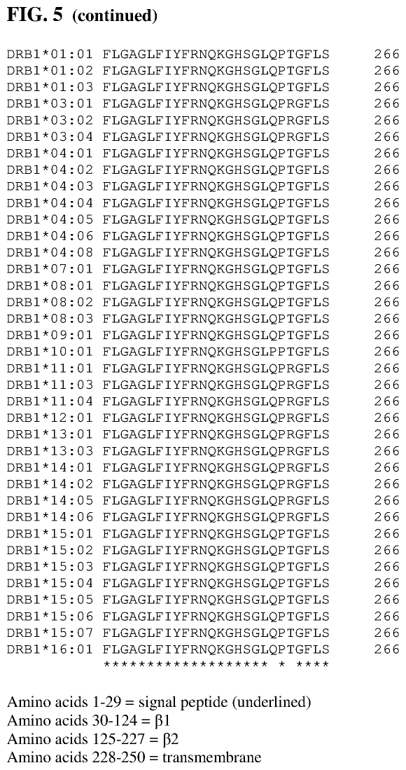

In some cases, a suitable MHC Class II β chain polypeptide for inclusion in a MAPP is a DRB1 polypeptide. In an embodiment, a DRB1 polypeptide can have at least 60%, at least 70%, at least 80%, at least 90%, at least 95%, at least 98%, at least 99%, or 100%, aa sequence identity with at least 170, at least 180, or at least 190, contiguous aas of the sequence from aa 30 to aa 227 of any DRB1 aa sequence depicted in FIG. 5, including naturally occurring allelic variants. FIG. 5 displays the DRB1 precursor proteins in which aas 1-29 are the signal sequence (underlined), 30-124 the β1 region (bolded), 125-227 the β2 region (bolded and underlined), and 228-250 the transmembrane region. In some cases, a DRB1 polypeptide suitable for inclusion in a MAPP comprises an aa substitution, relative to a wild-type DRB1 polypeptide, where the aa substitution replaces an aa (other than a Cys) with a Cys (e.g., for forming a disulfide bond that stabilizes the MAPP).

A suitable MHC Class II β chain polypeptide suitable for incorporation into a MAPP may be a DRB1 polypeptide, wherein the DRB1 polypeptide has at least 60%, at least 70%, at least 80%, at least 90%, at least 95%, at least 98%, at least 99%, or 100% aa sequence identity with at least 170, at least 180, or at least 190, contiguous aas of the sequence from aa 30 to aa 227 (the β1 and β2 domain region) of a DRB1 sequence provided in FIG. 5, including one of the following DRB1 polypeptides:
(i) the DRB1-1 (DRB1*01:01) beta chain aa sequence Swiss-Prot/UniProt reference (“sp”) P04229.2 in FIG. 5;
(ii) the DRB1-3 (DRB1*03:01) beta chain aa sequence sp P01912.2 in FIG. 5;
(iii) the DRB1-4 (DRB1*04:01) beta chain aa sequence sp P13760.1 in FIG. 5;
(iv) the DRB1-7 (DRB1*07:01) beta chain aa sequence sp P13761.1 in FIG. 5;
(v) the DRB1-8 (DRB1*08:01) beta chain aa sequence sp Q30134.2 in FIG. 5;
(vi) the DRB1-9 (DRB1*09:01) beta chain aa sequence sp Q9TQE0.1 in FIG. 5;
(vii) the DRB1-10 (DRB1*10:01) beta chain aa sequence sp Q30167.2 in FIG. 5;
(viii) the DRB1-11 (DRB1*11:01) beta chain aa sequence sp P20039.1 in FIG. 5;
(ix) the DRB1-12 (DRB1*12:01) beta chain aa sequence sp Q95IE3.1 in FIG. 5;
(x) the DRB1-13 (DRB1*13:01) beta chain aa sequence sp Q5Y7A7.1 in FIG. 5;
(xi) the DRB1-14 (DRB1*14:01) beta chain aa sequence sp Q9GIY3.1 in FIG. 5;
(xii) the DRB1-15 (DRB1*15:01) beta chain aa sequence sp P01911 in FIG. 5; and
(xiii) the DRB1-16 (DRB1*16:01) beta chain aa sequence sp Q29974.1 in FIG. 5.
   In some cases, the DRB1 β chain polypeptide has a length of about 198 aas, including, e.g., 195, 196, 197, 198, 199, 200, 201, or 202 aas.

A suitable “DRB1 polypeptide” for incorporation into a MAPP includes allelic variants, e.g., naturally occurring allelic variants. Thus, in some cases, a suitable DRB1 polypeptide comprises aas 31-227 of DRB1*04:01 (DRB1-4) provided in FIG. 5 (SEQ ID NO:24) or an allelic variant thereof.

Another suitable DRB1 polypeptide may comprise a sequence having at least 70%, at least 80%, at least 90%, at least 95%, at least 98%, at least 99%, or 100% aa sequence identity to at least 170, at least 180, or at least 190 contiguous aas of the following DRB1*04:01 aa sequence: GDTRPRFLEQVKHECHFFNGTERVRFLDRYFYHQEEYVRFDSDVGEYRAVTELGRPDAEYWNS QKDLLEQKRAAVDTYCRHNYGVGESFTVQRRVYPEVTVYPAKTQPLQHHNLLVCSVNGFYPAS IEVRWFRNGQEEKTGVVSTGLIQNGDWTFQTLVMLETVPRSGEVYTCQVEHPSLTSPLTVEWRA RSESAQSKM (SEQ ID NO:84), which may bear one or more cysteine substitutions. In an embodiment the cysteine substitution is a P5C substitution. In an embodiment the cysteine substitution is a G151C substitution. In an embodiment the cysteine substitution is a W153C substitution.

A suitable DRB1 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: DTRPRFLEQVKHECHFFNGTERVRFLDRYFYHQEEYVRFDSDVGEYRAVTELGRPDAEYWNSQ KDLLEQKRAAVDTYCRHNYGVGESFTVQRRV (SEQ ID NO:85); and can have a length of about 95 aas, including, e.g., 92, 93, 94, 95, 96, 97, or 98 aas.

A suitable DRB1 β1 domain can comprise the following amino acid sequence: GDTRCRFLEQVKHECHFFNGTERVRFLDRYFYHQEEYVRFDSDVGEYRAVTELGRPDAEYWNS QKDLLEQKRAAVDTYCRHNYGVGESFTVQRRV (SEQ ID NO:86), where P5 is substituted with a Cys (shown in bold and italics text).

A suitable DRB1 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: YPEVTVYPAKTQPLQHHNLLVCSVNGFYPGSIEVRWFRNGQEEKTGVVSTGLIQNGDWTFQTLV MLETVPRSGEVYTCQVEHPSLTSPLTVEWRARSESAQSK (SEQ ID NO:87); and can have a length of about 103 aas, including, e.g., 100, 101, 102, 103, 104, 105, or 106 aas.

A suitable DRB1 β2 domain can comprise the following amino acid sequence: YPEVTVYPAKTQPLQHHNLLVCSVNGFYPASIEVRWFRNGQEEKTGVVSTGLIQNGDCTFQTLV MLETVPRSGEVYTCQVEHPSLTSPLTVEWRARSESAQSKM (SEQ ID NO:88), where W153 is substituted with a Cys (shown in bold and italics text).

### (2) DRB3 Polypeptides

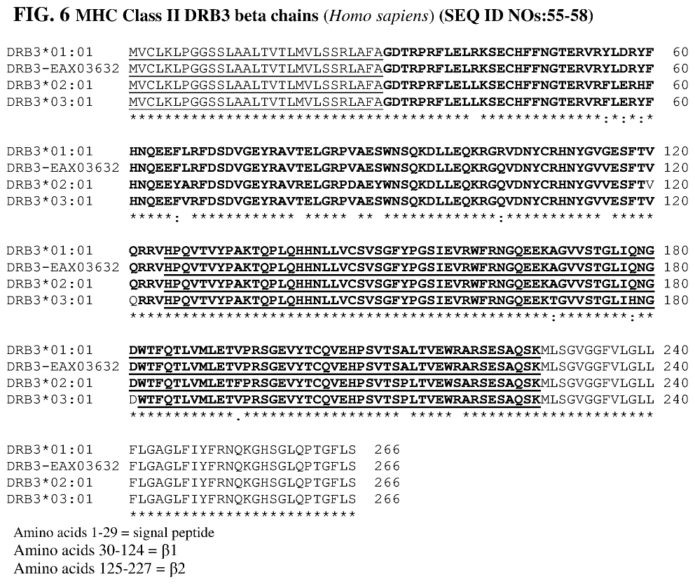

In some cases, a suitable MHC Class II β chain polypeptide for inclusion in a MAPP is a DRB3 polypeptide. In an embodiment, a DRB3 polypeptide can have at least 60%, at least 70%, at least 80%, at least 90%, at least 95%, at least 98%, at least 99%, or 100% aa sequence identity with aas 30-227 of any DRB3 aa sequence depicted in FIG. 6, which displays the DRB3 precursor proteins in which aas 1-29 are the signal sequence (underlined), 30-124 form the β1 region (shown bolded), 125-227 form the β2 region, and 228-250, the transmembrane region. A DRB3 β chain polypeptide suitable for incorporation into a MAPP may have at least 60%, at least 70%, at least 80%, at least 90%, at least 95%, at least 98%, at least 99%, or 100% aa sequence identity with aas 30-227 (the β1 and β2 domain region) of one of the following DRB3 polypeptides:
(i) the DRB1-3 (DRB3*01:01) beta chain aa sequence GenBank NP_072049.1 in FIG. 6;
(ii) the DRB1-3 beta chain aa sequence in GenBank accession EAX03632.1 in FIG. 6;
(iii) the DRB1-3 (DRB3*02:01) beta chain aa sequence GenBank CAA23781.1 in FIG. 6; and
(iv) the DRB1-3 (DRB3*03:01) beta chain aa sequence GenBank AAN15205.1 in FIG. 6.
   A DRB3 polypeptide suitable for inclusion in a MAPP may comprise an aa substitution, relative to a wild-type DRB3 polypeptide, where the aa substitution replaces an aa (other than a Cys) with a Cys (e.g., for forming a disulfide bond that stabilizes the MAPP).

As used herein, the term “DRB3 polypeptide” includes allelic variants, e.g., naturally occurring allelic variants. Thus, in some cases, a suitable DRB3 polypeptide comprises aas 30 to 227 of DRB3*01:01 provided in FIG. 6 (SEQ ID NO:55), or an allelic variant thereof. Thus, in some cases, a suitable DRB3 polypeptide comprises a sequence having at least 80%, at least 90%, at least 95%, at least 98%, at least 99%, or 100% sequence identity to at least 170, at least 180, or at least 190 contiguous aas of the following sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:89), or an allelic variant thereof. In some cases, a DRB3 polypeptide suitable for inclusion in a MAPP comprises an aa substitution, relative to a wild-type DRB3 polypeptide, where the aa substitution replaces an aa (other than a Cys) with a Cys. Thus, e.g., in some cases, the MHC Class II β chain polypeptide is a variant DRB3 MHC Class II polypeptide that comprises a non-naturally occurring Cys at an aa selected from the group consisting of P5C, F7C, L10C, N19C, G20C, N33C, G151C, D152C, and W153C (of a mature DRB3 polypeptide (lacking the N-terminal signal peptide MVCLKLPGGSSLAALTVTLMVLSSRLAFA (SEQ ID NO:90) depicted in FIG. 6).

A suitable DRB3 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: DTRPRFLELR KSECHFENGT ERVRYLDRYF HNQEEFLRFD SDVGEYRAVT ELGRPVAESW NSQKDLLEQK RGRVDNYCRH NYGVGESFTV QRRV (SEQ ID NO:91); and can have a length of about 95 aas (e.g., 93, 94, 95, 96, 97, or 98 aas). A suitable DRB3 β1 domain can comprise the following aa sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:91), or a naturally-occurring allelic variant A suitable DRB3 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:92); and can have a length of about 103 aas (e.g., 100, 101, 102, 103, 104, or 105 aas). A suitable DRB3 β2 domain can comprise the following aa sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:92), or a naturally-occurring allelic variant thereof.

### (3) DRB4 Polypeptides

In some cases, a suitable MHC Class II β chain polypeptide for inclusion in a MAPP is a DRB4 polypeptide. A DRB4 polypeptide can have at least 60%, at least 70%, at least 80%, at least 90%, at least 95%, at least 98%, at least 99%, or 100% aa sequence identity with aas 30-227 (the β1 and β2 domain region) of a DRB4 aa sequence depicted in FIG. 7. In some cases, the DRB4 polypeptide has a length of about 198 aas, including, e.g., 195, 196, 197, 198, 199, 200, 201, or 202 aas. In some cases, a DRB4 polypeptide suitable for inclusion in a MAPP comprises an amino acid substitution, relative to a wild-type DRB4 polypeptide, where the amino acid substitution replaces an amino acid (other than a Cys) with a Cys.

As used herein the term “DRB4 polypeptide” includes allelic variants, e.g., naturally occurring allelic variants. Thus, in some cases, a suitable DRB4 polypeptide comprises aas 30 to 227 of DRB4*01:03 (SEQ ID NO:60) provided in FIG. 7, or an allelic variant thereof. In some cases, a DRB4 polypeptide suitable for inclusion in a MAPP comprises an amino acid substitution, relative to a wild-type DRB4 polypeptide, where the amino acid substitution replaces an amino acid (other than a Cys) with a Cys. Thus, e.g., in some cases, the MHC Class II β chain polypeptide is a variant DRB4 MHC Class II polypeptide that comprises a non-naturally occurring Cys residue; e.g., where the variant DRB4 MHC class II polypeptide comprises an amino acid substitution selected from the group consisting of P15C. F17C, Q20C, N29C, G30C, N43C, G161C, D162C, and W163C of a mature DRB4 polypeptide (lacking the N-terminal signal peptide MVCLKLPGGSCMAALTVTL (SEQ ID NO:93) depicted in FIG. 7.

A suitable DRB4 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:94); and can have a length of about 95 aas (e.g., 93, 94, 95, 96, 97, or 98 aas).

A suitable DRB4 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:95); and can have a length of about 103 aas (e.g., 100, 101, 102, 103, 104, or 105 aas).

### (4) DRB5 Polypeptides

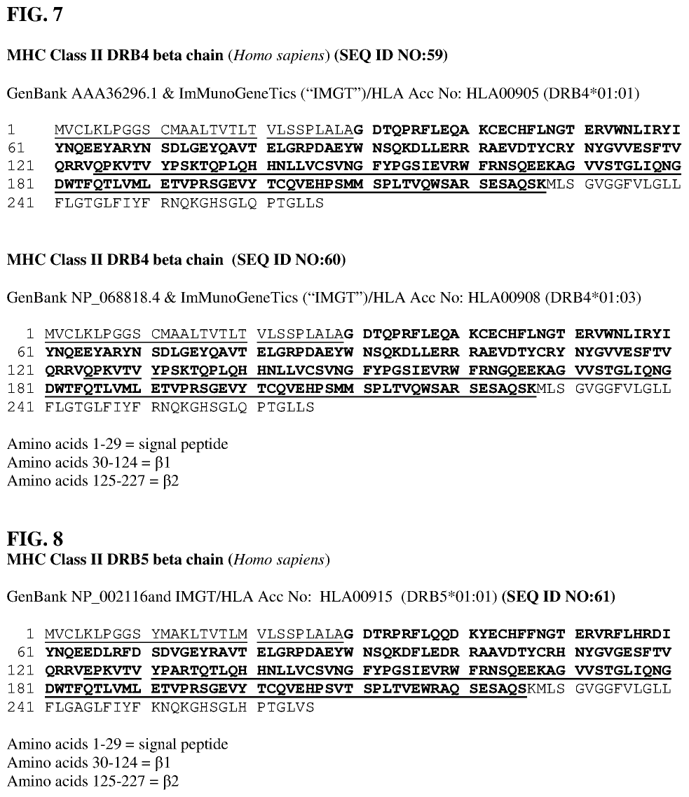

In some cases, a suitable MHC Class II β chain polypeptide for inclusion in a MAPP is a DRB5 polypeptide. A DRB5 polypeptide can have at least 60%, at least 70%, at least 80%, at least 90%, at least 95%, at least 98%, at least 99%, or 100% aa sequence identity with aas 30-227 (the β1 and β2 domain region) of the DRB5 aa sequence depicted in FIG. 8. In some cases, the DRB5 polypeptide has a length of about 198 aas (including, e.g., 195, 196, 197, 198, 199, 200, 201, or 202 aas). In some cases, a DRB5 polypeptide suitable for inclusion in a MAPP comprises an aa substitution, relative to a wild-type DRB5 polypeptide, where the aa substitution replaces an aa (other than a Cys) with a Cys (e.g., for forming a disulfide bond that stabilizes the MAPP).

As used herein, the term “DRB5 polypeptide” includes allelic variants, e.g., naturally occurring allelic variants. Thus, in some cases, a suitable DRB4 polypeptide comprises aas 30 to 227 of DRB5*01:01 (SEQ ID NO:61) provided in FIG. 8, or an allelic variant thereof.

A suitable DRB5 β1 domain, including-naturally occurring allelic variants thereof, may comprise an aa sequence having at least 70%, at least 80%, at least 90%, at least 95%, at least 98%, at least 99%, or 100% aa sequence identity to the following aa sequence: M VLSSPLALAG DTRPRFLQQD KYECHFENGT ERVRFLHRDI YNQEEDLRFD SDVGEYRAVT ELGRPDAEYW NSQKDFLEDR RAAVDTYCRH NYGVGESFTV QRRV (SEQ ID NO:96); and can have a length of about 95 aas (e.g., 93, 94, 95, 96, 97, or 98 aas).

A suitable DRB5 β2 domain, including-naturally occurring allelic variants thereof, may comprise an aa sequence having at least 70%, at least 80%, at least 90%, at least 95%, at least 98%, at least 99%, or 100% aa sequence identity to the following aa sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:97); and can have a length of about 103 aas (e.g., 100, 101, 102, 103, 104, or 105 aas).

### (b) DQB Polypeptides

A suitable MHC Class II a DQB polypeptide for inclusion in a MAPP may have at least 60%, at least 70%, at least 80%, at least 90%, at least 95%, at least 98%, at least 99%, or 100% aa sequence identity with the α1, α2, β1, and/or the β2 domain(s) of DQB1*0201, DQB1*0302, DQB1*0303, DQB1*0402, or DQB1*0501. In some cases, the DQA polypeptide has a length of about 178 aas (e.g., 175, 176, 177, 178, 179, or 180 aas). The sequences of those HLA polypeptides are available, for example on the world wide web at hla.alleles.org/nomenclature/index.html, which is run by the Anthony Nolan Research Institute, and at www.ncbi.nlm.nih.gov (National Center for Biotechnical Information or “NCBI”) operated by the U.S. National Library of Medicine.

c. Individual Disease Risk-Associated Alleles

### (i) DRB1

### (a) DRB1*01:01

A MAPP may comprise a DRB1*01:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*01:01 aa sequence provided in FIG. 5. In some cases, a MAPP comprises a DRB1*01:01 polypeptide comprising an aa sequence having at least 70%, at least 80%, at least 90%, at least 95%, at least 98%, at least 99%, or 100%, aa sequence identity to aas 30-124 of the DRB1*01:01 aa sequence provided in FIG. 5. In some cases, a MAPP comprises a DRB1*01:01 polypeptide comprising an aa sequence having at least 70%, at least 80%, at least 90%, at least 95%, at least 98%, at least 99%, or 100%, aa sequence identity to aas 125-227 of the DRB1*01:01 aa sequence depicted in FIG. 5.

### (b) DRB1*01:02

A MAPP may comprise a DRB1*01:02 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*01:02 aa sequence provided in FIG. 5. In some cases, a MAPP comprises a DRB1*01:02 polypeptide comprising an aa sequence having at least 70%, at least 80%, at least 90%, at least 95%, at least 98%, at least 99%, or 100%, aa sequence identity to aas 30-124 of the DRB1*01:02 aa sequence provided in FIG. 5. In some cases, a MAPP comprises a DRB1*01:02 polypeptide comprising an aa sequence having at least 70%, at least 80%, at least 90%, at least 95%, at least 98%, at least 99%, or 100%, aa sequence identity to aas 125-227 of the DRB1*01:02 aa sequence depicted in FIG. 5.

### (c) DRB1*01:03

A MAPP may comprise a DRB1*01:03 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*01:03 aa sequence provided in FIG. 5. In some cases, a MAPP comprises a DRB1*01:03 polypeptide comprising an aa sequence having at least 70%, at least 80%, at least 90%, at least 95%, at least 98%, at least 99%, or 100%, aa sequence identity to aas 30-124 of the DRB1*01:03 aa sequence provided in FIG. 5. In some cases, a MAPP comprises a DRB1*01:03 polypeptide comprising an aa sequence having at least 70%, at least 80%, at least 90%, at least 95%, at least 98%, at least 99%, or 100%, aa sequence identity to aas 125-227 of the DRB1*01:03 aa depicted in FIG. 5.

### (d) DRB1*03:01

DRB1*0301 (“DRB1*03:01” in FIG. 5) is associated with increased risk of developing T1D. Thus, a MAPP may comprise a DRB1*03:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*03:01 aa sequence provided in FIG. 5. In some cases, a MAPP comprises a DRB1*03:01 polypeptide comprising an aa sequence having at least 70%, at least 80%, at least 90%, at least 95%, at least 98%, at least 99%, or 100%, aa sequence identity to aas 30-124 of the DRB1*03:01 aa sequence depicted in FIG. 5. In some cases, a MAPP comprises a DRB1*03:01 polypeptide comprising an aa sequence having at least 70%, at least 80%, at least 90%, at least 95%, at least 98%, at least 99%, or 100%, aa sequence identity to aas 125-227 of the DRB1*03:01 aa sequence depicted in FIG. 5.

### (e) DRB1*03:02

A MAPP may comprise a DRB1*03:02 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*03:02 aa sequence provided in FIG. 5. In some cases, a MAPP comprises a DRB1*03:02 polypeptide comprising an aa sequence having at least 70%, at least 80%, at least 90%, at least 95%, at least 98%, at least 99%, or 100%, aa sequence identity to aa 30-124 of the DRB1*03:02 aa sequence provided in FIG. 5. In some cases, a MAPP comprises a DRB1*03:02 polypeptide comprising an aa sequence having at least 70%, at least 80%, at least 90%, at least 95%, at least 98%, at least 99%, or 100%, aa sequence identity to aas 125-227 of the DRB1*03:02 aa sequence depicted in FIG. 5.

### (f) DRB1*03:04:

A MAPP may comprise a DRB1*03:04 polypeptide comprising an aa sequence having at least 70%, at least 80% at least 90%, at least 95%, at least 98%, at least 99%, or 100%, aa sequence identity to at least 170, at least 180, or at least 190, contiguous aas of the sequence from aa 30 to aa 227 of the DRB1*03:04 aa sequence provided in FIG. 5. In some cases, a MAPP comprises a DRB1*03:04 polypeptide comprising an aa sequence having at least 70%, at least 80%, at least 90%, at least 95%, at least 98%, at least 99%, or 100%, aa sequence identity to aas 30-124 of the DRB1*03:04 aa sequence provided in FIG. 5. In some cases, a MAPP comprises a DRB1*03:04 polypeptide comprising an aa sequence having at least 70%, at least 80%, at least 90%, at least 95%, at least 98%, at least 99%, or 100%, aa sequence identity to aas 125-227 of the DRB1*03:04 aa sequence depicted in FIG. 5.

### (g) DRB1*04:01

A MAPP may comprise a DRB1*04:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*04:01 aa sequence depicted in FIG. 5. In some cases, a MAPP comprises a DRB1*04:01 polypeptide comprising an aa sequence having at least 70%, at least 80%, at least 90%, at least 95%, at least 98%, at least 99%, or 100%, aa sequence identity to aas 30-124 of the DRB1*04:01 aa sequence depicted in FIG. 5. In some cases, a MAPP comprises a DRB1*04:01 polypeptide comprising an aa sequence having at least 70%, at least 80%, at least 90%, at least 95%, at least 98%, at least 99%, or 100%, aa sequence identity to aas 125-227 of the DRB1*04:01 aa sequence depicted in FIG. 5.

### (h) DRB1*04:02

A MAPP may comprise a DRB1*04:02 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*04:02 aa sequence provided in FIG. 5. In some cases, a MAPP comprises a DRB1*04:02 polypeptide comprising an aa sequence having at least 70%, at least 80%, at least 90%, at least 95%, at least 98%, at least 99%, or 100%, aa sequence identity to aas 30-124 of the DRB1*04:02 aa sequence provided in FIG. 5. In some cases, a MAPP comprises a DRB1*04:02 polypeptide comprising an aa sequence having at least 70%, at least 80%, at least 90%, at least 95%, at least 98%, at least 99%, or 100%, aa sequence identity to aas 125-227 of the DRB1*04:02 aa sequence provided in FIG. 5.

### (i) DRB1*08:01

A MAPP may comprise a DRB1*08:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*08:01 aa sequence provided in FIG. 5 In some cases, a MAPP comprises a DRB1*08:01 polypeptide comprising an aa sequence having at least 70%, at least 80%, at least 90%, at least 95%, at least 98%, at least 99%, or 100%, aa sequence identity to aas 30-124 of the DRB1*08:01 aa sequence provided in FIG. 5. In some cases, a MAPP comprises a DRB1*08:01 polypeptide comprising an aa sequence having at least 70%, at least 80%, at least 90%, at least 95%, at least 98%, at least 99%, or 100%, aa sequence identity to aas 125-227 of the DRB1*08:01 aa sequence depicted in FIG. 5.

### (j) DRB1*09:01

A MAPP may comprise a DRB1*09:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB1*09:01 aa sequence provided in FIG. 5. In some cases, a MAPP comprises a DRB1*09:01 polypeptide comprising an aa sequence having at least 70%, at least 80%, at least 90%, at least 95%, at least 98%, at least 99%, or 100%, aa sequence identity to aas 30-124 of the DRB1*09:01 aa sequence provided in FIG. 5. In some cases, a MAPP comprises a DRB1*09:01 polypeptide comprising an aa sequence having at least 70%, at least 80%, at least 90%, at least 95%, at least 98%, at least 99%, or 100%, aa sequence identity to aas 125-227 of the DRB1*09:01 aa sequence depicted in FIG. 5.

### (ii) DRB3

In some cases, a MAPP comprises an MHC Class II β chain polypeptide of a DRB3 allele.

### (a) DRB3*03:01

A MAPP may comprise a DRB3*03:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB3*03:01 aa sequence provided in FIG. 6. In some cases, a MAPP comprises a DRB3*03:01 polypeptide comprising an aa sequence having at least 70%, at least 80%, at least 90%, at least 95%, at least 98%, at least 99%, or 100%, aa sequence identity to aas 30-124 of the DRB3*03:01 aa sequence provided in FIG. 6. In some cases, a MAPP comprises a DRB3*03:01 polypeptide comprising an aa sequence having at least 70%, at least 80%, at least 90%, at least 95%, at least 98%, at least 99%, or 100%, aa sequence identity to aas 125-227 of the DRB3*03:01 aa acid sequence depicted in FIG. 6.

(iii) DRB4

In some cases, a MAPP comprises an MHC Class II β chain polypeptide of a DRB4 allele.

A MAPP may comprise a DRB4*01:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB4*01:01 aa sequence provided in FIG. 7. In some cases, a MAPP comprises a DRB4*01:01 polypeptide comprising an aa sequence having at least 70%, at least 80%, at least 90%, at least 95%, at least 98%, at least 99%, or 100%, aa sequence identity to aas 30-124 of the DRB4*01:01 aa sequence provided in FIG. 7. In some cases, a MAPP comprises a DRB3*03:01 polypeptide comprising an aa sequence having at least 70%, at least 80%, at least 90%, at least 95%, at least 98%, at least 99%, or 100%, aa sequence identity to aas 125-227 of the DRB4*01:01 aa sequence provided in FIG. 7.

### (iv) DRB5

A MAPP may comprise a MHC Class II β chain polypeptide of a DRB5 allele. In some cases, the DRB5 polypeptide has a length of about 198 aas (e.g., 195, 196, 197, 198, 199, 200, 201, or 202 aas). In some cases, a DRB5 polypeptide suitable for inclusion in a MAPP comprises an aa substitution, relative to a wild-type DRB5 polypeptide, where the aa substitution replaces an aa (other than a Cys) with a Cys.

### (a) DRB5*01:01

A MAPP may comprise a DRB5*01:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB5*01:01 aa sequence provided in FIG. 8. In some cases, a DRB5 polypeptide suitable for inclusion in a MAPP comprises an aa substitution, relative to a wild-type DRB5 polypeptide, where the aa substitution replaces an aa (other than a Cys) with a Cys. Thus, e.g., in some cases, the MHC Class II B chain polypeptide is a variant DRB5 MHC Class II polypeptide that comprises a non-naturally occurring Cys residue; e.g., where the variant DRB5 MHC Class II polypeptide comprises an aa substitution selected from the group consisting of P15C, F17C, Q20C, N29C, G30C, N43C, G161C, D162C, and W163C (of a mature DRB5 polypeptide (lacking the N-terminal signal peptide MVCLKLPGGSYMAKLTVTL (SEQ ID NO:98) depicted in FIG. 8), or a naturally-occurring allelic variant thereof.

A MAPP may comprise a DRB5*01:01 β1 domain polypeptide comprising an aa sequence having at least 70%, at least 80%, at least 90%, at least 95%, at least 98%, at least 99%, or 100%, aa sequence identity to aas 30-124 of the DRB5*01:01 aa sequence provided in FIG. 8. In some cases, a MAPP comprises a DRB5*01:01 polypeptide comprising an aa sequence having at least 70%, at least 80%, at least 90%, at least 95%, at least 98%, at least 99%, or 100%, aa sequence identity to at least 170, at least 180, or at least 190, contiguous aas of the sequence from aa 30 to aa 227 of the DRB5*01:01 β2 domain aa sequence depicted in FIG. 8, or a naturally-occurring allelic variant thereof.

d. Disulfide Bonds and the Presenting Sequences and Presenting Complexes

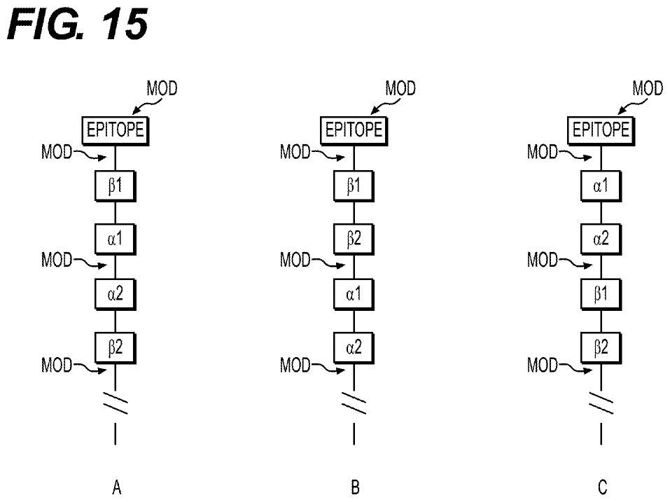

Disulfide bonds involving an MHC peptide sequence may be included in a presenting sequence or complex of a MAPP. The disulfide bonds may increase the stability (e.g., thermal stability) and/or assist in positioning a peptide epitope in the binding pocket/groove of the MHC formed by its α and β chain sequences. The disulfide bonds may be between two MHC peptide sequences (e.g., a cysteine located in an α chain and a cysteine located in a β chain sequence). Disulfide bonds, and particularly disulfide bonds made to position a peptide epitope may be between two MHC peptide sequences or, alternatively, between a MHC peptide sequence and a linker attaching the peptide epitope and an MHC sequence (e.g., the linker between the epitope and β1 domain sequence in FIG. 15 structures A and B). Disulfide bonds for the stabilization and/or positioning an epitope may be made using cysteines found within the MHC sequences and/or cysteines that have been provided in one or more MHC sequences using the techniques of molecular biology and protein engineering. As discussed above, the α chain may include, e.g., a cysteine at position 3, 4, 12, 28, 29, 72, 75, 80, 81, 82, 93, 94, or 95 of the mature α chain (lacking its signal sequence). For the DRA polypeptides cysteines substitutions include, e.g., those at E3C, E4C, F12C, G28C, D29C, I72C, K75C, T80C, P81C, 182C, T93C, N94C, and S95C (see FIG. 4). The β chain may include a cysteine, e.g., at position 5, 7, 10, 19, 20, 33, 151, 152, or 153 of the mature β chain (lacking its signal sequence). For the DB1 polypeptides, possible cysteines substitutions include those at positions P5C, F7C, Q10C (may be Y10C or E10C for some DRB1 alleles), N19C, G20C, H33C (may be N33C for some DRB1alleles), G151C, D152C, and W153C.

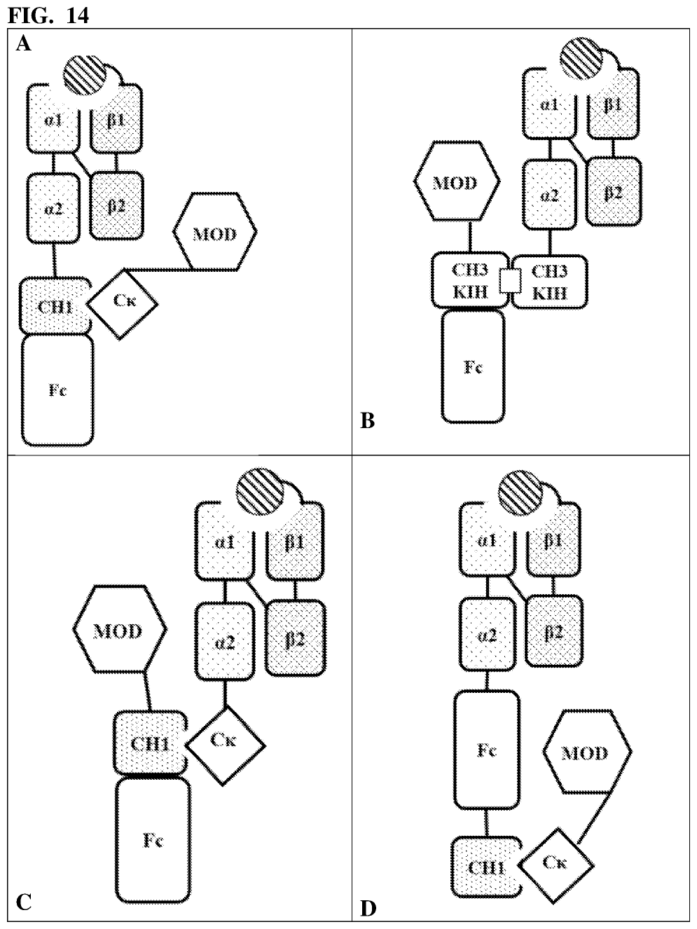

Stabilizing disulfide bonds between α and β chain sequences in the body of the MHC complex (body disulfides) include those between the α and β chain positions set forth in Table 3, which also provides the specific cysteine substitutions for HLA DRA*01:02 and DRB*0401 sequences. The stabilizing disulfide bonds between the MHC (e.g., HLA) α and β chains may be incorporated into any of the MAPP structures described herein. For example, such disulfide bonds may be incorporated into presenting sequences such as those shown in FIG. 15 and the presenting sequences shown in the MAPPs of FIG. 14. Stabilizing disulfide bonds may, for example be incorporated into a presenting sequence having, in order from the N- to C-terminus β1, β2, α1, and α2 domains (see e.g., FIG. 15 structure B).

TABLE 3

| | | DRA*01:02 | DRB*0401 |
|---|---|---|---|
| α chain | β chain | α chain | β chain |
| position | position | substitution | substitution |
| 3 | 19 | E3C | N19C |
| 3 | 20 | E3C | G20C |
| 4 | 19 | E4C | N19C |
| 4 | 20 | E4C | G20C |
| 28 | 151 | G28C | G151C |
| 28 | 152 | G28C | D152C |
| 28 | 153 | G28C | W153 |
| 29 | 151 | D29C | G151C |
| 29 | 152 | D29C | D152C |
| 29 | 153 | D29C | W153 |
| 80 | 33 | T80C | H33C |
| 81 | 33 | P81C | H33C |
| 82 | 33 | I82C | H33C |
| 93 | 156 | T93C | Q156C |
| 93 | 153 | T93C | W153C |
| 94 | 156 | N94C | Q156C |
| 94 | 120 | N94C | N120C |
| 95 | 156 | S95C | Q156C |
| 95 | 120 | S95C | N120C |

Disulfide bonds between the MHC α and β chain sequences that assist in positioning the peptide epitope and/or stabilizing the structure of the presenting sequence or complex are formed between a first aa and second aa of the MAPP. The first aa is either (i) an aa position proximate to the point where a peptide epitope (or a peptide epitope and linker) are attached to an MHC peptide sequence or (ii) an aa (a cysteine) in a linker attached to the peptide epitope, while the second aa is position elsewhere in the MHC peptide sequence. By way of example, where a presenting sequence comprises from N-terminus to C-terminus a peptide epitope, β1 domain, β2 domain, α1 domain, and α2 domain aa sequences, a cysteine substituted within the first ten amino acids (e.g., aas 5-10) of the β1 domain can serve as a first aa and provide a point to anchor the peptide epitope and/or stabilize the MAPP when bonded to a with second cysteine located in, for example, the α1 domain, or α2 domain of the presenting sequence. Some examples of disulfide bonds between the MHC α and β chain sequences that assist in positioning the peptide epitope and/or stabilizing the structure of a presenting sequence or complex include those set forth in Table 4.

TABLE 4

| | | DRA*01:02 | DRB*0401 |
|---|---|---|---|
| α chain | β chain | α chain | β chain |
| position | position | substitution | substitution |
| 12 | 7 | F12C | F7C |
| 12 | 10 | F12C | Q10C |
| 80 | 5 | T80C | P5C |
| 80 | 7 | T80C | F7C |
| 81 | 5 | P81C | P5C |
| 81 | 7 | P81C | F7C |
| 82 | 5 | I82C | P5C |
| 82 | 7 | I82C | F7C |

Thus, for example, when a presenting sequence of complex comprises in the N-terminal to C-terminal direction a peptide epitope bound to a β1 domain, then a disulfide bond between a cysteine substituted at one of position 5-7 of the β chain, and a cysteine at one of aa positions 80-82 of the α chain may be use for positioning the peptide epitope or stabilizing the structure of a presenting sequence. By way of example a disulfide bond between a β chain P5C substitution and an α chain P81C substitution may be used for positioning of the peptide epitope and or stabilization of a presenting sequence. The same type of disulfide bonding is applicable to presenting complexes, and both presenting complexes and presenting sequences may have additional disulfide bonds (e.g., as in Table 3) for stabilization.

Where a cysteine residue in a linker attached to the peptide epitope is employed to position the peptide epitope and/or stabilize the structure of a presenting sequence or complex, the cysteine is typically located at an aa proximate to the point where the linker and peptide epitope meet. For example, where the MAPP comprises an epitope place on the N-terminal side of a linker peptide sequence the cysteine may be within about 6 aas of the position were the linker and peptide epitope meet, that is to say at one of amino acids 1-5 (aa1, aa2, aa3, aa4, or aa5) of a MAPP comprising the construct epitope-aa1-aa2, aa3-aa4-aa5-(remainder of the linker/MAPP). Where the linker comprises repeats of the sequence GGGGS (SEQ ID NO:99), aa1 to aa5 are G1, G2, G3, G4, and S5, and the linker substitutions may be referred to as, for example a “G2C.” This is exemplified by SEQ ID NO: 100, that has four repeats of GGGGS in which the aa at position 2 of the linker (aa2), is a glycine substituted by a cysteine: GCGGSGGGGSGGGGSGGGGS (SEQ ID NO:100). Examples of cysteine containing linkers suitable for forming disulfide bonds with a cysteine in an MHC peptide (e.g., an α chain peptide sequence such a DRA peptide) in a presenting sequence or complex comprising an epitope placed on the N-terminal side of a linker bound to an MHC β chain, such as a DRB polypeptide (i.e., the MAPP comprises the structure epitope-aa1-aa2-aa3-aa4-aa5-[remainder of linker if present]-MHC β1 domain, such as a DRB β1 domain) are set forth in Table 5. Also provided in Table 5 is the location for a cysteine substituted in a DRA polypeptide (see e.g., FIG. 4) that will form the disulfide bond for positioning the peptide epitope and/or stabilizing the structure of a presenting sequence or complex.

TABLE 5
Exemplary Linker disulfide bonds for MAPPS comprising the
structure epitope-aa1-aa2-aa3-aa4-aa5-(remainder of linker
if present)-MHC β1 domain e.g., of a DRB peptide

| | | | α chain |
|---|---|---|---|
| | | | position, (DRA |
| Cysteine | Cysteine substitution in | Cysteine substitution in a | Cysteine |
| position | the general structure | GGGGS containing linker | substitution) |
| aa1 | epitope-C-aa2-aa3-aa4-aa5- | epitope-CGGGS-(remainder of | 72 (I72C) |
| | (remainder of linker)-DRB | linker)-DRB | |
| | | (i.e., a linker G1C substitution) | |
| aa2 | epitope-aa1-C-aa3-aa4-aa5- | epitope-GCGGS-(remainder of | 72 (I72C) |
| | (remainder of linker)-DRB | linker)-DRB | |
| | | (i.e., a linker G2C substitution) | |
| aa3 | epitope-aa1-aa2-C-aa4-aa5- | epitope-GGCGS-(remainder of | 72 (I72C) |
| | (remainder of linker)-DRB | linker)-DRB | |
| | | (i.e., a linker G3C substitution) | |
| aa4 | epitope-aa1-aa2-aa3-C-aa5- | epitope-GGGCS-(remainder of | 72 (I72C) |
| | (remainder of linker)-DRB | linker)-DRB | |
| | | (i.e., a linker G4C substitution) | |
| aa5 | epitope-aa1-aa2-aa3-aa4-C- | epitope-GCGGGC-(remainder | 72 (I72C) |
| | (remainder of linker)-DRB | of linker)-DRB | |
| | | (i.e., a linker S5C substitution) | |
| aal | epitope-C-aa2-aa3-aa4-aa5- | epitope-CGGGS-(remainder of | 75 (K75C) |
| | (remainder of linker)-DRB | linker)-DRB | |
| aa2 | epitope-aa1-C-aa3-aa4-aa5- | epitope-GCGGS-(remainder of | 75 (K75C) |
| | (remainder of linker)-DRB | linker)-DRB | |
| aa3 | epitope-aa1-aa2-C-aa4-aa5- | epitope-GGCGS-(remainder of | 75 (K75C) |
| | (remainder of linker)-DRB | linker)-DRB | |
| aa4 | epitope-aa1-aa2-aa3-C-aa5- | epitope-GGGCS-(remainder of | 75 (K75C) |
| | (remainder of linker)-DRB | linker)-DRB | |
| aa5 | epitope-aal-aa2-aa3-aa4-C- | epitope-GCGGGC-(remainder | 75 (K75C) |
| | (remainder of linker)-DRB | of linker)-DRB | |

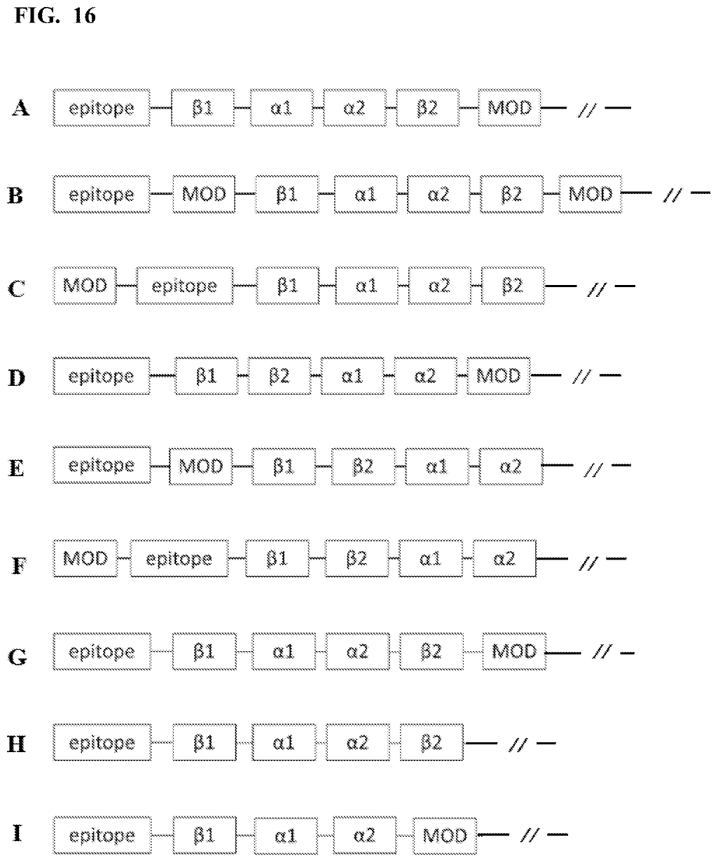

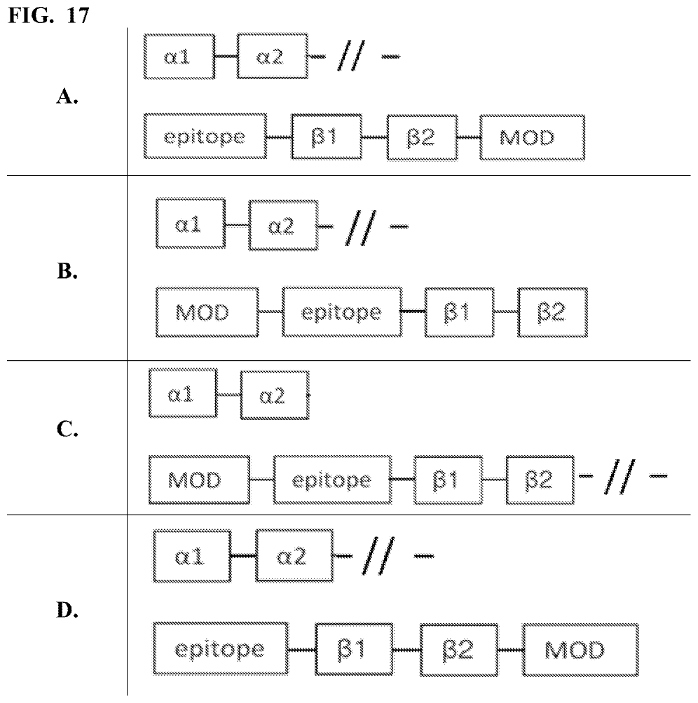

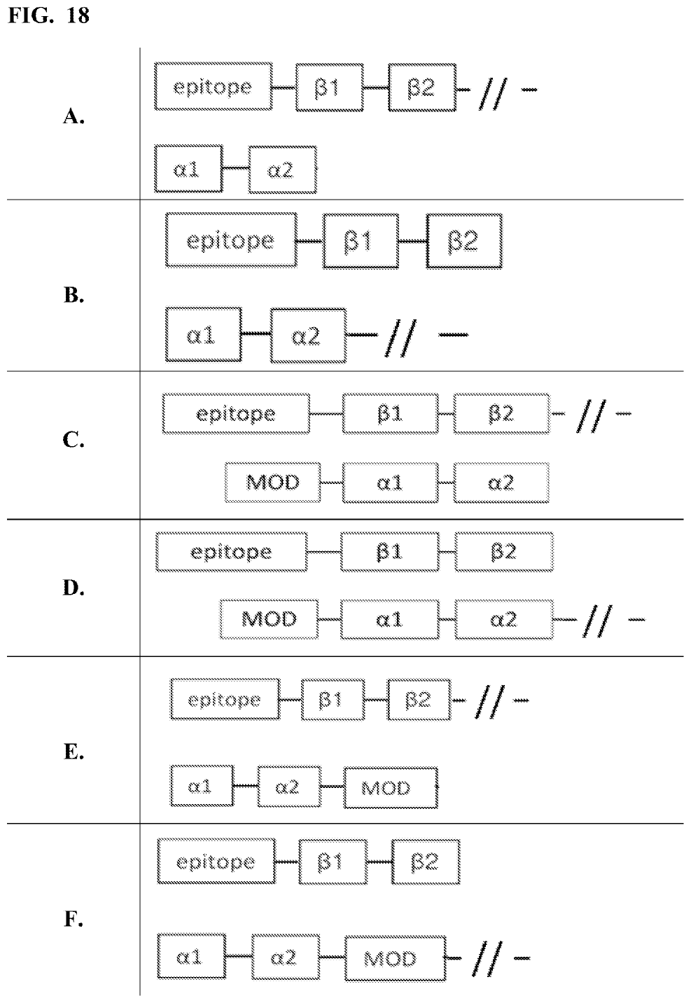

MAPPs with presenting sequences or complexes comprising an epitope-linker-DRB structure recited in Table 5 (see, e.g.: FIG. 14; FIG. 15 structures A and B; FIG. 16 structures A, D, F and H-I; FIG. 17; FIG. 18, and FIG. 19A-F) may have for example a disulfide bond for positioning the peptide epitope and/or stabilizing the structure of a presenting sequence or complex. The disulfide may be formed between linker aa2 (e.g., a G2C) and a cysteine at DRA aa 72 (e.g., I72C). The disulfide may be formed between linker aa2 (e.g., a G2C) and a cysteine at DRA aa 72 (e.g., K75C).

Where a disulfide bond is formed between the linker and an MHC polypeptide of a presenting sequence or presenting complex, the presenting sequence or presenting complex may have additional disulfide bonds (e.g., as in Table 3) for stabilization.

### 5. Immunomodulatory Polypeptides (“MODs”)

A MAPP may comprise one or more immunomodulatory polypeptides or “MODs”. MODs that are suitable for inclusion in a MAPP include, but are not limited to, IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL, and fragments of any thereof, such as ectodomain fragments, capable of engaging and signaling through their cognate receptor. Some MOD polypeptides suitable for inclusion in a MAPP, and their “co-MODS (“co-immunomodulatory polypeptides” or cognate costimulatory receptors) include polypeptide sequences with T cell modulatory activity from the protein pairs recited in the following table:

### Exemplary Pairs of MODs and Co-MODs

a) 4-1BBL (MOD) and 4-1BB (Co-MOD);
b) PD-L1 (MOD) and PD1 (Co-MOD);
c) IL-2 (MOD) and IL-2 receptor (Co-MOD);
d) CD80 (MOD) and CD28 (Co-MOD);
e) CD86 (MOD) and CD28 (Co-MOD);
f) OX40L (CD252) (MOD) and OX40 (CD134) (Co-MOD);
g) Fas ligand (MOD) and Fas (Co-MOD);
h) ICOS-L (MOD) and ICOS (Co-MOD);
i) ICAM (MOD) and LFA-1 (Co-MOD);
j) CD30L (MOD) and CD30 (Co-MOD);
k) CD40 (MOD) and CD40L (Co-MOD);
l) CD83 (MOD) and CD83L (Co-MOD);
m) HVEM (CD270) (MOD) and CD160 (Co-MOD);
n) JAG1 (CD339) (MOD) and Notch (Co-MOD);
o) JAG1 (CD339) (MOD) and CD46 (Co-MOD);
p) CD70 (MOD) and CD27 (Co-MOD);
q) CD80 (MOD) and CTLA4 (Co-MOD);
r) CD86 (MOD) and CTLA4 (Co-MOD);
s) PD-L1(MOD) and CD-80 (Co-MOD); and
t) TGF-β1, TGF-β2, and/or TGF-β3 (MODs) and TGF-β Receptor
(e.g., TGFBR1 and/or TGFBR2) (Co-MOD)

In some cases, the MOD is selected from an IL-2 polypeptide, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide. In some cases, the MAPP or duplex MAPP comprises two different MODs, such as an IL-2 MOD or IL-2 variant MOD polypeptide and either a CD80 or CD86 MOD polypeptide. In another instance, the MAPP or duplex MAPP comprises an IL-2 MOD or IL-2 variant MOD polypeptide and a PD-L1 MOD polypeptide. In some case MODs, which may be the same or different, are present in a MAPP or duplex MAPP in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD polypeptide may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a MAPP or duplex MAPP does not comprise the signal peptide, intracellular domain, or a sufficient portion of the transmembrane domain to anchor a substantial amount (e.g., more than 5% or 10%) of a MAPP or duplex MAPP into a mammalian cell membrane.

In some cases, a MOD suitable for inclusion in a MAPP comprises all or a portion of (e.g., an extracellular portion of) the aa sequence of a naturally-occurring MOD. In other instances, a MOD suitable for inclusion in a MAPP is a variant MOD that comprises at least one aa substitution compared to the aa sequence of a naturally-occurring MOD. In some instances, a variant MOD exhibits a binding affinity for a co-MOD that is lower than the affinity of a corresponding naturally-occurring MOD (e.g., a MOD not comprising the aa substitution(s) present in the variant) for the co-MOD. Suitable variations in MOD polypeptide sequence that alter affinity may be identified by scanning (making aa substitution e.g., alanine substitutions or “alanine scanning” or charged residue changes) along the length of a peptide and testing its affinity. Once key aa positions altering affinity are identified those positions can be subject to a vertical scan in which the effect of one or more aa substitutions other than alanine are tested.

a. MODs and Variant MODs with Reduced Affinity

A MOD can comprise a wild-type amino acid sequence, or can comprise one or more amino acid substitutions, insertions, and/or deletions relative to a wild-type amino acid sequence. The immunomodulatory polypeptide can comprise only the extracellular portion of a full-length immunomodulatory polypeptide. Alternatively, a MOD can comprise all or a portion of (e.g., an extracellular portion of) the amino acid sequence of a naturally-occurring MOD polypeptide.

Variant MODs comprise at least one amino acid substitution, addition and/or deletion as compared to the amino acid sequence of a naturally-occurring immunomodulatory polypeptide. As noted above, in some instances a variant MOD exhibits a binding affinity for a co-MOD that is lower than the affinity of a corresponding naturally-occurring MOD (e.g., an immunomodulatory polypeptide not comprising the amino acid substitution(s) present in the variant) for the co-MOD.

MOD polypeptides and variants, including reduced affinity variants, of proteins such as PD-L1, CD80, CD86, 4-1BBL and IL-2 are described in the published literature, e.g., published PCT application WO2020132138A1, the disclosure of which as it pertains to immunomodulatory polypeptides and specific variant immunomodulatory polypeptides of PD-L1, CD80, CD86, 4-1BBL, IL-2 are expressly incorporated herein by reference, including specifically paragraphs [00260]-[00455] of WO2020132138A1.

Suitable immunomodulatory domains that exhibit reduced affinity for a co-immunomodulatory domain can have from 1 aa to 20 aa differences from a wild-type immunomodulatory domain. For example, in some cases, a variant MOD present in a MAPP may include a single aa substitution compared to a corresponding reference (e.g., wild-type) MOD. A variant MOD present in a MAPP may include 2 aa substitutions compared to a corresponding reference (e.g., wild-type) MOD. A variant MOD present in a MAPP may include 3 or 4 aa substitutions compared to a corresponding reference (e.g., wild-type) MOD. A variant MOD present in a MAPP may include 5 or 6 aa substitutions compared to a corresponding reference (e.g., wild-type) MOD. A variant MOD present in a MAPP may include 7, 8, 9 or 10 aa substitutions compared to a corresponding reference (e.g., wild-type) MOD. A variant MOD present in a MAPP may include 11-15 or 15-20 aa substitutions compared to a corresponding reference (e.g., wild-type) MOD.

As discussed above, a variant MOD suitable for inclusion in a MAPP may exhibit reduced affinity for a cognate co-MOD, compared to the affinity of a corresponding wild-type MOD for the cognate co-MOD.

Binding affinity between a MOD polypeptide sequence and its cognate co-MOD polypeptide can be determined by bio-layer interferometry (BLI) using the purified MOD polypeptide sequence and purified cognate co-MOD polypeptide, following the procedure set forth in published PCT Application WO 2020/132138 A1.

b. Masked TGF-β MODS

As discussed above, a MAPP of the present disclosure comprises at least one TGF-β polypeptide reversibly masked by a polypeptide (a “masking polypeptide”) that binds to the TGF-β polypeptide, which together form a masked TGF-β MOD. The masking polypeptide can be, for instance, a TGF-β receptor polypeptide or an antibody that functions to reversibly mask the TGF-β polypeptide present in the MAPP, where the TGF-β polypeptide is otherwise capable of acting as an agonist of a cellular TGF receptor. The masked TGF-β MODs provide active TGF-β polypeptides (e.g., TGF-β signaling pathway agonists). The TGF-β polypeptides and masking polypeptides (e.g., a TGF-β receptor fragment) interact with each other to reversibly mask the TGF-β polypeptide, thereby permitting the TGF-β polypeptide to interact with its cellular receptor. In addition, the masking sequence competes with cellular receptors that can scavenge TGF-β, such as the non-signaling TβRIII, thereby permitting the TGF-β MOD (and thus the MAPP) to effectively deliver active TGF-β agonist to target cells. While the MAPP constructs discussed herein permit epitope-specific presentation of a reversibly masked TGF-β to a target T cell, they also provide sites for the presentation of one or more additional MODs. The ability of the MAPP construct to include one or more additional MODs thus permits the combined presentation of TGF-β and the additional MOD(s) to direct a target T cell's response in a substantially epitope-specific/selective manner in order to provide modulation of the target T cell. The MAPP thereby permits delivery of one or more masked TGF-β MODs in an epitope-selective (e.g., dependent/specific) manner that permits (i) formation of an active immune synapse with a target T cell, such as a CD4+ cell selective for the epitope, and (ii) modulation (e.g., control/regulation) of the target T cell's response to the epitope. Once engaged with the TCR of a T cell, the effect of a masked TGF-β MOD-containing MAPP on the T cell will depend on whether any additional MODs are present as part of the MAPP and, if so, which additional MOD(s) is/are present.

Further, although the MAPPs of this disclosure may comprise both one or more masked TGF-β MODs and one or more additional MODs such as a wt. or variant IL-2, PD-L1 and/or a 4-1BBL MOD (as discussed above), if desired, the MAPPs of this disclosure may comprise only one or more masked TGF-β MODs. That is, the one or more additional MODs such as the wt. or variant IL-2. PD-L1 and/or a 4-1BBL MOD need not be included in a MAPP of this disclosure. The masked TGF-β MOD-containing MAPPs of the present disclosure can function as a means of producing TGF-β-driven T cell responses. For example, TGF-β by itself can inhibit the development of effector cell functions of T cells, activate macrophages, and/or promote tissue the repair after local immune and inflammatory actions subside.

Although masked TGF-β MODs comprise a TGF-β polypeptide that is masked, the TGF-β polypeptide can still act as TβR agonist because the TGF-β polypeptide-mask complex is reversible and “breathes” between an open state where the TGF-beta polypeptide is available to cellular receptors, and a closed state where the mask engages the TGF-β polypeptide. Accordingly, the masking polypeptide functions to bind TGF-β polypeptide and prevent it from entering into tight complexes with, for example, ubiquitous non-signaling TβR3 molecules that can scavenge otherwise free TGF-β. Moreover, because the active forms of TGF-β are dimers that have higher affinity for TβR3, substitutions that limit dimerization (e.g., a C77Ssubstitution of the cysteine at position 77 with a serine) can be incorporated into TGF-β sequences in order to avoid scavenging by that receptor.

One effect of the masking sequence is to reduce the effective affinity of TGF-β1. TGF-β2, and TGF-β3 polypeptides for TβRs. At the same time, the affinity of the masking polypeptide for the TGF-β polypeptide can be altered so that it dissociates more readily from the TGF-β polypeptide, making the TGF-β polypeptide more available to cellular TβR proteins. That is, where the affinity of a masking polypeptide for a TGF-β polypeptide is reduced, the masked TGF-β MOD will spend more time in the open state. Although in the open state with the TGF-β polypeptide available for binding to cellular receptors, because the TβRII protein is generally the first peptide of the heteromeric TβR1/TβR2 signaling complex to interact with TGF-β, control of the affinity of the TGF-β polypeptide for TβRII effectively controls entry of TGF-β into active signaling complexes. The incorporation of substitution at, for example, one or more, two or more, or all three of Lys 25, Ile 92, and/or Lys 94 of TGF-2 (or the corresponding positions of TGF-β1. TGF-β3) reduces affinity for TβRII polypeptides. The reduced affinity permits interactions between the target cell's TCR and the MAPPs MHC polypeptides and epitope to effectively control binding and allows for target cell-specific interactions.

When a TβRII polypeptide is used as the masking polypeptide, the possibility of direct interactions with cellular TβRI receptors and off-target signaling can be addressed by appropriate modifications of the masking sequence. Where it is desirable to block/limit signaling by the masked TGF-β polypeptide through TβRI and/or modify (e.g., reduce) the affinity of a masking TβRII polypeptide for TGF-β, it is possible to incorporate N-terminal deletions and/or aa substitutions in the masking TβRII polypeptide. Modifications that can be made include deletions of N-terminal amino acids (e.g., N-terminal Δ14 or Δ25 deletions), and/or substitutions at one or more of L27, F30, D32, S49, I50, T51, S52, I53, E55, V77, D118, and/or E119. Some specific TβRII modifications resulting in a reduction in TβRI association with TβRII and reduced affinity for TGF-β include any one or more of L27A, F30A, D32A, D32N, S49A, 150A, T51A, S52A, S52L, 153A, E55A, V77A, D118A, D118R, E119A, and/or E119Q.

The TGF-β polypeptide present in a MAPP is in some cases a variant TGF-β polypeptide, including a variant TGF-β polypeptide that has a lower affinity for at least one class of TGF-β receptors, or is selective for at least one class of TGF-β receptors, compared to a wild-type TGF-β polypeptide.

While a TGF-β1 polypeptide, a TGF-β2 polypeptide, or a TGF-β3 polypeptide can be incorporated into a MAPP as part of a masked TGF-β polypeptide, a variety of factors may influence the choice of the specific TGF-β polypeptide, and the specific sequence and aa substitutions that will be employed. For example, TGF-β1 and TGF-β3 polypeptides are subject to “clipping” of their amino acid sequences when expressed in a certain mammalian cell lines (e.g., CHO cells). In addition, dimerized TGF-β (e.g., TGF-β2) has a higher affinity for the TβR3 (beta glycan receptor) than for the TβR2 receptor, which could lead to off target binding and loss of biologically active masked protein to the large in vivo pool of non-signaling TβR3 molecules. To minimize high-affinity off target binding to TβR3, it may be desirable to substitute the residues leading to dimeric TGF-β molecules, which are joined by a disulfide bond. Accordingly, cysteine 77 (C77) may be substituted by an amino acid other than cysteine (e.g., a serine forming a C77S substitution).

Amino acid sequences of TGF-β polypeptides are known in the art. In some cases, the TGF-β polypeptide present in a masked TGF-β polypeptide is a TGF-β1 polypeptide. In some cases, the TGF-β polypeptide present in a masked TGF-β polypeptide is a TGF-β2 polypeptide. In some cases, the TGF-β polypeptide present in a masked TGF-β polypeptide is a TGF-β3 polypeptide.

A suitable TGF-β polypeptide can have a length from about 70 aas to about 125 aas; for example, a suitable TGF-β polypeptide can have a length from about 70 aas to about 80 aas from about 80 aas to about 90 aas; from about 90 aas to about 100 aas; from about 100 aas to about 105 aas, from about 105 aas to about 110 aas, from about 110 aas to about 112 aas, from about 113 aas to about 120 aas, or from about 120 aas to about 125 aas. A suitable TGF-β polypeptide can comprise an amino acid sequence having at least 60%, at least 70%, at least 80%, at least 80%, at least 85%, at least 90%, at least 95%, at least 98%, at least 99%, or 100%, aa sequence identity to at least 80, at least 90, at least 100, or at least 110 contiguous aas of the mature form of a human TGF-β1 polypeptide, a human TGF-β2 polypeptide, or a human TGF-β3 polypeptide.

### (i) TGF-β1 Polypeptides

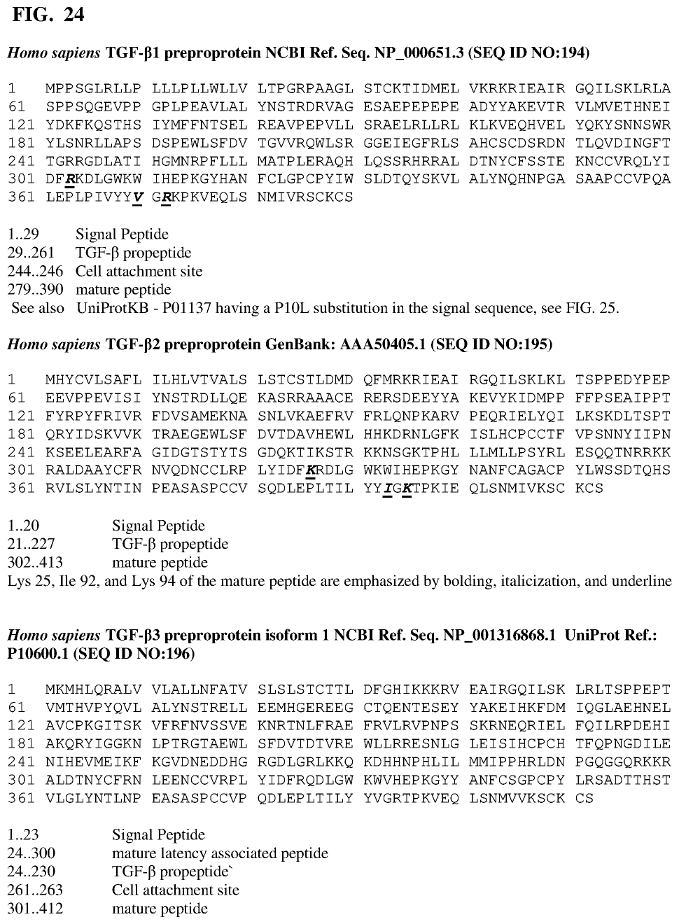

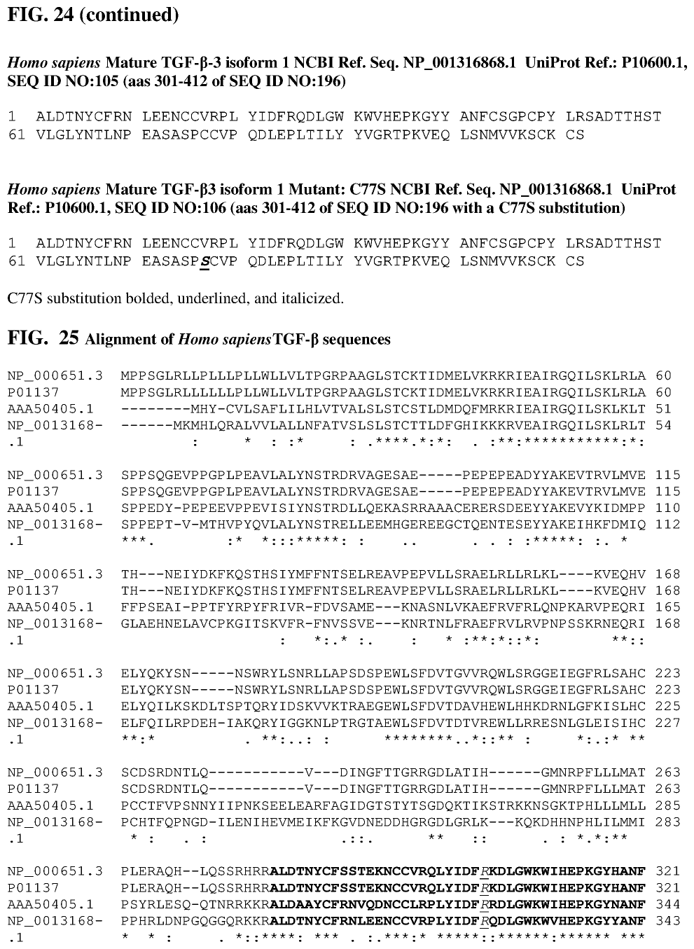

A suitable TGF-β1 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β1 amino acid sequence: AL DTNYCESSTE KNCCVRQLYI DERKDLGWKW IHEPKGYHAN FCLGPCPYIW SLDTQYSKVL ALYNQHNPGA SAAPCCVPQA LEPLPIVYYV GRKPKVEQLS NMIVRSCKCS (SEQ ID NO:101), 112 aas in length); where the TGF-β1 polypeptide has a length of about 112 aas. A TGF-β1 preproprotein is provided in FIG. 24 as SEQ ID NO: 194. Amino acids R25, C77, V92 and R94 are bolded and italicized. See FIG. 24.

In some cases, a suitable TGF-β1 polypeptide comprises a C77S substitution. Thus, in some cases, a suitable TGF-1 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β1 amino acid sequence: AL DTNYCFSSTE KNCCVRQLYI DFRKDLGWKW IHEPKGYHAN FCLGPCPYIW SLDTQYSKVL ALYNQHNPGA SAAPSCVPQA LEPLPIVYYV GRKPKVEQLS NMIVRSCKCS (SEQ ID NO:102), where amino acid 77 is Ser. Positions 25, 77, 92 and 94 are bolded and italicized.

### (ii) TGF-β2 Polypeptides

A suitable TGF-β2 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β2 amino acid sequence: ALDAAYCFR NVQDNCCLRP LYIDFKRDLG WKWIHEPKGY NANFCAGACP YLWSSDTQHS RVLSLYNTIN PEASASPCCV SQDLEPLTIL YYIGKTPKIE QLSNMIVKSC KCS (SEQ ID NO: 103), where the TGF-β2 polypeptide has a length of about 112 aas. A TGF-β2 preproprotein is provided in FIG. //34 as SEQ ID NO: 195. Residues Lys 25. Cys 77, Ile 92, and Lys 94 are bolded and italicized.

In some cases, a suitable TGF-β2 polypeptide comprises a C77S substitution. Thus, in some cases, a suitable TGF-β2 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-2 amino acid sequence: ALDAAYCFR NVQDNCCLRP LYIDFKRDLG WKWIHEPKGY NANFCAGACP YLWSSDTQHS RVLSLYNTIN PEASASPSCV SQDLEPLTIL YYIGKTPKIE QLSNMIVKSC KCS (SEQ ID NO:104), where amino acid 77 is substituted by a Ser that is bolded and italicized.

(iii) TGF-β3 Polypeptides

A suitable TGF-β3 polypeptide can comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β3 amino acid sequence: ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO:105), where the TGF-β3 polypeptide has a length of about 112 aas. A TGF-β3 isoform 1 preproprotein is provided in FIG. 34 as SEQ ID NO: 196. Positions 25, 77, 92 and 94 are bolded and italicized.

In some cases, a suitable TGF-β3 polypeptide comprises a C77S substitution. In some cases, a suitable TGF-β3 polypeptide comprises an amino acid sequence having at least 60%, at least 70%, at least 80%, at least 90%, at least 95%, at least 98%, at least 99%, or 100%, aa sequence identity to at least 70, at least 80, at least 90, at least 100, at least 110, or 112 aas of the following TGF-β3 amino acid sequence: ALDTNYCFRN LEENCCVRPL YIDFRQDLGW KWVHEPKGYY ANFCSGPCPY LRSADTTHST VLGLYNTLNP EASASPSCVP QDLEPLTILY YVGRTPKVEQ LSNMVVKSCK CS (SEQ ID NO:106), where amino acid 77 is Ser. Positions 25, 77, 92 and 94 are bolded and italicized.

### (iv) Additional TGF-β Polypeptide Sequence Variations

In addition to sequence variations that alter TGF-β molecule dimerization (e.g., cysteine 77 substitutions such as C77S), TGF-β1, TGF-β2, and TGF-β3 polypeptides having sequence variations that affect affinity and other properties may be incorporated into a masked TGF-β MOD. When a variant TGF-β with reduced affinity for the masking polypeptide (e.g., a TβR polypeptide such as a TβRII polypeptide) is present in the masked TGF-β MOD those components dissociate more readily, making the TGF-β polypeptide more available to cellular TβR proteins. Because the TβRII protein is generally the first peptide of the heteromeric TβR signaling complex to interact with TGF-β, interactions with TβRII effectively controls entry of TGF-β into active signaling complexes. Accordingly, variants controlling the affinity of TGF-β for TβRII may effectively control entry of masked TGF-β MODs into active signaling complexes.

The present disclosure includes and provides for masked TGF-β MODs comprising a variant masking TβR (e.g., TβRII) polypeptide sequence and/or a variant TGF-β polypeptide having altered (e.g., reduced) affinity for each other (relative to an otherwise identical masked TGF-β MOD without the sequence variation(s)). Affinity between a TGF-β polypeptide and a TβR (e.g., TβRII) polypeptide may be determined using (BLI) as described above for MODs and their co-MODs.

### (a) Additional TGF-β2 Sequence Variants

The present disclosure includes and provides for masked TGF-β2 MODs comprising a masking TβR (e.g., TβRII) polypeptide sequence and either a wt. or a variant TGF-β2 polypeptide; where the variant polypeptide has a reduced affinity for the masking TβR (relative to an otherwise identical wt. TGF-β polypeptide sequence without the sequence variations).

The disclosure provides for a masked TGF-β MODs that comprise a masking TβRII receptor sequence and a variant TGF-2 polypeptide having greater than 85% (e.g., greater than 90%, 95%, 98% or 99%) sequence identity to at least 100 contiguous aa of SEQ ID NO: 103, and comprising a substitution reducing the affinity of the variant TGF-β2 polypeptide for the TβRII receptor sequence.

In some cases, a masked TGF-β MOD comprises a masking TβRII polypeptide and a variant TGF-β (e.g., TGF-β2) polypeptide comprising a substitution at one or more, two or more, or all three of Lys 25, Ile 92, and/or Lys 94 (see SEQ ID NO: 103 for the location of the residues, and FIG. 25 for the corresponding residues in TGF-β1 and TGF-β3). Those aa residues have been shown to affect the affinity of TGF-β2 for TβRII polypeptides (see Crescenzo et al., J. Mol. Biol. 355: 47-62 (2006)). The MAPP optionally comprises one or more independently selected MODs such as IL-2 or a variant thereof. In one instance, the masked TGF-β MOD comprises a masking TβRII polypeptide and a TGF-β2 polypeptide having an aa other than Lys or Arg at position 25 of SEQ ID NO: 103; with the MAPP optionally comprising one or more additional independently selected MODs (e.g., one or more IL-2 MOD polypeptide or reduced affinity variant thereof). A masked TGF-β MOD with a masking TβRII polypeptide may comprises a TGF-β2 polypeptide having an aa other than Ile or Val at position 92 of SEQ ID NO: 103 (or an aa other than Ile. Val, or Leu at position 92); with the MAPP optionally comprising one or more additional independently selected MODs (e.g., one or more IL-2 MOD polypeptide or reduced affinity variant thereof). A masked TGF-β MOD with a masking TβRII polypeptide may comprise a TGF-β2 polypeptide having an aa other than Lys or Arg at position 94 of SEQ ID NO:103); with the MAPP optionally comprising one or more additional independently selected MODs (e.g., one or more IL-2 MOD polypeptide or reduced affinity variant thereof). A masked TGF-β MOD with a masking TβRII polypeptide may comprise a TGF-β2 polypeptide comprising a substitution at one or more, two or more or all three of Lys 25, Ile 92, and/or Lys 94); with the MAPP optionally comprising one or more additional independently selected MODs. A masked TGF-β MOD with a masking TβRII polypeptide may comprise a TGF-β2 polypeptide comprising a substitution at one or more, two or more or all three of Lys 25, Ile 92, and/or Lys 94); with the MAPP optionally comprising one or more additional independently selected IL-2 MODs or reduced affinity variants thereof.

### (b) Additional TGF-β1 and TGF-β3 Sequence Variants and Placement in Tandem

In some cases, a masked TGF-β MOD comprises a masking TβRII polypeptide and a variant TGF-β1 or TGF-β3 polypeptide comprising a substitution at one or more, two or more or all three aa positions corresponding to Lys 25, Ile 92, and/or Lys 94 in TGF-β2 SEQ ID NO: 103. In TGF-β1 or TGF-3, the aa that corresponds to: Lys 25 is an Arg 25, Ile 92 is Val 92, and Lys 94 is Arg 94, each of which is a conservative substitution. See e.g., SEQ ID NOs: 194 and 102 for TGF-β1 and SEQ ID NOs: 196 and 106 for // TGF-β3.

As noted above, the masked TGF-β MOD optionally comprises one or more independently selected MODs such as IL-2 or a variant thereof. In one instance, the masked TGF-β MOD with a masking TβRII polypeptide comprises a TGF-β1 or β3 polypeptide having an aa other than Arg or Lys at position 25; and optionally comprises one or more independently selected MODs (e.g., one or more IL-2 MOD polypeptide or reduced affinity variant thereof). In one instance, the masked TGF-β MOD with a masking TβRII polypeptide comprises a TGF-β1 or β3 polypeptide having an aa other than Val or Ile at position 92 (or an aa other than Ile, Val, or Leu at position 92); and optionally comprises one or more independently selected MODs (e.g., one or more IL-2 MOD polypeptide or reduced affinity variant thereof). In another instance, the masked TGF-β MOD with a masking TβRII polypeptide comprises a TGF-β2 polypeptide having an aa other than Arg or Lys; and optionally comprises one or more independently selected MODs (e.g., one or more IL-2 MOD polypeptide or reduced affinity variant thereof). In one specific instance, a masked TGF-β MOD with a masking TβRII polypeptide comprises a TGF-β1 or β3 polypeptide comprising a substitution at one or more, two or more or all three of Arg 25. Val 92, and/or Arg 94, and further comprises one or more independently selected MODs (e.g., IL-2 or variant IL-2 MODs). In another specific instance, a masked TGF-β MOD with a masking TβRII polypeptide comprises a TGF-β1 or β3 polypeptide comprising a substitution at one or more, two or more or all three of Arg 25, Val 92, and/or Arg 94, and further comprises one or more independently selected IL-2 MODs, or reduced affinity variants thereof.

(v) TGF-β Receptor Polypeptides and Other Polypeptides that Bind and Mask TGF-β

In any of the above-mentioned TGF-β polypeptides or polypeptide complexes the polypeptide that binds to and masks the TGF-β polypeptide (the “masking polypeptide”) can take a variety of forms, including fragments of TβRI, TβRII, TβRIII and anti-TGF-β antibodies or antibody-related molecules (e.g., antigen binding fragment of an antibody. Fab, Fab′, single chain antibody, scFv, peptide aptamer, or nanobody).

### (a) TGF-β Receptor Polypeptides

The masking of TGF-β in masked TGF-β MODs may be accomplished by utilizing a TGF-β receptor fragment (e.g., the ectodomain sequences of TβRI, TβRII or TβRIII) that comprises polypeptide sequences sufficient to bind a TGF-β polypeptide (e.g., TGF-β1, TGF-β2 or TGF-β3). In an embodiment, the masking sequence comprises all or part of the TβRI, TβRII, or TβRIII ectodomain.

### (1) TGF-β Receptor I (TβRI)

The polypeptide sequence masking TGF-β in a masked TGF-β MODs may be derived from a TβRI (e.g., isoform 1 SEQ ID NO:197) and may comprises all or part of the TβRI ectodomain (aas 34-126). A suitable TβRI polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, or 103 aas of the following TβRI ectodomain aa sequence: LQCFCHL CTKDNFTCVT DGLCFVSVTE TTDKVIHNSM CIAEIDLIPR DRPFVCAPSS KTGSVTTTYC CNQDHCNKIE LPTTVKSSPG LGPVEL (SEQ ID NO:107).

### (2) TGF-β Receptor II (TβRII)

A polypeptide sequence masking TGF-β in a masked TGF-β MOD may be derived from a TβRII (e.g., isoform A SEQ ID NO:198), and may comprises all or part of the TβRII ectodomain sequence (aas 24 to 177). A suitable TβRII isoform A polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, at least 150 or at least 154 aas of the following TβRII isoform A ectodomain aa sequence: IPPHVQK SDVEMEAQKD EIICPSCNRT AHPLRHINND MIVTDNNGAV KFPQLCKFCD VRESTCDNQK SCMSNCSITS ICEKPQEVCV AVWRKNDENI TLETVCHDPK LPYHDFILED AASPKCIMKE KKKPGETFFM CSCSSDECND NIIFSEE (SEQ ID NO: 108). The location of the aspartic acid residue corresponding to D118 in the B isoform is bolded and italicized.

A polypeptide sequence masking TGF-β in a masked TGF-β MOD may be derived from TβRII isoform B SEQ ID NO: 199) and may comprises all or part of the TβRII ectodomain sequence (aas 24 to 166). A suitable TβRII isoform B polypeptide for masking TGF-β may comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, or 143 aas of the TβRII isoform B ectodomain aa sequence: IPPHVQKSVN NDMIVTDNNG AVKFPQLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEEY NTSNPDLLLV IFQ (SEQ ID NO: 109). As discussed below, any one or more of F30, D32, S52, E55, or D118 (italicized and bolded) may be substituted by an amino acid other than the naturally occurring aa at those positions (e.g., alanine). A polypeptide sequence masking TGF-β may comprise the polypeptide of SEQ ID NO:109 bearing a D118A or D118R substitution. A sequence masking TGF-β may comprise the peptide of SEQ ID NO: 109 bearing a D118A or D118R substitution and one or more of a F30A, D32N, S52L and/or E55A substitution.

Although TβRII's ectodomain may be utilized as a masking polypeptide, that region of the protein has charged and hydrophobic patches that can lead to an unfavorable pI and can be toxic to cells expressing the polypeptide. In addition, combining a TβRII ectodomain with the an active TGF-β polypeptide can result in a complex that could combine with cell surface TβRI and cause activation of that signaling receptor (e.g., signaling through the Smad pathway). Modifying TβRII ectodomain sequences used to mask TGF-β by removing or altering sequences involved in TβRI association can avoid the unintentional stimulation of cells by the masked TGF-β except through their own cell surface heterodimeric TβRI/TβRII complex. Modifications of TβRII may also alter (e.g., reduce) the affinity of the TβRII for TGF-β (e.g., TGF-β3), thereby permitting control of TGF-β unmasking and its availability as a signaling molecule. Masked TGF-β MODs comprising TβR (e.g., TβRII) peptides with the highest affinity for TGF-β (e.g., TGF-β3) most tightly mask the TGF-β sequence and require higher doses to achieve the same effect. In contrast, aa substitutions in TβRII that lower the affinity unmask the TGF-β polypeptide and are biologically effective at lower doses.

Accordingly, where it is desirable to block/limit signaling by the masked TGF-β polypeptide through TβRI and/or modify (e.g., reduce) the affinity of a masking TβRII polypeptide for TGF-β a number of alterations to TβRII may be incorporated into the TβRII polypeptide sequence. Modifications that can be made include the above-mentioned deletions of N-terminal amino acids, such as 14 or 25 N-terminal amino acids (from 1 to 14aas or from 1 to 25 aas; Δ14, Δ25 modifications), and/or substitutions at one or more of L27, F30, D32, S49, I50, T51, S52, I53, E55, V77, D118, and/or E119. Some specific TβRII modifications resulting in a reduction in TβRI association with TβRII and reduced affinity for TGF-β include any one or more of L27A, F30A, D32A, D32N, S49A, 150A, T51A, S52A, S52L, 153A, E55A, V77A, D118A, D118R, E119A, and/or E119Q based on SEQ ID NO: 109. See e.g., J. Groppe et al. Mol Cell 29, 157-168, (2008) and De Crescenzo et al. JMB 355, 47-62 (2006) for the effects of those substitutions on TGF-β3-TβRII and TβRI-TβRII complexes. Modifications of TβRII the including an N-terminal Δ25 deletion and/or substitutions at F24 (e.g., an F24A substitution) substantially or completely block signal through the canonical SMAD signaling pathway). In one aspect, the aspartic acid at position 118 (D118) of the mature TβRII B isoform (SEQ ID NO: 109) is replaced by an amino acid other than Asp or Glu, such as Ala giving rise to a “D118A” substitution or by an Arg giving rise to a D118R substitution. The Asp residues corresponding D118 are indicated SEQ ID Nos: 108, 199, 109, 110, 111, 112, 113 (with bold and underlining in FIG. 26B), N-terminal deletions of from 1 to 25 aa in length (e.g., a Δ25 deletions) and/or substitutions at F24 (e.g., an F24A substitution) may be combined with D118 substitutions (e.g., D118A or D118R). N-terminal deletions of from 1 to 25 aa in length (e.g., a Δ25 deletions) and/or substitutions at F24 (e.g., an F24A substitution) may also be combined with substitutions at any of L27, F30, D32, S49, I50, T51, S52, I53, E55, V77, D118, and/or E119 (e.g., D118A) substitutions, and particularly any of the specific substitutions recited for those locations in SEQ ID NO:109 described above to alter the affinity.

Deletions of the N-terminus of the TβRII polypeptides may also result in loss of TβRI interactions and prevent masked TGF-β MODs comprising a TβRII polypeptide from acting as a constitutively active complex that engages and activates TβRI signaling. A 14 aa deletion (Δ14) of the TβRII polypeptide substantively reduces the interaction of the protein with TβRI, and a Δ25 aa deletion of TβRII appears to completely abrogate the interaction with TβRI. N-terminal deletions also substantially alter the pI of the protein, with the Δ14 TβRII ectodomain mutant displaying a pI of about 4.5-5.0 (e.g., about 4.74). Accordingly. TGF-β MODs may comprise TβRII ectodomain polypeptides (e.g., polypeptides of SEQ ID NOs: 108 or 199) with N-terminal deletions, such as from 14 to 25 aas (e.g., 14, 15, 16, 17, 18, 19, 20, 21, 22, 23, 24, or 25 aa). Modified ectodomain sequences, including those that limit interactions with TβRI, that may be utilized to mask TGF-β polypeptides in a masked TGF-β MOD are described in the paragraphs that follow.

In an embodiment, the sequence masking TGF-β in a masked TGF-β MOD comprises sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, or 142 aas of the TβRII isoform B ectodomain sequence: IPPHVQKSVN NDMIVTDNNG AVKFPQLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:110). Any one or more of F30, D32, S52, E55, or D118 (italicized and bolded) may be substituted by an amino acid other than the naturally occurring aa at those positions (e.g., alanine). In an embodiment, the sequence masking TGF-β comprises the peptide of SEQ ID NO:110 bearing a D118A substitution. In an embodiment, the sequence masking TGF-β comprises the polypeptide of SEQ ID NO:110 bearing a D118A substitution and one or more of a F30A, D32N, S52L and/or E55A substitution.

Combinations of N-terminal deletions of TβRII, such as from 14 to 25 aas (e.g., 14, 15, 16, 17, 18, 19, 20, 21, 22, 23, 24, or 25 aa), that block inadvertent cell signaling due to the masked TGF-β/TβRII complex interacting with TβRI may be combined with other TβRII ectodomain substitutions, including those at any one or more of F30, D32, S52, E55, and/or D118. The combination of deletions and substitutions ensures the masked TGF-β MOD does not cause cell signaling except through the cell's membrane bound TβRI & TβRII receptors.

In an embodiment, the sequence masking TGF-β in a masked TGF-β MOD comprises sequence having at least 60%, at least 70%, at least 80%, at least 90%, at least 95%, at least 98%, at least 99%, or 100%, aa sequence identity to at least 70, at least 80, at least 90, at least 100, at least 110, or 114 aas of the TβRII isoform B ectodomain sequence: VTDNNG AVKFPQLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:111), which has aas 1-14 (Δ14) deleted. Any one or more of F30, D32, S52, E55, or D118 (italicized and bolded) may be substituted by an amino acid other than the naturally occurring aa at those positions (e.g., alanine). In an embodiment, the sequence masking TGF-β comprises the peptide of SEQ ID NO: 111 bearing a D118A substitution. In an embodiment, the sequence masking TGF-β comprises the polypeptide of SEQ ID NO: 111 bearing a D118A substitution and one or more of a F30A, D32N, S52L and/or E55A substitution.

In an embodiment, the sequence masking TGF-β in a masked TGF-β MOD comprises sequence having at least 60%, at least 70%, at least 80%, at least 90%, at least 95%, at least 98%, at least 99%, or 100%, aa sequence identity to at least 70, at least 80, at least 90, at least 100, or 104 aas of the TβRII isoform B ectodomain sequence: QLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO: 112), which has aas 1-25 (Δ25) deleted. Any one or more of F30, D32, S52, E55, or D118 (italicized and bolded) may be substituted by an amino acid other than the naturally occurring aa at those positions (e.g., alanine). In an embodiment, the sequence masking TGF-β comprises the polypeptide of SEQ ID NO: 112 bearing a D118A substitution (shown as SEQ ID NO:113 in FIG. 26B). In an embodiment, the sequence masking TGF-β in a masked TGF-β MOD comprises the peptide of SEQ ID NO: 112 bearing a D118A substitution and one or more of a F30A, D32N, S52L and/or E55A substitution. In an embodiment, the sequence masking TGF-β in a masked TGF-β MOD comprises the peptide of SEQ ID NO:112 (see FIG. 5B) bearing D118A and F30A substitutions. In an embodiment, the sequence masking TGF-β in a masked TGF-β MOD comprises the peptide of SEQ ID NO:112 (see FIG. 26B) bearing D118A and D32N substitutions. In an embodiment, the sequence masking TGF-β in a masked TGF-β MOD comprises the peptide of SEQ ID NO:112 (see FIG. 26B) bearing D118A and S52L substitutions. In an embodiment, the sequence masking TGF-β in a masked TGF-β MOD comprises the peptide of SEQ ID NO: 112 (see FIG. 26B) bearing D118A and E55A.

### (3) TGF-β Receptor III (TβRIII)

In an embodiment, the polypeptide sequence masking TGF-β in a masked TGF-β MOD may be derived from a TβRIII (e.g., isoform A SEQ ID NO:200 and isoform B 125), and may comprises all or part of a TβRIII ectodomain (aas 27-787 of the A isoform or 27-786 of the B isoform). In some cases, a suitable TβRIII polypeptide for masking TGF-β comprises an amino acid sequence having at least 60%, at least 70%, at least 80%, at least 90%, at least 95%, at least 98%, at least 99%, or 100%, aa sequence identity to at least 70, at least 80, at least 90, at least 100, or 120 aas of a TβRIII A isoform or B isoform ectodomain sequences (e.g., provided in FIG. 26C as SEQ ID NO:200 or SEQ ID NO:201).

### (b) Antibodies

Although TGF-β receptor polypeptides (e.g., ectodomain sequences) can function to bind and mask TGF-β polypeptides in masked TGF-β MODs, other polypeptide sequences (protein sequences) that bind to TGF-β sequences can also be employed as masking polypeptides. Among the suitable polypeptide or protein sequences that can be used to mask TGF-β are antibodies with affinity for TGF-β (e.g., antibodies specific for an one or more of TGF-β1, TGF-β2, or TGF-β3) or antibody-related molecules such as anti-TGF-β antibody fragments, nanobodies with affinity for TGF-β polypeptides, and particularly single chain anti-TGF-β antibodies (e.g., any of which may be humanized). Some antibodies, including scFV antibodies, that bind and neutralize TGF-β have been described. See e.g., U.S. Pat. No. 9,090,685. Throughout the embodiments and/or aspects of the invention described in this disclosure. TβR (e.g., TβRII) sequences used to mask TGF-β polypeptides may be replaced with masking antibody sequences (e.g., a scFV or a nanobody) with affinity for the TGF-β polypeptide. For instance, in each of the masked TGF-β MODs in FIG. 1 where a TGF-β receptor sequence is used to mask a TGF-β polypeptide, the receptor polypeptide may be replaced with a masking antibody polypeptide (e.g., scFV or a nanobody) with affinity for the TGF-β polypeptide.

One potential advantage of using an antibody (e.g., a single chain antibody) as a masking polypeptide is the ability to limit it to the isoform of the TGF-β polypeptide(s) to be masked. By way of example, single chain antibody sequences based on Metelimumab (CAT192) directed against TGF-β1 (e.g., Lord et al., mAbs 10(3): 444-452 (2018)) can be used to mask that TGF-β isoform when present in TGF-β MODs. In another embodiment, a single chain antibody sequence specific for TGF-β2 is used to mask that TGF-β isoform when present in TGF-β MODs. In another embodiment, a single chain antibody sequence specific for TGF-β3 is used to mask that TGF-β isoform when present in TGF-β MODs. Single chain antibodies can also be specific for a combination of TGF-β isoforms (e.g., ectodomain sequences appearing in masked TGF-β MODs selected from the group consisting of: TGF-β1 & TGF-β2; TGF-β1 & TGF-β3; and TGF-β2 & TGF-β3. The single chain antibodies may also be pan-specific for TGF-β1. TGF-β2, and TGF-β3 ectodomain sequences appearing in masked TGF-β MODs See e.g., WO 2014/164709. Antibodies and single chain antibodies that have the desired specificity and affinity for TGF-β isoforms can be prepared by a variety of methods, including screening hybridomas and/or modification (e.g., combinatorial modification) to the variable region sequence of antibodies that have affinity for a target TGF-β polypeptide sequence.

In an embodiment, a masked TGF-β MOD comprises a single chain antibody to mask a TGF-β sequence (e.g., a TGF-β3 sequence). In one such embodiment the single chain amino acid sequence is specific for the TGF-β3 set forth in SEQ ID NO: 105 comprising a C77S substitution (see SEQ ID NO:196).

### (i) Placement of TGF-β and TGF-β Masking Sequence in MAPPs

The masking sequence (e.g., a TGF-β receptor sequence) of a masked TGF-β MOD may either be part of the same polypeptide as the TGF-β sequence, that is both the masking and TGF-β sequences are present in “cis.” Alternatively, the masking sequence (e.g., a TGF-β receptor sequence) and the TGF-β sequence may be part of a different polypeptides, that is to say they are present in “trans.”

When the masking sequence and the TGF-β sequence of a masked TGF-β MOD are present in a single aa sequence (single polypeptide) of a MAPP (placed in cis), the aa sequence may be arranged in the N-terminal to C-terminal direction as either: a) TGF-β receptor sequence(s) followed by TGF-β sequence(s), or b) TGF-β sequence(s) followed by TGF-β receptor sequence(s). Regardless of the order from N-terminus to C-terminus, the polypeptide sequence of a masked TGF-β MOD may be linked to any other MAPP polypeptide at its N-terminus or C-terminus. Independently selected linker polypeptide (e.g., Gly₄Ser repeats) may be used to join the masking sequence (e.g., a TGF-β receptor sequence) and the TGF-β sequence, and also to join the TGF-β MOD to a polypeptide of the MAPP (e.g., a framework or dimerization polypeptide). As an example, a cis-masked TGF-β MOD may be linked to the C terminus of a MAPP polypeptide and have the order from N-terminus to C-terminus a) TGF-β receptor sequence (e.g., a TβRII sequence) followed by TGF-β sequence (e.g., TGF-β3). To further that example, the cis-masked TGF-β MOD may be linked to the framework polypeptide (e.g., at its C-terminus) and the cis-masked TGF-β MOD may optionally be followed by another MOD such as IL-2.

One example of a masked TGF-β MOD with the TβR and TGF-β in cis (a cis-masked TGF-β MOD) is the sequence QLCKFCDVRFSTCDNQKSCMSNCSITSICEKPQEVCVAVWRKNDENITLETVCHDPKLPYHDFIL EDAASPKCIMKEKKKPGETFFMCSCSSAECNDNIIFSEEYNTSNPDGGGGSGGGGSGGGGSGGGG SGGGGSALDTNYCFRNLEENCCVRPLYIDFRQDLGWKWVHEPKGYYANFCSGPCPYLRSADTT HSTVLGLYNTLNPEASASPSCVPQDLEPLTILYYVGRTPKVEQLSNMVVKSCKCS (SEQ ID NO:209), where: aas 1-111 are a human TβRII masking sequence with the N-terminal 25 aas removed (Δ25) and a D118A substitution; aas 112-136 are a linker (five Gly₄Ser repeats); and 137-248 is a human TGF-β3 sequence with a C77S substitution. Such a sequence may be attached, for example, by its N-terminus, directly or indirectly, via an independently selected linker to the C-terminus of a MAPP polypeptide (e.g., a framework polypeptide). In addition, the cis masked TGF-β MOD sequence may have appended to it another MOD sequence (e.g., a human IL-2 or variant IL-2 MOD polypeptide sequence).

When the masking sequence (e.g., TGF-β receptor sequence) and the TGF-β sequence of a masked TGF-β MODs are present as part of different MAPP polypeptides (placed in trans), those polypeptide sequences are attached to different (separate) MAPP polypeptides that interact, thereby pairing TGF-β sequence with masking polypeptide (e.g., a TGF-β receptor sequence). The TGF-β sequence and masking sequence may be located at the N-terminus or C-terminus of MAPP polypeptides (e.g., framework or dimerization polypeptides). Independently selected linker polypeptide (e.g., Gly₄Ser repeats) may be used to join the masking sequence (e.g., TGF-β receptor sequence) or the TGF-β sequence to other MAPP polypeptides. As an example, in a trans-masked TGF-β MOD a TGF-β receptor sequence (e.g., TβRII) may be part of one framework polypeptide and the TGF-β sequence (e.g., TGF-β3) part of a second framework polypeptide, where the first and second framework polypeptides associate through interspecific multimerization sequences. To further that example, the TGF-β sequence and TGF-β receptor sequences may be located at the C-terminus of the framework polypeptides and may optionally be followed by another MOD such as IL-2. By way of example, a MAPP having first and second framework polypeptides with interspecific multimerization sequences may have a masking TβR sequence located at the C-terminus of a first framework polypeptide, and a TGF-β polypeptide located at the C-terminus of the second framework polypeptides (positions 3 and 3′ (see e.g., FIGS. 1A and 1B). The masking TβR sequence may, for example, be a TβRII sequence lacking its N-terminal 25 aas (Δ25) and bearing a D118A substitution: SQLCKFCDVRFSTCDNQKSCMSNCSITSICEKPQEVCVAVWRKNDENITLETVCHDPKLPYHDFI LEDAASPKCIMKEKKKPGETFFMCSCSSAECNDNIIFSEEYNTSNPD (SEQ ID NO:114). The TGF-β polypeptide may be a human TGF-β3 polypeptide bearing a C77S substitution:

(SEQ ID NO: 115)
ALDTNYCFRNLEENCCVRPLYIDFRQDLGWKWVHEPKGYYANFCSGP
CPYLRSADTTHSTVLGLYNTLNPEASASPSCVPQDLEPLTILYYVGR
TPKVEQLSNMVVKSCKCS.

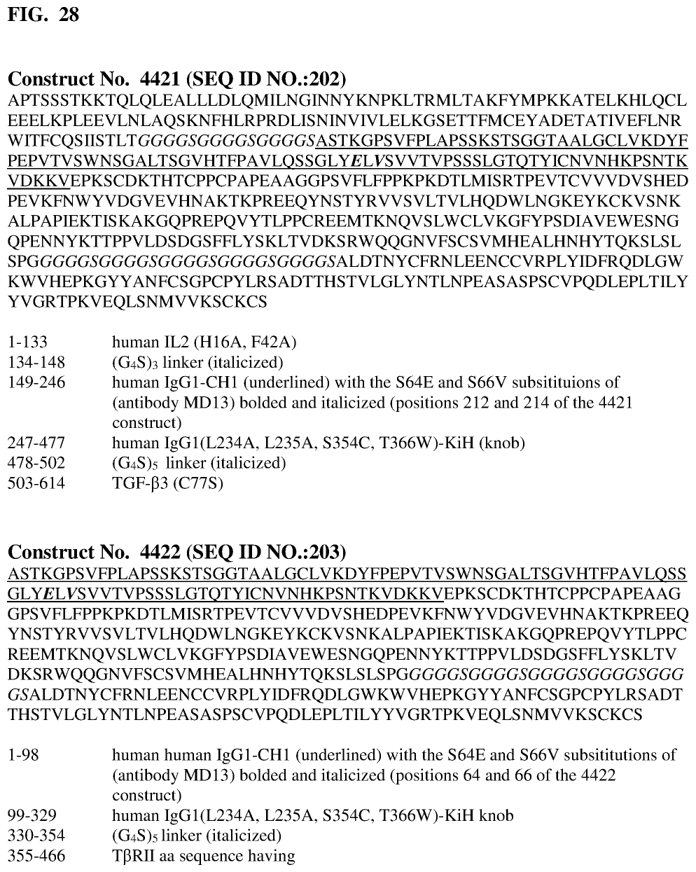

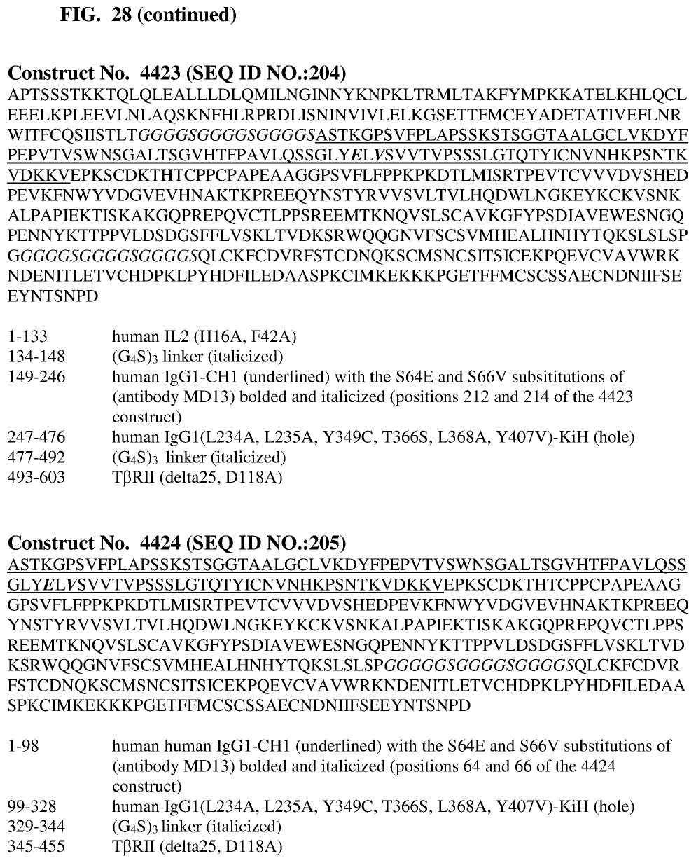

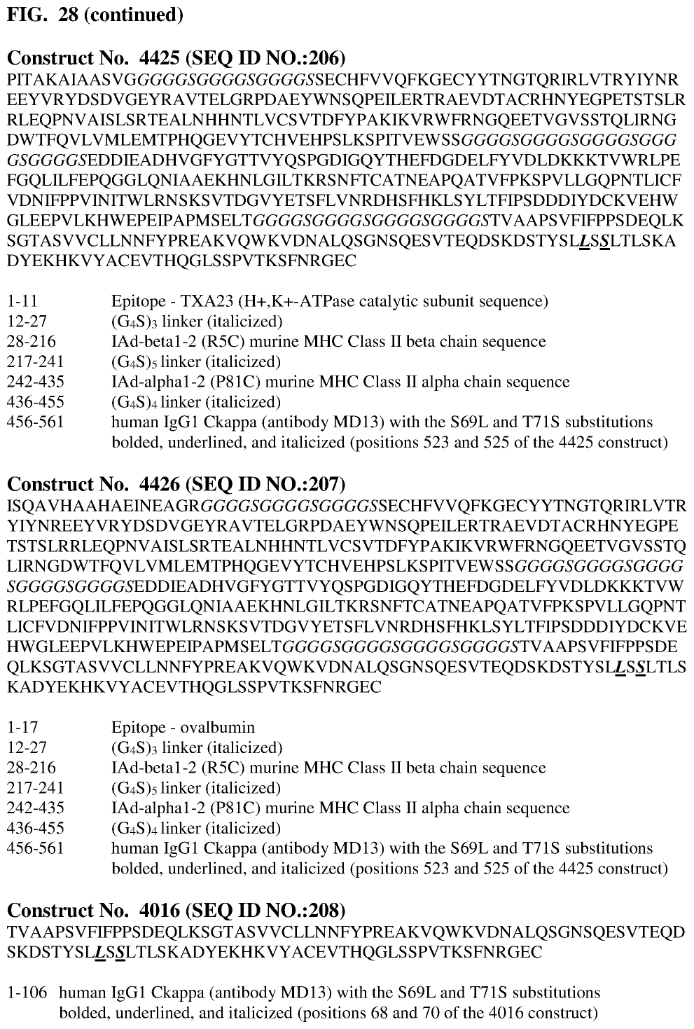

Linkers that are selected independently may be used to join the TGF-β and TβR sequences to the framework polypeptides. See e.g., Example 1, FIGS. 27 and 28.
c. IL-2 and its Variants

As one non-limiting example, a MOD or variant MOD present in a MAPP is an IL-2 or variant IL-2 polypeptide. In some cases, a variant MOD present in a MAPP is a variant IL-2 polypeptide. Wild-type IL-2 binds to an IL-2 receptor (IL-2R). A wild-type IL-2 aa sequence can be as follows:

(aa 21-153 of UniProt P60568, SEQ ID NO: 116)
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT.

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ are provided in the accompanying sequence listing as SEQ ID NO:117, SEQ ID NO:118 and SEQ ID NO:119 respectively, and are also provided in, for example, U.S. Patent Pub. No. 20200407416.

In some cases, a variant IL-2 polypeptide exhibits reduced binding affinity to one or more of the IL-2Rα, IL2Rβ, and/or IL-2Rγ chains of human IL-2R, compared to the binding affinity of an IL-2 polypeptide comprising the aa sequence set forth in SEQ ID NO:116. For example, in some cases, a variant IL-2 polypeptide binds to one or more of the IL-2Rα, IL2Rβ, and/or IL-2Rγ chains of human IL-2R with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an IL-2 polypeptide comprising the aa sequence set forth in SEQ ID NO: 116 for the α, β, and/or γ chains of IL-2R (e.g., an IL-2R comprising polypeptides comprising the aa sequence set forth in SEQ ID NOs: 117-119), when assayed under the same conditions.

For example, IL-2 variants with a substitution of phenylalanine at position 42 (e.g., with an alanine), exhibit substantially reduced binding to the IL-2Rα chain, in which case the variant may reduce the activation of Tregs. IL-2 variants with a substitution of histidine at position 16 (e.g., with an alanine) exhibit reduced binding to the IL2Rβ chain, thereby reducing the likelihood of a MAPP binding to non-target T cells by virtue of off-target binding of the IL-2 MOD. Some IL-2 variants, e.g., those with substitutions of the F42 and H16 amino acids, exhibit substantially reduced binding to the IL-2Rα chain and also reduced binding to the IL2Rβ chain. See, e.g., Quayle, et al., Clin Cancer Res; 26(8) Apr. 15, 2020.

In some cases, a variant IL-2 polypeptide has a single aa substitution compared to the IL-2 aa sequence set forth in SEQ ID NO:116. In some cases, a variant IL-2 polypeptide has from 2 to 10 aa substitutions compared to the IL-2 aa sequence set forth in SEQ ID NO:116. In some cases, a variant IL-2 polypeptide has 2, 3, 4, 5, 6, 7, 8, 9 or 10 aa substitutions compared to the IL-2 aa sequence set forth in SEQ ID NO:116. In some cases, a variant IL-2 polypeptide has 2 or 3 aa substitutions compared to the IL-2 aa sequence set forth in SEQ ID NO:116.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:116. Potential amino acids where substitutions may be introduced include one or more of the following positions:
(i) position 15, where the aa is other than E (e.g., A);
(ii) position 16, where the aa is other than H (e.g., A, T, N, C, Q, M, V or W);
(iii) position 20 is an aa other than D (e.g., A);
(iv) position 42, where the aa is other than F (e.g., A, M, P, S, T, Y, V or H);
(v) position 45, where the aa is other than Y (e.g., A);
(vi) position 88, where the aa is other than N (e.g., A or R);
(vii) position 126, where the aa is other than Q (e.g., A);

Combinations of the above substitutions include (H16X, F42X), (D20X, F42X), (E15X, D20X, F42X), (an H16X, D20X, F42X), (H16X, F42X, R88X), (H16X, F42X, Q126X), (D20X, F42X, Q126X), (D20X, F42X, and Y4X), (H16X, D20X, F42X, and Y45X), (D20X, F42X, Y45X, Q126X), (H16X, D20X, F42X, Y45X, Q126X), where X is the substituted aa, optionally chosen from the following: positions 15, 20, 45, 126—A; position 16—A or T, or also N, C, Q, M, V or W; position 42—A, or also M, P, S, T, Y, V or H; position 88—A or R.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:116, wherein the aa at position 16 is an aa other than H. In one case, the position of H16 is substituted by Asn, Cys, Gln, Met, Val, or Trp. In one case, the position of H16 is substituted by Ala. In another case, the position of H16 is substituted by Thr. Additionally, or alternatively, IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO: 116, wherein the aa at position 42 is an aa other than F. In one case, the position of F42 is substituted by Met, Pro, Ser, Thr, Trp, Tyr, Val, or His. In one case, the position of F42 is substituted by Ala.

IL-2 variants include polypeptides comprising an aa sequence comprising all or part of human IL-2 polypeptide having a substitution at position H16 and/or F42 (e.g., H16A and/or F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:116, wherein the aa at position 16 is an aa other than H and the aa at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In a second case, the position of H16 is substituted by Thr and the position of F42 is substituted by Ala (an H16T and F42A variant). In a third case, the position of H16 is substituted by Ala and the position of F42 is substituted by Thr (an H16A and F42T variant). In a fourth case, the position of H16 is substituted by Thr and the position of F42 is substituted Thr Ala (an H16T and F42T variant). As noted above, such variants will exhibit reduced binding to both the human IL-2Rα chain and IL2Rβ chain.

In any of the wild-type or variant IL-2 sequences provided herein, the cysteine at position 125 may be substituted with an aa other than cystine, such as alanine (a C125A substitution). In addition to any stability provided by the substitution, it may be employed where, for example, an epitope containing peptide or additional peptide is to be conjugated to a cysteine residue elsewhere in a MAPP, thereby avoiding competition from the C125 of the IL-2 MOD sequence.

d. PD-L1 and its Variants

As one non-limiting example, a MOD or variant MOD present in a MAPP is a PD-L1 or variant PD-L1 polypeptide. Wild-type PD-L1 binds to PD1.

A wild-type human PD-L1 polypeptide can comprise the following aa sequence: MRIFAVFIFM TYWHLLNAFT VTVPKDLYVV EYGSNMTIEC KFPVEKQLDL AALIVYWEME DKNIIQFVHG EEDLKVQHSS YRORARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO:120); where aas 1-18 form the signal sequence, aas 19-127 form the Ig-like V-type or IgV domain, and 133-225 for the Ig-like C2 type domain.

A wild-type human PD-L1 ectodomain aa sequence can comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO: 121); where aas 1-109 form the Ig-like V-type or “IgV” domain, and aas 115-207 for the Ig-like C2 type domain.

A wild-type human PD-L1 ectodomain aa sequence can also comprise the following aa sequence: FT VTVPKDLYVV EYGSNMTIEC KFPVEKQLDL AALIVYWEME DKNIIQFVHG EEDLKVQHSS YRORARLLKD QLSLGNAALQ ITDVKLQDAG VYRCMISYGG ADYKRITVKV NAPYNKINQR ILVVDPVTSE HELTCQAEGY PKAEVIWISS DHQVLSGKTT TTNSKREEKL FNVTSTLRIN TTTNEIFYCT FRRLDPEENH TAELVIPELP LAHPPNER LNVSIKI (SEQ ID NO: 122); where aas 1-109 form the Ig-like V-type or “IgV” domain, and aas 115-207 for the Ig-like C2 type domain. See e.g., NCBI Accession and version 3BIK_A, which includes an N-terminal alanine as its first aa.

A wild-type PD-L1 IgV domain, suitable for use as a MOD may comprise aa 18 and aas IgV aas 19-127 of SEQ ID NO:120, and a carboxyl terminal stabilization sequences, such as for instance the last seven aas (bolded and italicized) of the sequence: A FTVTVPKDLY VVEYGSNMTI ECKFPVEKQL DLAALIVYWE MEDKNIIQFV HGEEDLKTQH SSYRQRARLL KDQLSLGNAA LOITDVKLQD AGVYRCMISY GGADYKRITV KVNAPYAAAL HEH (SEQ ID NO:123). Where the carboxyl stabilizing sequence comprises a histidine (e.g., a histidine approximately 5 residues to the C-terminal side of the Tyr (Y) appearing as aa 117 of SEQ ID NO:123) to about aa 122, the histidine may form a stabilizing electrostatic bond with the backbone amide at aas 82 and 83 (bolded and italicized in SEQ ID NO:120 (Q107 and L106 of SEQ ID NO:120). As an alternative, a stabilizing disulfide bond may be formed by substituting one of aas 82 or 83) (Q107 and L106 of SEQ ID NO: 120) and one of aa residues 121, 122, or 123 (equivalent to aa positions 139-141 of SEQ ID NO:120).

A wild-type PD-1 polypeptide can comprise the following aa sequence: PGWFLDSPDR PWNPPTFSPA LLVVTEGDNA TFTCSESNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:124).

In some cases, a variant PD-L1 polypeptide (e.g., a variant of SEQ ID NO:121 or PD-L1's IgV domain) exhibits reduced binding affinity to PD-1 (e.g., a PD-1 polypeptide comprising the aa sequence set forth in SEQ ID NO:124), compared to the binding affinity of a PD-L1 polypeptide comprising the aa sequence set forth in SEQ ID NO:120 or SEQ ID NO: 121. For example, in some cases, a variant PD-L1 polypeptide binds PD-1 (e.g., a PD-1 polypeptide comprising the aa sequence set forth in SEQ ID NO:124) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:120 or SEQ ID NO:121.

### e. 4-1BBL and its Variants

In some cases, a wild-type and/or a variant 4-1BBL MOD polypeptide sequence is present as a MOD in a MAPP. Wild-type 4-1BBL binds to 4-1BB (CD137).

A wild-type 4-1BBL aa sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWOLTQ GATVLGLFRV TPEIPAGLPS PRSE (SEQ ID NO:125). NCBI Reference Sequence: NP_003802.1, where aas 29-49 are a transmembrane region.

In some cases, a variant 4-1BBL polypeptide is a variant of the tumor necrosis factor (TNF) homology domain (THD) of human 4-1BBL. A wild-type aa sequence of the THD of human 4-1BBL can comprise, e.g., one of SEQ ID NOs:126-128, as follows:

(SEQ ID NO: 126)
PAGLLDLROG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
(SEQ ID NO: 127)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
and
(SEQ ID NO: 128)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB aa sequence can be as follows: MGNSCYNIVA TLLLVLNFER TRSLQDPCSN CPAGTFCDNN RNQICSPCPP NSFSSAGGQR TCDICROCKG VERTRKECSS TSNAECDCTP GFHCLGAGCS MCEQDCKQGQ ELTKKGCKDC CFGTENDOKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKOPFMR PVQTTQEEDG CSCRFPEEEE GGCEL (SEQ ID NO:129).

A variant 4-1BBL polypeptide exhibits reduced binding affinity to 4-1BB, compared to the binding affinity of a 4-1BBL polypeptide comprising the aa sequence set forth in one of SEQ ID NOs:126-128. For example, a variant 4-1BBL polypeptide may bind 4-1BB with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a 4-1BBL polypeptide comprising the aa sequence set forth in one of SEQ ID NOs:126-128 for a 4-1BB polypeptide (e.g., a 4-1BB polypeptide comprising the aa sequence set forth in SEQ ID NO: 129), when assayed under the same conditions.

4-1BBL variants suitable for use as a MOD in a MAPP include those polypeptides with at least one aa substitution having at least 90%, at least 95%, at least 98%, or at least 99% aa sequence identity to one of SEQ ID NOs: 126, 127 or 128.

4-1BBL variants suitable for inclusion in a MAPP include those with at least one aa substitution (e.g., two, three, or four substitutions) include those having at least 90%, at least 95%, at least 98%, or at least 99% aa sequence identity to at least 140 (e.g., at least 160, 175, 180, or 181) contiguous aas of SEQ ID NO:126.

### 6. Linkers

As noted above, a MAPP can include a linker sequence (aa, peptide, or polypeptide linker sequence) or “linker” interposed between any two elements of a MAPP, e.g., an epitope and an MHC polypeptide; between an MHC polypeptide and an Ig Fc polypeptide; between a first MHC polypeptide and a second MHC polypeptide; etc. Although termed “linkers,” sequences employed for linkers may also be placed at the N- and/or C-terminus of a MAPP polypeptide to, for example, stabilize the MAPP polypeptide or protect it from proteolytic degradation.

Suitable polypeptide linkers (also referred to as “spacers”) are known in the art and can be readily selected and can be of any of a number of suitable lengths, e.g., from 2 to 50 aa in length, e.g., from 2 aa to 10 aa, from 10aa to 20 aa, 20 aa to 30 aa, from 30 aa to 40aa, from 40aa to 50aa, or longer than 50aa. In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 aa in length. Linkers can be generally classified into three groups, i.e., flexible, rigid and cleavable. See, e.g., Chen et al. (2013) Adv. Drug Deliv. Rev. 65:1357; and Klein et al. (2014) Protein Engineering, Design & Selection 27:325. Unless stated otherwise, the linkers employed in the MAPPs of this disclosure are not the cleavable linkers generally known in the art.

Polypeptide linkers in the MAPP may include, for example, polypeptides that comprise, consist essentially of, or consists of: i) Gly and Ser; ii) Ala and Ser; iii) Gly, Ala, and Ser; iv) Gly, Ser, and Cys (e.g., a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers (including, for example polymers comprising the sequences GSGGS (SEQ ID NO:130) or GGGS (SEQ ID NO:131), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used; both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:132), GGSGG (SEQ ID NO:133), GSGSG (SEQ ID NO:134), GSGGG (SEQ ID NO:135), GGGSG (SEQ ID NO:136), GSSSG (SEQ ID NO:137), any which may be repeated from 1 to 10 times (e.g., repeated 1, 2, 3, 4, 5, 6, 7, 8, 9, or 10 times), or combinations thereof, and the like. Linkers can also comprise the sequence Gly(Ser)₄ (SEQ ID NO:138) or (Gly)₄Ser (SEQ ID NO:99), either of which may be repeated from 1 to 10 times (e.g., 1, 2, 3, 4, 5, 6, 7, 8, 9, or 10 times). In one embodiment the linker comprises the aa sequence AAAGG (SEQ ID NO:139), which may be repeated from 1 to 10 times.

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the MAPP. Rigid peptide linkers include peptide linkers rich in proline, and peptide linkers having an inflexible helical structure, such as an α-helical structure. Examples of rigid peptide linkers include, e.g., (EAAAK)n (SEQ ID NO:140), A(EAAAK)nA (SEQ ID NO:141), A(EAAAK)nALEA(EAAAK)nA (SEQ ID NO:142), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:143) include EAAAK (SEQ ID NO:143). (EAAAK): (SEQ ID NO:144), (EAAAK); (SEQ ID NO:145). A(EAAAK)₄ALEA(EAAAK)₄A (SEQ ID NO:146), and AEAAAKEAAAKA (SEQ ID NO:147). Non-limiting examples of suitable rigid linkers comprising (AP)n include PAPAP (SEQ ID NO: 148; also referred to herein as “(AP)2”); APAPAPAP (SEQ ID NO:149; also referred to herein as “(AP)4”); APAPAPAPAPAP (SEQ ID NO:150; also referred to herein as “(AP)6”); APAPAPAPAPAPAPAP (SEQ ID NO:151; also referred to herein as “(AP)8”); and APAPAPAPAPAPAPAPAPAP (SEQ ID NO: 152; also referred to herein as “(AP)10”). Non-limiting examples of suitable rigid linkers comprising (KP)n include KPKP (SEQ ID NO:153; also referred to herein as “(KP)2”); KPKPKPKP (SEQ ID NO:154; also referred to herein as “(KP)4”); KPKPKPKPKPKP (SEQ ID NO:155; also referred to herein as “(KP)6”); KPKPKPKPKPKPKPKP (SEQ ID NO:156; also referred to herein as “(KP)8”); and KPKPKPKPKPKPKPKPKPKP (SEQ ID NO:157; also referred to herein as “(KP)10”). Non-limiting examples of suitable rigid linkers comprising (EP)n include EPEP (SEQ ID NO: 158; also referred to herein as “(EP)2”); EPEPEPEP (SEQ ID NO:159; also referred to herein as “(EP)4”); EPEPEPEPEPEP (SEQ ID NO: 160; also referred to herein as “(EP)6”); EPEPEPEPEPEPEPEP (SEQ ID NO:161; also referred to herein as “(EP)8”); and EPEPEPEPEPEPEPEPEPEP (SEQ ID NO:162; also referred to herein as “(EP)10”).

In some cases, a linker polypeptide, present in a polypeptide of a MAPP includes a cysteine residue that can form a disulfide bond with a cysteine residue present in another polypeptide of the MAPP. In some cases, for example, the linker comprises an aa sequence selected from (CGGGS), (GCGGS), (GGCGS), (GGGCS), and (GGGGC) with the rest of the linker comprised of Gly and Ser residues (e.g., GGGGS units that may be repeated from 1 to 10 times, such as 1, 2, 3, 4, 5, 6, 7, 8, 9, or 10 times). Cysteine containing linkers may also be selected from the sequences GCGASGGGGSGGGGS (SEQ ID NO:163), GCGGSGGGGSGGGGSGGGGS (SEQ ID NO:100), and GCGGSGGGGSGGGGS (SEQ ID NO:164).

Accordingly, the linker to which an epitope is attached may be from about 5 to about 50 aas in length. The linker to which an epitope may be attached may, for example be from about 5 to about 50 aas in length and comprise more than 50% Gly and Ser residues with one cysteine residue. The linker to which an epitope may be attached may be from about 5 to about 50 aas in length and comprise more than 50% (Gly)₄S repeats with one optional cysteine residue. The linker to which an epitope may be attached may be a (Gly)₄S sequence repeated from 3 to 8 (e.g., 3 to 7) times, optionally having one aa replaced by a cysteine residue.

### 7. Epitopes

A variety of peptide epitopes (also referred to herein as “epitopes”) may be present in a MAPP or higher order complexes of MAPPs (such as duplex MAPPs), and presentable to a TCR on the surface of a T cell.

A peptide epitope present in a MAPP (e.g., a duplex MAPP) is designed to be specifically bound by a target T cell that has a T cell receptor (“TCR”) that is specific for the epitope and which specifically binds the peptide epitope of the MAPP. An epitope-specific T cell thus binds a peptide epitope having a reference aa sequence, but substantially does not bind an epitope that differs from the reference aa sequence.

a. Peptide Epitopes in MAPPs with Class II MHC Presenting Sequences and Presenting Complexes

Among the epitopes that may be bound and presented to a TCR by a MAPP with Class II MHC presenting sequences or Class II MHC presenting complexes are T1D-associated peptide epitopes derived from of self antigens associated with T1D. A Type 1 Diabetes-associated epitope (also referred to herein as a “T1D peptide epitope” or “T1D epitope”) present in a MAPP presents a T1D-associated peptide epitope to a TCR on the surface of a T cell.

A T1D peptide epitope can have a length of from about 4 aas to about 25 aas (aa), e.g., the epitope can have a length of from 5 aa to 10 aa, from 10 aa to 15 aa, from 15 aa to 20 aa, or from 20 aa to 25 aa. For example, a T1D epitope present in a MAPP can have a length of 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, a T1D peptide epitope present in a MAPP has a length of from 10 aa to 20 aa, e.g., 10 aa, 11 aa, 12 aa, 13 aa, 14 aa, 15 aa, 16 aa, 17 aa, 18 aa, 19 aa and 20 aa.

Antigens associated with type 1 diabetes (T1D) include, e.g., preproinsulin, proinsulin, insulin, insulin B chain, insulin A chain, proinsulin C peptide, 65 kilodaltons (kDa) isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. An antigen “associated with” a particular autoimmune disorder is an antigen that is a target of autoantibodies and/or autoreactive T cells present in individuals with that autoimmune disorder, T1D in this instance, where such autoantibodies and/or autoreactive T cells mediate a pathological state associated with the autoimmune disorder. A suitable T1D peptide epitope for inclusion in a MAPP can be a peptide epitope of from 4 aas to about 25 aas in length of any one of the aforementioned T1D-associated antigens.

As one non-limiting example, a T1D peptide epitope is proinsulin 73-90 (GAGSLQPLALEGSLQKR; SEQ ID NO:165). As another non-limiting example, a T1D peptide epitope is the following insulin (InsA (1-15) peptide: GIVDQCCTSICSLYQ (SEQ ID NO: 166). As another non-limiting example, a T1D peptide epitope is the following insulin (InsA(1-15; D4E) peptide: GIVEQCCTSICSLYQ (SEQ ID NO:167). As another non-limiting example, a T1D peptide epitope is the following GAD65 (555-567) peptide: NFFRMVISNPAAT (SEQ ID NO:168). As another non-limiting example, a T1D peptide epitope is the following GAD65 (555-567; F5571) peptide: NFIRMVISNPAAT (SEQ ID NO:169). As another non-limiting example, a T1D peptide epitope is the following islet antigen 2 (IA2) peptide: SFYLKNVQTQETRTLTQFHF (SEQ ID NO: 170). As another non-limiting example, a T1D peptide epitope is the following proinsulin peptide: SLQPLALEGSLQSRG (SEQ ID NO:171). As another non-limiting example, a T1D peptide epitope is the following proinsulin peptide GSLQPLALEGSLQSRGIV (SEQ ID NO:172; proIns 75-92(K88S)).

In some cases, a suitable T1D peptide epitope comprises from 4 to 25 contiguous aas of an aa sequence having at least 90%, at least 95%, at least 98%, at least 99%, or 100%, aa sequence identity to aas 25-110 of the following human preproinsulin aa sequence (wherein italicized aas 1-24 form the signal peptide): MALWMRLLPL LALLALWGPD PAAA FVNQHL CGSHLVEALY LVCGERGFFY TPKTRREAED LQVGQVELGG GPGAGSLQPL ALEGSLQKRG IVEQCCTSIC SLYQLENYCN (SEQ ID NO:173); where the T1D peptide epitope has a length of 4 aas, 5 aas, 6 aas, 7, aas, 8 aas, 9 aas, 10 aas, 11 aas, 12 aas, 13 aas, 14 aas, 15 aas, 16 aas, 17 aas, 18 aas, 19 aas, 20 aas, 21 aas, 22 aas, 23 aas, 24 aas, or 25 aas. In some cases, the T1D peptide epitope has the aa sequence: GAGSLQPLALEGSLQKRG (SEQ ID NO:174) (proIns 73-90). In some cases, the T1D peptide epitope has the aa sequence: SLQPLALEGSLQKRG (SEQ ID NO:175) (proIns 76-90). In some cases, the T1D peptide epitope has the aa sequence: SLQPLALEGSLQSRG (SEQ ID NO:176) (proIns 76-90; K88S). In some cases, the T1D peptide epitope has the aa sequence: QPLALEGSLQKRG (SEQ ID NO:177). In some cases, the T1D peptide epitope has the aa sequence: QPLALEGSLQSRG (SEQ ID NO:178).

### 8. Additional Polypeptides

A polypeptide chain of a MAPP (e.g., a dimerization or framework polypeptide) may include one or more polypeptides in addition to those described above. Suitable additional polypeptides include epitope tags and affinity domains. The one or more additional polypeptides can be included at the N-terminus of a polypeptide chain of a MAPP, at the C-terminus of a polypeptide chain of a MAPP, or within (internal to) a polypeptide chain of a MAPP.

a. Affinity Tags and Affinity Domains

Suitable affinity tags/polypeptide affinity domains include, but are not limited to, hemagglutinin (HA; e.g., YPYDVPDYA (SEQ ID NO:179); FLAG (e.g., DYKDDDDK (SEQ ID NO:180); c-myc (e.g., EQKLISEEDL; SEQ ID NO:181), and the like.

Affinity tags/domains include peptide sequences that can interact with a binding partner, e.g., such as one immobilized on a solid support, useful for identification or purification. DNA sequences encoding multiple consecutive single aas, such as histidine, when fused to the expressed protein, may be used for one-step purification of the recombinant protein by high affinity binding to a resin column, such as nickel Sepharose. Exemplary affinity tags/domains include HisX5 (HHHHH) (SEQ ID NO:182). HisX6 (HHHHHH) (SEQ ID NO: 183), C-myc (EQKLISEEDL) (SEQ ID NO:181). Flag (DYKDDDDK) (SEQ ID NO:180). StrepTag (WSHPQFEK) (SEQ ID NO:184, hemagglutinin, e.g., HA Tag (YPYDVPDYA) (SEQ ID NO:179), glutathione-S-transferase (GST), thioredoxin, cellulose binding domains. RYIRS (SEQ ID NO:185). FHHT (SEQ ID NO:186), chitin binding domains. S-peptide. T7 peptide. SH2 domains. C-end RNA tag. WEAAAREACCRECCARA (SEQ ID NO:187), metal binding domains, e.g., zinc binding domains or calcium binding domains such as those from calcium-binding proteins, e.g., calmodulin, troponin C, calcineurin B, myosin light chain, recoverin. S-modulin, visinin, VILIP, neurocalcin, hippocalcin, frequenin, caltractin, calpain large-subunit. S100 proteins, parvalbumin. calbindin D9K, calbindin D28K, calretinin, inteins, biotin, streptavidin, MyoD, Id, leucine zipper sequences, and maltose binding protein.

### D. Nucleic Acids

The present disclosure provides a nucleic acid comprising a nucleotide sequence encoding one or more polypeptides of a MAPP bearing a T1D associated epitope. In some cases, the nucleic acid is a recombinant expression vector; thus, the present disclosure provides a recombinant expression vector comprising a nucleotide sequence encoding a.

1. Nucleic Acids Encoding a MAPP or MAPP Forming a Higher Order Complex, Such as a Duplex MAPP, that Comprises at Least One Dimerization Sequence and a Multimerization Sequence

The present disclosure provides nucleic acids comprising a nucleotide sequence encoding a MAPP having a framework polypeptide that comprises at least one dimerization sequence and at least one multimerization sequence that permits two molecules of the framework polypeptide to form dimers or higher order complexes. The nucleic acids may additionally comprise a nucleotide sequence encoding a dimerization peptide. Where the MAPP comprises a presenting sequence the nucleic acids encoding either or both of the framework polypeptide and/or dimerization peptide may include a sequence encoding a presenting sequence. Where the MAPP comprises a presenting complex, the nucleic acids encoding either or both of the framework polypeptide and/or dimerization peptide may further comprise sequences encoding a presenting complex 1^{st} sequence and/or a presenting complex 2^{nd} sequence. The nucleic acid sequences encoding MAPPs may also encode a peptide epitope. The nucleotide sequence(s) comprising any of the MAPP polypeptides can be operably linked to a transcription control element(s). e.g., a promoter. It will be apparent that individual polypeptides of a MAPP (e.g., a framework polypeptide and dimerization polypeptide) may be encoded on a single nucleic acid (e.g., under the control of separate promoters), or alternatively, may be located on two or more separate nucleic acids (e.g., plasmids).

### 2. Recombinant Expression Vectors

The present disclosure provides recombinant expression vectors comprising nucleic acids encoding one or more polypeptides of a MAPP or its higher order complexes. In some cases, the recombinant expression vector is a non-viral vector. In some cases, the recombinant expression vector is a viral construct, such as a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (sec, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (sec, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol. 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In some cases, a nucleotide sequence encoding one or more polypeptides of a MAPP is operably linked to a control element, e.g., a transcriptional control element, such as a promoter. The transcriptional control element may be functional in either a eukaryotic cell, e.g., a mammalian cell such as a human, hamster, or mouse cell; or a prokaryotic cell (e.g., bacterial). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include the cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### E. Genetically Modified Host Cells

The present disclosure provides a genetically modified host cell, where the host cell is genetically modified with a nucleic acid(s) that encode, or encode and express, MAPP proteins or higher order complexes of MAPPs (e.g., duplex MAPPs).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2),™), CHO cells (e.g., ATCC Nos. CRL9618, CCL61, CRL-9618™, CCL-61™, CRL9096), 293 cells (e.g., ATCC No. CRL-1573),™), Vero cells, NIH 3T3 cells (e.g., ATCC No. CRL-1658), Huh-7 cells, BHK cells (e.g., ATCC No. CCL10), CCL-10™), PC12 cells (ATCC No. CRL1721), CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. In some cases, the host cell is a mammalian cell that has been genetically modified such that it does not synthesize endogenous MHC Class II heavy chains (MHC-H).

Genetically modified host cells can be used to produce a MAPP and higher order complexes of MAPPs. For example, a genetically modified host cell can be used to produce a duplex MAPP. For example, an expression vector(s) comprising nucleotide sequences encoding the MAPP polypeptide(s) is/are introduced into a host cell, generating a genetically modified host cell, which genetically modified host cell produces the polypeptide(s) (e.g., as an excreted soluble protein).

### F. Methods of Producing MAPPs

The present disclosure provides methods of producing MAPPs (e.g., duplex MAPPs) with at least one masked TGF-β MOD presenting a T1D-associated peptide epitope. The methods generally involve culturing, in a culture medium, a host cell that is genetically modified with a recombinant expression vector(s) comprising a nucleotide sequence(s) encoding the MAPP (e.g., a genetically modified host cell of the present disclosure); and isolating the MAPP from the genetically modified host cell and/or the culture medium. As noted above, in some cases, the individual polypeptide chains of a MAPP are encoded in separate nucleic acids (e.g., recombinant expression vectors). In some cases, all polypeptide chains of a MAPP are encoded in a single recombinant expression vector.

Isolation of the MAPP from the host cell employed for expression (e.g., from a lysate of the expression host cell) and/or the culture medium in which the host cell is cultured, can be carried out using standard methods of protein purification. For example, a lysate of the host cell may be prepared, and the MAPP purified from the lysate using high performance liquid chromatography (HPLC), exclusion chromatography (e.g., size exclusion chromatography), gel electrophoresis, affinity chromatography, or other purification technique. Alternatively, where the MAPP is secreted from the expression host cell into the culture medium, the MAPP can be purified from the culture medium using HPLC, exclusion chromatography, gel electrophoresis, affinity chromatography, or other purification technique. In some cases, the MAPP is purified, e.g., a composition is generated that comprises at least 80% by weight, at least about 85% by weight, at least about 95% by weight, or at least about 99.5% by weight, of the MAPP in relation to contaminants related to the method of preparation of the product and its purification. The percentages can be based upon total protein.

In some cases, e.g., where the expressed MAPP comprises an affinity tag or affinity domain, the MAPP can be purified using an immobilized binding partner of the affinity tag. For example, where a MAPP comprises an Ig Fc polypeptide, the MAPP can be isolated from genetically modified mammalian host cell and/or from culture medium comprising the MAPP by affinity chromatography, e.g., on a Protein A column, a Protein G column, or the like. An example of a suitable mammalian cell is a CHO cell; e.g., an Expi-CHO-S™ cell (e.g., ThermoFisher Scientific, Catalog #A29127).

The polypeptides of the MAPP will self-assemble into heterodimers, and where applicable, spontaneously form disulfide bonds between, for example, framework polypeptides, or framework and dimerization polypeptides. As also noted above, when both framework polypeptides include Ig Fc polypeptides with suitable cysteines residues, disulfide bonds will spontaneously form between the respective Ig Fc polypeptides to covalently link the two heterodimers of framework and dimerization polypeptides to one another to form a covalently linked duplex MAPP.

### G. Compositions

### 1. Compositions Comprising a MAPP

The present disclosure provides compositions, including pharmaceutical compositions, comprising a MAPP and/or higher order complexes of MAPPs (e.g., duplex MAPPs). Pharmaceutical composition can comprise, in addition to a MAPP, one or more known carriers, excipients, diluents, buffers, salts, surfactants (e.g., non-ionic surfactants), amino acids (e.g., arginine), etc., a variety of which are known in the art and need not be discussed in detail herein. For example, see “Remington: The Science and Practice of Pharmacy”, 19^{th} Ed. (1995), or latest edition, Mack Publishing Co.

In some cases, a subject pharmaceutical composition will be suitable for administration to a subject, e.g., will be sterile and/or substantially free of pyrogens. For example, in some embodiments, a subject pharmaceutical composition will be suitable for administration to a human subject, e.g., where the composition is sterile and is substantially free of detectable pyrogens and/or other toxins, or such detectable pyrogens and/or other toxins are below a permissible limit.

The compositions may, for example, be in the form of aqueous or other solutions, powders, granules, tablets, pills, suppositories, capsules, suspensions, sprays, and the like. The composition may be formulated according to the various routes of administration described below.

Where a MAPP or higher order MAPP complex (e.g., duplex MAPP) is administered as an injectable (e.g., subcutaneously, intraperitoneally, intramuscularly, intralymphatically, and/or intravenously) directly into a tissue, a formulation can be provided as a ready-to-use dosage form, or as non-aqueous form (e.g., a reconstitutable storage-stable powder) or an aqueous form, such as liquid composed of pharmaceutically acceptable carriers and excipients. MAPPs may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a MAPP composition comprises: a) a MAPP higher order MAPP complex (e.g., a duplex MAPP); and b) saline (e.g., 0.9% NaCl). In some cases, the composition is sterile and/or substantially pyrogen free, or the amount of detectable pyrogens and/or other toxins are below a permissible limit. In some cases, the composition is suitable for administration to a human subject, e.g., where the composition is sterile and is free of detectable pyrogens and/or other toxins, or the amount of detectable pyrogens and/or other toxins are below a permissible limit. Thus, the present disclosure provides a composition comprising: a) a MAPP or higher order MAPP complex (e.g., duplex MAPP); and b) saline (e.g., 0.9% NaCl), where the composition is sterile and is substantially free of detectable pyrogens and/or other toxins, or such detectable pyrogens and/or other toxins are below a permissible limit.

Other examples of components suitable for inclusion in formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. A pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a MAPP in a formulation can vary widely. For example, a MAPP or higher order MAPP complex (e.g., duplex MAPP) may be present from less than about 0.1% (usually at least about 2%) to as much as 20% to 50% or more by weight (e.g., from 1% to 10%, 5% to 15%, 10% to 20% by weight, or 20-50% by weight) by weight. The concentration will usually be selected primarily based on fluid volumes, viscosities, and patient-based factors in accordance with the particular mode of administration selected and the patient's needs.

The present disclosure provides a container comprising a composition, e.g., a liquid composition. The container can be, e.g., a syringe, an ampoule, and the like. In some cases, the container is sterile. In some cases, both the container and the composition are sterile and substantially free of detectable pyrogens and/or other toxins, or such detectable pyrogens and/or other toxins are below a permissible limit. A pharmaceutical composition or a container comprising a composition (e.g., pharmaceutical composition) set forth herein may be packaged as a kit. The kit may comprise, for example, the composition or the container comprising a composition along with instructions for use of those materials. Materials packaged as a kit may be sterile and/or substantially free of detectable pyrogens and/or other toxins, or such detectable pyrogens and/or other toxins are below a permissible limit.

### H. Methods of Utilizing MAPPs

MAPPs and higher order MAPP complexes (e.g., duplex MAPP) are useful for modulating an activity of a T cell. Thus, the present disclosure provides methods of modulating an activity of a T cell, the methods generally involving contacting a target T cell with a MAPP or a higher order MAPP complex (e.g., duplex MAPP).

### 1. Methods of Modulating T Cell Activity

The present disclosure provides a method of selectively modulating the activity of an epitope-specific T cell, the method comprising contacting the T cell with a MAPP comprising a T1D peptide epitope, where contacting the T cell with a MAPP selectively modulates the activity of the epitope-specific T cell. In some cases, the contacting occurs in vivo (e.g., in a mammal such as a human, rat, mouse, dog, cat, pig, horse, or primate). In some cases, the contacting occurs in vitro. In some cases, the contacting occurs in vivo.

In some cases, a MAPP reduces activity of an autoreactive T cell and/or an autoreactive B cell. In some cases, a MAPP increases the number and/or activity of a regulator T cell (Treg), resulting in reduced activity of an autoreactive T cell and/or an autoreactive B cell.

In some cases, a MAPP is contacted with an epitope-specific CD4⁺ T cell. In some cases, the epitope-specific T cell is a CD4⁺CD8⁺ (double positive) T cell (see e.g., Boher et al Front. Immunol., 29 Mar. 2019 on the www at: doi.org/10.3389/fimmu.2019.00622 and Matsuzaki et al. J. Immuno. Therapy of Cancer 7: Article number: 7 (2019)). In some cases, the epitope-specific T cell is a NK-T cell (see, e.g., Nakamura et al. J, Immunol. 2003 Aug. 1; 171(3):1266-71). In some cases, the epitope-specific T cell is a T (Treg). The contacting may result in modulating the activity of a T cell, which can result in, but is not limited to proliferation and/or maintenance of regulatory T cells (e.g., when IL-2 MOD polypeptides are present, the effect of which may be amplified by the presence of retinoic acids such as all trans retinoic acid).

In some cases, a MAPP is contacted with an epitope-specific CD4⁺ T cell. In some cases, the CD4⁺ T cell is a Th1 that produces, among other things, interferon gamma, and which may be a target for inhibition in autoimmunity. In some cases, the CD4⁺ T cell is a Th2 cell that produces, among other things, IL-4. Th2 cells may be inhibited to suppress autoimmune diseases such T1D. In some cases, the CD4⁺ T cell is a Th17 cell that produces, among other things, IL-17, and which may be inhibited to suppress autoimmune diseases such as T1D. In some cases, the CD4⁺ T cell is a Th9 cell that produces, among other things, IL-9, and which may be inhibited to suppress its actions in autoimmune conditions such as T1D. In some cases, the CD4⁺ T cell is a Tfh cell that produces, among other things, IL-21 and IL-4, and which may be inhibited to suppress autoimmune diseases such as T1D.

In some cases, the T cell being contacted with a MAPP is a regulatory T cell (Treg) that is CD4⁺, FOXP3⁺, and CD25⁺. Tregs can suppress autoreactive T cells.

The present disclosure provides a method of increasing proliferation of Tregs, the method comprising contacting Tregs with a MAPP, where the contacting increases proliferation of Tregs specific/selective for epitope presented by the MAPP. The present disclosure provides a method of increasing the number of epitope specific Tregs in an individual, the method comprising administering to the individual a MAPP, where the administering results in an increase in the number of Tregs specific to the epitope presented by the MAPP in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

In some cases, the cell being contacted with a MAPP is a helper T cell, where contacting the helper T cell with a MAPP inhibits or blocks the proliferation and/or differentiation of Th1 and/or Th2 cells specific/selective for the epitope presented by the MAPP by, for example, inhibiting the expression of the transcription factors T-bet and/or GATA3. The suppression of Th1 and/or Th2 cells results in the decreased activity and/or number effector cells such as CD8⁺ cytotoxic T cells specific to the epitope.

In some cases a MAPP interacts with T cells that are subject to IL-2 receptor activation provided either by an IL-2 MOD of the MAPP or IL-2 in the T cell environment resulting in: (i) activation, proliferation, or maintenance of T reg cells specific for the epitope presented by the MAPP; and/or (ii) suppression of epitope specific Th1 cell development; and/or (iii) suppression of epitope specific Th2 cell development; and/or (iv) suppression of epitope specific cytotoxic T lymphocyte (CTL) development. The addition of retinoic acid (e.g., all trans retinoic acid) may potentiate the action of the TGF-β-bearing MAPPs described herein a MAPP in any of those functions, particularly activation, proliferation, or maintenance of T reg cells where the MAPP bears one or more IL-2 MODs. Where the epitope is an TD1-associated epitope the MAPP can be utilized to suppress responses to the epitope and effect treatment of T1D.

MAPPs may interact with T cells in the presence of IL-2 and PD1 receptor agonist, either or both of which may be provided by IL-2 or PD-L1 MODs of the MAPP and/or IL-2 or PD-L1present in the T cell's environment during the interaction. Under such conditions the MAPP along with agonist of the IL-2 and PD1 receptors may regulate the development, maintenance, and function of Treg cells (e.g., induced regulatory T cells) specific for the epitope presented by the MAPP. See, e.g., Franciso et al., J Exp Med., 206(13):3015-3029 (2009). Accordingly, masked TGF-β MOD-bearing MAPPs along with agonist of the IL-2 receptor and PD1 receptor (e.g., a MAPP bearing one or more masked TGF-β MODs and additionally one or more IL-2 MODs and one or more PD-L1 MODs) may be employed to suppress immune responses to epitopes of autoantigens associate with T1D.

### 2. Treatment Methods

The present disclosure provides treatment methods, the methods comprising administering to the individual composition comprising an amount of a MAPP or higher order MAPP such as duplex MAPP, effective to selectively modulate the activity of a T1D epitope-specific T cell in an individual and to treat the individual. In some cases, a treatment method comprises administering to an individual in need thereof a pharmaceutical composition comprising an effective amount of a MAPP or higher order MAPP such as duplex MAPP useful for treating type 1 diabetes (T1D) occurring in human patients and in experimental animal models (e.g., non-obese diabetic (NOD) mouse and the Biobreeding (BB) rat).

The present disclosure provides a method of selectively modulating the activity of a T1D epitope-specific T cell in an individual, the method comprising administering to the individual a pharmaceutical composition comprising an effective amount of a MAPP or higher order MAPP such as duplex MAPP, where the MAPP selectively modulates the activity of the epitope-specific T cell in the individual. Selectively modulating the activity of an epitope-specific T cell can treat T1D in the individual. Thus, the present disclosure provides a treatment method comprising administering to an individual in need thereof a pharmaceutical composition comprising an effective amount of a MAPP or higher order MAPP such as duplex MAPP for the purpose of treating T1D.

The present disclosure provides a method of treating T1D in an individual, the method comprising administering to the individual a pharmaceutical composition comprising an effective amount of a MAPP or higher order MAPP such as a duplex MAPP, where the MAPP comprises a T1D peptide epitope (as described above), and where the MAPP comprises PD-L1. In some cases, an “effective amount” of a MAPP or higher order MAPP is an amount that, when administered in one or more doses to an individual in need thereof, reduces the number of self-reactive (i.e., reactive with aT1D-associated antigen) CD4+ and/or CD8+ T cells by, for example, at least 10%, at least 15%, at least 20%, at least 25%, at least 30%, at least 40%, at least 50%, at least 60%, at least 70%, at least 80%, at least 90%, or at least 95% (e.g., from 10% to 50%, or from 50% to 95%) compared to number of self-reactive T cells in the individual before administration of the MAPP, or in the absence of administration with the MAPP. In some cases, an “effective amount” of a MAPP is an amount that, when administered in one or more doses to an individual in need thereof, reduces production of Th1 cytokines (e.g., IL-2, IL-10, and TNF-alpha/beta) in the individual. In some cases, an “effective amount” of a MAPP is an amount that, when administered in one or more doses to an individual in need thereof, reduces production of Th2 cytokines (e.g., IL-4, IL-5, and/or IL-13) in the individual. In some cases, an “effective amount” of a MAPP is an amount that, when administered in one or more doses to an individual in need thereof, reduces production of Th17 cytokines (e.g., IL-17A, IL-17F, and/or IL-22) in the individual. In some cases, an “effective amount” of a MAPP is an amount that, when administered in one or more doses to an individual in need thereof, ameliorates one or more symptoms associated with T1D in the individual. In some instances, the MAPP reduces the number of CD4+ self-reactive T cells (i.e., the number of CD4+ T cells reactive with a T1D-associated antigen), which in turn leads to a reduction in CD8+ self-reactive T cells. In some instances, the MAPP increases the number or activity (e.g., IL-10 and/or TGF-β production) of CD4+ Tregs specific for a T1D peptide epitope presented by the MAPP, which in turn may reduce the number or activity of CD4+ self-reactive T cells, B cells, and/or CD8+ T self-reactive T cells specific for that epitopc.

The present disclosure provides a method of reducing elevated blood sugar (e.g., glucose) in an individual (e.g., a mammal such as a human) having or suspected of having T1D, the method comprising administering to the individual an effective amount of a MAPP, or one or more nucleic acids comprising nucleotide sequences encoding the MAPP, where the MAPP comprises a T1D peptide epitope (as described above). The individual may be a human having a fasting blood sugar in excess of 130 or about 140 mg/dL, or postprandial blood sugar in excess of 180 or about 200 mg/dL, and wherein the treatment reduces fasting blood sugar (e.g., such as to a level below 130 mg/dL) or post prandial blood sugar (e.g., to less than 180 mg/dL) in the individual relative to the level prior to receiving the MAPP. The reduction in blood sugar may be maintained for a period of at least about a week, at least about two weeks, at least about a month (30 days), or more than one month.

The present disclosure provides a method of reducing prediabetic glycosylated hemoglobin referred to as hemoglobin A1C (also referred to as hemoglobin A1c or HbA1c) levels (in the range of 5.7% to 6.4%) or diabetic hemoglobin A1C levels (above 6.4%) in an individual (e.g., a mammal such as a human) having or suspected of having T1D, the method comprising administering to the individual an effective amount of a MAPP, or one or more nucleic acids comprising nucleotide sequences encoding the MAPP, where the MAPP comprises a T1D peptide epitope (as described above). The treatment reduces diabetic hemoglobin A1C (e.g., to less than 6.4%, and preferably to less than 5.7%), or prediabetic A1C (e.g., such as to less than 5.7% and into the normal range) in the individual relative to the level prior to receiving the MAPP. The reduction in hemoglobin A1C may be maintained for a period of at least about a week, at least about two weeks, at least about a month (30 days), or more than one month.

### 3. Methods of Selectively Delivering a MOD

The present disclosure provides a method of delivering TGF-β either alone or in combination with a MOD polypeptide such as IL-2, 4-1BBL, PD-L1, or a reduced-affinity variant of any thereof (e.g., PD-L1 and/or an IL-2 variant disclosed herein) to a selected T cell or a selected T cell population, e.g., in a manner such that a TCR specific for a given T1D-associated epitope. As used herein, the phrases “selectively delivers” and “selectively provides” means that the majority of T cells for which the MAPP provides detectable TGF-β modulation comprise a TCR that specifically or preferentially binds the epitope of the MAPP.

The present disclosure thus provides a method of delivering TGF-β (masked TGF-β) and a MOD polypeptide such as a PD-L1 polypeptide, or a reduced-affinity variant of a naturally occurring MOD polypeptide such as a PD-L1 variant, selectively to a target T cell bearing a TCR specific for the T1D epitope presented by a MAPP (e.g., duplex MAPP). The present disclosure provides a method of delivering a TGF-β and an IL-2 MOD polypeptide sequence, or a reduced-affinity variant of IL-2, selectively to a target T cell bearing a TCR specific for the T1D epitope presented by a MAPP (e.g., duplex MAPP). The method comprises contacting a population of T cells with a MAPP (e.g., duplex MAPP). The population of T cells can be a mixed population that comprises: i) the target T cell with a TCR specific to a target epitope; and ii) non-target T cells that are not specific for the target epitope presented by the MAPP-associated peptide epitope (e.g., T cells that are specific for an epitope(s) other than the epitope to which the epitope-specific T cell binds). Epitope-specific T cells specific for the peptide epitope present in the MAPP (e.g., duplex MAPP) bind to the peptide MHC complex provided by the MAPP thereby delivering the TGF-β and any other additional MOD polypeptide in the MAPP ((e.g., PD-L1 or a reduced-affinity variant of PD-L1) selectively to the bound T cells.

Thus, the present disclosure provides a method of delivering TGF-β and an IL-2 MOD. PD-L1 MOD, and/or a reduced-affinity variant of IL-2 and/or PD-L1, to T cell selective for the T1D epitope presented by the MAPP. Similarly, the disclosure provides a method of delivering TGF-β, and an IL-2. MOD polypeptide and/or a reduced-affinity variant of a naturally occurring IL-2 MOD polypeptide to a target T cell that is selective for the T1D epitope presented by the MAPP. In some cases, the IL-2 MOD bears a substitution at position H16 and/or F42 (e.g., H16 and F42 such as H16A and F42A) (see supra SEQ ID NO:116).

For example, a MAPP or higher order MAPP complex (e.g., duplex MAPP) is contacted with a population of T cells comprising: i) target T cells that are specific for the epitope present in the MAPP or a higher order MAPP complex; and ii) non-target T cells, e.g., a T cells that are specific for a second epitope(s) that is not the epitope present in the MAPP or a higher order MAPP complex. Contacting the population results in substantially selective delivery of the TGF-β and any other MOD polypeptide(s) present in the MAPP (e.g., naturally-occurring or variant MOD polypeptide(s)) to the target T cell. Less than 50%, less than 40%, less than 30%, less than 25%, less than 20%, less than 15%, less than 10%, less than 5%, or less than 4%, 3%, 2% or 1%, of the MAPP or higher order MAPP complex (e.g., duplex MAPP) may bind to non-target T cells and, as a result, the masked TGF-β MOD polypeptide along with any additional MODs (e.g., wt. or variant IL-2. PD-L1 and/or 4-1BBL MODs) present in the MAPP are selectively delivered to target T cell (and accordingly, not effectively delivered to the non-target T cells).

The population of T cells to which a MOD and/or variant MOD is selectively delivered may be in vivo. In some cases, the population of T cells to which a MOD and/or variant MOD is selectively delivered is in vitro.

In some cases, the population of T cells to which a MOD and/or variant MOD is selectively delivered may be in vivo. In some cases, the population of T cells is in vitro. For example, a mixed population of T cells is obtained from an individual, and is contacted with a MAPP (e.g., duplex MAPP) in vitro. Such contacting, which can comprise single or multiple exposures of the T cells to one or more defined doses and/or exposure schedules in the context of in vitro cell culture, can be used to determine whether the mixed population of T cells includes T cells that are specific for the epitope presented by the MAPP. The presence of T cells that are specific for the epitope presented by the MAPP can be determined by assaying a sample comprising a mixed population of T cells, which population of T cells comprises T cells that are not specific for the epitope (non-target T cells) and may comprise T cells that are specific for the epitope (target T cells). Known assays can be used to detect the desired modulation of the target T cells, thereby providing an in vitro assay that can determine whether a particular MAPP (e.g., duplex MAPP) possesses an epitope that binds to T cells present in the individual, and thus whether the MAPP has potential use as a therapeutic composition for that individual. Suitable known assays for detection of the desired modulation (e.g., activation/proliferation or inhibition/suppression) of target T cells include, e.g., flow cytometric characterization of T cell phenotype, numbers, and/or antigen specificity. Such an assay to detect the presence of epitope-specific T cells, e.g., a companion diagnostic, can further include additional assays (e.g., effector cytokine ELISpot assays) and/or appropriate controls (e.g., antigen-specific and antigen-nonspecific multimeric peptide-HLA staining reagents) to determine whether the MAPP or higher order MAPP complex is selectively binding, modulating (activating or inhibiting), and/or expanding the target T cells. Thus, for example, the present disclosure provides a method of detecting, in a mixed population of T cells obtained from an individual, the presence of a target T cell that binds an epitope of interest, the method comprising: a) contacting in vitro the mixed population of T cells with a MAPP (e.g., duplex MAPP) comprising an epitope of the present disclosure; and b) detecting modulation (activation or inhibition) and/or proliferation of T cells in response to said contacting, wherein modulation of and/or proliferation of T cells indicates the presence of the target T cell. Alternatively, and/or in addition, if activation and/or expansion (proliferation) of the desired T cell population (e.g., Tregs) is obtained using a MAPP (e.g., a duplex MAPP), then all or a portion of the population of T cells comprising the activated/expanded T cells can be administered back to the individual as a therapy.

The population of T cells to be targeted by a MAPP may be in vivo in an individual. In such instances, a method of the present disclosure for selectively delivering TGF-β an any other MOD polypeptide (e.g., wt. or variant IL-2 polypeptides) to an epitope-specific T cell comprises administering the MAPP (e.g., duplex MAPP) to the individual.

In some instances, the epitope-specific T cell to which TGF-β and any other MOD polypeptide sequence present in the MAPP (e.g., a wild-type or reduced affinity IL-2 and/or PD-L1 MOD) is being selectively delivered is referred to herein is a target regulatory T cell (Treg) that may inhibit or suppresses activity of an autoreactive T cell.

### I. Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A MAPP (whether as a single heterodimer or, as described above, as a higher order complex such as a duplex MAPP) may be administered in amounts between 1 ng/kg body weight and 20 mg/kg body weight per dose; for example from 0.1 μg/kg body weight to 1.0 mg/kg body weight, from 0.1 mg/kg body weight to 0.5 mg/kg body weight, from 0.5 mg/kg body weight to 1 mg/kg body weight, from 1.0 mg/kg body weight to 5 mg/kg body weight, from 5 mg/kg body weight to 10 mg/kg body weight, from 10 mg/kg body weight to 15 mg/kg body weight, and from 15 mg/kg body weight to 20 mg/kg body weight. Doses below 0.1 mg/kg body weight or above 20 mg/kg are envisioned, especially considering the aforementioned factors. Amounts thus include from about 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight.

Those of skill will readily appreciate that dose levels can vary as a function of the MAPP or higher order MAPP complex (e.g., duplex MAPP), the severity of the symptoms and the susceptibility of the subject to side effects. Preferred dosages for a given compound are readily determinable by those of skill in the art by a variety of means.

In some cases, multiple doses of a MAPP or higher order MAPP complex (e.g., duplex MAPP) are administered. The frequency of administration of a MAPP or higher order MAPP complex (e.g., duplex MAPP) can vary depending on any of a variety of factors, e.g., severity of the symptoms, patient response, etc. For example, in some cases, a MAPP or higher order MAPP complex (e.g., duplex MAPP) is administered once per month, twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid).

The duration of administration of a MAPP, e.g., the period of time over which a MAPP is administered, can vary, depending on any of a variety of factors, e.g., patient response, etc. For example, a MAPP can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo periodic maintenance therapy to prevent the recurrence of the T1D disease state, wherein a MAPP is administered in maintenance doses, ranging from those recited above. i.e., 0.1 mg/kg body weight to about 0.5 mg/kg body weight, from about 0.5 mg/kg body weight to about 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight. The periodic maintenance therapy can be once per month, once every two months, once every three months, once every four months, once every five months, once every six months, or less frequently than once every six months.

### J. Routes of Administration

A MAPP or higher order MAPP complex is administered to an individual using any available method and route suitable for drug delivery, including in vivo and in vitro methods, as well as systemic and localized routes of administration. A MAPP or higher order MAPP complex can be administered to a host using any available conventional methods and routes suitable for delivery of conventional drugs, including systemic or localized routes. In general, routes of administration contemplated for use in a method include, but are not necessarily limited to, enteral, parenteral, and inhalational routes.

Conventional and pharmaceutically acceptable routes of administration include intramuscular, intratracheal, subcutaneous, intradermal, topical application, intravenous, intraarterial, rectal, nasal, oral, and other enteral and parenteral routes of administration. Of these, intravenous, intramuscular and subcutaneous may be more commonly employed. Routes of administration may be combined, if desired, or adjusted depending upon, for example, the MAPP or higher order MAPP complex (e.g., duplex MAPP) and/or the desired effect. A MAPP or higher order MAPP complex can be administered in a single dose or in multiple doses.

### K. Subjects Suitable for Treatment

Subjects suitable for treatment with a method include individuals who have T1D, including individuals who have been diagnosed as having T1D, and individuals who have been treated for T1D but who failed to respond to the treatment. Suitable subjects also may include individuals who have been diagnosed as being likely to develop T1D or who have symptoms indicating the imminent onset of T1D.

Subjects suitable for treatment include those with T1D or a genetic disposition to develop T1D including a family history of T1D (e.g., a grandparent, parent, or sibling with T1D). As discussed above, a number of serotypes have been associated with a substantial risk of developing T1D (e.g., DR3, DR4, DQ2.5 and DQ 8.1), and others with a moderate risk of developing T1D (e.g., DR1, DR8, DR9 and DQ5.

Subjects suitable for treatment include those who have T1D, including individuals who have been diagnosed as having T1D, and individuals who have been treated for T1D but who failed to respond to the treatment who have fasting blood sugars (blood sugar after not eating or drinking for 8 hours) in excess of about 130 mg/dL (or about 140 mg dL), and/or a blood sugar higher than about 180 mg/dl (or about 200 m/dL) 2 hours after a meal (postprandial hyperglycemia). This includes individuals with (i) a genetic disposition to T1D such as a family history of T1D and/or a serotype associated with T1D (e.g., DR4), and (ii) either an elevated fasting blood sugar in excess of 130 or about 140 mg/dL, or postprandial blood sugar in excess of 180 or about 200 mg/dL. See, e.g., www.cdc.gov/diabetes/managing/managing-blood-sugar/bloodglucosemonitoring.html.

Subjects suitable for treatment include those who have T1D, including individuals who have been diagnosed as having T1D, and individuals who have been treated for T1D but who failed to respond to the treatment who have hemoglobin A1C levels from 5.7 to 6.4% (prediabetic levels) or hemoglobin A1C levels above 6.4% (diabetic levels). See, e.g., www.cdc.gov/diabetes/managing/managing-blood-sugar/alc.html. This includes individuals with both prediabetic or diabetic A1C levels and a genetic disposition to T1D such as a family history of T1D and/or a serotype associated with T1D (e.g., DR4).

### V. CERTAIN ASPECTS

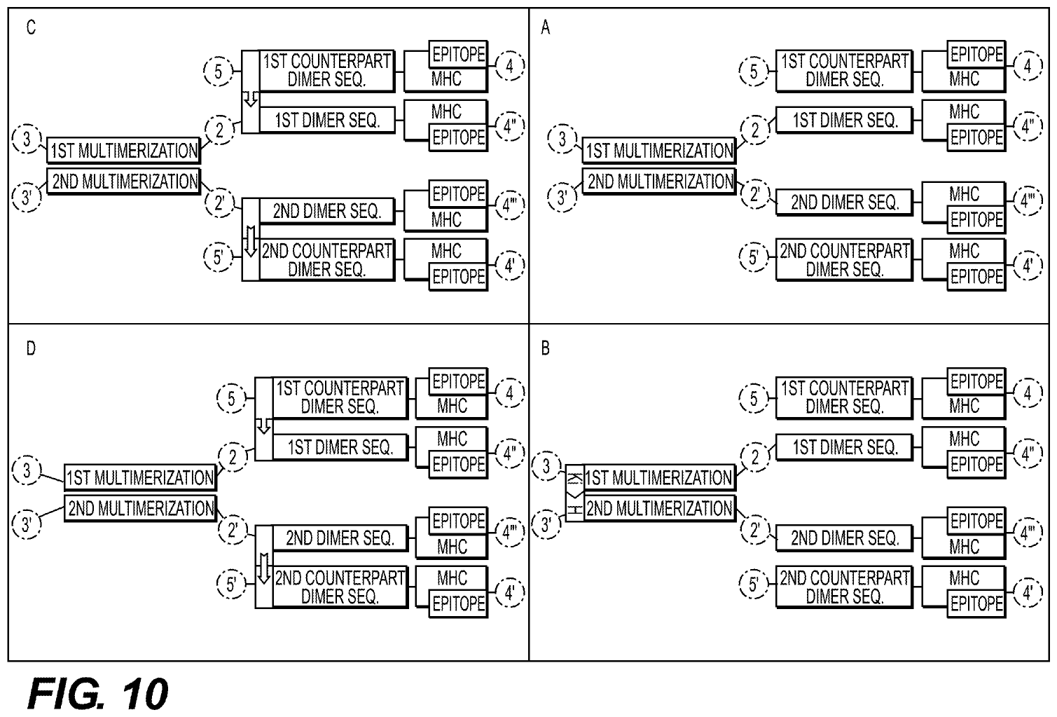

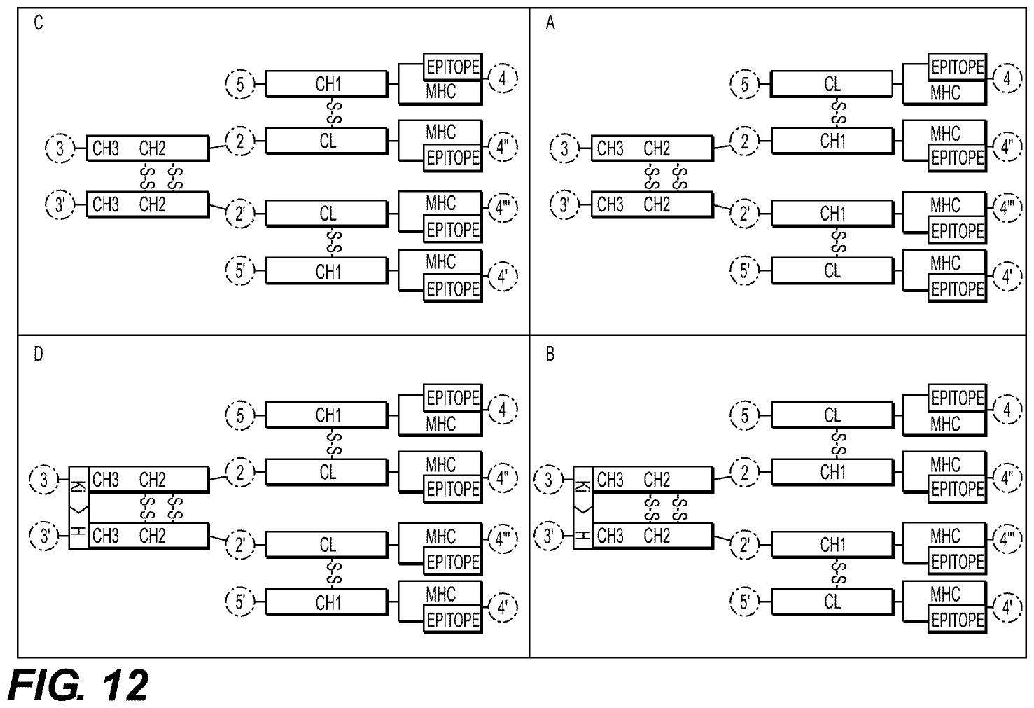

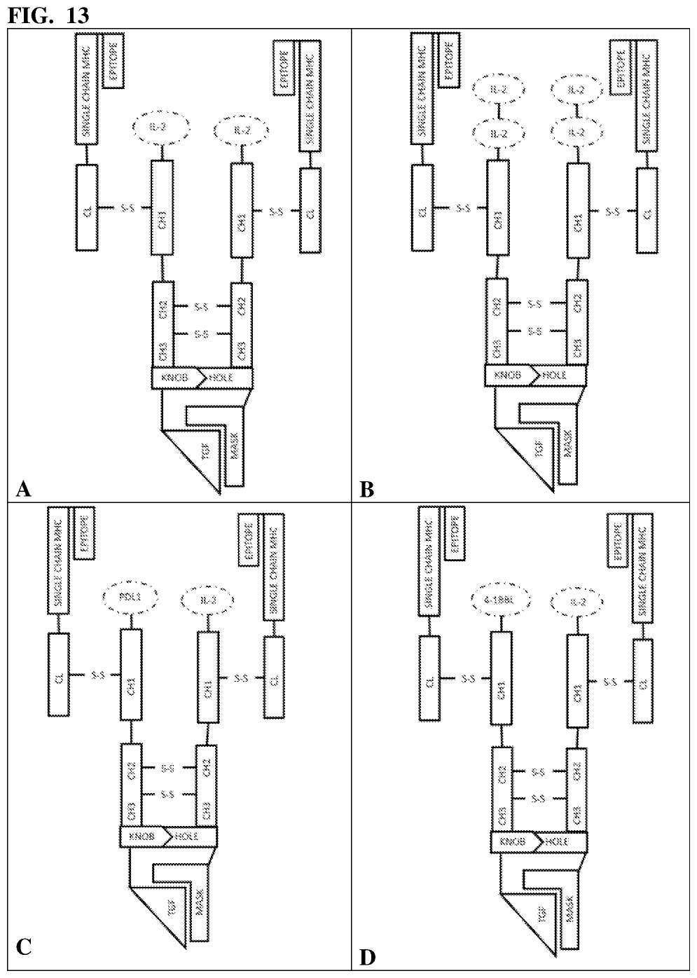

Certain aspects, including embodiments/aspects of the present subject matter described above, may be beneficial alone or in combination, with one or more other aspects recited hereinbelow. In addition, while the present subject matter has been disclosed with reference to certain aspects recited below and, in the claims, numerous modifications, alterations, and changes to the described aspects/embodiments are possible without departing from the sphere and scope of the present disclosure. Accordingly, it is intended that the present disclosure not be limited to the described embodiments, aspects, and claims, but that it has the full scope defined by the language of this disclosure and equivalents thereof.
1. A multimeric antigen-presenting polypeptide complex (MAPP) comprising:
   a framework polypeptide comprising (e.g., from N-terminus to C-terminus) a dimerization sequence and a multimerization sequence;
   a dimerization polypeptide comprising a counterpart dimerization sequence complementary to the dimerization sequence of the framework polypeptide, and dimerizing therewith through covalent (e.g., disulfide bonds) and/or non-covalent interactions to form a MAPP heterodimer; and
   at least one (e.g., at least two) presenting sequence and/or presenting complex,
   wherein each presenting sequence comprises a T1D peptide epitope, and MHC Class II α1, α2, β1, and β2 domain polypeptide sequences;
   wherein each presenting complex comprises a presenting complex 1^{st} sequence and a presenting complex 2^{nd} sequence that together comprise a T1D peptide epitope, and MHC Class II α1, α2, β1, and β2 domain polypeptide sequences, where the peptide epitope is part of the presenting complex 1^{st} sequence or presenting complex 2^{nd} sequence along with at least one of the α1, α2, β1, or β2 domain polypeptide sequences;
   wherein at least one or both of the dimerization polypeptide and/or the framework polypeptide (e.g., either the framework polypeptide, dimerization polypeptide, or both polypeptides) comprises a presenting sequence or a presenting complex 1^{st} sequence (e.g., located on the N-terminal side of the framework polypeptide's dimerization sequence, or the N-terminal side of the dimerization polypeptide′ counterpart dimerization sequence);
   wherein the framework polypeptide, dimerization polypeptide, presenting sequence, or presenting complex comprises (i) a TGF-β sequence, (ii) a masking sequence, or (iii) at least one (e.g., at least two) masked TGF-β immunomodulatory polypeptide(s) (masked TGF-β MOD), each masked TGF-β MOD comprising a masking sequence and TGF-β sequence;
   wherein the framework polypeptide, or dimerization peptide (including the presenting sequence(s) or presenting complex(s) that are present) optionally comprise at least one (e.g., at least two, at least three or more) additional MOD (wt. and/or variant) or pair of additional MODs in tandem (both wt., both variant, or one wt. and one variant) (e.g., located at the N-terminus or C-terminus of the dimerization polypeptide or framework polypeptide, and/or on the C-terminal side of the dimerization sequences); and
   wherein the framework polypeptide, dimerization polypeptide, presenting sequence, presenting complex 1^{st} sequence and/or presenting complex 2^{nd} sequence optionally comprise one or more linker sequences that are selected independently. (See, e.g., FIGS. 1A and 1B).
      It is understood that the dimerization sequence and multimerization sequences are different polypeptide sequences and do not bind in any substantial manner to each other, e.g., the framework polypeptides do not, to any substantial extent, form hair pin structures, self-polymerize, or self-aggregate. Similarly, this aspect may be subject to the proviso that neither the dimerization sequence nor the multimerization sequence of the framework polypeptide comprises an MHC-Class II polypeptide sequence having at least 85% (e.g., 90%, 95% or 98%) sequence identity to at least 20 (e.g., at least 30, 40, 50, 60 or 70) contiguous aas of a MHC-Class II polypeptide in any of FIGS. 4 through 8B. It is also understood that none of the α1, α2, β1 and β2 domain polypeptide sequences include a transmembrane domain, or a portion thereof, that will anchor the MAPP in a cell membrane.
2. The MAPP of aspect 1, wherein the MHC Class II α1 and α2 domain polypeptide sequences comprise human Class II α1 and α2 domain polypeptide sequences having at least 90% or at least 95% (e.g., at least 98% or 100%) sequence identity to an HLA DR alpha (DRA), DQ alpha 1 (DQA1), or DQ alpha 2 (DQA2) α1 and α2 domain polypeptide sequences.
3. The MAPP of aspect 1 or 2, wherein the MHC Class II β1 and β2 domain polypeptide sequences comprises human class β1 and β2 domain polypeptide sequences selected having at least 90% or at least 95% (e.g., at least 98% or 100%) sequence identity to an HLA DR beta 1 (DRB1), DR beta 3 (DRB3), DR beta 4 (DRB4), DR beta 5 (DRB5), DQ beta 1 (DQB1), and DQ beta 2 (DQB2) β1 and β2 domain polypeptide sequences.
4. The MAPP of any preceding aspect, wherein at least one presenting sequence or presenting complex (e.g., at least two, three, or all presenting sequences and/or complexes) comprises:
   a. α1 and α2 domain polypeptide sequences each having at least 90% or at least 95% (e.g., at least 98% or 100%) sequence identity to all or at least about 50 (e.g., at least about 60, 70, 80, 85, or 90) contiguous aas of the HLA DR alpha (DRA) polypeptide sequence provided in FIG. 4; and
   b. β1 and β2 domain polypeptide sequences each having at least 90% or at least 95% (e.g., at least 98% or 100%) sequence identity to all or at least about 50 (e.g., at least about 60, 70, 80, 85, or 90) contiguous aas of an HLA DR beta 1 (DRB1), DR beta 3 (DRB3), DR beta 4 (DRB4), or DR beta 5 (DRB5) polypeptide sequence provided in any one of FIG. 5, 6, 7, or 8.
   For example, a MAPP of aspect 4, may be a MAPP wherein at least one presenting sequence or presenting complex (e.g., at least two or all presenting sequences and/or complexes) comprises:
   α1 and α2 domain polypeptide sequences each having at least 98% or 100% sequence identity to an α1 or α2 domain of a HLA DR alpha (DRA) polypeptide sequence provided in FIG. 4; and
   β1 and β2 domain polypeptide sequences each having at least 98% or 100% sequence identity to a β1 or β2 domain of a HLA DR beta 1 (DRB1), DR beta 3 (DRB3), DR beta 4 (DRB4), or DR beta 5 (DRB5) 31 polypeptide sequences provided in any one of FIG. 5, 6, 7, or 8.
5. The MAPP of any of aspects 1-4, wherein:
   the β1 and β2 domain polypeptide sequences each have at least 90% or at least 95% (e.g., at least 98% or 100%) sequence identity to all or at least about 50 (e.g., at least about 60, 70, 80, 85, or 90) contiguous aas of an HLA DR beta 1 (DRB1) polypeptide sequence provided in FIG. 5 (e.g., DRB1*0301, DRB1*0401, DRB1*0402, DRB1*0405, DRB1*0801, or DRB1*0901).
   For example, MAPP of aspect 5, may be a MAPP wherein the β1 and β2 domain polypeptide sequences each have at least 98% or 100% sequence identity to a β1 or β2 domain of an HLA DR beta 1 (DRB1) polypeptide sequence provided in FIG. 5 (e.g., DRB1*0301, DRB1*0401, DRB1*0402, DRB1*0405, DRB1*0801, or DRB1*0901).
6. The MAPP of aspect 5, wherein:
   the β1 and β2 domain polypeptide sequences each have at least 90% or at least 95% (e.g., at least 98% or 100%) sequence identity to all or at least about 50 (e.g., at least about 60, 70, 80, 85, or 90) contiguous aas of an HLA DR beta 1 (DRB1), DRB1*0301, DRB1*0401, DRB1*0402, or DRB1*0405, polypeptide sequence provided in FIG. 5.
   For example, a MAPP of aspect 6, may be a MAPP wherein the β1 and β2 domain polypeptide sequences each have at least 98% or 100% sequence identity to a β1 or β2 domain of a, DRB1*0301, DRB1*0401, DRB1*0402, or DRB1*0405, polypeptide sequence provided in FIG. 5.
7. The MAPP of any of claims 1 to 3, wherein at least one presenting sequence or presenting complex comprises:
   a. α1 and α2 domain polypeptide sequences each having at least 90% or at least 95% (e.g., at least 98% or 100%) sequence identity to all or at least about 60 (e.g., at least about 70, 80, 85, or 90) contiguous aas of an HLA DQ alpha 1 or DQ alpha 2 (DQA1 or DQA2) polypeptide sequence; and
   b. β1 and β2 domain polypeptide sequences each having at least 90% or at least 95% (e.g., at least 98% or 100%) sequence identity to all or at least about 60 (e.g., at least about 70, 80, 85, or 90) contiguous aas of a HLA DQ beta 1 or DQ beta 2 (DQB1 or DQB2) polypeptide sequence).
   For example, a MAPP of aspect 7, may be a MAPP wherein at least one presenting sequence or presenting complex (e.g., at least two or all presenting sequences and/or complexes) comprises:
   a α1 and α2 domain polypeptide sequences each have at least 98% or 100% sequence identity to an α1 or α2 domain of HLA DQ alpha 1 or DQ alpha 2 (DQA1 or DQA2) polypeptide sequence; and
   a β1 and β2 domain polypeptide sequences each have at least 98% or 100% sequence identity to a β1 or β2 domain of a HLA DQ beta 1 (DQB1) polypeptide sequence.
8. The MAPP of any of claims 1 to 3, wherein at least one presenting sequence or presenting complex comprises either:
   (i) α1 and α2 domain polypeptide sequences each having at least 90% or at least 95% (e.g., at least 98% or 100%) sequence identity to all or at least about 60 (e.g., at least about 70, 80, 85, or 90) contiguous aas of a HLA DQ alpha 1 (DQA1) sequence, and β1 and β2 domain polypeptide sequences each having at least 90% or at least 95% (e.g., at least 98% or 100%) sequence identity to all or at least about 60 (e.g., at least about 70, 80, 85, or 90) contiguous aas of a HLA DQ beta 1 (DQB1) β1 or β2 domain polypeptide sequences; or
   (ii) α1 and α2 domain polypeptide sequences each having at least 90% or at least 95% (e.g., at least 98% or 100%) sequence identity to all or at least about 60 (e.g., at least about 70, 80, 85, or 90) contiguous aas of a HLA DQ alpha 2 (DQA2) polypeptide sequence, and β1 and β2 domain polypeptide sequences each having at least 90% or at least 95% (e.g., at least 98% or 100%) sequence identity to all or at least about 60 (e.g., at least about 70, 80, 85, or 90) contiguous aas of a HLA DQ beta 2 (DQB2) polypeptide sequences.
   For example, a MAPP of aspect 8, may be a MAPP wherein at least one presenting sequence or presenting complex comprises either:
   (i) α1 and α2 domain polypeptide sequences each having at least at least 98% or 100% sequence identity to an α1 or α2 domain of a DQA1polypeptide sequence, and β1 and β2 domain polypeptide sequences each having at least 98% or 100% sequence identity to a β1 or β2 of a DQB1polypeptide sequence; or
   (ii) α1 and α2 domain polypeptide sequences each having at least at least 98% or 100% sequence identity to an α1 or α2 domain of a DQA2 polypeptide sequence, and β1 and β2 domain polypeptide sequences each having at least 98% or 100% sequence identity to a β1 or β2 of a DQB2 polypeptide sequence.
9. The MAPP of any of claims 1 to 3, wherein at least one presenting sequence or presenting complex comprises either:
   (i) α1 and α2 domain polypeptide sequences each having at least 90% or at least 95% (e.g., at least 98% or 100%) sequence identity to all or at least about 60 (e.g., at least about 70, 80, 85, or 90) contiguous aas of a DQA1*0501 polypeptide sequence, and β1 and β2 domain polypeptide sequences each having at least 90% or at least 95% (e.g., at least 98% or 100%) sequence identity to all or at least about 60 (e.g., at least about 70, 80, 85, or 90) contiguous aas of a DQB1*0201 polypeptide sequence; or
   (ii) α1 and α2 domain polypeptide sequences each having at least 90% or at least 95% (e.g., at least 98% or 100%) sequence identity to all or at least about 60 (e.g., at least about 70, 80, 85, or 90) contiguous aas of a DQA1*0301 sequence, and β1 and β2 domain polypeptide sequences each having at least 90% or at least 95% (e.g., at least 98% or 100%) sequence identity to all or at least about 60 (e.g., at least about 70, 80, 85, or 90) contiguous aas of a DQB1*0302 polypeptide sequence.
   For example, a MAPP of aspect 9, may be a MAPP wherein at least one presenting sequence or presenting complex comprises either:
   (i) α1 and α2 domain polypeptide sequences each having at least at least 98% or 100% sequence identity to an α1 or α2 domain of a DQA1*0501 polypeptide sequence, and β1 and β2 domain polypeptide sequences each having at least 98% or 100% sequence identity to a β1 or β2 of a DQB1*0201 polypeptide sequence; or
   (ii) α1 and α2 domain polypeptide sequences each having at least at least 98% or 100% sequence identity to an α1 or α2 domain of a DQA1*0301 sequence, and β1 and β2 domain polypeptide sequences each having at least 98% or 100% sequence identity to a β1 or β2 of a DQB1*0302 polypeptide sequence.
10. The MAPP of any preceding aspect, wherein the MAPP comprises at least one:
   (i) linker comprising Gly (polyG or polyglycine), Gly and Ala (e.g., GA or AG), Ala and Ser (e.g., AS or SA), Gly and Ser (e.g., GS, GSGGS, GGGS, GGSG, GGSGG, GSGSG, GSGGG, GGGSG, GSSSG, GGGGS), or Ala and Gly (e.g., AAAGG), any of which may be repeated 1, 2, 3, 4, 5, 6, 7, 8, 9, or 10 times;
   (ii) linker comprising a cysteine-containing linker sequence selected from CGGGS, GCGGS, GGCGS, GGGCS, and GGGGC, with the remainder of the linker comprised of Gly and Ser residues (e.g., GGGGS units that may be repeated from 1 to 10 times); or
   (iii) at least one rigid peptide linker.
11. The MAPP of any preceding aspect, wherein the MAPP comprises one or more linker aa sequences independently selected from GCGASGGGGSGGGGS, GCGGSGGGGSGGGGSGGGGS, GCGGSGGGGSGGGGS, and GCGGS(G₄S) where the G₄S unit may be repeated from 1 to 10 times (e.g., repeated 1, 2, 3, 4, 5, 6, 7, 8, 9, or 10 times); wherein the linker cysteine residue optionally forms a disulfide bond (e.g., with another peptide sequence of the MAPP).
12. The MAPP of any preceding aspect, wherein the at least one (e.g., at least two) presenting sequence comprises one or more MOD polypeptide sequences, and wherein the presenting sequence has a structure selected from those FIG. 15 or in FIG. 16.
13. The MAPP of any preceding aspect, wherein when the MAPP comprises a presenting sequence, and wherein the presenting sequence comprises, in the N-terminal to C-terminal direction:
   a) the T1D peptide epitope, the β1, α1, α2 and β2 domain polypeptide sequences (see, e.g., FIG. 15 structure A);
   b) the T1D peptide epitope, the β1, β2, α1, and α2 domain polypeptide sequences (see, e.g., FIG. 15 structure B); or
   c) the T1D peptide epitope, the α1, α2, β1, and β2, domain polypeptide sequences (see, e.g., FIG. structure C); and
   wherein the presenting sequence optionally comprises one or more MOD or variant MOD polypeptide sequences; and
   wherein said presenting sequence optionally comprises one or more independently selected linker sequences (e.g., joining any one or more of the peptide epitope, α1, α2, β1, and/or β2 domains and/or at the N- or C-terminus).
14. The MAPP of any preceding aspect, wherein the at least one (e.g., at least two) presenting sequence comprises one or more MOD polypeptide sequences.
15. The MAPP of any preceding aspect, wherein the at least one presenting sequence (e.g., at least two presenting sequences) comprises one or more MOD polypeptide sequences, and wherein the presenting sequence has a structure selected from those set forth in FIG. 15A-C or FIG. 16A-I.
16. The MAPP of any of aspects 1 to 11, comprising at least one presenting complex (e.g., at least two presenting complexes), wherein the at least one presenting complex comprises a presenting complex 1^{st} sequence and presenting complex 2^{nd} sequence wherein:
   (i) the presenting complex 1^{st} sequence comprises the α1 domain polypeptide sequence, and its associated presenting complex 2^{nd} sequence comprises the T1D peptide epitope sequence and the β1 domain polypeptide sequence (e.g., the T1D epitope is placed N-terminal to the β1) (See, e.g., FIG. 17, structures A, B, and D);
   (ii) the presenting complex 1^{st} sequence comprises the α2 domain polypeptide sequence, and its associated presenting complex 2^{nd} sequence comprises the T1D peptide epitope sequence and the β2 domain polypeptide sequence (e.g., the T1D epitope is placed N-terminal to the β2) (See, e.g., FIG. 17, structures A, B, and D);
   (iii) the presenting complex 1^{st} sequence comprises the β1 domain polypeptide sequence, and its associated presenting complex 2^{nd} sequence comprises the T1D peptide epitope sequence and the α1 domain polypeptide sequence (e.g., the T1D epitope is placed N-terminal to the α1);
   (iv) the presenting complex 1^{st} sequence comprises the β2 domain polypeptide sequence, and its associated presenting complex 2^{nd} sequence comprises the T1D peptide epitope sequence and the α2 domain polypeptide sequence (e.g., the T1D epitope is placed N-terminal to the α2);
   (v) the presenting complex 1^{st} sequence comprises the α1 domain polypeptide sequence, and its associated presenting complex 2^{nd} sequence comprises the T1D peptide epitope sequence and the β1 and β2 domain polypeptide sequences (e.g., the T1D epitope is placed N-terminal to the β1 and/or β2);
   (vi) the presenting complex 1^{st} sequence comprises the α2 domain polypeptide sequence, and its associated presenting complex 2^{nd} sequence comprises the T1D peptide epitope sequence and the β1 and β2 domain polypeptide sequences (e.g., the T1D epitope is placed N-terminal to the β1 and/or β2);
   (vii) the presenting complex 15′ sequence comprises the α1 and/or α2 domain polypeptide sequences, and its associated presenting complex 2^{nd} sequence comprises the T1D peptide epitope sequence and the β1 and β2 domain polypeptide sequences (e.g., the T1D epitope is placed N-terminal to the β1 and/or β2) (See, e.g., FIG. 17 structures A, B, and D, FIG. 18 structures B, D, and F, and FIG. 19 structure B); or
   (viii) the presenting complex 1^{st} sequence comprises the β1 and/or β2 domain polypeptide sequences, and its associated presenting complex 2^{nd} sequence comprises the T1D peptide epitope sequence and the α1 and α2 domain polypeptide sequences (e.g., the T1D epitope is placed N-terminal to the α1 and/or α2) (See, e.g., FIG. 19 structures D, E, F, G, and H), and wherein the at least one presenting complex optionally comprises one or more, or two more MODs or variant MODs.
17. The MAPP of any of aspects 1 to 11, comprising at least one presenting complex (e.g., at least two presenting complexes), wherein the at least one presenting complex comprises a presenting complex 1^{st} sequence and presenting complex 2^{nd} sequence, and wherein:
   (i) the presenting complex 1^{st} sequence comprises the peptide epitope sequence and the al domain polypeptide sequence (e.g., the T1D epitope is placed N-terminal to the α1 sequence), and its associated presenting complex 2^{nd} sequence comprises the β1 domain polypeptide sequence;
   (ii) the presenting complex 1^{st} sequence comprises the T1D peptide epitope sequence and the α2 domain polypeptide sequence (e.g., the T1D epitope is placed N-terminal to the α2), and its associated presenting complex 2^{nd} sequence comprises the β2 domain polypeptide sequence (See, e.g., FIG. 17, structure C, FIG. 19, structure C);
   (iii) the presenting complex 1^{st} sequence comprises the T1D peptide epitope sequence and the B1 domain polypeptide sequence (e.g., the T1D epitope is placed N-terminal to the β1), and its associated presenting complex 2^{nd} sequence comprises the α1 domain polypeptide sequence (See, e.g., FIG. 17, structure C, FIG., 28 structures A, C, and E, and FIG. 19 structure A);
   (iv) the presenting complex 1^{st} sequence comprises the T1D peptide epitope sequence and the β2 domain polypeptide sequence(e.g., the T1D epitope is placed N-terminal to the β2), and its associated presenting complex 2^{nd} sequence comprises the α2 domain polypeptide sequence (e.g., the T1D epitope is placed N-terminal to the α2) (See, e.g., FIG. 27, structure C, FIG., 28 structures A, C, and E, and FIG. 19, structures A, G and C);
   (v) the presenting complex 151 sequence comprises the T1D peptide epitope sequence and the al domain polypeptide sequence (e.g., the T1D epitope is placed N-terminal to the al sequence), and its associated presenting complex 2^{nd} sequence comprises the β1 and 2 domain polypeptide sequences (e.g., the T1D epitope is placed N-terminal to the β1 and/or β2);
   (vi) the presenting complex 1^{st} sequence comprises the T1D peptide epitope sequence and the α2 domain polypeptide sequence (e.g., the T1D epitope is placed N-terminal to the α2 sequence), and its associated presenting complex 2^{nd} sequence comprises the β1 and β2 domain polypeptide sequences (e.g., the T1D epitope is placed N-terminal to the β1 and/or β2);
   (vii) the presenting complex 1^{st} sequence comprises the T1D peptide epitope sequence and the al and/or α2 domain polypeptide sequences (e.g., the T1D epitope is placed N-terminal to the α1 and/or α2), and its associated presenting complex 2^{nd} sequence comprises the β1 and β2 domain polypeptide sequences (e.g., the T1D epitope is placed N-terminal to the β1 and/or β2) (See, e.g., FIG. 19 structure C); or
   (viii) the presenting complex 151 sequence comprises the T1D peptide epitope sequence and the β1 and/or β2 domain polypeptide sequences (e.g., the T1D epitope is placed N-terminal to the β1 and/or β2), and its associated presenting complex 2^{nd} sequence comprises the α1 and α2 domain polypeptide sequences (See, e.g., FIG. 17 structure C, FIG. 18 structures A, B, C, and E, and FIG. 19 structure A, D, E, and F), and
      wherein the at least one presenting complex optionally comprises one or more, or two more MODs or variant MODs.
18. The MAPP of any of aspects 1 to 11, comprising at least one presenting complex that comprises one or more (e.g., two or more) MOD or variant MOD polypeptide sequences.
19. The MAPP of any of aspects 1 to 11, comprising at least one (e.g., at least two) presenting complex, wherein the at least one presenting complex has a structure selected from those in FIG. 17, 18, or 19.
20. The MAPP of any of aspects 1 to 11, comprising at least one (e.g., at least two) presenting complex, wherein the at least one presenting complex has a structure selected from those in FIGS. 20 to 22.
21. The MAPP of any preceding aspect, wherein, the at least one presenting sequence, presenting complex 1^{st} sequence, or presenting complex 2^{nd} sequence has the T1D peptide epitope sequence within 10 aa, 15 aa, 20 aa, or 25 aa of the N-terminus of the presenting sequence, presenting complex 1^{st} sequence, or presenting complex 2^{nd} sequence.
22. The MAPP of any preceding aspect, comprising a presenting sequence or presenting complex that comprises a cysteine-containing linker, wherein the cysteine residue in the cysteine-containing linker forms a disulfide bond between a between the presenting sequence and another polypeptide of the MAPP, or between the presenting complex 1^{st} sequence and another polypeptide of the MAPP (e.g., with a presenting complex 2^{nd} sequence).
23. The MAPP of any preceding aspect, comprising at least one presenting sequence or a presenting complex comprising a disulfide bond formed between one of MHC α1 or α2 domain polypeptide sequence and one of the β1 or β2 domain polypeptide sequences.
24. The MAPP of any preceding aspect, comprising:
   at least one presenting sequence or a presenting complex comprising a disulfide bond formed between cysteines positioned at:
      α chain position 3 and β chain position 19 or 20,
      α chain position 4 and β chain position 19 or 20,
      α chain position 28 and β chain position 151, 152, or 153,
      α chain position 29 and β chain position 151, 152, or 153,
      α chain position 80, 81, or 82 and β chain position 33,
      α chain position 93 and β chain position 153 of 156,
      α chain position 94 and β chain position 120 or 156, or
      α chain position 95 and β chain position 120 or 156.
25. The MAPP of any preceding aspect, comprising at least one presenting sequence or a presenting complex comprising a disulfide bond formed between cysteines positioned at:
   α chain position 12 and β chain position 7 or 10,
   α chain position 80 and β chain position 5 or 7,
   α chain position 81 and β chain position 5 or 7, or
   α chain position 82 and β chain position 5 or 7.
26. The MAPP of any preceding aspect, comprising:
   at least one presenting sequence or at least one presenting complex that comprises a cysteine-containing polypeptide linker having the structure (aa1-aa2-aa3-aa4-aa5-[remainder of linker if present]) located between the peptide epitope (e.g., an N-terminal peptide epitope) and a β1 domain polypeptide sequence such that the at least one presenting sequence or at least one presenting complex comprises a substructure of the form {epitope-aa1-aa2-aa3-aa4-aa5-[remainder of linker if present or bond]-β1 domain};
   wherein the presenting sequence or presenting complex comprises a disulfide bond between a cysteine located at any of aa1 to aa5 and a cysteine located in the MHC α chain polypeptide sequence (e.g., an α1 or α2 domain polypeptide sequence).
27. The MAPP of aspect 26, wherein the cysteine located in the MHC α chain is located at position 72 or 75 of the mature α chain (e.g., position 72 or 75 of the mature DRA polypeptide sequence lacking its signal sequence, or the equivalent positions in other MHC alpha chain sequence, see Table 5 and associated text).
28. The MAPP of aspect 26 or 27, wherein aa3 of the polypeptide linker is the cysteine (Cys) located at any of aa1 to aa5.
29. The MAPP of any of aspects 27 to 28, wherein aa1 to aa5 are the amino acid sequence Gly Gly Cys Gly Ser.
30. The MAPP of any of aspects 27 to 29, wherein the presenting sequence comprises polypeptide having greater than 90% or greater than 95% sequence identity to aas 26-203 of the mature DRA polypeptide of SEQ ID NO: 17 (see FIG. 4), and the disulfide bond between a cysteine located at any of aa1 to aa5 and a cysteine located in the MHC α chain polypeptide sequence is formed between a cysteine at position 3 of the polypeptide linker (i.e. aa3) and a cysteine substituted at position 72 or 75 (corresponding to positions 97 and 100 of the sequence in FIG. 4 with the 25aa signal peptide) of the DRA polypeptide (i.e., I72C or K75C substitutions).
31. The MAPP of any preceding aspect, comprising at least one presenting sequence or a presenting complex that comprises a disulfide bond formed between cysteines positioned at:
   α chain position 80 and β chain position 5 or 7; or
   α chain position 81 and β chain position 5 or 7.
32. The MAPP of any preceding aspect, wherein the dimerization and multimerization sequences are independently selected from non-interspecific sequences or interspecific sequences.
33. The MAPP of aspect 32, wherein the interspecific and non-interspecific sequences are selected from the group consisting of: immunoglobulin heavy chain constant regions (Ig Fc, e.g., CH2-CH3); collectin polypeptides, coiled-coil domains, leucine-zipper domains; Fos polypeptides; Jun polypeptides; Ig CH1; Ig C_{L} κ; Ig C_{L} λ; knob-in-hole without disulfide (“KiH”); knob-in hole with a stabilizing disulfide bond (“KiHs-s”); HA-TF; ZW-1; 7.8.60; DD-KK; EW-RVT; EW-RVTs-s; and A107 sequences.
34. The MAPP of any preceding aspect, complexed to form a duplex or higher order MAPP comprising at least a first MAPP heterodimer and a second MAPP heterodimer of any of aspects 1-33, wherein:
   (i) the first MAPP comprises a first framework polypeptide having a first multimerization sequence and a first dimerization sequence, and a first dimerization polypeptide having first counterpart dimerization sequence complementary to the first dimerization sequence; and
   (ii) the second MAPP comprises a second framework polypeptide having a second multimerization sequence and a second dimerization sequence, and a second dimerization polypeptide having second counterpart dimerization sequence complementary to the second dimerization sequence;
   wherein the first and second framework polypeptides are associated by binding interactions between the first and second multimerization sequences optionally including one or more interchain covalent bonds (e.g., one or two disulfide bonds), and the multimerization sequences are not the same (e.g., not the same type and/or not identical to), and do not substantially associate with or bind to, the dimerization sequences or counterpart dimerization sequences. See, e.g., the duplexes in FIGS. 9 to 13; and
   wherein the duplex or higher order MAPP comprises at least one masked TGF-β MOD with the masking sequence and the TGF-β sequence present in cis or in trans.
35. The duplex MAPP of aspect 34, wherein the first and second dimerization sequence are identical, and the first and second counterpart dimerization sequences are identical. See, e.g., FIGS. 11, 12, and FIG. 13 structures A-H.
36. The duplex MAPP of aspect 34, wherein the first and second dimerization sequences do not substantially associate with or bind to each other.
37. The duplex MAPP of aspect 34, wherein the first and second multimerization sequences are interspecific multimerization sequences that form an interspecific pair, the first and second dimerization sequence are identical, and the first and second counterpart dimerization sequences are identical. See, e.g., FIG. 9 structure B and FIG. 11, structures B and D.
38. The duplex MAPP of aspect 37, wherein the first and second dimerization sequences do not substantially associate with or bind each other.
39. The duplex MAPP of any of aspects 37 to 38, wherein the first or second framework polypeptide comprises at least one MOD (e.g., two or three MODs) that is/are not present on the other framework polypeptide, optionally wherein the at least one MOD comprises a polypeptide sequence that is a variant of a wild-type MOD sequence.
40. The duplex MAPP of aspect 34, wherein the first and second multimerization sequences are identical, the first dimerization sequence and the first counterpart dimerization sequence are interspecific dimerization sequences forming a first interspecific pair, and the second dimerization sequence and second counterpart dimerization sequence are interspecific dimerization sequences forming a second interspecific pair. See, e.g., FIG. 9 structure C.
41. The duplex MAPP of aspect 40, wherein the first and second dimerization sequences are identical and the first and second counterpart dimerization sequences are identical. See, e.g., FIG. 11 structure A.
42. The duplex MAPP of aspect 40, wherein the first and second dimerization sequences are not identical do not substantially associate with or bind with each other.
43. The duplex MAPP of aspect 40, wherein the polypeptides of the first interspecific pair are different from (not identical to), and do not bind or interact with the polypeptides of the second interspecific pair.
44. The duplex MAPP of any of aspects 42 to 43, wherein the first or second dimerization polypeptide comprises at least one MOD (e.g., two or three MODs or variant MODs or variant MODs) that is/are not present on the other dimerization polypeptide, optionally wherein the at least one MOD comprises a polypeptide sequence that is a variant of a wild-type MOD sequence.
45. The duplex MAPP of aspect 34, wherein
   the first and second multimerization sequences are interspecific multimerization sequences that form an interspecific multimerization pair,
   the first dimerization sequence and the first counterpart dimerization sequence are interspecific dimerization sequences forming a first interspecific pair, and
   the second dimerization sequence and second counterpart dimerization sequence are interspecific dimerization sequences forming a second interspecific pair. See, e.g., FIG. 9 structure D.
46. The duplex MAPP of aspect 45, wherein the first and second dimerization sequences are identical and the first and second counterpart dimerization sequences are identical. See, e.g., FIG. 11 structure D.
47. The duplex MAPP of aspect 45, wherein the first and second dimerization sequences do not substantially associate with or bind with each other.
48. The duplex MAPP of aspect 45, wherein the polypeptides of the first interspecific pair of polypeptides are different from (not identical to), and do not bind or interact with the polypeptides of the second interspecific pair.
49. The duplex MAPP of any of aspects 45 to 48, wherein the first or second dimerization polypeptide comprises at least one MOD (e.g., two or three MODs or variant MODs) that is/are not present on the other dimerization polypeptide, optionally wherein the at least one MOD comprises a polypeptide sequence that is a variant of a wild-type MOD sequence.
50. The duplex MAPP of any of aspects 45 to 49, wherein the first or second framework polypeptide comprises at least one MOD (e.g., two or three MODs and/or variant MODs) that is/are not present on the other framework polypeptide, optionally wherein the at least one MOD comprises a polypeptide sequence that is a variant of a wild-type MOD sequence.
51. The duplex MAPP of any of aspects 34 to 50, wherein:
   (i) when the first and second multimerization sequences are not an interspecific multimerization pair, the multimerization sequence(s) is/are selected from the group consisting of immunoglobulin heavy chain constant regions (e.g., Ig CH2-CH3), collectin family dimerization sequences, coiled-coil domains, and leucine-zipper domains; and
   (ii) when the first and second multimerization sequences are an interspecific multimerization pair, the multimerization sequence(s) is/are selected from the group consisting of a Fos and Jun polypeptide pair, Ig CH1 and Ig CL κ or λ constant region polypeptide pair, and interspecific Ig Fc pairs (e.g., a KiH pair, a KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, or an A107 polypeptide pair).
52. The duplex MAPP of aspect 51, wherein the first and second multimerization sequences, the first dimerization sequence and its counterpart first dimerization sequence, and second dimerization sequence and its counterpart dimerization sequence are each selected from the group consisting of: immunoglobulin heavy chain constant regions (e.g., IgFc CH2-CH3), collectin family dimerization sequences, coiled-coil domains, and leucine-zipper domains, and
   wherein the first and second dimerization sequences, which are selected independently and may be the same or different.
53. The duplex MAPP of aspect 51, wherein the first and second multimerization sequences are selected from the group consisting of immunoglobulin heavy chain constant regions (e.g., Ig CH2-CH3 regions), collectin family dimerization sequences, coiled-coil domains, and leucine-zipper domains, and
   wherein the first dimerization sequence and its counterpart dimerization sequence are independently selected from the group consisting of a Fos and Jun polypeptide pair, Ig CH1 and Ig C_{L} κ or λ constant region polypeptide pair, a KiH pair, KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair; and
   wherein the dimerization sequence and counterpart dimerization pairs present in the MAPP may be the same or different.
54. The duplex MAPP of aspect 51, wherein the first and second multimerization sequences are selected from the group consisting of a Fos and Jun polypeptide pair, Ig CH1 and Ig C_{L} κ or λ constant region polypeptide pair, a KiH pair, a KiHs-s pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair,
   wherein the first and second dimerization sequences and their counterpart dimerization sequences are independently selected from the group consisting of immunoglobulin heavy chain constant regions (e.g., Ig CH2-CH3), collectin family dimerization sequences, coiled-coil domains, and leucine-zipper domains; and
   wherein the dimerization sequence and counterpart dimerization pairs present in the MAPP may be the same or different.
55. The duplex MAPP of aspect 51, wherein the first and second multimerization sequences, the first dimerization sequence and its counterpart first dimerization sequence, and second dimerization sequence and its counterpart dimerization sequence are each selected as a pair from the group consisting of: Fos and Jun polypeptide pairs, Ig CH1 (see e.g., FIGS. 2A, 2B, and 2E-2I) and Ig C_{L} κ or λ constant region polypeptide pairs, KiH pairs, KiHs-s pairs, HA-TF polypeptide pairs, ZW-1 polypeptide pairs, 7.8.60 polypeptide pairs, a DD-KK polypeptide pairs, EW-RVT polypeptide pairs, EW-RVTs-s polypeptide pairs, and A107 polypeptide pairs, and
   wherein the pairs comprising the first and second dimerization sequences may be the same or different.
56. The duplex MAPP of aspect 51, wherein the first and second multimerization sequences comprise Ig Fc regions comprising CH2-CH3 domains and the first and second dimerization sequences comprise independently selected Ig CH1, Ig C_{L} κ or λ, leucine zipper, Fos or Jun domains.
57. The duplex MAPP of aspect 51, wherein the first and second multimerization sequences comprise Ig Fc regions and the first and second dimerization sequences comprise independently selected Ig CH1 or Ig C_{L} κ or λ domains.
58. The duplex MAPP of any of aspects 56 to 57, wherein the Ig CH2-CH3 domains are selected from the group consisting of IgA, IgD, IgE, IgG and IgM Fc regions.
59. The duplex MAPP of any of aspects 56 to 58, wherein the Ig Fc regions are selected from IgG1, IgG2, IgG3, and IgG4 CH2-CH3 domains (e.g., having at least about 85%, at least about 90%, at least about 95%, at least about 98%, at least about 99%, or 100% aa sequence identity to an aa sequence of the CH2 and/or CH3 domains of an Fc region of SEQ ID NOs: 4-12).
60. The duplex MAPP of any of aspects 56 to 59, wherein the Ig Fc regions are IgG1CH2-CH3 domains.
61. The duplex MAPP of aspect 51, wherein the first and second multimerization sequences are an interspecific Ig Fc pair.
62. The duplex MAPP of aspect 61, wherein the interspecific Ig Fc pair is selected from the group consisting of KiH pairs, KiHs-s pairs, HA-TF polypeptide pairs, ZW-1 polypeptide pairs, 7.8.60 polypeptide pairs, a DD-KK polypeptide pairs, EW-RVT polypeptide pairs, EW-RVTs-s polypeptide pairs, and A107 polypeptide pairs.
63. The duplex MAPP of aspect 61 or 62, wherein the pair of interspecific Ig Fc pair is a KiH pair.
64. The duplex MAPP of any of aspects 61 to 63, wherein the knob-in hole pair further comprises at least one stabilizing disulfide bond (e.g., a KiHs-s pair).
65. The duplex MAPP of any of aspects 61 to 64, wherein the first and second dimerization sequences comprise independently selected Ig CH1, Ig C_{L} κ or λ, leucine zipper, Fos or Jun domains.
66. The duplex MAPP of any of aspects 61 to 64, wherein the first and second dimerization sequences comprise Ig CH1 or Ig C_{L} κ or λ domains.
67. The duplex MAPP of any of aspects 61 to 64, wherein the first and second dimerization sequences comprise Ig CH1 domains.
68. The duplex MAPP of any of aspects 63 to 64, wherein the first and/or second dimerization sequences do not comprise Ig CH1 domains.
69. The MAPP of any of aspects 1 to 68, wherein the dimerization sequence and its counterpart dimerization sequence are covalently linked by at least one (e.g., two) disulfide bond(s); or
70. the duplex MAPP of any of aspects 34-69, wherein the dimerization sequence and counterpart dimerization sequence of the first MAPP and/or the dimerization sequence and counterpart dimerization sequence of the second MAPP are covalently linked by at least one (e.g., two) disulfide bond(s).
71. The duplex MAPP of any of aspects 34 to 70, wherein the multimerization sequences of the first and second framework polypeptides of the first and second MAPP are covalently linked by at least one (e.g., two) disulfide bond(s). See e.g., FIGS. 11 to 13.
72. The duplex MAPP of any of aspects 34 to 71, wherein the first dimerization sequence and its counterpart dimerization sequence and/or the second dimerization sequence and its counterpart dimerization sequence are covalently linked by at least one (e.g., two) disulfide bond(s), and the multimerization sequences of the first and second framework polypeptides of both the first and second MAPP are covalently linked by at least one (e.g., two) disulfide bond(s). See e.g., FIGS. 12 and 13.
73. The MAPP or duplex MAPP of any preceding aspect, wherein the MAPP comprises only one presenting sequence or one presenting complex, or the duplex MAPP comprises only two presenting sequences or two presenting complexes. See, e.g., FIGS. 1A and 1B. See, e.g., the 1^{st} or 2^{nd} MAPP heterodimers.
74. The MAPP or duplex MAPP of aspect 73, wherein the one presenting sequence or presenting complex of the MAPP, or the two presenting sequences or presenting complexes of the duplex MAPP, is/are provided on the dimerization polypeptide(s), and further, when the MAPP or duplex MAPP comprises a presenting complex, the presenting complex 1^{st} sequence(s) is/are located on the dimerization polypeptide(s) (e.g., located on the N-terminal side of the counterpart dimerization sequence). See, e.g., FIGS. 1A and 1B.
75. The MAPP or duplex MAPP of aspect 73, wherein the one presenting sequence or the presenting complex 1^{st} sequence of the MAPP, or the two presenting sequences or presenting complex 1^{st} sequences of the duplex MAPP, is/are provided on (is part of) the framework polypeptide(s) (e.g., located on the N-terminal side of the dimerization sequence).
76. The duplex MAPP of any of aspects 34 to 72, wherein the duplex MAPP comprises only one presenting sequence or one presenting complex.
77. The duplex MAPP of aspect 76, wherein the one presenting sequence or the presenting complex 1^{st} sequence of the one presenting complex is an aa sequence of (is part of) a dimerization polypeptide (e.g., located on the N-terminal side of the counterpart dimerization sequence of the one dimerization polypeptide).
78. The duplex MAPP of aspect 76, wherein the one presenting sequence or the presenting complex 1^{st} sequence of the one presenting complex is an aa sequence of (is part of) a framework polypeptide (e.g., located on the N-terminal side of the dimerization sequence).
79. The duplex MAPP of any of aspects 34 to 72, wherein the duplex MAPP comprises at least two presenting sequences or at least two presenting complexes. See, e.g., the duplex in FIGS. 1A and 1B.
80. The duplex MAPP of aspect 79, wherein one of the at least two presenting sequences or presenting complex 1^{st} sequences of the at least two presenting complexes is part of the first dimerization polypeptide, and the second of the at least two presenting sequences or presenting complex 1^{st} sequences is part of the second dimerization polypeptides (e.g., located on the N-terminal side of their counterpart dimerization sequences). See, e.g., the duplex in FIGS. 1A and 1B.
81. The duplex MAPP of aspect 79, wherein one of the at least two presenting sequences or each of the presenting complex 1^{st} sequences of the at least two presenting complexes is part of the first framework polypeptide, and the second of the at least two presenting sequences or presenting complex 1^{st} sequences is part of the second framework polypeptide (e.g., located on the N-terminal side of their dimerization sequence).
82. The duplex MAPP of any of aspects 34 to 72, wherein the duplex MAPP comprises at least four presenting sequences or four presenting complexes. See, e.g., FIG. 10 structures A-D.
83. The duplex MAPP of aspect 74, wherein each one of the four presenting sequences or each one of the presenting complex 1^{st} sequences of the four presenting complexes are each part of a different one of the first dimerization polypeptide, second dimerization polypeptide, first framework polypeptide and second framework polypeptide (e.g., located on the N-terminal side of their dimerization sequence or counterpart dimerization sequence). See, e.g., FIG. 10 structures A-D.
84. The MAPP or duplex MAPP of any preceding aspect, wherein when at least one framework or dimerization polypeptide comprises one or more IgFc regions, and wherein at least one of the one or more IgFc regions comprises one or more substitutions that limit complement-dependent cytotoxicity (CDC) and/or antibody-dependent cell cytotoxicity (ADCC).
85. The MAPP or duplex MAPP of any preceding aspect, wherein when a framework or dimerization polypeptides of the MAPP or duplex MAPP comprises one or more IgFc regions, and wherein at least one of the one or more IgFc regions comprises one or more substitutions at L234, L235, G236, G237, P238, S239, D270, N297, K322, P329, and/or P331 (respectively, aas L14, L15, G16, G17, P18, S19, N77, D50, K102, P109, and P111 of the wt. IgG1 aa sequence in FIG. 2D).
86. The MAPP or the duplex MAPP of aspect 85, wherein when a framework or dimerization polypeptide comprises an IgFc region comprising a substitution at N297 (e.g., N297A).
87. The MAPP or the duplex MAPP of aspect 85, wherein when a framework or dimerization polypeptide comprises an IgFc region comprising a substitution at L234, and/or L235 (e.g., L234A, and/or L235A).
88. The MAPP or the duplex MAPP of aspect 85, wherein when a framework or dimerization polypeptide comprises an IgFc region comprising a substitution at P331 (e.g., P331A or P331S.
89. The MAPP or the duplex MAPP of aspect 85, wherein when a framework or dimerization polypeptide comprises an Ig Fc region that comprises: (i) one or more substitutions selected from the group consisting of L234, L235, and P331 (e.g., L234F, L235E, and/or P331S substitution(s)); or (ii) any one or more of D270, K322, and P329 (e.g., D270, K322, and/or P329 substitution(s)).
90. The MAPP or the duplex MAPP of aspect 85, wherein when a framework or dimerization polypeptide comprises an IgFc region that comprises either (i) one or more substitutions selected from the group consisting of L234, L235, and P331, or (ii) one or more substitutions selected from the group consisting of D270, K322, and P329.
91. The MAPP of any of aspects 1 to 90, complexed to form a duplex MAPP of two heterodimers, a triplex MAPP of three heterodimers, a quadraplex MAPP of four heterodimers, a pentaplex MAPP of five heterodimers, or a hexaplex MAPP of six heterodimers.
92. The MAPP or duplex MAPP of any preceding aspect, wherein the masking sequence and TGF-β sequence of the at least one masked TGF-β MOD (e.g., all masked TGF-β MODs) are present in “trans” with the masking sequence and TGF-β sequence part of different MAPP polypeptides (such as different framework or dimerization polypeptides) that interact by an interspecific multimerization sequences or interspecific dimerization sequences (see, e.g., FIG. 13A-D, wherein interaction of the IgG KIH sequences bring a masking sequence into proximity to a TGF-β amino acid sequence).
93. The MAPP or duplex MAPP of aspect 92, wherein one of the masking sequence or the TGF-β sequence is present at the carboxyl terminus of a dimerization polypeptide (e.g., position 5 or 5′ in FIG. 1A).
94. The MAPP or duplex MAPP of aspect 92, wherein one of masking sequence or the TGF-β sequence is present at the amino terminus of a dimerization polypeptide or a framework polypeptide (e.g., position 1, 1′ 4, 4′, 4″ or 4″ of FIG. 1A, 1B, or 9-12).
95. The duplexed MAPP of aspect 92, wherein one of the masking sequence or the TGF-β sequence is present at the carboxyl terminus of the first framework polypeptide and the other of the masking sequence and the TGF-β amino acid sequence is present at the carboxy terminus of the second framework polypeptide (e.g., positions 3 and 3′ of FIG. 1A or FIG. 1C or 1D at (a) or (b)).
96. The MAPP or duplex MAPP of any of aspects 1 to 91, wherein the at least one masked TGF-β MOD (e.g., all masked TGF-β MODs) comprises a masking sequence and a TGF-β sequence present in “cis” as part of a single polypeptide amino acid sequence (see, e.g., FIG. 13 E-H).
97. The MAPP duplex MAPP of aspect 96, wherein the at least one masked TGF-β MOD (e.g., each masked TGF-β MODs) comprising a masking sequence and a TGF-β sequence present in “cis” is present at the carboxyl terminus of a dimerization polypeptide (e.g., position 5 or 5′ in FIG. 1A).
98. The MAPP duplex MAPP 96, wherein the at least one masked TGF-β MOD (e.g., each masked TGF-β MODs) comprising a masking sequence and a TGF-β sequence present in “cis” is present at the amino terminus of a dimerization or framework polypeptide (e.g., position 1, 1′ 4, 4′, 4″ or 4′″ of FIG. 1A, 1B, or 9-12).
99. The MAPP duplex MAPP 96, wherein the at least one masked TGF-β MOD (e.g., each masked TGF-β MODs) comprises a masking sequence and a TGF-β sequence present in “cis” is present at the carboxyl terminus of a framework polypeptide (e.g., positions 3 or 3′ of FIG. 1A or FIG. 1C or 1D at (c) or (d).
100. The MAPP or duplex MAPP of any preceding aspect, wherein the TGF-β sequence present in the at least one masked TGF-β MOD is a TGF-β1 polypeptide optionally comprising a substitution of C77.
101. The MAPP or duplex MAPP of aspect 100, wherein the TGF-β1 polypeptide comprises an amino acid sequence having at least 90%, at least 95%, at least 98%, at least 99%, or 100%, aa sequence identity to at least 70, at least 80, at least 90, at least 100, at least 110, or 112 aas of the mature TGF-β1 aa sequence set forth in FIG. 24 (SEQ ID NO: 194), and optionally comprising a substitution of C77.
102. The MAPP or duplex MAPP of aspect 100, wherein the TGF-β1 polypeptide comprises an amino acid sequence having at least 90%, at least 95%, at least 98%, at least 99%, or 100%, aa sequence identity to at least 70, at least 80, at least 90, at least 100, at least 110, or contiguous 112 aas of the TGF-β1 amino acid sequence AL DTNYCFSSTE KNCCVRQLYI DERKDLGWKW IHEPKGYHAN FCLGPCPYIW SLDTQYSKVL ALYNQHNPGA SAAPCCVPQA LEPLPIVYYV GRKPKVEQLS NMIVRSCKCS (SEQ ID NO:101), and optionally comprising a substitution of C77. For example, a MAPP or duplex MAPP of aspect 154 may comprises a TGF-β1 aa sequence having at least 90%, or at least 95%, (e.g., at least 98% or 100%) aa sequence identity to the mature TGF-β1 amino acid sequence of SEQ ID NO: 101) optionally comprising a substitution of C77.
103. The MAPP or duplex MAPP of any of aspects 100 to 102, wherein the TGF-β1 aa sequence comprises a C77S substitution.
104. The MAPP or duplex MAPP of any of aspects 1 to 99, wherein the TGF-β sequence present in the at least one masked TGF-β MOD is a TGF-β2 polypeptide optionally comprising a substitution of C77.
105. The MAPP or duplex MAPP of aspect 104, wherein the TGF-β2 polypeptide comprises an amino acid sequence having at least 90%, at least 95%, at least 98%, at least 99%, or 100%, aa sequence identity to at least 70, at least 80, at least 90, at least 100, at least 110, or 112 aas of the mature TGF-β2 aa sequence set forth in FIG. 24 (SEQ ID NO: 109), and optionally comprising a substitution of C77.
106. The MAPP or duplex MAPP of aspect 104, wherein the TGF-β2 polypeptide comprises an amino acid sequence having at least 90%, at least 95%, at least 98%, at least 99%, or 100%, aa sequence identity to at least 70, at least 80, at least 90, at least 100, at least 110, or 112 contiguous aas of the TGF-β2 amino acid sequence ALDAAYCFRN VQDNCCLRPL YIDFKRDLG WKWIHEPKGY NANFCAGACP YLWSSDTQHS RVLSLYNTIN PEASASPCCV SQDLEPLTIL YYIGKTPKIE QLSNMIVKSC KCS (SEQ ID NO:103), and optionally comprising a substitution of C77. For example, a MAPP or duplex MAPP of aspect 158 may comprises a TGF-β2 aa sequence having at least 90%, or at least 95%, (e.g., at least 98% or 100%) aa sequence identity to the TGF-β2 amino acid sequence of SEQ ID NO: 104) optionally comprising a substitution of C77.
107. The MAPP or duplex MAPP of any of aspects 104 to 106, wherein the TGF-β2 aa sequence comprises a C77S substitution.
108. The MAPP or duplex MAPP of any of aspects 1 to 99, wherein the TGF-β sequence present in the at least one masked TGF-β MOD is a TGF-β3 polypeptide optionally comprising a substitution of C77.
109. The MAPP or duplex MAPP of aspect 108, wherein the TGF-β3 polypeptide comprises an amino acid sequence having at least 90%, at least 95%, at least 98%, at least 99%, or 100%, aa sequence identity to at least 70, at least 80, at least 90, at least 100, at least 110, or 112 aas of the TGF-β3 aa sequence set forth in FIG. 24 (SEQ ID NO:196), and optionally comprising a substitution of C77.
110. The MAPP or duplex MAPP of aspect 108, wherein the TGF-β3 polypeptide comprises an amino acid sequence having at least 90%, at least 95%, at least 98%, at least 99%, or 100%, aa sequence identity to at least 70, at least 80, at least 90, at least 100, at least 110, or 112 contiguous aas of the TGF-3 amino acid sequence ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO:105), and optionally comprising a substitution of C77. For example, a MAPP or duplex MAPP of aspect 162 may comprises a TGF-β3 aa sequence having at least 90%, or at least 95%, (e.g., at least 98% or 100%) aa sequence identity to the TGF-β3 amino acid sequence of SEQ ID NO: 105) optionally comprising a substitution of C77.
111. The MAPP or duplex MAPP of any of aspects 108 to 110, wherein the TGF-β3 aa sequence comprises a C77S substitution.
112. The MAPP or duplex MAPP of any of aspects 100 to 111, wherein the TGF-β sequence comprising a substitution at one or more of position 25, 92, and/or 94.
113. The MAPP or duplex MAPP of any preceding aspect, wherein the masking sequence present in the at least one masked TGF-β MOD is a TGF-β receptor (TβR) polypeptide, anti-TGF-β antibody (e.g., anti-TGF-β1, anti-TGF-β2, and/or anti-TGF-β3) or antibody-related polypeptide/aa sequence (e.g., antigen binding fragment, Fab, Fab′, single chain antibody, scFv, peptide aptamer, or nanobody aa sequence).
114. The MAPP or duplex MAPP of aspect 113, wherein the masking sequence comprises all or part of a TβRI ectodomain.
115. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, or 103 aas of the following TβRI ectodomain aa sequence: LQCFCHL CTKDNFTCVT DGLCFVSVTE TTDKVIHNSM CIAEIDLIPR DRPFVCAPSS KTGSVTTTYC CNQDHCNKIE LPTTVKSSPG LGPVEL (SEQ ID NO:107) (See e.g., FIG. 26A). For example, a MAPP or duplex MAPP of aspect 167 may comprise a masking sequence having at least 90%, or at least 95% (e.g., at least 98% or 100%) aa sequence identity to the TβRI ectodomain aa sequence of SEQ ID NO:107).
116 The MAPP or duplex MAPP of aspect 113, wherein the masking sequence comprises all or part of a TβRII ectodomain (See e.g., FIG. 26B).
117. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, at least 110, at least 120, at least 130, at least 140, at least 150 or at least 154 aas of TβRII isoform A ectodomain aa sequence: IPPHVQK SDVEMEAQKD EIICPSCNRT AHPLRHINND MIVTDNNGAV KFPQLCKFCD VRFSTCDNQK SCMSNCSITS ICEKPQEVCV AVWRKNDENI TLETVCHDPK LPYHDFILED AASPKCIMKE KKKPGETFFM CSCSSDECND NIIFSEE (SEQ ID NO:108) optionally comprising a substitution at any one or more of F55, D57, S77, E80, and D143, which correspond to F30, D32, S52, E55 and D118 of the mature isoform B. For example, a MAPP or duplex MAPP of aspect 169, may comprise a masking sequence having at least 90%, or at least 95% (e.g., at least 98% or 100%) aa sequence identity to the TβRII isoform A ectodomain aa sequence of SEQ ID NO: 108), and optionally comprise a substitution at any one or more of F55, D57, S77, E80, and D143.
118. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, at least 110, at least 120, at least 130, at least 140, or 143 aas of TβRII isoform B ectodomain aa sequence: IPPHVQKSVN NDMIVTDNNG AVKFPQLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEEY NTSNPDLLLV IFQ (SEQ ID NO: 109), optionally comprising a substitution at any one or more of F30, D32, S52, E55 and D118 of the mature isoform B. For example, a MAPP or duplex MAPP of aspect 170, may comprise a masking sequence having at least 90%, or at least 95% (e.g., at least 98% or 100%) aa sequence identity to the TβRII isoform B ectodomain aa sequence of SEQ ID NO:109).
119. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, at least 110, at least 120, at least 130, at least 140, or 142 aas of TβRII isoform B ectodomain aa sequence: IPPHVQKSVN NDMIVTDNNG AVKFPQLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:110), optionally comprising a substitution at any one or more of F30, D32, S52, E55 and D118 of the mature isoform B. For example, a MAPP or duplex MAPP of aspect 171, may comprise a masking sequence having at least 90%, or at least 95% (e.g., at least 98% or 100%) aa sequence identity to the TβRII isoform B ectodomain aa sequence of SEQ ID NO:110).
120. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, at least 110, or 114 aas of TβRII isoform B 414 ectodomain aa sequence: VTDNNG AVKFPQLCKF CDVRESTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:111), optionally comprising a substitution at any one or more of F30, D32, S52, E55 and D118 of the mature isoform B. For example, a MAPP or duplex MAPP of aspect 172, may comprise a masking sequence having at least 90%, or at least 95% (e.g., at least 98% or 100%) aa sequence identity to the TβRII isoform B Δ14 ectodomain aa sequence of SEQ ID NO:111).
121. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, or 104 aas of TβRII isoform B Δ25 ectodomain aa sequence: QLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO: 112), optionally comprising a substitution at any one or more of F55, D57, S77, E80, and D143, which correspond to F30, D32, S52, E55 and D118 of the mature isoform B. For example, a MAPP or duplex MAPP of aspect 173, may comprise a masking sequence having at least 90%, or at least 95% (e.g., at least 98% or 100%) aa sequence identity to the TβRII isoform B A25 ectodomain aa sequence of SEQ ID NO:112).
122. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, at least 110, or 111 aas of a TβRII isoform B Δ25 ectodomain containing aa sequence: QLCKE CDVRFSTCDN QKSCMSNCSI TSICEKPQEV CVAVWRKNDE NITLETVCHD PKLPYHDFIL EDAASPKCIM KEKKKPGETF FMCSCSSAEC NDNIIFSEEY NTSNPD (SEQ ID NO: 188), optionally comprising a substitution at any one or more of F30, D32, S52, and E55 of the mature isoform B. For example, a MAPP or duplex MAPP of aspect 174, may comprise a masking sequence having at least 90%, or at least 95% (e.g., at least 98% or 100%) aa sequence identity to the TβRII isoform B Δ25 ectodomain aa sequence of SEQ ID NO:188).
123. The MAPP or duplex MAPP of aspect 117 comprising a substitution in the TβRII isoform A sequence of at least one aa (e.g., at least two aas) selected from the groups consisting of L52, F55, D57, S74, 175, T76, S77, 178, E80, V102, D143, and E144 for isoform A.
124. The MAPP or duplex MAPP of aspect 123, wherein the at least one aa is (e.g., at least two aas are) selected from the group consisting of L52A, F55A, D57A, D57N, S74A, 175A, T76A, S77A, S77L, I78A, E80A, V102A, D143A, D143R, E144A, and/or E144Q.
125. The MAPP or duplex MAPP of any of aspects 118-122, comprising a substitution in the TβRII isoform B sequence of at least one aa (e.g., at least two aas) selected from the groups consisting of substitutions at L27, F30, D32, S49, 150, T51, S52, 153, E55, V77, D118, and/or E119.
126. The MAPP or duplex MAPP of aspect 125, comprising a substitution in the TβRII isoform B sequence of at least one aa is (e.g., at least two aas are) selected from the groups consisting L27A, F30A, D32A, D32N, S49A, 150A, T51A, S52A, S52L, I53A, E55A, V77A, D118A, D118R, E119A, and/or E119Q.
127. The MAPP or duplex MAPP of aspect 125 or 126 comprising, a D118A or D118R substitution in the TβRII isoform B sequence.
128. The MAPP or duplex MAPP of aspect 127, comprising more of a F30A, D32N, S52L and/or E55A substitution in the TβRII isoform B sequence.
129. The MAPP or duplex MAPP of any of aspects 118-128, comprising an N-terminal deletion in the TβRII receptor mature polypeptide aa sequence.
130. The MAPP or duplex MAPP of aspect 129, wherein from 1 to 14, or 1 to 25 amino acids of the mature TβRII polypeptide sequence have been deleted from the N-terminus of the mature polypeptide (see, e.g., FIG. 26B SEQ ID NOs: 111 and 112).
131. The MAPP or duplex MAPP of aspect 113, wherein the masking sequence comprises all or part of a TβRIII ectodomain (See e.g., FIG. 26C).
132. The MAPP or duplex MAPP of aspect 113, wherein the masking sequence comprise an amino acid sequence having at least 90%, at least 95%, at least 98%, at least 99%, or 100%, aa sequence identity to at least 70, at least 80, at least 90, at least 100, or 120 aas of a TβRIII A isoform or B isoform ectodomain sequences (e.g., provided in FIG. 26C as SEQ ID NO:200 or SEQ ID NO:201). For example, a MAPP or duplex MAPP of aspect 173, may comprise a masking sequence having at least 90%, or at least 95% (e.g., at least 98% or 100%) aa sequence identity to the TβRIII isoform A or B ectodomain aa sequence of SEQ ID NO:200 or 201).
133. The MAPP or duplex MAPP of aspect 113, wherein the masking sequence comprise an anti-TGF-β antibody or antibody-related polypeptide/aa sequence
134. The MAPP or duplex MAPP of any preceding aspect, comprising: at least one additional MOD (wt. or variant), at least one pair of additional MODs in tandem (both wt., both variant, or one wt. and one variant), wherein the at least one MOD or at least one pair of MODs is located at one or more positions selected independently from positions 1, 1′, 2, 2′, 3, 3′, 4, 4′, 4″, 4″, 5, and/or 5′ (see FIGS. 1A and 1B) subject to the proviso that those independently selected positions selected are not occupied by a masked TGF-β MOD masking sequence or TGF-β sequence).
135. The MAPP or duplex MAPP of any preceding aspect, wherein:
   at least one additional MOD (wt. or variant), or pair of additional MODs in tandem (both wt., both variant, or one wt. and one variant), is located:
      (i) on the N-terminal side (e.g., at the N-terminus) of at least one framework polypeptide dimerization sequence (see, e.g., position 1 and 1′ in any of FIGS. 9 and 11);
      (ii) on the N-terminal side (e.g., at the N-terminus) of at least one framework polypeptide dimerization sequence and any MHC Class II polypeptide sequences that may be part of the framework polypeptide (see, e.g., positions 4″ and 4″ in FIGS. 10 and 12); and/or
      (iii) on the C-terminal side (e.g., at the C-terminus) of at least one framework polypeptide framework multimerization sequence (see, e.g., position 3 and 3′ in any of FIGS. 1 and 9 to 13).
136. The MAPP or duplex MAPP of any preceding aspect, wherein:
   at least one additional MOD (wt. or variant), or pair of additional MODs in tandem (both wt., both variant, or one wt. and one variant), located:
      (i) on the N-terminal side (e.g., at the N-terminus) of each framework polypeptide dimerization sequence (see, e.g., position 1 and 1′ in any of FIGS. 9 and 11);
      (ii) on the N-terminal side (e.g., at the N-terminus) of each framework polypeptide dimerization sequence and any MHC Class II polypeptide sequences that may be part of the framework polypeptide (see, e.g., positions 4″ and 4″ in FIGS. 10 and 12); and/or
      (iii) on the C-terminal side (e.g., at the C-terminus) of each framework polypeptide framework multimerization sequence (see, e.g., position 3 and 3′ in any of FIGS. 1 and 9 to 13).
137. The MAPP or duplex MAPP of aspect 134 to 136 comprising:
   (i) at least one additional MOD (wt. or variant), or pair of additional MODs in tandem (both wt., both variant, or one wt. and one variant) located on the N-terminal side (e.g., at the N-terminus) of at least one (e.g., each) framework polypeptide dimerization sequence (see, e.g., position 1 and 1′ in any of FIGS. 9 and 11), or on the N-terminal side (e.g., at the N-terminus) of at least one (e.g., each) framework polypeptide dimerization sequence and any MHC Class II polypeptide sequences that may be part of the framework polypeptide (see, e.g., positions 4″and 4′″ in FIGS. 10 and 12); and/or
   (ii) at least one additional MOD (wt. or variant), or pair of additional MODs in tandem (both wt., both variant, or one wt. and one variant) located on the C-terminal side (e.g., at the C-terminus) of at least one (e.g., each) framework polypeptide framework multimerization sequence (see, e.g., position 3 and 3′ in any of FIGS. 1, 9 to 13).
138. The MAPP or duplex MAPP of any preceding aspect, comprising: at least one additional MOD (wt. or variant), or pair of additional MODs in tandem (both wt., both variant, or one wt. and one variant) located:
   (i) on the N-terminal side (e.g., at the N-terminus) of at least one (e.g., two or each) dimerization polypeptide counterpart dimerization sequence (see, e.g., positions 4 and 4′ in FIGS. 1, and 10 to 12); and/or
   (ii) on the C-terminal side (e.g., at the C-terminus) of at least one (e.g., two or each) dimerization polypeptide counterpart dimerization sequence (see, e.g., position 5 and 5′ in any of FIGS. 1 and 9 to 12).
139. The MAPP or duplex MAPP of any of aspects 134 to 138, wherein when the at least one (e.g., at least two or each) dimerization polypeptide comprises a presenting sequence, the at least one additional MOD (wt. or variant), or pair of additional MODs in tandem (both wt., both variant, or one wt. and one variant) may be located:
   (i) between the counterpart dimerization sequence and the α1, α2, β1, β2 sequences and T1D epitope sequence;
   (ii) between any of the α1, α2, β1, β2 and T1D epitope sequences;
   (iii) between the T1D epitope and either the α1 and α2 or the β1 and β2 sequence; and/or
   (iv) at the N-terminus of the presenting sequence. See FIG. 15.
140. The MAPP or duplex MAPP of any of aspects 134 to 138, wherein when the dimerization polypeptide comprises a presenting complex, the at least one additional MOD (wt. or variant), or pair of additional MODs in tandem (both wt., both variant, or one wt. and one variant) may be located:
   (i) between the counterpart dimerization sequence and any of the α1, α2, β1, and/or β2 or T1D epitope sequences present in the presenting complex 1^{st} sequence;
   (ii) between any of the α1, α2, β1, and/or β2 or T1D epitope sequences present in the presenting complex 1^{st} sequence;
   (iii) at the N-terminus of the presenting complex 1^{st} sequence;
   (iv) at the N-terminus of the presenting complex 2^{st} sequence;
   (v) between any of the α1, α2, β1, and/or β2 and T1D epitope sequences present in the presenting complex 251 sequence; and/or
   (vi) at the C-terminus of the presenting complex 2^{st} sequence. See, e.g., FIGS. 17A-D, 18C-F, 19A-H, 20C-L, and 21A-F.
141. The duplex MAPP of any of aspects 34 to 140, comprising: at least one additional MOD (wt. or variant), or pair of additional MODs in tandem (both wt., both variant, or one wt. and one variant) at position 1 and/or 1′.
142. The duplex MAPP of any of aspects 34 to 140, comprising: at least one additional MOD (wt. or variant), or pair of additional MODs in tandem (both wt., both variant, or one wt. and one variant) at position 1 and/or 1′, and at least one additional MOD (wt. or variant), or pair of additional MODs in tandem (both wt., both variant, or one wt. and one variant) at position 3 and/or 3′.
143. The duplex MAPP of any of aspects 34 to 140, comprising: at least one additional MOD (wt. or variant), or pair of additional MODs in tandem (both wt., both variant, or one wt. and one variant) at position 1 and/or 1′, and at least one MOD (wt. or variant), or pair of MODs in tandem (both wt., both variant, or one wt. and one variant) at position 5 and/or 5′.
144. The MAPP or duplex MAPP of any preceding aspect, wherein the additional the MOD (wt. or variant) or the additional pair of MODs (wt. or variant) are selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4(immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-1, TGF-β2, TGF-β3, and 4-1BBL polypeptide sequences.
145. The MAPP or duplex MAPP of any preceding aspect, wherein the additional the MOD (wt. or variant) or the additional pair of MODs (wt. or variant) are selected independently from the group consisting of: 4-1BBL, PD-L1, IL-2, OX40L (CD252), ICOS-L, ICAM, CD30L, CD40, CD83, HVEM (CD270), JAG1 (CD339), CD70, CD80, and CD86, polypeptide sequences.
146. The MAPP or duplex MAPP of any preceding aspect, wherein the additional the MOD (wt. or variant) or the additional pair of MODs (wt. or variant) are selected independently from the group consisting of IL-2. PD-L1, 4-1BBL polypeptide sequences and variants of any thereof. For example, the MAPP or duplex MAPP may comprise at least one IL-2 MOD (wt. or variant) and/or at least one PD-L1 (wt. or variant) polypeptide sequence(s).
147. The MAPP or duplex MAPP of any preceding aspect, wherein the additional the MOD (wt. or variant) or the additional pair of MODs (wt. or variant) comprise at least one IL-2 MOD (wt. or variant) polypeptide sequence, or at least one pair of IL-2 MOD (wt. or variant) polypeptide sequences in tandem (optionally located at position 1 or 1′).
148. The MAPP or duplex MAPP of any preceding aspect, wherein the additional the MOD (wt. or variant) or the additional pair of MODs (wt. or variant) comprise at least one PD-L1 MOD (wt. or variant) or variant MOD polypeptide sequence, which is optionally located at position 1 or 1′.
149. The MAPP or duplex MAPP of any preceding aspect, wherein the additional the MOD (wt. or variant) or the additional pair of MODs (wt. or variant) comprise at least one 4-1BBL MOD (wt. or variant) polypeptide sequence, which is optionally located at position 1 or 1′.
150. The MAPP or duplex MAPP of any preceding aspect further, comprising an additional polypeptide, or a payload covalently attached to one or more framework polypeptides and/or dimerization polypeptides.
151. The MAPP or duplex MAPP of aspect 150, wherein the additional peptide is an affinity tag.
152. The MAPP or duplex MAPP of aspect 150, wherein the additional peptide is selected from the group consisting of: antibody or antigen binding fragment/portion thereof (e.g., an scFv or a nanobody such as a heavy chain nanobody or a light chain nanobody).
153. The MAPP of aspect 1, wherein the MAPP comprises:
   a framework polypeptide comprising, from N-terminus to C-terminus, aa sequences of (i) a T1D peptide epitope, (ii) human MHC class II β1 and β2 domains, (iii) human Class II α1 and α2 domains, (iv) a human or humanized IgG CH1, and (v) an Ig Fc region (e.g., CH2 CH3 regions with optional “LALA” substitutions), each of (i) to (v) optionally separated by linker peptide sequences that are selected independently; and
   a dimerization polypeptide comprising from N-terminus to C-terminus aa sequences of (i) a human PD-L1 extracellular domain polypeptide, and (ii) a human or humanized kappa light constant region, with (i) and (ii) optionally separated by a linker peptide sequence. See, e.g., FIG. 14 structure A.
154. The MAPP of aspect 1, wherein the MAPP comprises:
   a framework polypeptide comprising, from N-terminus to C-terminus, aa sequences of (i) a human PD-L1 extracellular domain polypeptide, (ii) a human or humanized CH3 KIH _{S-S} hole, and (iii) a human IgG Fc sequence (e.g., IgG1 CH2 and CH3 regions with optional “LALA” substitutions), each of (i) to (iii) optionally separated by linker peptide sequences that are selected independently; and
   a dimerization polypeptide comprising from N-terminus to C-terminus aa sequences of (i) a T1D peptide epitope, (ii) human MHC class II β1 and β2 domains, (iii) human Class II α1 and α2 domains, and (iv) a human or humanized IgG1 with CH3 KIH _{S-S} knob, each of (i) to (iv) optionally separated by linker peptide sequences that are selected independently. See, e.g., FIG. 14 structure B
155. The MAPP of any of aspect 1, wherein the MAPP comprises:
   a framework polypeptide comprising, from N-terminus to C-terminus, aa sequences of (i) a human PD-L1 extracellular domain polypeptide, (ii) a human or humanized CH1 (optionally with M13 stabilization substitutions), and (iii) a human Ig Fc sequence (e.g., IgG1 CH2 and CH3 regions with optional “LALA” substitutions), each of (i) to (iii) optionally separated by linker peptide sequences that are selected independently; and
   a dimerization polypeptide comprising from N-terminus to C-terminus aa sequences of (i) a T1D peptide epitope, (ii) human MHC class II β1 and β2 domains, (iii) human Class II α1 and α2 domains, and (iv) a human or humanized kappa light chain sequence (optionally with MD13 stabilization substitutions), each of (i) to (iv) optionally separated by linker peptide sequences that are selected independently. See, e.g., FIG. 14 structure C
156. The MAPP of any of aspect 1, wherein the MAPP comprises:
   a framework polypeptide comprising, from N-terminus to C-terminus, aa sequences of (i) a T1D peptide epitope, (ii) human MHC Class II β1 and β2 domains, (iii) human Class II α1 and α2 domains, (iv) an IgG Fc region (e.g., CH2 and CH3 regions with optional “LALA” substitutions), (v) a human or humanized IgG CH1, each of (i) to (v) optionally separated by linker peptide sequences that are selected independently; and
   a dimerization polypeptide comprising from N-terminus to C-terminus aa sequences of (i) a human PD-L1 extracellular domain polypeptide, and (ii) a human or humanized kappa light constant region, with (i) and (ii) optionally separated by a linker peptide sequence. See, e.g., FIG. 14 structure D.
157. A duplex of the MAPPs of any of aspects 153 to 156, wherein the duplex is formed by interactions between the IgFc multimerization sequences.
158. The MAPP or duplex MAPP of any of aspects 153 to 157, wherein the immunoglobulin (Ig) sequences are Ig G sequences (e.g., Ig G1 sequences)
159. The MAPP or duplex MAPP of any of aspects 153 to 158, wherein the MHC Class II α1 and α2 are DRA α1 and α2 domains.
160. The MAPP or duplex MAPP of any of aspects 153 to 159, wherein the MHC Class II α1 and α2 domains have greater than 95% identity to DRA*0101 α1 and α2 domains.
161. The MAPP or duplex MAPP of any of aspects 153 to 160, wherein the MHC Class II β1 and β2 are DRB1 β1 and β2 domains.
162. The MAPP or duplex MAPP of any of aspects 153 to 161, wherein the MHC Class II β1 and β2 domains have greater than 95% identity to DRB1*01:01, DRB1*0301, DRB1*0401, DRB1*0402, DRB1*0405, DRB1*0801, or DRB1*0901 β1 and β2 domains.
163. The MAPP or duplex MAPP of any of aspects 153 to 162, wherein the MHC Class II β1 and 2 domains have greater than 95% identity to DRB1*03:01 or DRB1*04:01.
164. The MAPP or duplex MAPP of any of aspects 153 to 163, wherein the MHC Class II α1 and α2 domains have greater than 95% identity to DRA*01:01 and the MHC Class II β1 and 2 domains have greater than 95% identity to DRB1*03:01 or DRB1*04:01.
165. The MAPP or duplex MAPP of any of aspects 153 to 158, wherein the MHC Class II α1 and α2 are DQA1 α1 and α2 domains.
166. The MAPP or duplex MAPP of any of aspects 153 to 158, wherein the MHC Class II α1 and α2 domains have greater than 95% identity to DQA*01:01, DQA*03:01, DQA*04:01, or DQA*05:01,
167. The MAPP or duplex MAPP of any of aspects 165 to 166, wherein the MHC Class II β1 and β2 are DQB1 β1 and β2 domains.
168. The MAPP or duplex MAPP of any of aspects 165 to 167, wherein the MHC Class II β1 and β2 domains have greater than 95% identity to DQB1*02:01, DQB1*03:02, DQB1*03:03, DQB1*04:02, or DQB1*05:01 β1 and 2 domains.
169. The MAPP or duplex MAPP of any of aspects 165 to 168, wherein the MHC Class II β1 and β2 domains have greater than 95% identity to DQB1*02:01 or DQB1*03:02.
170. The MAPP or duplex MAPP of any of aspects 165 to 169, wherein the MHC Class II α1 and α2 domains have greater than 95% identity to DQA*03:01 or DQA*05:01, and the MHC Class II β1 and β2 domains have greater than 95% identity to DQB1*02:01 or DQB1*03:02.
171. The MAPP, or higher order MAPP (e.g., duplex MAPP) of any preceding aspect, wherein the T1D peptide epitope is selected from an epitope of preproinsulin, proinsulin, insulin, insulin β chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8).
172. The MAPP, or higher order MAPP (e.g., duplex MAPP) of aspect 171, wherein the T1D peptide epitope is selected from the group consisting of proinsulin 73-90 (GAGSLQPLALEGSLQKR; SEQ ID NO:165), insulin (InsA (1-15) peptide GIVDQCCTSICSLYQ (SEQ ID NO:166), insulin (InsA(1-15; D4E) peptide GIVEQCCTSICSLYQ (SEQ ID NO:167), GAD65 (555-567) peptide NFFRMVISNPAAT (SEQ ID NO:168), GAD65 (555-567; F5571) peptide NFIRMVISNPAAT (SEQ ID NO:169), islet antigen 2 (IA2) peptide SFYLKNVQTQETRTLTQFHF (SEQ ID NO:170), proinsulin peptide SLQPLALEGSLQSRG (SEQ ID NO:171), and proinsulin peptide GSLQPLALEGSLQSRGIV (SEQ ID NO:172; proIns 75-92(K88S)).
173. The MAPP, or higher order MAPP (e.g., duplex MAPP) of aspect 171, wherein the T1D peptide comprises from 4 to 25 contiguous aas of an aa sequence having at least 90%, at least 95%, at least 98%, at least 99%, or 100%, aa sequence identity to aas 25-110 of the human preproinsulin aa sequence (wherein italicized aas 1-24 form the signal peptide): MALWMRLLPL LALLALWGPD PAAA FVNQHL CGSHLVEALY LVCGERGFFY TPKTRREAED LQVGQVELGG GPGAGSLQPL ALEGSLQKRG IVEQCCTSIC SLYQLENYCN (SEQ ID NO:173).
174. The MAPP, or higher order MAPP (e.g., duplex MAPP) of aspect 171, wherein the T1D peptide epitope has the aa sequence: GAGSLQPLALEGSLQKRG (SEQ ID NO:174) (proIns 73-90), SLQPLALEGSLQKRG (SEQ ID NO:175) (proIns 76-90), SLQPLALEGSLQSRG (SEQ ID NO:176) (proIns 76-90; K88S), QPLALEGSLQKRG (SEQ ID NO:177), or the sequence QPLALEGSLQSRG (SEQ ID NO:178).
175. The MAPP or duplex MAPP of any preceding aspect, wherein the T1D peptide epitope is from about 4 aas (aa) to about 25 aa (e.g., the epitope can have a length of from 4 aa to 10 aa, from about 6 aa to about 12 aa, from 8 aa to 20 aa, from 10 aa to 15 aa, from 10 aa to 20 aa, from 15 aa to 20 aa, or from 20 aa to 25 aa).
176. The MAPP or duplex MAPP of any preceding aspect wherein the peptide epitope is from about 810 aa to about 20 aa.
177. A pharmaceutical composition comprising a MAPP or duplex MAPP of any of the preceding aspects.
178. A method of treatment or prophylaxis of a T1D comprising administering to a patient/subject (e.g., a diabetic or prediabetic patient in need thereof) a pharmaceutical composition comprising an effective amount of one or more MAPPs or higher order MAPP complexes (e.g., duplex MAPPs) of any of aspects 1 to 176.
179. The method of aspect 178, wherein the MAPP or duplex MAPP reduces hemoglobin A1C or blood sugar (e.g., glucose) levels in a diabetic or prediabetic patient/subject relative to the hemoglobin A1C or blood sugar levels prior to administration of the MAPP or duplex MAPP.
180. The method of any of aspects 178 to 179, wherein the one or more MAPPs or duplex MAPPs are administered to a mammalian patient or subject.
181. The method of any of aspects 178 to 180, wherein the subject is human.
182. The method of any of aspects 178 to 180, wherein the subject non-human (e.g., rodent, lagomorph, bovine, canine, feline, rodent, murine, caprine, simian, ovine, equine, lapine, porcine, etc.).
183. A framework polypeptide of a MAPP or duplex MAPP according to any of aspects 1 to 176, optionally comprising an additional polypeptide.
184. A dimerization polypeptide of a MAPP or duplex MAPP, according to any of aspects 1 to 176, optionally comprising an additional polypeptide.
185. A nucleic acid sequence encoding the framework polypeptide of any of aspects 1 to 176, wherein the framework polypeptide optionally comprises an additional polypeptide.
186. A nucleic acid sequence encoding the dimerization polypeptide of any of aspects 1 to 176, wherein the dimerization polypeptide optionally comprises an additional polypeptide.
187. One or more nucleic acids comprising one or more nucleic acid sequences encoding a MAPP or higher order MAPP complex (e.g., a duplex MAPP) according to any of aspects 1 to 176.
188. The one or more nucleic acids of aspect 187, comprising a nucleic acid sequence encoding the framework polypeptide operably linked to a promoter, and/or a nucleic acid sequence encoding the dimerization polypeptide operably linked to a promoter.
189. One or more recombinant expression vectors comprising one or more nucleic acid molecules comprising sequences encoding a MAPP of any of embodiments 1 to 176.
190. A method of producing cells expressing a MAPP or duplex MAPP, the method comprising introducing one or more nucleic acid molecules according to aspect 187 or 188, or one or more expression vectors according to aspect 189, into the cells in vitro; selecting for cells that produce the MAPP or duplex MAPP; and optionally selecting for cells comprising all or part of the one or more nucleic acids either unintegrated or integrated into at least one cellular chromosome.
191. The method of aspect 190, wherein the cell is a cell of a mammalian cell line selected from the group consisting of: HeLa cells, CHO cells, 293 cells, Vero cells, NIH 3T3 cells, Huh-7 cells, BHK cells, PC12, COS cells, COS-7 cells, RAT1 cells, mouse L cells, human embryonic kidney (HEK) cells, and HLHepG2 cells.
192. A cell transiently or stably expressing a MAPP or duplex MAPP prepared by the method of aspect 190 or 191.
193. The cell of aspect 192, wherein the cell express from about 25 to about 350 (e.g., 20-50, 50-100, 100-200, 200-300, 300-350) mg/liter or more of the MAPP or duplex MAPP without a substantial reduction (e.g., less than a 5%, 10%, or 15% reduction) in viability relative to otherwise identical cells not expressing the MAPP or duplex MAPP.
194. A method of selectively delivering one or more MOD polypeptides and/or variant MOD polypeptides to a cell, tissue, patient or subject having or suspected of having T1D, the method comprising administering to a patient/subject having or suspected of having T1D an effective amount of one or more MAPPs or higher order MAPPS (e.g., duplex MAPPs) of any of aspects 1 to 176.

### VI. EXAMPLES

### Example 1

Example 1 illustrates the preparation of 8 MAPP and 4 control proteins in the form of heterodimers each comprising a framework and dimerization polypeptide that have formed higher order duplex structures through interactions between their interspecific IgG1 CH2-CH3 “KIH” sequences. MAPP proteins 1-8 comprise a dimerization polypeptide comprising from N terminus to C terminus a peptide epitope sequence, a presenting sequence fused to an Ig C kappa (Cκ) light chain sequence with intervening linkers. See e.g., FIG. 1A generally, and more specifically FIG. 1C at “(a) and (b)” showing a duplex MAPP with presenting sequences at the terminus of the dimerization peptide and bearing a MAPP with the masking peptide and masked TGF-β peptide placed in “trans” at the carboxyl terminus of the framework polypeptides (positions 3 and 3′ of the MAPP). The overall structure of the presenting sequence attached to the C kappa (Cκ) polypeptide sequence is shown in FIG. 24 at (b). FIG. 37 provides at (a) a more detailed presentation of the constructs with variant IL-2 MODS having H16A F42A substitutions at positions 1 and 1′ of the MAPP. An intrachain disulfide bond between is used to stabilize the MHC alpha and beta polypeptide sequences, in this case between the R5C and P81C substitutions). Similarly, interchain disulfide bonds (shown in red) between the CH1 and C kappa (Cκ and kappa light chain or κL) stabilize their interactions. The CH1 and C kappa sequence comprise substitutions found to stabilize their interactions based on the MD13 antibody to HIV gp140 described by Chen et al., in MAbs. 8(4): 761-774 (2016) (see e.g., FIGS. 21 and 3A). The framework polypeptide interspecific multimerization Knob-In-Hole (KIH) sequence interactions are stabilized by the pair of hinge region disulfides and an additional disulfide bond shown as dashed lines in the multimerization sequence, making the KIH an interspecific KIHs-s sequence. The four control proteins share the structure of the eight MAPPs described above, however, the epitope, presenting sequences, and associated linkers are not present on the dimerization peptide.

Each of the eight MAPP and four control proteins are detailed in the table that follows. The peptide epitope sequences are noted as TXA23 and OVA. TXA23 represents aas 630-641 of the human H+/K+ ATPase a subunit (see e.g., GenBank: accession BAD96979.1 where those aa residues are at positions 632-643). OVA represents an peptide epitope from gallus (chicken) ovalbumin, specifically aas 324-340 (see, e.g., Genbank accession AUD54707.1 aas 324-340). The presenting sequence in the MAPPs are murine Class II MHC sequences. More specifically the alpha and beta chain sequences are derived from the murine Class II I-ad alpha chain with a P81C substitution (see e.g., GenBank: AAR19089.1); and the Class II I-ad beta chain with an R5C substitution (see, e.g., NCBI accession GI: 4139969). The P81C and R5C substitutions permit the formation of the intrachain disulfide bond that stabilizes the MHC sequences. IL-2 MOD sequences appear either at one (1X) or both (2X) of the framework polypeptides N-termini and are indicated as being on the framework polypeptide with the knob (position “1”) or the with the hole (position 1′) as depicted in FIG. 1A and FIG. 1C. The sequences of each polypeptide in the MAPPs and control proteins is provided in FIG. 28.

A reducing SDS PAGE gel of the proteins expressed in culture (e.g., in CHO cells) and purified by size separation and affinity chromatography on immobilized protein A is provided in FIG. 27 at (b). The left side of the gel provides the molecular weight of the protein standards appearing in the left-most lane. The numbers across the top of the gel correspond to the “Protein No.” in the table that follows. The theoretical molecular weights of the peptides in each of the MAPPS and control proteins are provided in the Table that follows, along with the retention time of the duplex MAPPs and control proteins on size exclusion chromatography. Also provided is the amount of MAPP and control proteins as unaggregated duplex MAPP and control protein (% monomer) based on area under the curve from size exclusion chromatography analysis.

Other epitopes, including T1D-associated epitopes, and human and mouse MHC sequences specific for those epitopes may be used to replace the exemplified epitopes and murine MHC sequences employed in this example.

| | | | | | Un- | |
|---|---|---|---|---|---|---|
| | | | | | aggregated | |
| | Poly- | | | | duplex | Theo- |
| | peptides | | | Retention | MAPP | retical |
| Protein | in the | | IL-2 | time | or control | M.W. |
| No. | constructs | Epitope | (position) | (minutes) | protein (%) | (kDa) |
| 1 | 4421- | TXA23 | | 6.899 | 33 | 66 |
| | 4423- | | 2X | | | 65 |
| | 4425 | | | | | 61 |
| 2 | 4421- | TXA23 | 1X (1) | 6.97 | 46 | 66 |
| | 4424- | | | | | 50 |
| | 4425 | | | | | 61 |
| 3 | 4421- | OVA | 2X | 6.911 | 53 | 66 |
| | 4423- | | | | | 65 |
| | 4426 | | | | | 61 |
| 4 | 4421- | OVA | 1X (1) | 6.966 | 57.5 | 66 |
| | 4424- | | | | | 50 |
| | 4426 | | | | | 61 |
| 5 | 4422- | TXA23 | 1X (1') | 6.965 | 50 | 50 |
| | 4423- | | | | | 65 |
| | 4425 | | | | | 61 |
| 6 | 4422- | TXA23 | — | 7.015 | 54 | 50 |
| | 4424- | | | | | 50 |
| | 4425 | | | | | 61 |
| 7 | 4422- | OVA | 1X (1') | 6.962 | 60.8 | 50 |
| | 4423- | | | | | 65 |
| | 4426 | | | | | 61 |
| 8 | 4422- | OVA | — | 7.045 | 85 | 50 |
| | 4424- | | | | | 50 |
| | 4426 | | | | | 61 |
| 9 | 4421- | None | 2X | 7.729 | 50 | 66 |
| | 4423- | | | | | 65 |
| | 4016 | | | | | 15 |
| 10 | 4421- | None | 1X (1) | 7.95 | 60.5 | 66 |
| | 4424- | | | | | 50 |
| | 4016 | | | | | 15 |
| 11 | 4422- | None | 1X (1') | 7.967 | 60.6 | 50 |
| | 4423- | | | | | 65 |
| | 4016 | | | | | 15 |
| 12 | 4422- | None | — | 8.241 | 83.9 | 50 |
| | 4424- | | | | | 50 |
| | 4016 | | | | | 15 |

1. A multimeric antigen-presenting polypeptide (MAPP) comprising:
(i) a framework polypeptide comprising a dimerization sequence and a multimerization sequence,
(ii) a dimerization polypeptide comprising a counterpart dimerization sequence complementary to the dimerization sequence of the framework polypeptide, the dimerization sequence and counterpart dimerization sequence dimerizing through covalent and/or non-covalent interactions to form a MAPP heterodimer, and
(iii) at least one presenting sequence and/or presenting complex;
wherein
(a) each presenting sequence comprises (i) a T1D peptide epitope, and (ii) MHC Class II α1, α2, β1, and β2 domain polypeptide sequences;
(b) each presenting complex comprises a presenting complex 1^{st} sequence and a presenting complex 2^{nd} sequence, wherein
the presenting complex 1^{st} sequence or presenting complex 2^{nd} sequence comprises the peptide epitope and at least one of the α1, α2, β1, and β2 polypeptide sequences, and
the presenting complex 1^{st} sequence and presenting complex 2^{nd} sequence together comprise a T1D peptide epitope and MHC Class II α1, α2, β1, and β2 domain polypeptide sequences,
(c) one or both of the dimerization polypeptide and/or the framework polypeptide comprises a presenting sequence or a presenting complex 1^{st} sequence, and
(d) the framework polypeptide, dimerization polypeptide, presenting sequence, or presenting complex comprises (i) a TGF-β sequence, (ii) a masking sequence, or (iii) at least one masked TGF-β immunomodulatory polypeptide(s) (“masked TGF-β MOD”), each masked TGF-β MOD comprising a masking sequence and TGF-β sequence; and
(e) at least one framework polypeptide, dimerization peptide, presenting sequence, or presenting complex comprises one or more independently selected additional MOD and/or additional variant MOD polypeptide sequences; and
wherein the framework polypeptide, dimerization polypeptide, presenting sequence, presenting complex 1^{st} sequence and/or presenting complex 2^{nd} sequence optionally comprise one or more linker sequences that are selected independently.

2. The MAPP of claim 1, wherein at least one presenting sequence or presenting complex comprises:
(i) α1 and α2 domain polypeptide sequences each having at least 90% or at least 95% (e.g., at least 98% or 100%) sequence identity to all or at least about 50 contiguous aas of a HLA DRA*01*01 or HLA DRA*01*02 polypeptide sequence; and
(ii) β1 and β2 domain polypeptide sequences each having at least 90% or at least 95% (e.g., at least 98% or 100%) sequence identity to at least about 60 contiguous aas of an DRB1*0301, DRB1*0401, DRB1*0402, DRB1*0405, DRB1*0801, or DRB1*0901 polypeptide sequence.

3. The MAPP of claim 1, wherein at least one presenting sequence or presenting complex comprises:
(i) α1 and α2 domain polypeptide sequences each having at least 90% or at least 95% (e.g., at least 98% or 100%) sequence identity to at least 60 contiguous aas of a HLA DQ alpha 1 or DQ alpha 2 (DQA1 or DQA2) polypeptide sequence; and
(ii) β1 and β2 domain polypeptide sequences each having 90% or at least 95% (e.g., at least 98% or 100%) sequence identity to at least 60 contiguous aas of an HLA DQ beta 1 or DQ beta 2 (DQB1 or DQB2) polypeptide sequence.

4. The MAPP of claim 3, wherein at least one presenting sequence or presenting complex comprises either:
(i) α1 and α2 domain polypeptide sequences each having at least 90% or at least 95% (e.g., at least 98% or 100%) sequence identity to at least 60 contiguous aas of a DQA1*0501 polypeptide sequence, and
β1 and β2 domain polypeptide sequences each having at least 90% or at least 95% (e.g., at least 98% or 100%) sequence identity to at least 60 contiguous aas of a DQB1*0201 polypeptide sequence; or
(ii) α1 and α2 domain polypeptide sequences each having at least 90% or at least 95% (e.g., at least 98% or 100%) sequence identity to at least 60 contiguous aas of a DQA1*0301 polypeptide sequence, and
β1 and β2 domain polypeptide sequences each having at least 90% or at least 95% (e.g., at least 98% or 100%) sequence identity to at least 60 contiguous aas of a DQB1*0302 polypeptide sequence.

5. The MAPP of claim 1, wherein the MAPP comprises at least one linker comprising:
(i) GSGGS, GGGS, GGSG, GGSGG, GSGSG, GSGGG, GGGSG, GSSSG, GGGGS sequences, any of which may be repeated 1, 2, 3, 4, 5, 6, 7, 8, 9, or 10 times; or
(ii) a cysteine-containing linker sequence selected from CGGGS, GCGGS, GGCGS, GGGCS, and GGGGC, with the remainder of the linker comprised of Gly and Ser residues.

6. The MAPP of claim 1, wherein when the MAPP comprises a presenting sequence, and wherein the presenting sequence comprises, in the N-terminal to C-terminal direction:
(i) the T1D peptide epitope, the β1, α1, α2 and β2 domain polypeptide sequences;
(ii) the T1D peptide epitope, the β1, β2, α1, and α2 domain polypeptide sequences; or
(iii) the T1D peptide epitope, the α1, α2, β1, and β2, domain polypeptide sequences,
wherein the presenting sequence optionally comprises one or more wt. or variant MOD polypeptide sequences, and
wherein said presenting sequence optionally comprises one or more independently selected linker sequences.

7. The MAPP of claim 1, comprising at least one presenting complex, wherein the at least one presenting complex comprises a presenting complex 1^{st} sequence and presenting complex 2^{nd} sequence, and wherein:
(i) the presenting complex 1^{st} sequence comprises the α1 domain polypeptide sequence, and its associated presenting complex 2^{nd} sequence comprises the T1D peptide epitope sequence and the β1 domain polypeptide sequence;
(ii) the presenting complex 1^{st} sequence comprises the α2 domain polypeptide sequence, and its associated presenting complex 2^{nd} sequence comprises the T1D peptide epitope sequence and the β2 domain polypeptide sequence;
(iii) the presenting complex 1^{st} sequence comprises the β1 domain polypeptide sequence, and its associated presenting complex 2^{nd} sequence comprises the T1D peptide epitope sequence and the α1 domain polypeptide sequence;
(iv) the presenting complex 1^{st} sequence comprises the β2 domain polypeptide sequence, and its associated presenting complex 2^{nd} sequence comprises the T1D peptide epitope sequence and the α2 domain polypeptide sequence;
(v) the presenting complex 1^{st} sequence comprises the α1 domain polypeptide sequence, and its associated presenting complex 2^{nd} sequence comprises the T1D peptide epitope sequence and the β1 and β2 domain polypeptide sequences;
(vi) the presenting complex 1^{st} sequence comprises the α2 domain polypeptide sequence, and its associated presenting complex 2^{nd} sequence comprises the T1D peptide epitope sequence and the β1 and β2 domain polypeptide sequences;
(vii) the presenting complex 1^{st} sequence comprises the α1 and/or α2 domain polypeptide sequences, and its associated presenting complex 2^{nd} sequence comprises the T1D peptide epitope sequence and the β1 and β2 domain polypeptide sequences;
(viii) the presenting complex 1^{st} sequence comprises the β1 and/or β2 domain polypeptide sequences, and its associated presenting complex 2^{nd} sequence comprises the T1D peptide epitope sequence and the α1 and α2 domain polypeptide sequences;
(ix) the presenting complex 1^{st} sequence comprises the peptide epitope sequence and the al domain polypeptide sequence, and its associated presenting complex 2^{nd} sequence comprises the β1 domain polypeptide sequence;
(x) the presenting complex 1^{st} sequence comprises the T1D peptide epitope sequence and the α2 domain polypeptide sequence, and its associated presenting complex 2^{nd} sequence comprises the β2 domain polypeptide sequence;
(xi) the presenting complex 1^{st} sequence comprises the T1D peptide epitope sequence and the β1 domain polypeptide sequence, and its associated presenting complex 2^{nd} sequence comprises the α1 domain polypeptide sequence;
(xii) the presenting complex 1^{st} sequence comprises the T1D peptide epitope sequence and the β2 domain polypeptide sequence, and its associated presenting complex 2^{nd} sequence comprises the α2 domain polypeptide sequence;
(xiii) the presenting complex 1^{st} sequence comprises the T1D peptide epitope sequence and the α1 domain polypeptide sequence, and its associated presenting complex 2^{nd} sequence comprises the β1 and β2 domain polypeptide sequences;
(xiv) the presenting complex 1^{st} sequence comprises the T1D peptide epitope sequence and the α2 domain polypeptide sequence, and its associated presenting complex 2^{nd} sequence comprises the β1 and β2 domain polypeptide sequences;
(xv) the presenting complex 1^{st} sequence comprises the T1D peptide epitope sequence and the α1 and/or α2 domain polypeptide sequences, and its associated presenting complex 2^{nd} sequence comprises the β1 and β2 domain polypeptide sequences; or
(xvi) the presenting complex 1^{st} sequence comprises the T1D peptide epitope sequence and the β1 and/or β2 domain polypeptide sequences, and its associated presenting complex 2^{nd} sequence comprises the α1 and α2 domain polypeptide sequences,
and further wherein the at least one presenting complex optionally comprises one or more, or two more wt. or variant MODs.

8. The MAPP of any preceding claim, complexed to form a duplex or higher order MAPP comprising at least a first MAPP heterodimer and a second MAPP heterodimer of any of claims 1-7, wherein:
(i) the first MAPP heterodimer comprises a first framework polypeptide having a first multimerization sequence and a first dimerization sequence, and a first dimerization polypeptide having first counterpart dimerization sequence complementary to the first dimerization sequence; and
(ii) the second MAPP heterodimer comprises a second framework polypeptide having a second multimerization sequence and a second dimerization sequence, and a second dimerization polypeptide having second counterpart dimerization sequence complementary to the second dimerization sequence;
wherein the first and second framework polypeptides are associated by binding interactions between the first and second multimerization sequences optionally including one or more interchain covalent bonds, and the multimerization sequences are not the same as, and do not substantially associate with or bind to, the dimerization sequences or counterpart dimerization sequences; and
wherein the duplex or higher order MAPP comprises at least one masked TGF-β MOD with the masking sequence and the TGF-β sequence present in cis or in trans.

9. The duplex MAPP of claim 8, wherein when the multimerization sequences are not an interspecific multimerization pair, the multimerization sequences are selected from the group consisting of immunoglobulin heavy chain constant regions, collectin family dimerization sequences, coiled-coil domains, and leucine-zipper domains; and
wherein when the multimerization sequences are an interspecific multimerization pair, the multimerization sequences are selected from the group consisting of a Fos and Jun polypeptide pair, Ig CH1 and Ig CL κ or λ constant region polypeptide pair, a knob-in-hole without disulfide (“KiH”) pair, a knob-in hole with a stabilizing disulfide bond (“KiHs-s”) pair, a HA-TF polypeptide pair, a ZW-1 polypeptide pair, a 7.8.60 polypeptide pair, a DD-KK polypeptide pair, an EW-RVT polypeptide pair, an EW-RVTs-s polypeptide pair, and an A107 polypeptide pair.

10. The duplex MAPP of claim 8, wherein the multimerization sequences comprise Ig Fc regions and the first and second dimerization sequences comprise independently selected Ig CH1, Ig C_{L} κ or λ, leucine zipper, Fos or Jun domains.

11. The duplex MAPP of claim 8, wherein:
(i) the multimerization sequences of the first and second framework polypeptides are covalently linked by at least one disulfide bond; or
(ii) the first dimerization sequence and its counterpart dimerization sequence and/or the second dimerization sequence and its counterpart dimerization sequence are covalently linked by at least one disulfide bond, and the multimerization sequences of the first and second framework polypeptides are covalently linked by at least one disulfide bond.

12. The duplex MAPP of claim 8, comprising at least one wt. or variant additional MOD polypeptide sequence, wherein each additional MOD is selected independently from the group consisting of IL-2, PD-L1, CD80, CD86 and 4-1BBL polypeptide sequences.

13. The duplex MAPP of claim 12, comprising at least one wt. or variant IL-2 polypeptide or at least one wt. or variant PD-L1 polypeptide.

14. The duplex MAPP of claim 13 wherein the peptide epitope is from about 10 aa to about 20 aa.

15. A method of treatment or prophylaxis of a T1D comprising administering to a patient/subject an effective amount of one or more MAPPs of any of claims 1-7 and/or duplex MAPPs of any of claims 8-14.

16. One or more nucleic acid molecules comprising sequences encoding a MAPP of any one of claims 1-7.

17. A method of producing cells expressing a MAPP or duplex MAPP, the method comprising introducing one or more nucleic acid molecules according to claim 16 into the cells in vitro; selecting for cells that produce the MAPP or duplex MAPP; and optionally selecting for cells comprising all or part of the one or more nucleic acids either unintegrated or integrated into at least one cellular chromosome.

18. One or more cells transiently or stably expressing the MAPP or duplex MAPP prepared by the method of claim 17; optionally, wherein the cells express from about 25 to about 350 mg/liter or more of the MAPP or duplex MAPP without a substantial reduction in viability relative to otherwise identical cells not expressing the MAPP or duplex MAPP.

19. A method of selectively delivering one or more MOD polypeptides and/or variant MOD polypeptides to a human patient having or suspected of having T1D, the method comprising administering to the patient an effective amount of one or more MAPPs of claims 1-7 and/or one more duplex MAPPs of claims 8-14.
